(19) United States
(12) Patent Application Publication
(10) Pub. No.: US 2022/0402998 A1
(43) Pub. Date: Dec. 22, 2022

(54) PROTEASE SWITCH FOR DUAL TARGETS CHIMERIC ANTIGEN RECEPTOR T CELL THERAPY

(71) Applicant: THE TEXAS A&M UNIVERSITY SYSTEM, COLLEGE STATION, TX (US)

(72) Inventors: WENSHE RAY LIU, COLLEGE STATION, TX (US); WENYUE CAO, COLLEGE STATION, TX (US)

(21) Appl. No.: 17/772,539

(22) PCT Filed: Oct. 30, 2020
(86) PCT No.: PCT/US2020/058185

§ 371 Date: Apr. 28, 2022

Related U.S. Application Data
(60) Provisional application No. 62/927,898, filed on Oct. 30, 2019.

(51) Int. Cl. C07K 14/725; C07K 14/705; A61K 35/17; A61P 35/00; C12N 9/50; C07K 16/28

(52) U.S. Cl. CPC C07K 14/7051; C07K 14/70521; A61K 35/17; A61P 35/00; C12N 9/50; C07K 16/2803; C07K 14/70578; C07K 2319/03; C07K 2317/622; C07K 2319/02; C07K 2319/33

(57) ABSTRACT

The subject invention pertains to a chimeric antigen receptor, a T cell comprising said CAR and methods of making and using said CAR and said T cell for the treatment of a cancer and/or an immune disease. In specific embodiments, the method comprises the use of T cells comprising CARs to target different antigens on target cells. In further specific embodiments, the CARs of the invention comprise NS3 protease domains and cleavage sites, which NS3 domains are inhibited by small molecule inhibitors for customized CAR-T cell therapy.

### CROSS-REFERENCE TO RELATED APPLICATION

This application claims the benefit of U.S. Provisional Application Ser. No. 62/927,898, filed Oct. 30, 2019, the disclosure of which is hereby incorporated by reference in its entirety, including all figures, tables and amino acid or nucleic acid sequences.

The Sequence Listing for this application is labeled “Seq-List.txt” which was created on Oct. 29, 2020 and is 1,053 KB. The entire content of the sequence listing is incorporated herein by reference in its entirety.

### BACKGROUND OF THE INVENTION

This section provides background information to facilitate a better understanding of the various aspects of the invention. It should be understood that the statements in this section of this document are to be read in this light, and not as admissions of prior art.

Adoptive T cell therapy utilizing autologous cells genetically engineered to target tumor antigens has revolutionized the treatment of hematological malignancies. Synthetic chimeric antigen receptors (CARs) targeting the tumor antigens have been used to transduce T cells (CAR-T cells) to target them towards tumors expressing the tumor antigen.

T cells expressing CARs are generated by genetic engineering and are designed to arm the immunocompetent T cells of a patient with an activating receptor consisting of (1) an extracytoplasmic variable fragment of an immunoglobulin, e.g., a single chain variable fragment (scFv), directed against a tumor target, (2) an intracellular T-cell receptor activation molecule (e.g., CD3 zeta (CD3ζ)), and (3) positive co-stimulation molecules, e.g., CD28 and/or 4-1BB. T cells obtained from a patient are transformed with a vector, e.g., a retro- or lentiviral vector that transfers the desired DNA sequences encoding the above elements into the genome of the transduced T cell, such that the elements of the exogenous CAR are expressed in the transduced T cell. Expression of these elements is generally controlled by promoters that are either constitutive providing a continuous expression of the CAR elements or inducible providing expression of the CAR elements only when the inducing agent is present. Alternatively, the promoters can be endogenously regulated and provide expression of the CAR elements whenever the protein that is normally controlled by the endogenous promoter is expressed. Generally, strong promoters that induce high levels of the proteins are desirable in genetically engineered cells.

Although mostly immunocompetent T cells are used for the expression of CARs, any immune cells capable of being activated by a CD3-zeta and costimulatory CD28 and/or 4-1BB molecules can be used to express CARs.

The genetically engineered T cells of a patient expressing a CAR are capable of recognizing, e.g., a tumor target without Major Histocompatibility Complex (MHC) restriction, and destroying a target through cytotoxic effector mechanisms.

Furthermore, allogenic CAR-T cells can be generated with lymphocytes from hematopoietic stem cell donors and used, e.g., in the context of post-allograft relapse.

Methods of treatment using CAR-T cells involve lympho-depletion of a patient's T cells to make room for the patient's newly generated CAR-T cells to home after i.v. infusion. The immune-depletion further provides potentially a decrease in residual tumor mass, an induction of inflammation, the release of tumor antigen, and the decrease in the number of regulatory T cells, the latter of which could suppress the function of newly infused engineered CAR-T cells. Furthermore, if any elements of the CAR are of non-human origin the lympho-depletion may also decrease the risk of immunization against this CAR element. The use of variable fragments of humanized immunoglobulin in the construction of CARs can also aid in lowering the risk of immunization against CARs.

The inability to control CAR-T cells after infusion into patients has raised safety concerns. For example, on-target/off-tumor and off-target antigen recognition were observed in some patients during clinical trials. Further, uncontrolled CAR-T cell action in vivo can result in severe adverse events involving cytokine release syndrome and CAR-T related encephalopathy syndrome. For example, two major safety concerns of CAR-T cell therapies are the cytokine release syndrome and neurological toxicity, such as, e.g., CAR-T cell-related encephalopathy syndrome. Unfortunately, the cytokine release syndrome is relatively frequent and can occur in about 50% to 100% of patients receiving CAR-T cell therapy. Furthermore, extensive activation of T cells, in general, and CAR-T cells specifically leads to T cell death and, thus, exhaustion of the CAR-T cell population, In order to extend the lifespan of CAR-T cells following infusion into a patient, it would be desirable to control CAR-T cell activation, These safety and efficacy concerns are related to the inability to control CAR-T activation, expansion and, more importantly, terminate the expression of CARs in therapeutic CAR-T cells. Therefore, temporal and spatial control of CARs in therapeutic CAR-T cells could improve the safety and efficacy profile of this important technology and could curb, e.g., an excessive cytokine release and CAR-T cell exhaustion by fine-tuning the expression levels of CARs on CAR-T cells. Further, by allowing CAR fine-tuning on CAR-T cells, CAR expression could be turned off during remission and turned back on in the case of disease relapse.

A further problem for CAR-T therapy is a lack of or low response and relapse after CAR-T cell therapy related to a loss of antigen expression on the target cells. The use of CAR-T cells that are targeting more than one antigen can, therefore, be useful to address this issue. Thus, there is a need to develop new CAR-T cell therapies that can address the safety and efficacy concerns and the potential target cell resistance.

### BRIEF SUMMARY OF THE INVENTION

The instant invention provides chimeric-antigen-receptor (CAR) T cell systems and methods of making and using these for therapies of diseases that can be treated with targeted immune cells. The CAR-T cell systems of the instant invention comprise an inducible switch design based on a non-structural protein 3 (NS3) protease domain of the hepatitis C virus that can be used to fine-tune CAR expression in genetically engineered CAR-T cells. With this design, the safety and potency of CAR T cell therapy can be substantially improved.

Advantageously, the NS3 protease domain of the instant CAR T cell system provides a default auto-proteolysis of the CAR in transduced T cells as a means for maximum control over CAR expression and avoidance of CAR presence in a patient when such presence is not desired.

In contrast, when CAR expression is desired, a small molecule inhibitor can be administered to inhibit the proteolytic activity of the NS3 domain and block CAR auto-proteolysis leading to expression of the CAR on the surface of engineered CAR-T cells. Moreover, dose-dependent administration of said small molecule inhibitor to a subject treated with engineered CAR-T cells allows a fine-tuning of the level of CAR expression by engineered CAR-T cells and can be adjusted, e.g., according to the remaining burden of target cells and/or the occurrence of adverse events such as a cytokine release syndrome.

Advantageously, the CAR-T cell systems of the invention can comprise a variety of single-chain variable fragments against a variety of disease-related antigens including, but not limited to, cancer- and/or immune disorder-related antigens to treat cancer and/or immune disorders.

Further provided are CAR-T cell systems that simultaneously target at least two target antigens on the same or different target cells to improve therapeutic efficacy and lower the risk for adverse events.

For example, in one embodiment the invention provides CD19-CAR and CD22-CAR “cocktails” and/or CD19-CAR-T cell and CD22-CAR-T cell “cocktails” to provide high efficiency treatment for B-cell leukemia.

Advantageously, the CAR-T cell systems of the instant invention combine safety switches and dual target designs. For example, this strategy targets both CD19 and CD22 simultaneously, enabling more extensive cover for B-cell malignancies, reducing relapse caused by antigen escape and improving therapeutic efficacy. Furthermore, the novel protease switch system provides a precise regulation of CAR-T cell activity and allows the avoidance of CAR-T cell exhaustion in vivo because CAR expression can be stopped to prevent CAR-T cell overactivation and prolong CAR-T cell life in patients. This novel CAR-T cell system, thus, increases the safety and potency of CAR-T cell therapies.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1C show drawings of the CAR-T cell system of the instant invention. FIG. 1A shows a target cell and T cell interacting through a CAR-tumor antigen (TAA) binding event. FIG. 1B shows a detailed drawing of the CAR of the invention in an “off” and “on” state in the absence and presence of the NS3 inhibitor asunaprevir (ASV). FIG. 1C shows NS3-CAR constructs of the invention with a first cleavage site (1CS), a second cleavage site (2CS), and both cleavage sites. The yellow lines indicate NS3 cleavage sites and the red arrows indicate cleavage action of NS3 protease in the absence of asunaprevir.

FIG. 2 shows the CAR cassettes of a control CAR (top) and a NS3 switch-CAR (bottom) used for transduction of T cells to generate CAR-T cells.

FIG. 3 shows flow cytometry analyses of CD19-CAR expression in untransduced cells, CD19-CAR transduced cells, cells transduced with NS3-CD19-CAR without asunaprevir treatment and cells transduced with NS3-CD19-CAR with asunaprevir treatment.

FIGS. 4A-4B show CAR expression levels in CAR-transduced cells in the presence of increasing asunaprevir concentrations. FIG. 4A shows CAR expression determined by flow cytometry. FIG. 4B shows percentage of CAR expressing cells at different time points and in the presence of different concentrations of asunaprevir.

FIGS. 5A-5B show CAR expression levels in CAR-transduced cells upon withdrawal of asunaprevir. FIG. 5A shows flow cytometric data on CAR expression at 24 hours and 48 hours after asunaprevir withdrawal. FIG. 5B shows flow cytometric data on CAR expression at 24 hours and 48 hours in the presence of asunaprevir.

FIGS. 6A-6B show cell death and CD4/CD8 profiles of CAR-transduced cells in the presence and absence of asunaprevir. FIG. 6A shows flow cytometric data on annexin V expression and propidium iodine staining of untransduced cells, CD19-CAR transduced cells and cells transduced with NS3-CD19 CAR in the presence and absence of asunaprevir.

FIG. 6B shows flow cytometric data on CD4 and CD8 expression on untransduced cells, CD19-CAR transduced cells and cells transduced with NS3-CD19 CAR in the presence and absence of asunaprevir.

FIG. 7 shows a map of plasmid PLVX-EF1a-NS3 WT.

FIG. 8 shows a map of plasmid PLVX-EF1a-NS3 T54A.

FIG. 9 shows a map of plasmid PLVX-EF1a-NS3 1CS.

FIG. 10 shows a map of plasmid PLVX-EF1a-NS3 2CS.

FIG. 11 shows a map of plasmid PLVX-EF1a-NS3 AI.

FIG. 12 shows a map of plasmid PLVX-EF1a-NS3 TI.

FIG. 13 shows a map of plasmid PLVX-EF1a-NS3 Double Switch 1.

FIG. 14 shows a map of plasmid PLVX-EF1a-NS3 Double Switch 2.

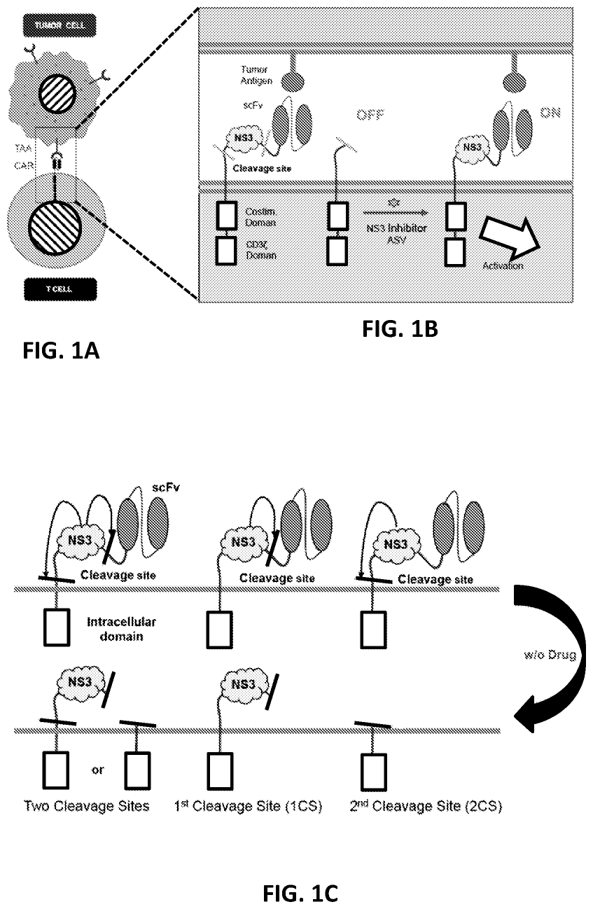

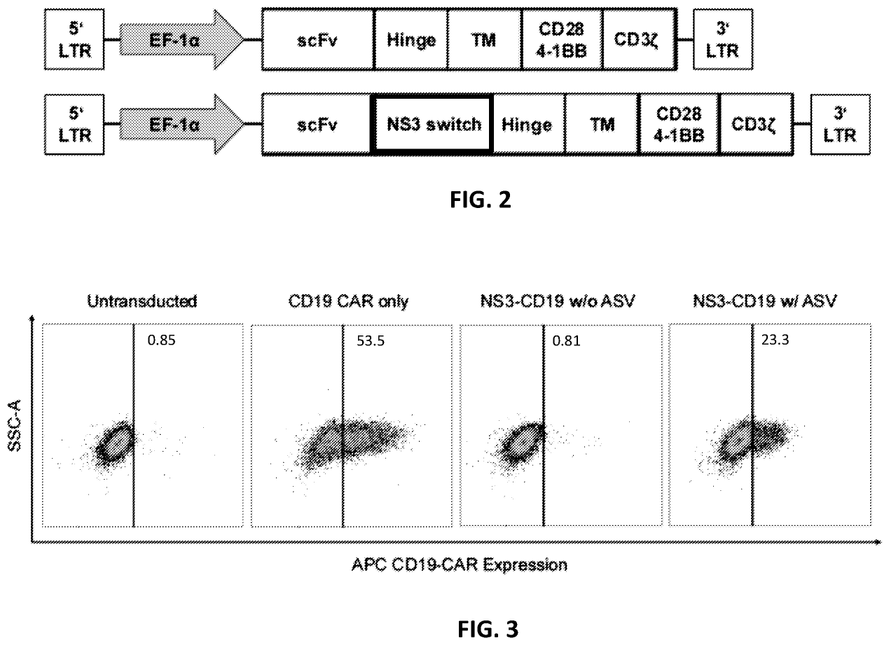

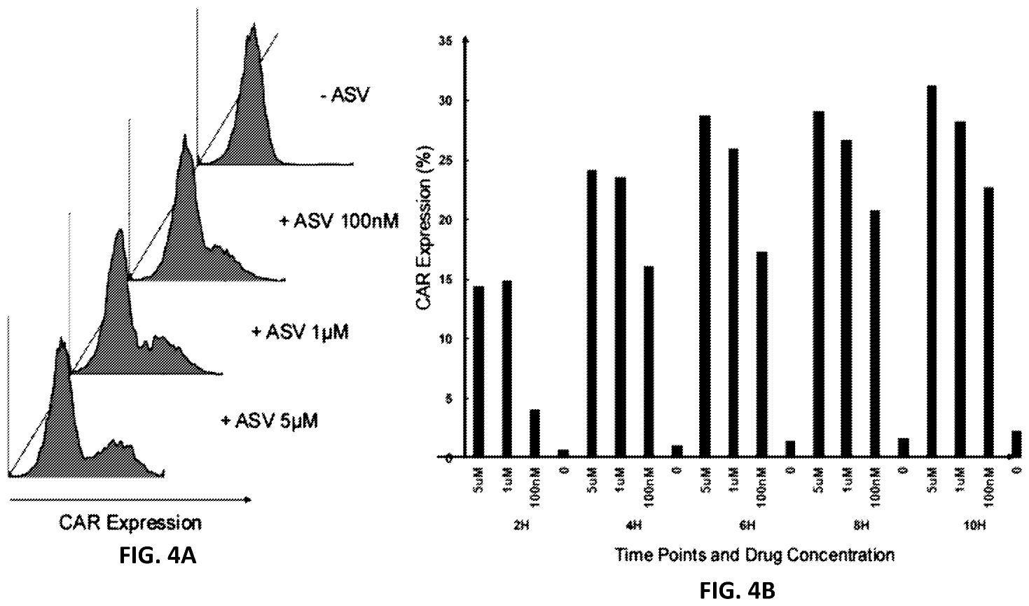

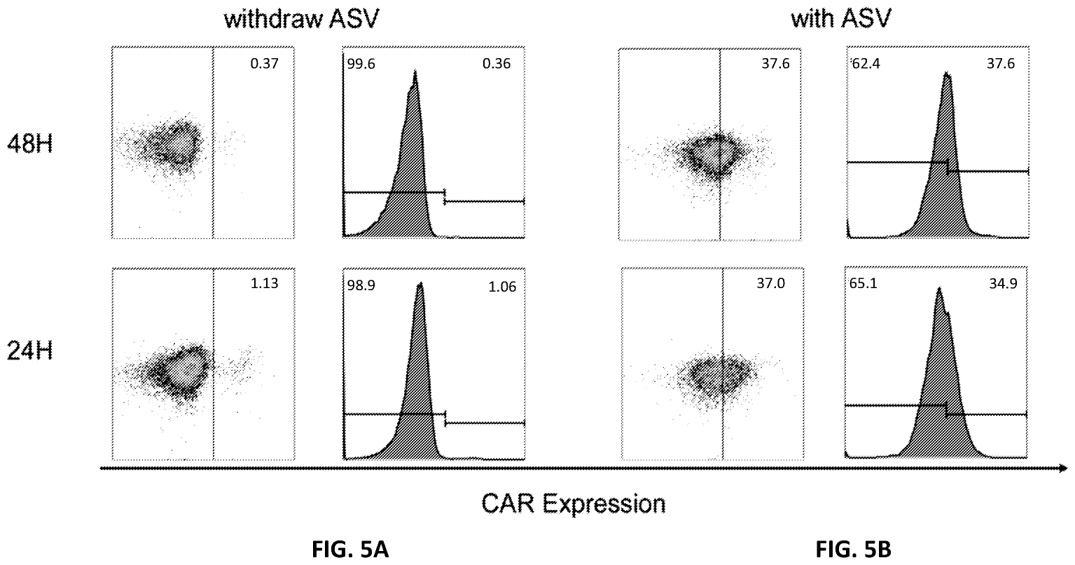

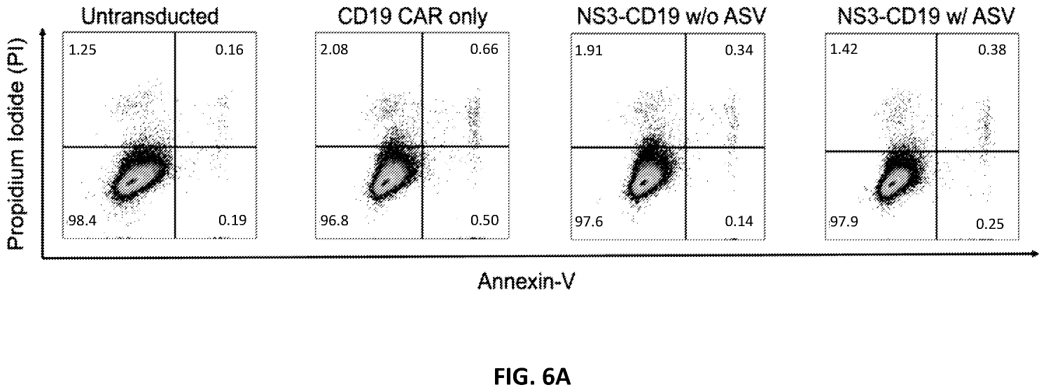

### BRIEF DESCRIPTION OF THE SEQUENCES

SEQ ID NO: 1 is the amino acid sequence of a CD8-alpha signal peptide of a CAR of the invention.

SEQ ID NO: 2 is the amino acid sequence of a CD19-single chain variable fragment of a CAR of the invention.

SEQ ID NO: 3 is the amino acid sequence of a CD22-single chain variable fragment of a CAR of the invention.

SEQ ID NO: 4 is the amino acid sequence of a CD8 alpha hinge region of a CAR of the invention.

SEQ ID NO: 5 is the amino acid sequence of a CD28 hinge region of a CAR of the invention.

SEQ ID NO: 6 is the amino acid sequence of an IgG4 hinge region of a CAR of the invention.

SEQ ID NO: 7 is the amino acid sequence of an IgG4m hinge region of a CAR of the invention.

SEQ ID NO: 8 is the amino acid sequence of an IgG1 hinge region of a CAR of the invention.

SEQ ID NO: 9 is the amino acid sequence of an IgG2 hinge region of a CAR of the invention.

SEQ ID NO: 10 is the amino acid sequence of an IgG4 CH2 CH3 hinge and spacer region of a CAR of the invention.

SEQ ID NO: 11 is the amino acid sequence of an IgG2 CH2 CH3 hinge and spacer region of a CAR of the invention.

SEQ ID NO: 12 is the amino acid sequence of an IgG1 CH2 CH3 hinge and spacer region of a CAR of the invention.

SEQ ID NO: 13 is the amino acid sequence of a CD28 transmembrane domain of a CAR of the invention.

SEQ ID NO: 14 is the amino acid sequence of a CD8-alpha transmembrane domain of a CAR of the invention.

SEQ ID NO: 15 is the amino acid sequence of a CD4 transmembrane domain of a CAR of the invention.

SEQ ID NO: 16 is the amino acid sequence of a CD3-zeta transmembrane domain of a CAR of the invention.

SEQ ID NO: 17 is the amino acid sequence of an ICOS transmembrane domain of a CAR of the invention.

SEQ ID NO: 18 is the amino acid sequence of a 4-1BB intracellular domain of a CAR of the invention.

SEQ ID NO: 19 is the amino acid sequence of a CD28 intracellular domain of a CAR of the invention.

SEQ ID NO: 20 is the amino acid sequence of a CD3-zeta intracellular domain of a CAR of the invention.

SEQ ID NOs: 21-979 are the amino acid sequences of light chain variable domains and heavy chain variable domains of the antibodies listed in Table 2.

SEQ ID NO: 980 is the amino acid sequence of a wild-type NS3 protease domain of a CAR of the invention.

SEQ ID NO: 981 is the amino acid sequence of a T54A mutant NS3 protease domain of a CAR of the invention.

SEQ ID NO: 982 is the amino acid sequence of an asunaprevir-inhibitable (AI) NS3 protease domain of a CAR of the invention.

SEQ ID NO: 983 is the amino acid sequence of a telaprevir-inhibitable (TI) NS3 protease domain of a CAR of the invention.

SEQ ID NO: 984 is the amino acid sequence of a NS4A domain of a CAR of the invention.

SEQ ID NO: 985 is the amino acid sequence of a first protease cleavage site of a CAR of the invention.

SEQ ID NO: 986 is the amino acid sequence of a second protease cleavage site of a CAR of the invention.

SEQ ID NO: 987 is the amino acid sequence of a third protease cleavage site of a CAR of the invention.

SEQ ID NO: 988 is the amino acid sequence of a fourth protease cleavage site of a CAR of the invention.

SEQ ID NO: 989 is the amino acid sequence of a first self-cleaving viral 2A peptide (T2A) of a CAR of the invention.

SEQ ID NO: 990 is the amino acid sequence of a second self-cleaving viral 2A peptide (P2A) of a CAR of the invention.

SEQ ID NO: 991 is the amino acid sequence of a CD19 light chain of a CAR of the invention.

SEQ ID NO: 992 is the amino acid sequence of a CD19 heavy chain of a CAR of the invention.

SEQ ID NO: 993 is the amino acid sequence of a CD22 heavy chain of a CAR of the invention.

SEQ ID NO: 994 is the amino acid sequence of a CD22 light chain of a CAR of the invention.

SEQ ID NO: 995 is the amino acid sequence of a first single chain variant fragment linker.

SEQ ID NO: 996 is the amino acid sequence of a second single chain variant fragment linker.

SEQ ID NO: 997 is the amino acid sequence of a third single chain variant fragment linker.

### DETAILED DISCLOSURE OF THE INVENTION

Provided are chimeric-antigen-receptor (CAR) T cell systems and methods of making and using these systems for therapies of oncological and other diseases. The CAR-T cell systems of the invention use chemogenetic control of protein function to achieve both customized and reliable on- and off-effects. The inducible switch design of the instant invention is based on the non-structural protein 3 (NS3) protease domain of the hepatitis C virus (HCV), which NS3 domain self-cleaves at specific cleavage sites.

The NS3 protease domain of the CAR-T cell system of the invention is localized within the CAR construct such that the auto-proteolytic activity of the NS3 protease cleaves the CAR construct by default. In contrast, in the presence of a small molecule inhibitor that blocks the NS3 proteolytic activity the engineered CARs of the invention are expressed on the surface of CAR transduced T cells. The CAR-T cell system of the invention allows dose-dependent induction of CAR expression on transduced T cells and removal of CARs from T cells upon withdrawal of the small molecule inhibitor. Therefore, this novel regulated CAR-T cell system allows customized CAR-T cell therapy though expression of CARs on transduced T cells and further enables the maintenance of CAR-transduced T cells.

In specific embodiments of the instant invention, T cells expressing CARs are generated by genetic engineering to arm the immunocompetent T cells of a patient with an activating receptor comprising (1) an extracytoplasmic variable fragment of an immunoglobulin, e.g., a single chain variable fragment (scFv), directed against a tumor target or an immune cell target, (2) a NS3 protease domain, (3) a hinge region, (4) a transmembrane domain, (5)) at least one positive co-stimulation molecule domain from, e.g., CD28 and/or 4-1BB, and (6) an intracellular T-cell receptor activation molecule domain from e.g., CD3-zeta.

In some embodiments, the CARs of the invention are engineered using the entire intracellular signaling domain of a T cell receptor. In other embodiments, parts of the entire intracellular signaling domain of a T cell receptor can be used. To the extent that a truncated portion of the intracellular signaling domain of a TCR is used, such truncated portion may be used in place of the intact chain as long as it transduces the effector function signal. In specific embodiments, the intracellular signaling domain of a CAR of the instant invention is a truncated portion of the intracellular signaling domain capable of transducing the effector function signal of a TCR.

Examples of intracellular signaling domains for use in the CAR of the invention include, but are not limited to, the cytoplasmic sequences of T cell receptors and co-receptors that initiate signal transduction following antigen receptor engagement, as well as any derivatives or variants of these sequences and any synthetic sequences that have equivalent functional capability.

In certain embodiments, the intracellular domain of the CAR further comprises a secondary or co-stimulatory signal. The costimulatory signaling portion refers to a region of the CAR comprising the intracellular domain of a costimulatory molecule. A costimulatory molecule is a cell surface molecule other than an antigen receptor or their ligands that is required for an efficient response of lymphocytes to an antigen.

In some embodiments, T cell activation is mediated by two distinct classes of intracellular signaling sequences: those that initiate antigen-dependent primary activation through the TCR (primary intracellular signaling sequences) and those that act in an antigen-independent manner to provide a secondary or co-stimulatory signal (secondary intracellular signaling sequences).

In some embodiments, the intracellular domain of a CAR of the invention comprises a CD3-zeta signaling domain, and optionally, any other desired cytoplasmic domain(s) useful in the context of a CAR. For example, in some embodiments, the intracellular domain of the CAR comprises a CD3-zeta chain portion and a costimulatory signaling domain. Examples of costimulatory molecules from which costimulatory signaling domains can be derived and used in the generation of CARs of the instant invention include, but are not limited to, CD27, CD28, 4-1BB (CD137), OX40, CD30, CD40, PD-1, ICOS (CD278), lymphocyte function-associated antigen-1 (LFA-1), CD2, CD7, TNFSF14, NKG2C, B7-H3, CD132, and ILRβ(CD122). While the use of CD28 and 4-1BB as co-stimulatory signaling elements (costimulatory signaling domains) are exemplified in the Examples, other costimulatory signaling domains (e.g., OX40, CD30, CD40, PD-1, ICOS (CD278), lymphocyte function-associated antigen-1 (LFA-1), CD2, CD7, TNFSF14, NKG2C, B7-H3, CD132, and ILRβ(CD122)) can be used and are within the scope of the invention.

In preferred embodiments, the intracellular domain of the CAR of the invention comprises one or more signaling domains including, but not limited to, signaling domains of CD3-zeta, CD28, and 4-1BB.

In some embodiments, the CARs of the invention comprise hinge regions including, but not limited to, a CD8-alpha hinge region, a CD28 hinge region, an IgG4 hinge region, an IgG4m hinge region, an IgG1 hinge region, an IgG2 hinge region. In some embodiments, the CARs of the invention comprise a hinge region and a spacer including but not limited to, an IgG4 CH2 CH3 hinge and spacer, an IgG2 CH2 CH3 hinge and spacer, or an IgG1 CH2 CH3 hinge and spacer.

In some embodiments, the CARs of the invention comprise transmembrane domain including, but not limited to, a CD28 transmembrane domain, a CD8-alpha transmembrane domain, a CD4 transmembrane domain, a CD3-zeta transmembrane domain, or an ICOS transmembrane domain,

CARs useful according to the present invention include first generation CARs, second generation CARs, and third generation CARs. First generation CARs comprise a binding moiety specifically recognizing an antigen of interest and a T cell activating signaling domain (without an intracellular costimulatory domain). Second and third generation CARs include signal sequences from various costimulatory molecules. Second generation CARs comprise a binding moiety specifically recognizing an antigen of interest, a T cell activating intracellular signaling domain, and one intracellular costimulatory domain. Third generation CARs comprise a binding moiety specifically recognizing an antigen of interest, a T cell activating intracellular signaling domain, and two or more intracellular costimulatory domains.

In some embodiments of first generation CARs, the intracellular domains comprise the signaling domain of CD3-zeta. In some embodiments of second generation CARs, the intracellular domains comprise the signaling domains of CD3-zeta and CD28. In some embodiments of third generation CARs, the intracellular domains comprise the signaling domains of CD3-zeta, CD28 and 4-1BB. In certain embodiments, the cytoplasmic domains of the CARs comprise any combination of CD3-zeta, CD28, and 4-1BB signaling domains or any combination of any other signaling domains useful in inducing T cell activation.

In preferred embodiments, the CARs of the invention comprise a single chain variable fragment of an antibody (scFv) a Non-Structural protein 3 (NS3) protease domain of hepatitis C virus (HCV), a human CD8 hinge and transmembrane domain, and human CD3 zeta, human CD28, and human 4-1BB signaling domains.

In some embodiments, the NS3 protease domain is integrated into the CAR at a specific location such that the NS3 protease activity cleaves the CAR protein construct at specific predetermined protease cleavage sites and prevents expression of an intact CAR protein construct on the cell surface.

In some embodiments, the NS3 protease domain further comprises NS4 protein sequences of HCV to ensure proper NS3 protease expression and function. In other embodiments, the CAR construct can comprise a protease domain originating from sequences of HCV NS2 and NS3 proteases. In preferred embodiments of the invention, the NS3 protease domain comprises NS4A sequences (NS3/4A protease domain).

In some embodiments, the CAR-T cells comprise an antigen-binding domain of an antibody that binds specifically to a cancer antigen and/or an immune cell antigen, a NS3/4A protease domain, cleavage sites, a hinge region, a transmembrane domain, and the intracellular signaling domain of CD3-zeta. In some embodiments, the CAR-T cells comprise an antigen-binding domain of an antibody that binds specifically to a cancer antigen and/or an immune cell antigen, a NS3/4A protease domain, cleavage sites, a hinge region, a transmembrane domain, the intracellular signaling domain of CD3-zeta, and the intracellular signaling domain of CD28. In some embodiments, the CAR-T cells comprise an antigen-binding domain of an antibody that binds specifically to a cancer antigen and/or an immune cell antigen, a NS3/4A protease domain, cleavage sites, a hinge region, a transmembrane domain, the intracellular signaling domain of CD3-zeta, the intracellular signaling domain of CD28, and the intracellular signaling domain of 4-1BB.

In some embodiments, the scFv comprise variable portions of the antibody heavy and light chains fused by a flexible linker. The linkers of the CARs of the invention include, but are not limited to, the single chain variable fragment linkers of SEQ ID NOs:995-997. In specific embodiments, the flexible linker allows the scFv to orient in different directions to enable antigen binding. In some embodiments, the CARs of the invention, when expressed in T cells, are able to direct antigen recognition based on the antigen binding specificity to activate a T cell-mediated immune response.

The single chain variable domain of the CAR of the instant invention can be engineered to target any one or more proteins present on the surface of a target cell. For example variable domain regions of antibodies that bind an antigen on a target cell can be engineered as single chain variable fragments of the CARs and can be used to generate CAR-T cells of the invention that target the specific antigen. The antigens that can be targeted by CAR-T cells of the invention include, but are not limited to, parasitic antigens, bacterial antigens, viral antigens and auto-antigens.

In some embodiments, the CARs of the invention comprise a single chain variable fragment of an anti-cancer antibody (anti-cancer scFv), a NS3/4A protease domain, cleavage sites, a human CD8 hinge and transmembrane domain, and human CD3-zeta, human CD28, and human 4-1BB signaling domains.

In some embodiments, the CARs of the invention comprise a single chain variable fragment of an anti-immune cell antibody (anti-immune cell scFv), a NS3/4A protease domain, cleavage sites, a human CD8 hinge and transmembrane domain, and human CD3-zeta, human CD28, and human 4-1BB signaling domains.

In some embodiments, the CARs of the invention comprise a single chain variable fragment of an antibody binds a parasitic antigen, a bacterial antigen and/or a viral antigen (anti-infectious scFv), a NS3/4A protease domain, cleavage sites, a human CD8 hinge and transmembrane domain, and human CD3-zeta, human CD28, and human 4-1BB signaling domains.

In some embodiments, the CARs of the invention comprise a single chain variable fragment of an antibody that binds an auto-immune-reactive cell (anti-auto-immune cell scFv), a NS3/4A protease domain, cleavage sites, a human CD8 hinge and transmembrane domain, and human CD3-zeta, human CD28, and human 4-1BB signaling domains. Advantageously, the CARs of the invention can be used to fine-tune CAR expression of CAR-T cells and, thus, allow the safe targeting of antigens on auto-reactive cells where the CAR expression can be turned off once the auto-reactive cells have been targeted to avoid targeting non-auto-reactive cells that might express similar antigens.

An anti-cancer antibody, as used herein, refers to any antibody that binds to any antigen that is present on a cancer cell.

An anti-immune cell antibody, as used herein, refers to any antibody that binds to any antigen that is present on an immune cell.

Examples of antibodies that comprise variable domains that can be used in the engineering of CARs of the instant invention include, but are not limited to, antibodies that specifically bind to alpha feto protein 1; anaplastic lymphoma kinase (CD246); V-set domain-containing T-cell activation inhibitor 1; B-cell CLL/lymphoma 2 (BCL-2); B cell receptor-Abl fusion protein (Bcr/Abl); Chorionic gonadotropin beta subunit 3 (beta-HCG); beta-2 microglobulin; B-raf proto-oncogene (BRAF); Breast cancer type 1 susceptibility protein (BRCA1); Breast cancer type 2 susceptibility protein (BRCA2); B-cell maturation antigen (BCMA); B7-like molecule H4 (B7-H4); Cancer antigen 15-3 (Ca 15-3); Cancer antigen 19-9 (Ca 19-9); calcitonin; calretinin; neprilysin (CD10); phagocytic glycoprotein-1 (CD44); protein tyrosine phosphatase receptor C (CD45); carcinoembryonic antigen (CEA); chromogranin A; tyrosine protein kinase Kit (c-kit); cytokeratin 19; epidermal growth factor receptor (EGFR); epithelial cell adhesion molecular (EpCAM); estrogen receptor-alpha; estrogen receptor-beta; folate receptor 1; epididymal secretory protein E4 (HE4); glypican-3 protein (GPC-3); immunoglobulin-associated beta (CD79b); v-erb-b2 erythroblastic leukemia viral oncogene homolog 2 (HER2); inhibin; integrin-associated protein (IAP/CD47); Interleukin-3 receptor (CD123); antigen identified by monoclonal antibody Ki-67 (Ki-67); Kirsten rat sarcoma viral oncogene homolog (KRAS); lysosomal-associated membrane protein 1 (LAMP1); leukocyte antigen CD37; melan-A (MART1); melanoma cell adhesion molecule (MCAM/CD146); mesothelin; mucin 1 (MUC1); mucin 16/ovarian carcinoma antigen 125 (MUC16/CA125); nuclear matric protein 22 (NMP22); neuron-specific enolase (NSE); neurotrophic tyrosine kinase receptor-related 1 (ROR1); nerve growth factor receptor (NGFR); tumor protein 53 (p53); cytoskeleton-associated protein 4 (p63); programmed cell death protein 1 (PD1); programmed death-ligand 1 (PD-L1/CD274); pyruvate kinase muscle (PKM); phospholipase A2-activating protein (PLAP); podoplanin; progesterone receptor; prostate-specific antigen (PSA); sialic acid binding Ig-like lectin 3 (CD33); S100 calcium binding protein A4 (S100A4); serpin peptidase inhibitor glade E (SERPINE1); secreted frizzled-related protein 1 (SFRP1) signaling lymphocyte activating-molecule-related receptor family including, but not limited to, signaling lymphocyte activating-molecule (CD150), 2B4 (CD244), CD84, NTB-A (Ly-108), and Ly-9 (CD229); syndecan-1 (CD138); tumor associated glycoprotein 72 (TAG-72); thymidine kinase; thyroglobulin; transthyretin; transcription termination factor 1 (TTF1); plasminogen activator urokinase (uPA); vascular endothelial growth factor receptor 2 (VEGR2); or vimentin.

In some embodiments of the instant invention, CARs comprise single chain variable fragments of any variable domains that bind to a desirable target antigen. Advantageously, any variable domain of any antibody binding a desired target antigen can be used to generate CARs of the instant invention because the amino acid sequences of antibody variable domains can readily be converted into single chain variable fragment sequences of the CARs of the instant invention. For example, in some embodiments, variable domains from bi-specific antibodies or multi-specific antibodies with variable domains that bind to more than two antigens can be used to generate CARs of the instant invention. In some embodiments, the heavy and light chain of a first variable domain of a bi- or multi-specific antibody can be used to generate one CAR and the heavy and light chain of a second variable domain of the bi- or multi-specific antibody can be used to generate a second CAR.

In some embodiments, a heavy and/or light chain domain of one variable domain of one antibody and a heavy and/or light chain of a second variable domain of a second antibody or of a second variable domain of a bi-specific or multi-specific antibody can be used to generate a CAR of the invention. In further embodiments, more than one heavy chain domain and more than one light chain domain can be combined by linkers to generate multi-specific CARs of the invention. The more than one heavy chain and more than one light chain domain can be from single variable domains of different antibodies or from different variable domains of bi- or multi-specific antibodies.

In further embodiments, heavy chain domains without light chain domains and light chain domains without heavy chain domains can be used to generate CARs of the invention. In some embodiments, a heavy chain from a first variable domain and a light chain from a second variable domain can be used to generate a CAR of the invention. In some embodiments, more than one heavy chain from more than one variable domain can be combined to generate a CAR of the invention. In some embodiments, more than one light chain from more than one variable domain can be combined to generate a CAR of the invention. In yet further embodiments, more than one heavy chain from more than one first group of variable domains and more than one light chain from more than one second group of variable domains can be combined to generate a CAR of the invention.

For example, the scFv of the CAR of the invention can comprise variable sequences from antibodies including, but not limited to, Abagovomab, Abelacimab, Abituzumab, Abrezekimab, Abrilumab, Actoxumab, Adalimumab, Aducanaumab, Afasevikumab, Alacizumab, Alemtuzumab, Alirocumab, Amatuximab, Andecaliximab, Anetumab, Anifrolumab, Anrukizumab, Apamistamab, Aprutumab, Ascrinvacumab, Atezolizumab, Atidortozumab, Atinumab, Atoltivimab, Avelumab, Axicabtagene Ciloleucel, Azintuxizumab, Balstilimab, Bapineuzumab, Basiliximab, Bavituximab, Bedinvetmab, Begelomab, Belantamab, Belimumab, Bemarituzumab, Benralizumab, Berlimatoxumab, Bermekimab, Bersanlimab, Bevacizumab, Bezlotoxumab, Bimagrumab, Bimekizumab, Birtamimab, Bleselumab, Blinatumomab, Blontuvetmab, Blosozumab, Bococizumab, Brazikumab, Brentuximab, Briakinumab, Brodalumab, Broluczumab, Brontictuzumab, Budigalimab, Burosumab, Cabiralizumab, Camidanlumab, Camrelizumab, Canakinumab, Cantuzumab, Caplacizumab, Carlumab, Carotuximab, Cemiplimab, Candakimab, Cergutuzumab, Certolizumab, Cetrelimab, Cetuximab, Cibisatamab, Cinpanemab, Citatuzumab, Cixutumumab, Clazakizumab, Clervonafusp, Clivatuzumab, Cobolimab, Codrituzumab, Cofetuzumab, Coltuximab, Conatumumab, Concizumab, Cosfroviximab, Crenezumab, Crizanlizumab, Crotedumab, Crovalimab, Cusatuzumab, Dacetuzumab, Daclizumab, Dalotuzumab, Dapirolizumab, Daratumumab, Dectrekumab, Demcizumab, Denintuzumab, Denosumab, Depatuxizumab, Derlotuximab, Dezamizumab, Dilpacimab, Dinutuximab, Diridavumab, Disitamab, Domagrozumab, Donanemab, Dostarlimab, Drozitumab, Duligotuzumab, Dupilumab, Durvalumab, Dusigitumab, Duvortuxizumab, Eculizumab, Edrecolomab, Efalizumab, Efungumab, Eldelumab, Elezanumab, Elgemtumab, Elipovimab, Elotuzumab, Emactuzumab, Emapalumab, Emicizumab, Emibetuzumab, Enapotamab, Enavatuzumab, Enfortumab, Enoblituzumab, Enokizumab, Enoticumab, Ensituximab, Envafolimab, Epratuzumab, Eptinezumab, Erenumab, Etaracizumab, Etigilimab, Etokimab, Etrolizumab, Evinacumab, Evolocumab, Faricimab, Farletuzumab, Fasinumab, Fezakinumab, Ficlatuzumab, Figitumumab, Firivumab, Flanvotumab, Fletikumab, Flotetuzumab, Fontolizumab, Foralumab, Foravirumab, Fremanezumab, Fresolimumab, Frovocimab, Frunevetmab, Fulranumab, Futuximab (Zatuximab), Galcanezumab, Galiximab, Gancotamab, Ganitumab, Gantenerumab, Garadacimab, Garetosmab, Gatipotuzumab, Gedivumab, Gemtuzumab, Gevokizumab, Gilvetmab, Gimsilumab, Girentuximab, Glembatumumab, Glenzocimab, Golimumab, Gosuranemab, Guselkumab, Ianalumab, Ibalizumab, Icrucumab, Idarucizumab, Ieramilimab, Ifabotuzumab, Iladatuzumab, Imalumab, Imaprelimab, Imgatuzumab, Inclacumab, Indatuximab, Indusatumab, Inebilizumab, Infliximab, Inotuzumab, Intetumumab, Ipilimumab, Iratumumab, Isatuximab, Iscalimab, Istiratumab, Itolizumab, Ixekizumab, Labetuzumab, Lacnotuzumab, Lacutamab, Ladiratuzumab, Lampalizumab, Lanadelumab, Landogrozumab, Laprituximab, Larcaviximab, Lebrikizumab, Lenvervimab, Lenzilumab, Leronlimab, Lesofavumab, Letolizumab, Levilimab, Lexatumumab, Lifastuzumab, Ligelizumab, Lilotomab, Lintuzumab, Lirilumab, Lodelcizumab, Lokivetmab, Loncastuximab, Lorukafusp, Lorvotuzumab, Losatuxizumab (Serclutamab), Lucatumumab, Lulizumab, Lumiliximab, Lumretuzumab, Lupartumab, Lutikizumab, Maftivimab, Magrolimab, Margetuximab, Marstacimab, Matuzumab, Mavrilimumab, Mepolizumab, Milatuzumab, Mirikizumab, Mirvetuximab, Mitazalimab (Vanalimab), Modotuximab, Mogamulizumab, Monalizumab, Mosunetuzumab, Motavizumab, Moxetumomab, Murlentamab, Muromonab (Zolimomab), Namilumab, Naptumomab, Naratuximab, Narnatumab, Natalizumab, Navivumab, Navicixizumab, Naxitamab, Necitumumab, Nemolizumab, Nesvacumab, Netakimab, Nidanilimab, Nimacimab, Nimotuzumab, Nirsevimab, Nivolumab, Obexelimab, Obiltoxaximab, Obinutuzumab (Afutuzumab), Ocaratuzumab, Ocrelizumab, Ofatumumab, Olaratumab, Oleclumab, Olendalizumab, Olinvacimab (Tanibirumab), Olokizumab, Omalizumab, Omburtamab, Onartuzumab, Ontamalimab, Ontuxizumab, Onvatilimab, Opicinumab, Oportuzumab, Orilanolimab, Orticumab, Osocimab, Otelixizumab, Otilimab, Otlertuzumab, Oxelumab, Ozanezumab, Ozoralizumab, Pabinafusp, Palivizumab, Pamrevlumab, Panitumumab, Panobacumab, Parsatuzumab, Pasotuxizumab, Pateclizumab, Patritumab, Pembrolizumab (Lambrolizumab), Pepinemab, Perakizumab, Pertuzumab, Pidilizumab, Pinatuzumab, Placulumab, Plamotamab, Plozalizumab, Polatuzumab, Ponezumab, Porgaviximab, Prasinezumab, Prezalumab, Pritoxaximab, Prolgolimab, Quetmolimab, Quilizumab, Racotumomab, Radretumab (Bifikafusp/Onfekafusp), Rafivirumab, Ralpancizumab, Ramucirumab, Ranevetmab, Ranibizumab, Ravagalimab, Ravulizumab, Refanezumab, Relatlimab, Relfovetmab, Remtolumab, Reslizumab, Rilotumumab, Rinucumab, Risankizumab, Rituximab, Rivabazumab, Robatumumab, Roledumab, Rolinsatamab, Romilkimab, Romosozumab, Rontalizumab, Rosmantuzumab, Rovalpituzumab, Rozanolixizumab, Rozipafusp, Ruplizumab, Sacituzumab, Samalizumab, Samrotamab, Sarilumab, Satralizumab (Sapelizumab), Secukinumab, Selicrelumab Semorinemab, Seribantumab, Setoxaximab, Setrusumab, Sifalimumab, Siltuximab, Simtuzumab, Sintilimab, Sirtratumab, Sirukumab, Sofituzumab, Solanezumab, Solitomab, Spartalizumab, Spesolimab, Suptavumab, Sutimlimab, Suvizumab, Suvratoxumab, Tabalumab, Tabituximab, Tadocizumab, Tafasitamab, Talacotuzumab, Tamrintamab, Tamtuvetmab, Tanezumab, Tarextumab, Tavolimab (Tavolixizumab), Tebentafusp, Teclistamab, Telisotuzumab, Temelimab, Tenatumomab, Teplizumab, Tepoditamab, Teprotumumab, Tesidolumab, Tezepelumab, Tibulizumab, Tidutamab, Tigatuzumab, Tilavonemab, Tildrakizumab, Timolumab, Tiragolumab, Tislelizumab, Tisotumab, Tocilizumab, Tomaralimab, Tomuzotuximab, Toripalimab, Tosatoxumab, Tovetumab, Tralokinumab, Trastuzumab (Timigutuzumab), Tregalizumab, Tremelimumab (Ticilimumab), Trevogrumab, Ublituximab, Ulocuplumab, Urelumab, Ustekinumab, Utomilumab, Vadastuximab, Valanafusp, Vantictumab, Vanucizumab, Varisacumab, Varlilumab, Vatelizumab, Vedolizumab, Veltuzumab, Vesencumab, Vibecotamab, Visilizumab, Vobarilizumab, Vofatamab, Volagidemab, Vonlerolizumab (Pogalizumab), Vopratelimab, Vorsetuzumab, Vunakizumab, Xentuzumab, Zalifrelimab, Zalutumumab, Zampilimab, Zanolimumab, Zenocutuzumab, Zolbetuximab (Claudiximab), and any combinations of variable light and/or variable heavy chain sequences of any of these antibodies. The amino acid sequences of the heavy chain and light chain variable regions of the above antibodies are available on the IMGT website (see Worldwide Website: imgt.org and are also provided in the attached sequence listing and Table 2).

In further embodiments, any variable region of any antibody discovered in the art at any time following the instant disclosure can be included in the CAR constructs of the instant invention because, based on the instant disclosure, a person or ordinary skill in the art can employ routine methodology to incorporate the variable region sequences into the CAR constructs and generate CAR-T cells according to the instant invention.

In preferred embodiments, the single chain variable fragment of a cell-binding antibody that is used in the construction of a CAR of the instant invention is a fragment that binds to a membrane proximal region of a target antigen such that the binding of the CAR generated from such binding fragment is efficient in physically blocking dimerization of the target antigen with any dimerization partner.

In some embodiments, the antigen binding fragment of the CAR of the invention is derived from a designed ankyrin repeat protein that binds an antigen on a target cell. Binding fragments of designed ankyrin repeat proteins useful in the CARs of the invention can contain between 1 and 20, preferably between 2 and 6, ankyrin repeats comprising both framework sequences and variable sequences and can be retrieved from ankyrin repeat protein libraries. Advantageously, ankyrin repeat proteins bind desirable target molecules on the cell surface of a target cell, have a high stability and low antigenicity, thus lowering the risk for adverse side effects.

The CARs of the instant invention comprise at least one cytoplasmic domain and/or intracellular signaling domain that activate at least one of the normal effector functions of the T cell in which the CAR is expressed. As used herein, the term “intracellular signaling domain” refers to the portion of a protein that transduces the effector function signal and directs the cell to perform a specialized function. The term “effector function” refers to a specialized function of a T cell. Effector function of a T cell can be, e.g., cytotoxic and immune-stimulatory activities including the secretion of inflammatory cytokines.

In some embodiments, the CARs of the invention further comprise a transmembrane domain. In preferred embodiments, the transmembrane domain is fused to an extracellular antigen-binding domain of the CAR. In certain embodiments, the transmembrane domain is derived from a molecule whose transmembrane domain is naturally associated with one of the domains of the CAR of the invention. In certain embodiments, the transmembrane domain is modified (such as by amino acid substitutions) to avoid binding to the transmembrane domains of other cell membrane molecules to minimize undesirable interactions with other membrane-associated molecules.

The transmembrane domain may be derived either from natural or synthetic sources. In some embodiments, the source is natural and the domain may be derived from any membrane-bound or transmembrane protein. Transmembrane regions useful according to the present invention can be derived from, or can comprise, at least one of the transmembrane region(s) of the alpha, beta or zeta chain of the T-cell receptor, CD4, CD8-alpha, CD28, CD3 epsilon, CD3-zeta, CD45, CD5, CDS, CD9, CD16, CD22, CD33, CD37, CD64, CD80, CD86, CD134, CD137, CD 154 and/or ICOS (inducible T-cell stimulator).

In some embodiments, the transmembrane domain useful according to the instant invention can be synthetic, and can comprise predominantly hydrophobic residues such as leucine and valine. Preferably, the N- and/or C-terminals of a synthetic transmembrane domain comprise a triplet of phenylalanine, tryptophan, and valine.

Optionally, the transmembrane domain and the intracellular signaling domain of the CAR of the invention can be linked by a short oligo- or polypeptide spacer, preferably between 2 and 10 amino acids in length.

As used herein, the terms “spacer” or “linker” mean any oligo- or polypeptide that functions to link the transmembrane domain to the extracellular domain or the intracellular domain in the polypeptide chain. A spacer may comprise up to 300 amino acids, preferably 10 to 100 amino acids, more preferably 25 to 50 amino acids, most preferably 5 to 20 amino acids.

The NS3/4A protease domains of the CARs of the instant invention cleave specific cleavage sites. In some embodiments, the cleavage site is a decapeptides comprising, e.g., six amino acids on the N-terminal side of the cleavage side and four resides on the C-terminal site. In other embodiments, the cleavage sites comprise between 1 and 100 amino acids and any number of amino acids in between and are cleaved at any location within the cleavage site peptide.

In preferred embodiments, the cleavage sites of the CARs of the invention comprise the amino acids DLEVVT/STWV; DEMEEC/SQHL; ECTTPC/SGSW; and/or EDVVCC/SMSY, wherein “/” indicates the cleaved peptide bond.

In preferred embodiments, the NS3/4A protease domain of the CARs of the instant invention is located between the single chain variable fragment and the hinge region,

In other embodiments, the NS3/4A protease domain of the CAR is located between the hinge region and the transmembrane domain. In yet other embodiments, the NS3/4A protease domain is located between the intracellular domains, e.g., between the CD28 domain and the 4-1BB domain, between the CD28 domain and the CD3-zeta domain; between the 4-1BB domain and the CD3-zeta domain; or within any portion of the CAR of the invention as long as its presence does not interfere with the expression, membrane location and/or function of the CAR.

In preferred embodiments, the CAR of the instant invention comprises a single NS3/4A cleavage site. In other preferred embodiments, the CAR of the instant invention comprises more than one NS3/4A cleavage site.

In preferred embodiments, the single NS3/4A cleavage site of the CAR of the instant invention is located at or adjacent to the N-terminus of the NS3/4A protease domain.

In other preferred embodiments, the single NS3/4A cleavage site of the CAR of the instant invention is located at or adjacent to the C-terminus of the NS3/4A protease domain.

In more preferred embodiments, the CAR of the invention comprises more than one NS3/4A cleavage site and at least one NS3/4A cleavage site is located at or adjacent to the N-terminus of the NS3/4A protease domain.

In further preferred embodiments, the CAR of the invention comprises more than one NS3/4A cleavage site and at least one NS3/4A cleavage site is located at or adjacent to the C-terminus of the NS3/4A protease domain.

In most preferred embodiments, the CAR of the invention comprises more than one NS3/4A cleavage site and at least one NS3/4A cleavage site is located at or adjacent to the N-terminus of the NS3/4A protease domain and at least one cleavage site is located at or adjacent to the C-terminus of the NS3/4A protease domain.

In further embodiments, the NS3/4A cleavage site(s) are located at any position within the CAR of the invention as long as their presence does not interfere with the expression, membrane location and/or function of the CAR.

In some embodiments, the NS3/4A protease of the CAR of the instant invention comprises a mutation, including an insertion, deletion or substitution of at least one amino acid within the NS3 and/or 4A domain sequence and/or cleavage site.

Although the at least one mutation can occur at any amino acid position within the NS3 and/or NS3/4A protease domain, preferred positions are amino acids 36, 43, 54, 80, 122, and 168 (numbering starting at A in position 1 of the HCV NS3 protease domain).

In some embodiments, the NS3/4A protease domain of the CAR of the invention comprises the amino acids sequence of the wild-type HCV NS3/4A protease domain (see, SEQ ID NO: 980).

In some embodiments, the NS3/4A protease domain of the CAR of the invention comprises a T54A substitution in the HCV NS3/4A protease domain (see, SEQ ID NO: 981).

In some embodiments, the NS3/4A protease domain of the CAR of the invention comprises a single cleavage site at or adjacent to the NS4A sequence of the NS3/4A protease domain.

In some embodiments, the NS3/4A protease domain of the CAR of the invention comprises a single cleavage site at or adjacent to the C-terminus of the CD8 hinge of the CAR construct of the invention.

In some embodiments, the NS3/4A protease domain of the CAR of the invention comprises at least one amino acid substitution that renders the NS3/4A protease of the invention sensitive to inhibition by a small molecule inhibitor. In a preferred embodiment, the NS3/4A protease domain of the CAR of the invention comprises at least one mutation that renders the NS3/4A protease sensitive for inhibition by asunaprevir (see, SEQ ID NO: 982).

In some embodiments, the NS3/4A protease domain of the CAR of the invention comprises at least one amino acid substitutions that render the NS3/4A protease of the invention sensitive to inhibition by the small molecule inhibitor telaprevir (see, SEQ ID NO: 983).

In preferred embodiments, the CAR of the instant invention comprises at least one NS3/4A protease domain that is inhibited in its protease activity by a small molecule inhibitor including, but not limited to, asunaprevir, telaprevir, simeprevir, faldaprevir, danoprevir, vaniprevir, narlaprevir/r, MK-5172, ABT-450/r, ACH-1625, ACH-2684, GS-9256, GS-9451, and IDX320.

Advantageously, the CARs of the invention are cleaved by the NS3/4A protease domain at the specific cleavage site(s) in the absence of a small molecule inhibitor. In contrast the CARs of the invention comprising at least one mutation in the NS3/4A protease domain that renders the NS3/4A domain sensitive to the binding of and inhibition by a small inhibitor molecule can be inhibited by contacting the T cell transduced with such CAR construct of the instant invention with an effective amount of the small molecule inhibitor.

As used herein, “an effective amount” of a small molecule inhibitor is any amount that results in the presence of CARs on the cell membrane of a cell transduced with a CAR construct of the invention.

When used in the context of the treatment or prevention of a disease, the term “effective amount,” as used herein, refers to an amount that is capable of treating or ameliorating a disease or condition or otherwise capable of producing an intended therapeutic effect (such as preventing or reducing the level of an auto-reactive cell).

For example, a therapeutically effective amount of CAR-T cells to be administered to a subject in need thereof can range from about 10 to about 10¹⁴ cells per administration, including but not limited to, about 10² to about 10¹³′ about 10³ to about 10¹², about 10⁴ to about 10¹¹, about 10⁵ to about 10¹⁰, about 10⁶ to about 10⁹, and any number in between, e.g., 1×10², 1.1×10², 1.2×10², 1.3×10², 1.4×10², 1.5×10², 1.6×102, 1.7×10², 1.8×10², 1.9×10², and 2×210² and so on.

The term “treatment” or any grammatical variation thereof (e.g. treat, treating, and treatment etc.), as used herein, includes but is not limited to, ameliorating or alleviating a symptom of a disease or condition, reducing, suppressing, inhibiting, lessening, or affecting the progression, severity, and/or scope of a condition.

The term “prevention” or any grammatical variation thereof (e.g., prevent, preventing, and prevention etc.), as used herein, includes but is not limited to, delaying the onset of symptoms, preventing relapse to a disease, decreasing the number or frequency of relapse episodes, increasing latency between symptomatic episodes, or a combination thereof.

In some embodiments, a mixed population of cells is extracted from a patient with a disease that is to be treated with the CAR-T cells of the invention or a donor subject. Subsequently, retrovirus- or lentivirus-mediated expression of a CAR of the invention in the isolated T cells is performed, and a therapeutically effective amount of CAR-T cells ranging from 1 to 10¹⁴ CAR-T cells are transfused into the patient. The CAR-T cells of the invention are able to replicate in vivo resulting in long-term persistence that can lead to sustained disease control. For example, the transfused CAR-T cells can persist in a patient for at least one month after administration. In some embodiments, the persisting population of genetically engineered CAR-T cells persists in the human for at least four months, five months, six months, seven months, eight months, nine months, ten months, eleven months, twelve months, two years, or three years after administration.

Further provided are dual switch CAR-T cell systems comprising T cells transduced with at least one CAR construct encoding a CAR having a single chain variable fragment that binds to a first antigen and T cells transduced with at least one CAR construct encoding a CAR having a single chain variable fragment that binds to a second antigen.

In some embodiments, the CAR constructs encoding a scFv that bind a first and a scFv that bind a second antigen are combined within a single vector and transferred into the same T cell.

In other embodiments, the CAR constructs encoding a scFv that binds a first and a scFv that binds a second antigen are contained on separate vectors and are transferred into different T cells.

In a preferred embodiment, the CAR encoding a scFv that binds to a first antigen comprises a NS3/4A protease domain that contain at least one mutation that renders the NS3/4A domain sensitive to inhibition with a small molecule inhibitor.

In a further preferred embodiment, the CAR encoding a scFv that binds to a second antigen comprises a NS3/4A protease domain that contain at least one mutation that renders the NS3/4A domain sensitive to inhibition with a different small molecule inhibitor.

In more preferred embodiments, the CAR constructs encoding a scFv that bind a first and a scFv that bind a second antigen comprise NS3/4A protease domains that are sensitive to inhibition by different small molecule inhibitors.

In most preferred embodiments, a first CAR construct of the invention encodes a scFv that binds a CD19 antigen and comprises a NS3/4A protease domain that is sensitive to inhibition by the small molecule inhibitor asunaprevir and a second CAR construct encodes a scFv that binds a CD22 antigen and comprises a NS3/4A protease domain that is sensitive to inhibition by the small molecule inhibitor telaprevir.

For example, in some embodiments, lentiviral vectors are constructed that encode both a switchable anti-CD19 CAR and a switchable anti-CD22 CAR (see, e.g., expression vector plasmid pLVX) using different variants of NS3/4A protease switches, which different NS3/4A variants are inhibited by different small molecule inhibitors. Co-expression of two CARs from a single lentivirus expression plasmid is achieved, e.g., by a self-cleaving viral 2A peptide sequence. Each CAR includes, e.g., a CD28 and 4-1-BB costimulatory domain in addition to a CD3 zeta activating domain (third-generation). The lentiviruses are packaged in 293T cells by co-transfecting an expression vector with a lentiviral envelope plasmid, e.g., plasmid pPAX2 and a lentiviral packaging plasmid, e.g., pMD2.G into 293T cells and the lentiviruses made by the transfected 293T cells are harvested. The lentiviruses thus produced comprise an anti-CD19 CAR construct and an anti-CD22 CAR construct and are used to transduce T cells obtained from patients in need of CAR-T therapy or T cells obtained from donors. The CAR-T cells are subsequently administered to the patient in need of CAR-T cell therapy by infusion.

Advantageously, the CAR-T cells of the instant invention when cultured in the absence of a small molecule inhibitor, e.g., asunaprevir do not express the CAR on the cell surface as measured by antibodies against the CD19-CAR and flow cytometry.

However, in the presence of asunaprevir, a significant portion of T cells express on their cell surface the CAR of the invention as measured by flow cytometry.

Furthermore, the expression levels of CARs of the invention present on the cell surface of transduced T cells increase with exposure of said T cells to increasing concentrations of asunaprevir.

The expression levels of CARs on CAR-T cells in the presence of asunaprevir are stable over time. For example, the expression levels are stable for at least 1 hour to about 4 weeks, e.g. for 2 hours, 4 hours, 6 hours, 8 hours. 10 hours, 12 hours, 14 hours, 16 hours, 18 hours, 20 hours, 24 hours. 30 hours. 36 hours, 40 hours, 48 hours, 72 hours, 96 hours, 120 hours, 144 hours, 1 week, 2, weeks, 3 weeks, and/or 4 weeks.

Importantly, the presence of a NS3/4A protease domain in the CAR of the instant invention does not have any negative effect on the killing ability of the T cells expressing the CARs of the invention. For example, the tumoricidal or cancer cell killing activity of T cells expressing a CAR without a NS3/4A protease domain is similar to the tumoricidal activity of CAR-T cells of the invention, which CAR-T cells express a NS3/4a protease domain.

Advantageously, when the small molecule inhibitor is withdrawn, the expression levels of CARs of the invention on the cell surface of CAR-T cells are significantly reduced or CARs are absent from the surface of inhibitor-treated CAR-T cells.

For example, withdrawal of asunaprevir for 24 hours results in a significant reduction of CARs on the surface of CAR-T cells and withdrawal of asunaprevir for 48 hours results in an absence of CARs on CAR-T cells.

However, CAR-T cells of the invention do not undergo apoptotic cell death, necrotic cells death, and/or autophagy in the presence or absence of the small molecule inhibitor.

Furthermore, CAR-T cells of the invention do not alter their CD4 and/or CD8 expression levels in the presence or absence of the small molecule inhibitor.

The efficient removal of CARs from the surface of CAR-T cells enables a return of the CAR-T cells of the invention to a non-activated state. Because extensive activation of T cells, in general, and CAR-T cells specifically leads to cell death and, thus, exhaustion of the CAR-T cell population, the on/off switches of the CARs of the instant invention provide for extended viability of CAR-T cells in patients and avoid activation-induced cell death and CAR-T cell exhaustion.

Nucleic Acid Molecules, Vectors, and Expression Constructs

In some embodiments, the instant invention provides nucleic acid molecules encoding antigen-specific CARs of the invention. The present invention also provides expression constructs and vectors comprising nucleic acid molecules encoding antigen-specific CARs of the invention.

In preferred embodiments, the CAR sequences are contained in a genetic construct that allows site-directed insertion of the CAR construct into the T cell genome to allow more consistent surface level expression of the CARs.

The nucleic acid sequences useful according to the instant invention can be obtained using recombinant methods known in the art, such as, e.g., by screening libraries from cells expressing a gene of interest, by deriving a gene of interest from a vector known to include the same, or by isolating directly from cells and tissues containing the same. Alternatively, the nucleic acid sequences of interest can be produced synthetically.

The expression of natural or synthetic nucleic acids encoding CARs can be achieved by operably linking a nucleic acid encoding the CAR polypeptide or portions thereof to a promoter, and incorporating the construct into an expression vector. The vectors can be suitable for replication and integration in eukaryotes. Typical cloning vectors contain initiation sequences, promoters useful for regulation of the expression of the desired nucleic acid sequence, and transcription and translation terminators.

As used herein, the term “expression construct” refers to a combination of nucleic acid sequences that provides for transcription of an operably linked nucleic acid sequence. Expression constructs of the invention also generally include regulatory elements that are functional in the intended host cell in which the expression construct is to be expressed. Regulatory elements include promoters, transcription termination sequences, translation termination sequences, enhancers, and polyadenylation elements.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In preferred embodiments, a promoter can be positioned about the same distance from the transcription start site as it is from the transcription start site in its natural genetic environment. Some variation in this distance is permitted without substantial decrease in promoter activity. A transcription start site is typically included in the expression construct.

As used herein, the term “operably linked” refers to a juxtaposition of the components described wherein the components are in a relationship that permits them to function in their intended manner. In general, operably linked components are in contiguous relation. Sequence(s) operably-linked to a coding sequence may be capable of effecting the replication, transcription and/or translation of the coding sequence. For example, a coding sequence is operably-linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In certain embodiments, promoters useful according to the current invention include, but are not limited to, cytomegalovirus (CMV) promoter, elongation growth factor-1a (EF-1a) promoter, simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, actin promoter, myosin promoter, hemoglobin promoter, and creatine kinase promoter.

Vectors useful according to the present invention include, but are not limited to, viral vectors, including but not limited to, retroviral vectors, adenoviral vectors, adeno-associated viral vectors (AAV), and lentiviral vectors.

In preferred embodiments, lentiviral vectors are provided that comprise a nucleic acid molecule encoding a CAR specific for a cancer antigen or immune disease antigen. If the CAR constructs of the invention have a size in excess of the packaging capacity of a viral vector, alternative methods including, but not limited to, piggyBac transposon systems can be used to transfer CAR constructs into target cells.

Transduction, Isolation, and Enrichment of T Cells

In some embodiments, T cells are transduced using a variety of viral vectors, including but not limited to, retroviral vectors, adenoviral vectors, adeno-associated viral vectors, and lentiviral vectors. In some embodiments, the viral vector comprises a nucleic acid molecule encoding a CAR of the present invention.

The term “transfected” or “transformed” or “transduced,” as used herein, refers to a process by which exogenous nucleic acid is transferred or introduced into a host cell.

In some embodiments, the instant invention provides the use of lentiviral vectors for transduction of T cells. Vectors derived from lentivirus are suitable tools to achieve long-term gene transfer since they allow long-term, stable integration of a transgene and its propagation in daughter cells. Lentiviral vectors have the added advantage over vectors derived, e.g., from retroviruses such as murine leukemia viruses in that they can transduce non-proliferating cells. Lentiviral vectors also have the added advantage of low immunogenicity.

Selected genes can be inserted into a vector and packaged in a virus (such as lentivirus) particle using techniques known in the art. The recombinant virus can then be isolated and delivered to cells, e.g., T cells of a subject either in vivo or ex vivo. In some embodiments, the lentivirus vectors of the invention comprise nucleic acid molecules encoding CARs of the instant invention.

In some embodiments, in order to assess the expression of a CAR polypeptide or portions thereof, the expression vector to be introduced into a cell can also contain either a selectable marker gene or a reporter gene. Reporter genes are used for identifying potentially transfected and/or infected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic or fluorescent activity. Expression of the reporter gene is assayed at a suitable time after the DNA has been introduced into the recipient cells. Suitable reporter genes include, but are not limited to, genes encoding luciferase, beta-galactosidase, chloramphenicol acetyl transferase, secreted alkaline phosphatase, and any fluorescent protein gene such as, e.g., green fluorescent protein. Suitable expression systems are well known in the art and may be prepared using techniques known in the art or may be obtained commercially.

The term “activation” or any grammatical variation thereof (e.g., activate, activating, activation etc.), as used herein, refers to the state of a T cell that has been sufficiently stimulated to induce detectable cellular proliferation. Activation can also be associated with induced cytokine production, and detectable effector functions.

The term “activated T cell,” as used herein, refers to, among other things, T cells that are undergoing cell division.

In one embodiment, prior to expansion and genetic modification of the T cells of the invention, a source of T cells can be obtained from a subject.

The term “isolating” or any grammatical variation thereof (e.g., isolate, isolating, isolation etc.), as used herein, refers to a cell that is removed from its natural environment (such as in peripheral blood) and is separated from the combined mixture of the blood to be at least about 75% free, and most preferably about 90% free, from other cells with which it is naturally present, but which lack the cell surface markers based on which the cells were isolated.

T cells can be obtained from a number of sources, including, but not limited to, peripheral blood mononuclear cells, bone marrow, lymph node tissue, cord blood, thymus tissues, tissues from sites of infection, ascites, pleural effusions, spleen tissues, and tumors. In certain embodiments of the present invention, any number of T cell lines available in the art, may be used.

In preferred embodiments, T cells are isolated and purified from blood or bone marrow of a subject into which the CAR-T cell-enriched composition is subsequently introduced. The subject can be a cancer patient or a patient suffering from an immune-related disease in whom suppression of an immune response against an immune cell-related antigen is desired. In some embodiments, the subject is a human afflicted with cancer or an auto-immune disease.

Alternatively, the T cells may be obtained from a donor distinct from the patient. In certain embodiments, T cells may be obtained from a unit of blood collected from a subject using any number of techniques known to the skilled artisan, such as Ficoll™ separation. In preferred embodiments, cells from the circulating blood of an individual are obtained by apheresis. The apheresis product typically contains lymphocytes, including T cells, monocytes, granulocytes, B cells, other nucleated white blood cells, red blood cells, and platelets.

In some embodiments, the cells collected by apheresis or Ficoll™ separation may be washed to remove the plasma fraction and to place the cells in an appropriate buffer or media for subsequent processing steps. In further embodiments, the cells are washed with phosphate buffered saline (PBS). In an alternative embodiment, the wash solution lacks calcium and may lack magnesium or may lack many, if not all, divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, e.g., Ca²⁺-free, Mg²⁺-free PBS, PlasmaLyte A, autoMACS Running Buffer or other saline solution with or without buffer. For example, autoMACS Running Buffer contains bovine serum albumin (BSA) and 0.09% azide. Alternatively, the undesirable components of the apheresis sample may be removed and the cells directly resuspended in culture media.

The harvested lymphocytes may be separated using the cell separation techniques based on T cell-specific cell markers such as those described herein. By selecting for phenotypic characteristics among the cells obtained from the blood sample, antibodies that recognize species-specific varieties of markers are used to enrich for and select T cells. For example, antibodies that recognize the species-specific varieties of CD4, CD25, and CD45RA, and other markers known in the art are used to enrich for or isolate T cells from a human (e.g., antibody to a human CD4 for human T cells).

In particular embodiments, the T cells are enriched from a population of cells using reagents that bind cell surface markers specific for T cells and separating these cells using cell sorting assays such as fluorescence-activated cell sorting (FACS), solid-phase magnetic beads, etc., as known in the art. In some embodiments, combinations of methods to sort the cells can be used, e.g., magnetic selection, followed by FACS. To enhance enrichment, positive selection, e.g., using surface markers that are expressed on T cells is combined with negative selection, e.g., using surface markers that are not expressed on T cells. It is intended that isolation/enrichment of T cells using cell surface markers can be performed in any order. Therefore, a positive selection step may immediately precede a negative selection step, or vice versa. It is also contemplated that isolation/enrichment be performed by grouping the positive selection and negative selection steps. Therefore, isolation/enrichment is done by first performing the positive selection steps of the method, followed by performing the negative selection steps of the method, or vice versa.

It is also possible to enrich for CD4⁺ and CD8⁺ cells by depleting non-CD4⁺ and non-CD8⁺ immune cells. Such cell types include, but are not limited to, B cells, natural killer cells, dendritic cells, monocytes, granulocytes and erythroid cells. Antibodies to surface markers that can used to deplete non-CD4⁺ and non-CD8⁺ cells are known in the art and include, but are not limited to, CD14, CD16, CD19, CD36, CD56, CD123, and glycophorin A.

For example, in one embodiment of the invention, a population of cells is first contacted with a first reagent or group of reagents that bind one or more of CD14, CD16, CD19, CD36, CD56, CD123, and glycophorin A, followed by reagents that respectively bind CD4 and/or CD8 and/or CD45RA.

Whether prior to or after genetic modification of the T cells to express a desirable CAR, the T cells can be activated and expanded generally using methods as known in the art.

Generally, the CAR-T cells of the invention are expanded by contact with a surface having attached thereto an agent that stimulates a CD3 TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore. For costimulation of an accessory molecule on the surface of T cells, a ligand that binds the accessory molecule is used. For example, a population of T cells can be contacted with anti-CD3 and anti-CD28 antibodies under conditions appropriate for stimulating proliferation of T cells.

In certain embodiments, the primary stimulatory signal and the costimulatory signal for the T cells may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent may be in solution. In one embodiment, the agent providing the costimulatory signal is bound to a cell surface and the agent providing the primary activation signal is in solution or coupled to a surface. In certain embodiments, both agents can be in solution. In other embodiments, the agents may be in soluble form, and then cross-linked to a surface, such as a cell expressing Fc receptors or an antibody or other binding agent which will bind to the agents.

In some embodiments, two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one embodiment, a 1:1 ratio of each antibody bound to the beads for T cell expansion and T cell growth is used. In certain aspects of the present invention, a ratio of anti CD3:CD28 antibodies bound to the beads is used such that an increase in T cell expansion is observed as compared to the expansion observed using a ratio of 1:1.

In some embodiments, the ratio of CD3:CD28 antibody bound to the beads ranges from 100:1 to 1:100 and all integer values there between. In some embodiments of the invention, more anti-CD28 antibodies are bound to the particles than anti-CD3 antibody such that the ratio of CD3:CD28 is less than one.

In certain embodiments, the ratio of anti-CD28 antibody to anti-CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells can range from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. In another embodiment, particles are added on a daily or every other day basis. One of skill in the art will appreciate that a variety of other ratios may be suitable for use in the present invention. In particular, ratios can vary depending on particle size.

In further embodiments, the T cells are combined with agent-coated beads and beads and cells are subsequently separated, and then the separated cells are cultured. In an alternative embodiment, prior to culture, the agent-coated beads and cells are not separated but are cultured together. In a further embodiment, beads and cells are first concentrated by application of a force, such as a magnetic force, resulting in increased ligation of cell surface markers, thereby inducing cell stimulation.

In some embodiments, the mixture of agent-coated beads and cells may be cultured for several hours (about 3 hours) to about 21 days or any hourly integer value in between. In preferred embodiments beads and T cells are cultured together for about eight days. In other preferred embodiments, beads and T cells are cultured together for 2-3 days. Several cycles of stimulation may also be desired such that culture time of T cells can be 60 days or more.

Conditions appropriate for T cell culture include an appropriate media (e.g., Minimal Essential Media or RPM1 Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, a-MEM, F-12, X-Vivo 1 5, X-Vivo 20, and Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, e.g., an appropriate temperature (e.g., 37° C.) and atmosphere (e.g., air plus 5% CO₂).

Antibody and Antibody Domain

An antibody that is contemplated for use in the present invention can be in any of a variety of forms, including a whole immunoglobulin, an antibody fragment such as Fv, Fab, and similar fragments, as well as a single chain antibody that includes the variable domain complementarity determining regions (CDR), and similar forms, all of which fall under the broad term “antibody,” as used herein.

The term “human antibody,” as used herein, is intended to include antibodies having variable and constant regions identical to, essentially identical to, or derived from human germ-line immunoglobulin sequences. Such human antibodies can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “antibody fragment” refers to a portion of a full-length antibody, generally the antigen binding or variable region. Examples of antibody fragments include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment of an antibody yields an F(ab′)₂ fragment that has two antigen binding fragments, which are capable of cross-linking antigen, and a residual other fragment (which is termed pFc′). Additional fragments can include diabodies, linear antibodies, single-chain antibody molecules, and multi-specific antibodies formed from antibody fragments. As used herein, “antigen binding fragment” with respect to antibodies, refers to, for example, Fv, F(ab) and F(ab′)₂ fragments. Of particular importance for binding are the first 110 to 130 amino acids at the N-terminus of the amino acid sequences exemplified herein. Thus, high identity in the N-terminus 110, 115, 120, 125, or 130 amino acids constituting the variable region is preferred. Variant sequences preferably have more than 75%, 90%, or even 95% identity in this region.

Fab is the fragment of an antibody that contains a monovalent antigen-binding fragment of an antibody molecule. A Fab fragment can be produced by digestion of whole antibody with the enzyme papain to yield an intact light chain and a portion of one heavy chain.

Fab′ is the fragment of an antibody molecule that can be obtained by treating whole antibody with pepsin, followed by reduction, to yield an intact light chain and a portion of the heavy chain. Two Fab′ fragments are obtained per antibody molecule. Fab′ fragments differ from Fab fragments by the addition of a few residues at the carboxyl terminus of the heavy chain CH1 domain including one or more cysteines from the antibody hinge region.

(Fab′)₂ is the fragment of an antibody that can be obtained by treating whole antibody with the enzyme pepsin without subsequent reduction. F(ab′)₂ is a dimer of two Fab′ fragments held together by two disulfide bonds.

Fv is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody.

However, even a single variable domain, or half of an Fv, comprising only three CDRs specific for an antigen has the ability to recognize and bind antigen, although potentially at a lower affinity than the entire binding site.

A single chain antibody is defined as a genetically engineered molecule containing the variable region of the light chain (VL), the variable region of the heavy chain (VH), linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding.

“Specific binding” or “specificity” refers to the ability of an antibody or other agent to detectably bind an epitope presented on an antigen, while having relatively little detectable reactivity with other proteins or structures. Specificity can be relatively determined by binding or competitive binding assays, using, e.g., Biacore instruments. Specificity can be exhibited by, e.g., an about 10:1, about 20:1, about 50:1, about 100:1, 10.000:1 or greater ratio of affinity/avidity in binding to the specific antigen versus nonspecific binding to other irrelevant molecules.

Strategies for antibody optimization are sometimes carried out using random mutagenesis. In these cases, positions are chosen randomly, or amino acid changes are made using simplistic rules such as a sequential change of all residues to alanine, Alanine scanning mutagenesis can also be used, for example, to map the antigen binding residues of an antibody. Sequence-based methods of affinity maturation may also be used to increase the binding affinities of antibodies.

Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

Formulations and Administration

In some embodiments, T cells expressing a CAR of the instant invention are administered to a subject. The term “subject,” as used herein, describes an organism, including a mammal such as a primate. Mammalian species that can benefit from the disclosed methods of treatment include, but are not limited to, apes, chimpanzees, orangutans, humans monkeys; and domesticated and laboratory animals such as dogs, cats, horses, cattle, pigs, sheep, goats, chicken, mice, rats, guinea pigs, and hamsters. In one specific embodiment, the subject is a human.

In some embodiments, effective amounts of CAR-expressing T cells engineered according to the instant invention are administered to a patient in need of treatment of a disease can be performed using methods well known in the art such as adoptive cell transfer.

In one embodiment, a mixed population of cells is extracted from a patient with a disease that is to be treated with the CAR-T cells of the invention or a donor subject. Subsequently, retrovirus- or lentivirus-mediated expression of a CAR of the invention in the isolated T cells is performed, and a therapeutically effective amount of CAR-T cells ranging from 1 to 10¹⁴ are transfused into the patient. The CAR-T cells of the invention are able to replicate in vivo resulting in long-term persistence that can lead to sustained disease control. For example, the transfused CAR-T cells can persist in a patient for at least one month after administration. In some embodiments, the persisting population of genetically engineered CAR-T cells persists in the human for at least four months, five months, six months, seven months, eight months, nine months, ten months, eleven months, twelve months, two years, or three years after administration.

The present invention also provides a pharmaceutical composition comprising one or more CAR-T cells of the present invention. In certain embodiments, the composition comprises at least 1, 10, 10², 10³, 10⁴, 10⁵, 10⁶, 10⁷, 10⁸, 10⁹, 10¹⁰, 10¹¹, 10¹², 10¹³, or 10¹⁴ CAR-T cells, or any amounts higher than 10¹⁴ of CAR-T cells.

In some embodiments, the persisting population of genetically engineered CAR-T cells comprises at least one cell selected from T cells that had been administered to a human, progenies of T cells that had been administered to a human, and a combination thereof.

In some embodiments, the CAR-T cells of the invention can undergo robust in vivo T cell expansion. In preferred embodiment, the CAR-T cells of the invention evolve into specific memory T cells that can be reactivated to inhibit any additional tumor cells or immune cells of the disease originally treated with the CAR-T cells.

In some embodiments, the CAR-T cells of the invention infused into a patient can eliminate cancer cells or immune cells in vivo in patients with cancer or an immune-related disease.

In some embodiments, the CAR-T cells of the invention infused into a patient can reduce the tumor burden or immunological response in vivo in patients suffering from cancer or an immune-related disease.

In other embodiments, the CAR-T cells of the invention infused into a patient can prevent the reoccurrence of cancer cells or immune cells that cause an immune disease.

In yet other embodiments, the CAR-T cells of the invention infused into a patient can prevent the occurrence of a cancer or an immune-related disease in vivo in subjects having a high risk of developing a cancer or an immune-related disease, wherein the risk can be based on prior history of the patient, family history, accumulation of a variety of risk factors or the presence of a precancerous lesion or an immune system alteration that is considered a precursor of an immune-related disease.

The CAR-T cells of the present invention may be administrated alone, but preferably, as a pharmaceutical composition, which usually comprises a suitable pharmaceutical excipient, diluent or carrier selected according to the intended administration route.

The CAR-T cells may be administrated to the patient in need thereof by any suitable route. The manner of application may vary widely, e.g., in certain embodiments, the CAR-T cell containing compositions of the invention will be administered intravenously, subcutaneously, peritoneally, intramuscularly and vaginally or at the site of a tumor or an inflammation directly. Regardless, any of the conventional methods for administration of a vaccine are applicable. The dosage of the vaccine will depend on the route of administration and will vary according to the size of the host.

In one embodiment, the composition of the present invention is administered via injection.

Some further suitable administration routes include, but are not limited to, oral, rectal, nasal, topical (including buccal and sublingual), subcutaneous, vaginal or parenteral (including subcutaneous, intramuscular, intravenous, intracutaneous, intrathecal and extradural) administration.

For intravenous injection and injection at the focal site, active ingredients are present in the form of a parenterally-acceptable aqueous solution, which is free of pyrogen and has appropriate pH value, isotonicity and stability.

A suitable solution may be formulated by the person skilled in the art using, e.g., isotonic excipients such as sodium chloride injection, Ringer's injection, Ringer's lactate injection. As required, preservative, stabilizer, buffering agent, antioxidant and/or some other additives may be added. The pharmaceutical composition orally administrated may be in a form of tablet, capsule, powder or oral liquid etc. Solid carrier, such as gelatin or adjuvant, may be comprised in a tablet. Liquid pharmaceutical composition usually comprises liquid carrier, such as water, petroleum, animal or vegetable oil, mineral oil or synthetic oil. Also included may be normal saline solution, glucose or other sugar solutions or glycols such as ethylene glycol, propylene glycol or polyethylene glycol.

### Materials and Methods

PBMCs are isolated from blood drawn from donors or from buffy coats obtained from a Blood Bank. Approximately 150 ml donor blood are drawn into heparinized blood collection tubes (VWR, West Chester, Pa.), then diluted 1:1 in PBS. Buffy coats are diluted to a final volume of 200 ml in PBS. Approximately 4 volumes of diluted sample are layered over 3 volumes of Ficoll-Paque PLUS (GE Healthcare Bio-Sciences, Pittsburgh, Pa.), then centrifuged at 800 rcf at room temperature for 30 minutes without brake. Cells at the interface are harvested, washed and resuspended in MACS Buffer for further separation.

For in vivo animal studies and in vitro experiments, cell lines, including, but not limited to, Raji, K562, Nalm-6, 293T are purchased from ATCC and maintained according to ATCC instructions. Cells lines are passaged every 2-3 days, and cells at logarithmic growth phase are used for experiments.

PBMC are isolated through density gradient centrifugation. After sorting T cells by CD3 magnetic microbeads from PBMC, these cells are cultured in complete T-cell medium with CD3/CD28 stimulant. T cells are transduced with concentrated lentivirus within twenty-four hours after isolation. After that, cell density will be adjusted with the culture media every day.

Cells are separated by MACS microbeads (Miltenyi Biotec, Auburn, Calif.), following the manufacturer's protocol, using LS columns (Miltenyi Biotec). In order to elute bound cells, the columns are removed from the magnetic field. 3 ml of MACS Buffer is added to the column, and the eluted cells are collected.

For flow cytometry, cells are stained using standard procedures. Briefly, cells are suspended at a concentration of 1×10⁷ cells/ml in PBS+3% FBS for analysis or in Sort Buffer (PBS, 25 mM HEPES, 1 mM EDTA, 0.1% BSA) for sorting. The amount of antibody added is in accordance to manufacturer's suggested volume, or is determined by titration. Cells are analyzed by flow cytometry using a CytoFLEX (Beckman Coulter Life Sciences, Indianapolis, Ind.) and operated under standard procedures. To enrich subpopulations of CD4+ and/or CD8+ cells, magnetically separated cells are stained with anti-CD4 and/or anti-CD8 antibodies and sorted on a FACScalibur Cell Sorter.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### Example 1—Vector Construction and Lentivirus Packaging

A lentiviral vector was constructed that encodes both a switchable anti-CD19 CAR and a switchable anti-CD22 CAR by applying different variants of NS3 protease switch. Co-expression of two targets was achieved by a self-cleaving viral 2A peptide sequence. Each CAR includes a CD28 and 4-1-BB costimulatory domain in addition to a CD3 zeta activating domain (Third-generation). For lentivirus packaging, 293T cells were co-transfected with the expression vector, psPAX2 and pMD2.G. The table below shows the CAR designations and respective CAR structures of CARs of the instant invention.

| | CAR Structure | | | | | | | |
|---|---|---|---|---|---|---|---|---|
| | signal | | | | | | | |
| CAR | peptide + | Cleavage | | | | Cleavage | | Intracellular |
| Designation | scFv | site | NS4A | NS3 | Hinge | site | TM | domain |
| NS3 T54A | + | + | + | + | + | + | + | + |
| NS3 WT | + | + | + | + | + | + | + | + |
| NS3 AI | + | + | + | + | + | + | + | + |
| NS3 TI | + | + | + | + | + | + | + | + |
| NS3 1CS | + | + | + | + | + | − | + | + |
| NS3 2CS | + | − | + | + | + | + | + | + |
| NS3 NCS | + | − | + | + | + | − | + | + |

### Example 2—In Vitro Functional Assays

After T cell transduction, different drugs were provided, CD19-, CD22-CAR were detected by CD19, CD22 protein through flow cytometry. Further, the biological characteristics of the dual-target switchable CAR-T cells were determined. For example, CD25 and CD69 antibodies were used to perform activation assays, CFSE for proliferation assays, CD4, CD8, CCR7 and CD45RA antibodies for determining T cell subsets, and Annexin-V for apoptosis assays. Further, cytotoxicity assays (Calcein-AM release), CD107a assay, and cytokine production detection were performed. These assays are of great importance in testing potency and defining how the switch system works.

### Example 3—Animal Experiments

Nalm-6/Raji cell lines were genetically edited to generate CD19 negative and/or CD22 negative cells by shRNA. Mutant versions of Luciferase were constructed into these cells, so that the cells that express different antigens react only with a unique luciferin [14]. This way, the activity of the dual-target switch can be determined by measuring luminescence intensities of different target cells.

Modified Nalm-6/Raji cell lines were intravenously (i.v.) injected into NSG mice (NOD.Cg-PrkdcscidIl2rgtm1Wj1/SzJ). Bioluminescence imaging was performed weekly after intraperitoneal injection of modified luciferins. Tumor burden was measured by flow cytometry of peripheral blood, bone marrow, and spleen.

### Example 4—Tumoricidal Assay

The positive target cells (Raji) and negative target cells (K562) were labeled with calcein-AM (Biolegend) and then cocultured with effector cells (CART cells with ASV) in 96-well plates at different ratios. The medium used in the cocultures was PBS+5% FBS. Wells with co-cultured target cells and PBS+5% FBS were used as spontaneous release wells, and wells with cocultured target cells and lysis solution were used as maximum release wells. The cultures were centrifuged after 3 h of incubation, and the supernatants were transferred to another 96-well plate. The fluorescence value of each well (F) was measured with a microplate reader, and the tumor-killing efficiency was calculated according to the following formula: lysis %=(F_{experimental well}−F_{spontaneous release})/(F_{maximum release}−F_{spontaneous release})×100%.

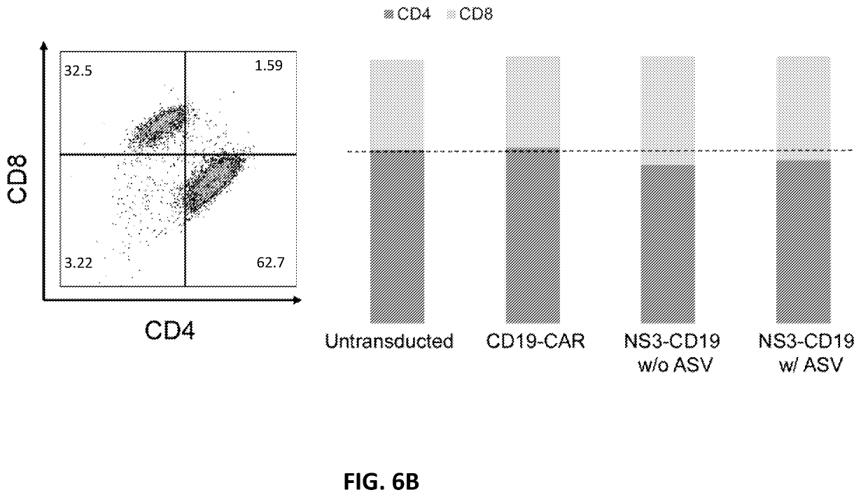

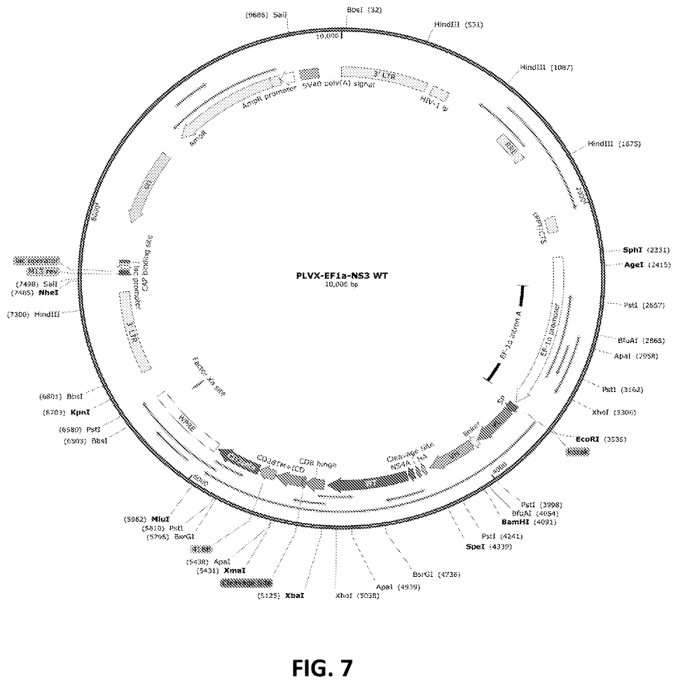

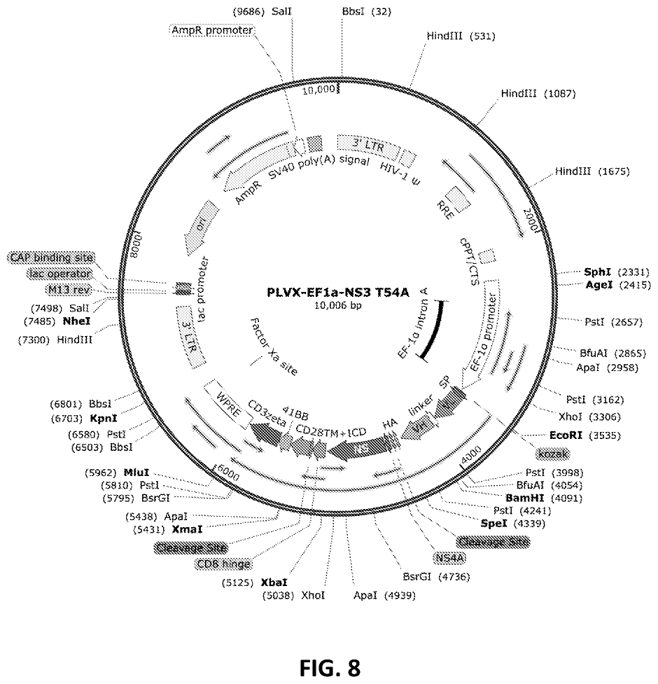

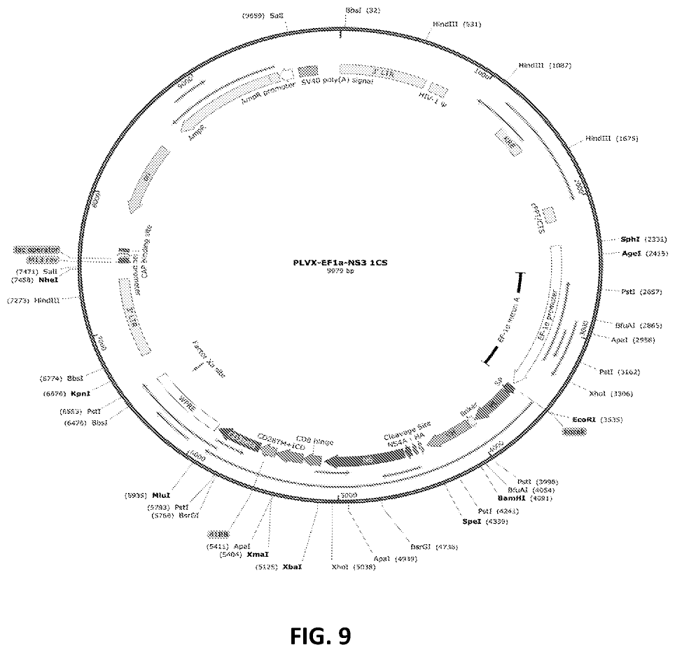

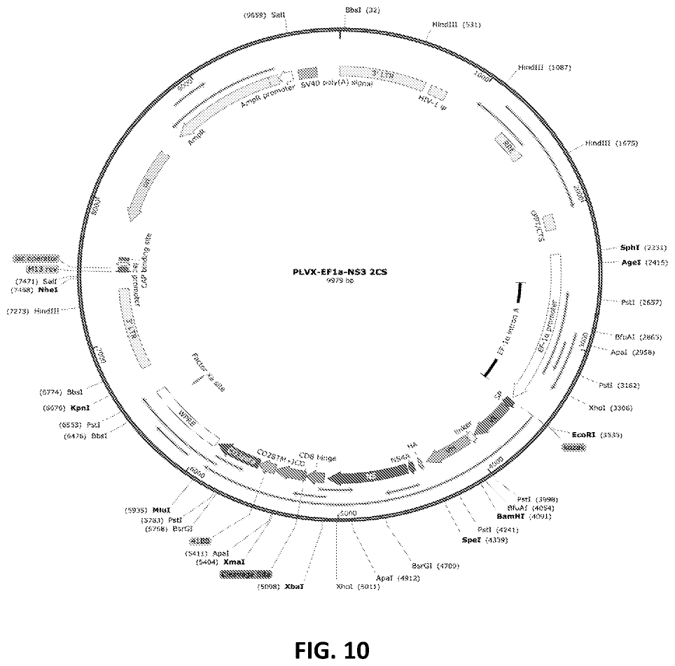

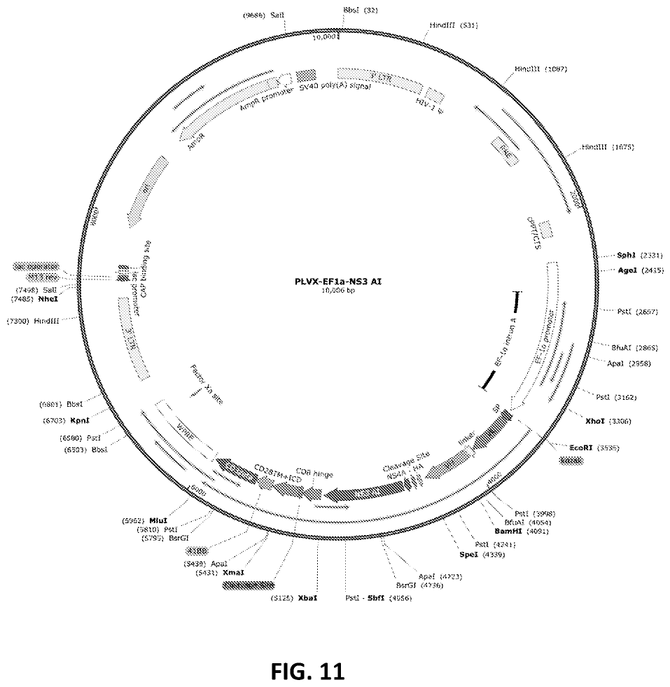

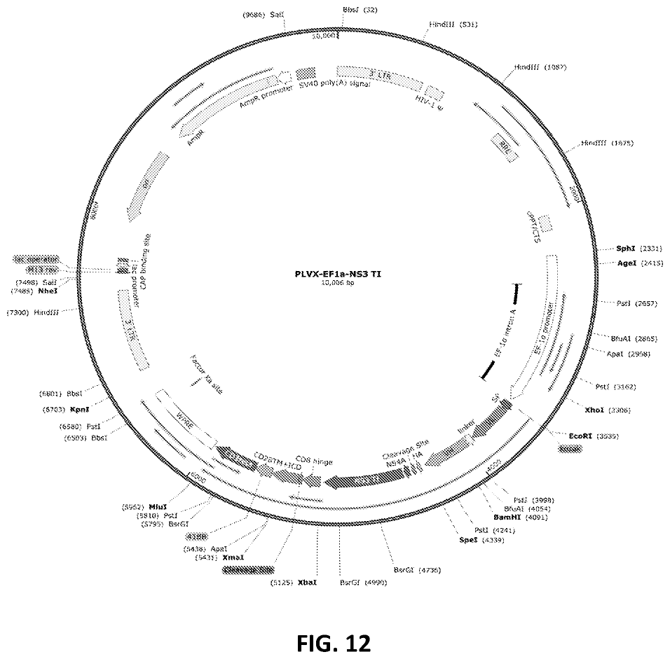

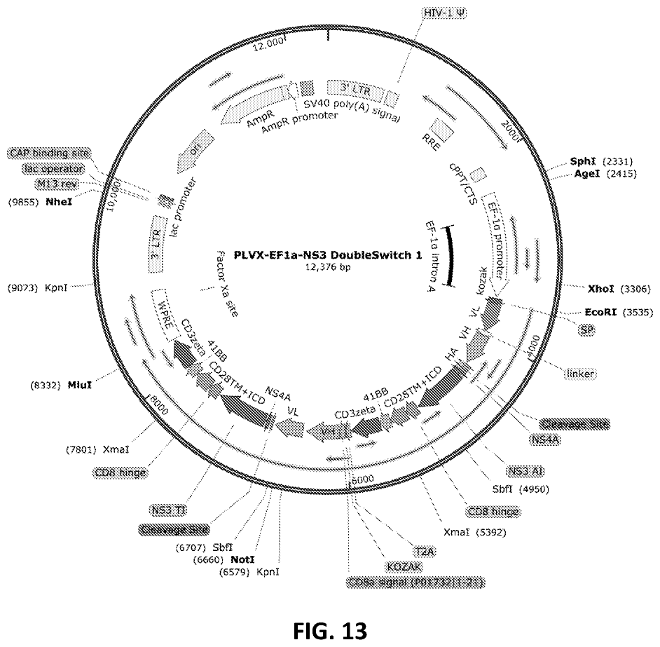

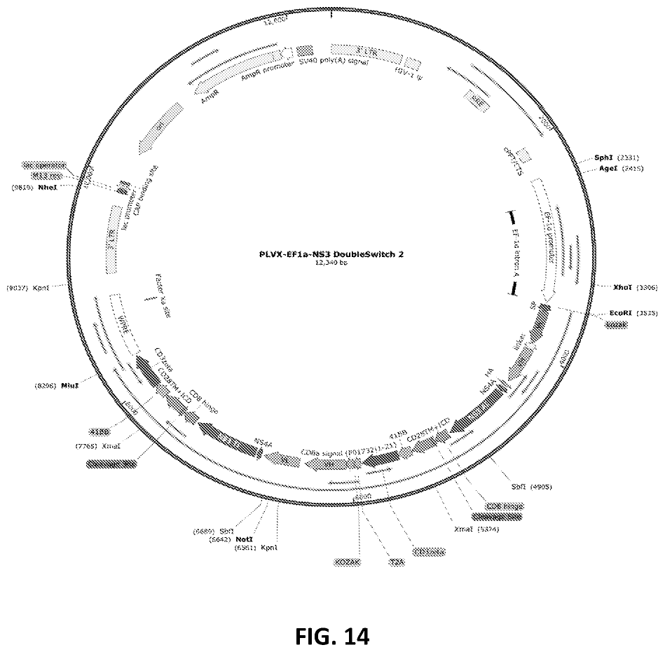

Table 1 shows the results of the tumoricidal in vitro assay. The results are also shown in FIGS. 6A-6B which show cell death and CD4/CD8 profiles of CAR-transduced cells in the presence and absence of asunaprevir.

TABLE 1

| E:T | 5:1 | 2:1 | 0.5:1 |
|---|---|---|---|
| Blank | 21% | 12% | 6% |
| CD19-only | 61% | 33% | 10% |
| NS3 T54A-CD19 | 60% | 32% | 10% |
| NS3 1CS-CD19 | 56% | 44% | 14% |

TABLE 2

| | | | Heavy | Light |
|---|---|---|---|---|
| | | | Sequence | Sequence |
| | | | (if | (if |
| Therapeutic | Heavy Sequence | Light Sequence | bispec) | bispec) |
| Abagovomab | QVKLQESGAELARPGASV | DIELTQSPASLSASVGETV | na | na |
| (Format: Whole | KLSCKASGYTFTNYWMQ | TITCQASENIYSYLAWHQ | | |
| mAb) | WVKQRPGQGLDWIGAIY | QKQGKSPQLLVYNAKTL | | |
| | PGDGNTRYTHKFKGKATL | AGGVSSRFSGSGSGTHFS | | |
| | TADKSSSTAYMQLSSLASE | LKIKSLQPEDFGIYYCQHH | | |
| | DSGVYYCARGEGNYAWF | YGILPTFGGGTKLEIK | | |
| | AYWGQGTTVTVSS (SEQ | (SEQ ID NO: 22) | | |
| | ID NO: 21) | | | |
| Abelacimab | QVQLLESGGGLVQPGGSL | QSVLTQPPSASGTPGQR | na | na |
| (Format: Whole | RLSCAASGFTFSTAAMSW | VTISCSGSSSNIGSNDVS | | |
| mAb) | VRQAPGKGLEWVSGISGS | WYQQLPGTAPKLLIYKNY | | |
| | GSSTYYADSVKGRFTISRD | NRPSGVPDRFSGSKSGTS | | |
| | NSKNTLYLQMNSLRATAV | ASLAISGLQSEDEADYYA | | |
| | YYCARELSYLYSGYYFDYW | WDQRQFDVVFGGGTKL | | |
| | GQGTLVTVSS (SEQ ID | TVL (SEQ ID NO: 24) | | |
| | NO: 23) | | | |
| Abituzumab | QVQLQQSGGELAKPGASV | DIQMTQSPSSLSASVGD | na | na |
| (Format: Whole | KVSCKASGYTFSSFWMH | RVTITCRASQDISNYLAW | | |
| mAb) | WVRQAPGQGLEWIGYIN | YQQKPGKAPKLLIYYTSKI | | |
| | PRSGYTEYNEIFRDKATMT | HSGVPSRFSGSGSGTDYT | | |
| | TDTSTSTAYMELSSLRSED | FTISSLQPEDIATYYCQQG | | |
| | TAVYYCASFLGRGAMDY | NTFPYTFGQGTKVEIK | | |
| | WGQGTTVTVSS (SEQ ID | (SEQ ID NO: 26) | | |
| | NO: 25) | | | |
| Abrezekimab | QVTLKESGPVLVKPTETLTL | DIQMTQSPSSLSASVGD | na | na |
| (Format: Fab) | TCTVSGFSLTNYHVQWIR | RVTITCLASEDISNYLAWY | | |
| | QPPGKALEWLGVMWSD | QQKPGKAPKLLIYHTSRL | | |
| | GDTSFNSVLKSRLTISRDTS | QDGVPSRFSGSGSGTDF | | |
| | KSQVVLTMTNMDPVATY | TLTISSLQPEDFATYYCGY | | |
| | YCARDGTIAAMDYFDYW | RFPLTFGGGTKVEIK | | |
| | GQGTLVTVSS (SEQ ID | (SEQ ID NO: 28) | | |
| | NO: 27) | | | |
| Abrilumab | QVQLVQSGAEVKKPGASV | DIQMTQSPSSVSASVGD | na | na |
| (Format: Whole | KVSCKVSGYTLSDLSIHWV | RVTITCRASQGISSWLAW | | |
| mAb) | RQAPGKGLEWMGGFDP | YQQKPGKAPKLLIYGASN | | |
| | QDGETIYAQKFQGRVTMT | LESGVPSRFSGSGSGTDF | | |
| | EDTSTDTAYMELSSLKSED | TLTISSLQPEDFANYYCQ | | |
| | TAVYYCATGSSSSWFDPW | QANSFPWTFGQGTKVEI | | |
| | GQGTLVTVSS (SEQ ID | K (SEQ ID NO: 30) | | |
| | NO: 29) | | | |
| Actoxumab | QVQLVESGGGVVQPGRSL | DIQMTQSPSSVSASVGD | na | na |
| (Format: Whole | RLSCAASGFSFSNYGMH | RVTITCRASQGISSWLAW | | |
| mAb) | WVRQAPGKGLEWVALIW | YQHKPGKAPKLLIYAASSL | | |
| | YDGSNEDYTDSVKGRFTIS | QSGVPSRFSGSGSGTDFT | | |
| | RDNSKNTLYLQMNSLRAE | LTISSLQPEDFATYYCQQ | | |
| | DTAVYYCARWGMVRGVI | ANSFPWTFGQGTKVEIK | | |
| | DVFDIWGQGTVVTVSS | (SEQ ID NO: 32) | | |
| | (SEQ ID NO: 31) | | | |
| Adalimumab | EVQLVESGGGLVQPGRSL | DIQMTQSPSSLSASVGD | na | na |
| (Format: Whole | RLSCAASGFTFDDYAMH | RVTITCRASQGIRNYLAW | | |
| mAb) | WVRQAPGKGLEWVSAIT | YQQKPGKAPKLLIYAAST | | |
| | WNSGHIDYADSVEGRFTIS | LQSGVPSRFSGSGSGTDF | | |
| | RDNAKNSLYLQMNSLRAE | TLTISSLQPEDVATYYCQR | | |
| | DTAVYYCAKVSYLSTASSL | YNRAPYTFGQGTKVEIK | | |
| | DYWGQGTLVTVSS (SEQ | (SEQ ID NO: 34) | | |
| | ID NO: 33) | | | |
| Aducanumab | QVQLVESGGGVVQPGRSL | DIQMTQSPSSLSASVGD | na | na |
| (Format: Whole | RLSCAASGFAFSSYGMHW | RVTITCRASQSISSYLNWY | | |
| mAb) | VRQAPGKGLEWVAVIWF | QQKPGKAPKLLIYAASSL | | |
| | DGTKKYYTDSVKGRFTISR | QSGVPSRFSGSGSGTDFT | | |
| | DNSKNTLYLQMNTLRAED | LTISSLQPEDFATYYCQQS | | |
| | TAVYYCARDRGIGARRGP | YSTPLTFGGGTKVEIK | | |
| | YYMDVWGKGTTVTVSS | (SEQ ID NO: 36) | | |
| | (SEQ ID NO: 35) | | | |
| Afasevikumab | EVQLVESGGGLVQPGRSL | EIVLTQSPATLSLSPGERA | na | na |
| (Format: Whole | RLSCAASGFTFDDYAMH | TLSCRASQSVRSYLAWYQ | | |
| mAb) | WVRQAPGKGLEWVSGIN | QKPGQAPRLLIYDASNRA | | |
| | WSSGGIGYADSVKGRFTIS | TGIPARFSGSGSGTDFTLT | | |
| | RDNAKNSLYLQMNSLRAE | ISSLEPEDFAVYYCQQRS | | |
| | DTALYYCARDIGGFGEFY | NWPPATFGGGTKVEIK | | |
| | WNFGLWGRGTLVTVSS | (SEQ ID NO: 38) | | |
| | (SEQ ID NO: 37) | | | |
| Alacizumab | EVQLVESGGGLVQPGGSL | DIQMTQSPSSLSASVGD | na | na |
| (Format: di-Fab) | RLSCAASGFTFSSYGMSW | RVTITCRASQDIAGSLNW | | |
| | VRQAPGKGLEWVATITSG | LQQKPGKAIKRLIYATSSL | | |
| | GSYTYYVDSVKGRFTISRD | DSGVPKRFSGSRSGSDYT | | |
| | NAKNTLYLQMNSLRAEDT | LTISSLQPEDFATYYCLQY | | |
| | AVYYCVRIGEDALDYWGQ | GSFPPTFGQGTKVEIK | | |
| | GTLVTVSS (SEQ ID NO: | (SEQ ID NO: 40) | | |
| | 39) | | | |
| Alemtuzumab | QVQLQESGPGLVRPSQTL | DIQMTQSPSSLSASVGD | na | na |
| (Format: Whole | SLTCTVSGFTFTDFYMNW | RVTITCKASQNIDKYLNW | | |
| mAb) | VRQPPGRGLEWIGFIRDK | YQQKPGKAPKLLIYNTNN | | |
| | AKGYTTEYNPSVKGRVTM | LQTGVPSRFSGSGSGTDF | | |
| | LVDTSKNQFSLRLSSVTAA | TFTISSLQPEDIATYYCLQ | | |
| | DTAVYYCAREGHTAAPFD | HISRPRTFGQGTKVEIK | | |
| | YWGQGSLVTVSS (SEQ ID | (SEQ ID NO: 42) | | |
| | NO: 41) | | | |
| Alirocumab | EVQLVESGGGLVQPGGSL | DIVMTQSPDSLAVSLGER | na | na |
| (Format: Whole | RLSCAASGFTFNNYAMN | ATINCKSSQSVLYRSNNR | | |
| mAb) | WVRQAPGKGLDWVSTIS | NFLGWYQQKPGQPPNL | | |
| | GSGGTTNYADSVKGRFIIS | LIYWASTRESGVPDRFSG | | |
| | RDSSKHTLYLQMNSLRAE | SGSGTDFTLTISSLQAEDV | | |
| | DTAVYYCAKDSNWGNFD | AVYYCQQYYTTPYTFGQ | | |
| | LWGRGTLVTVSS (SEQ ID | GTKLEIK (SEQ ID NO: | | |
| | NO: 43) | 44) | | |
| Amatuximab | QVQLQQSGPELEKPGASV | DIELTQSPAIMSASPGEK | na | na |
| (Format: Whole | KISCKASGYSFTGYTMNW | VTMTCSASSSVSYMHWY | | |
| mAb) | VKQSHGKSLEWIGLITPYN | QQKSGTSPKRWIYDTSKL | | |
| | GASSYNQKFRGKATLTVD | ASGVPGRFSGSGSGNSYS | | |
| | KSSSTAYMDLLSLTSEDSA | LTISSVEAEDDATYYCQQ | | |
| | VYFCARGGYDGRGFDYW | WSKHPLTFGSGTKVEIK | | |
| | GSGTPVTVSS (SEQ ID | (SEQ ID NO: 46) | | |
| | NO: 45) | | | |
| Andecaliximab | QVQLQESGPGLVKPSETLS | DIQMTQSPSSLSASVGD | na | na |
| (Format: Whole | LTCTVSGFSLLSYGVHWVR | RVTITCKASQDVRNTVA | | |
| mAb) | QPPGKGLEWLGVIWTGG | WYQQKPGKAPKLLIYSSS | | |
| | TTNYNSALMSRFTISKDDS | YRNTGVPDRFSGSGSGT | | |
| | KNTVYLKMNSLKTEDTAIY | DFTLTISSLQAEDVAVYYC | | |
| | YCARYYYGMDYWGQGTL | QQHYITPYTFGGGTKVEI | | |
| | VTVSS (SEQ ID NO: 47) | K (SEQ ID NO: 48) | | |
| Anetumab | QVELVQSGAEVKKPGESL | DIALTQPASVSGSPGQSIT | na | na |
| (Format: Whole | KISCKGSGYSFTSYWIGWV | ISCTGTSSDIGGYNSVSW | | |
| mAb ADC) | RQAPGKGLEWMGIIDPG | YQQHPGKAPKLMIYGVN | | |
| | DSRTRYSPSFQGQVTISAD | NRPSGVSNRFSGSKSGN | | |
| | KSISTAYLQWSSLKASDTA | TASLTISGLQAEDEADYY | | |
| | MYYCARGQLYGGTYMDG | CSSYDIESATPVFGGGTKL | | |
| | WGQGTLVTVSS (SEQ ID | TVL (SEQ ID NO: 50) | | |
| | NO: 49) | | | |
| Anifrolumab | EVQLVQSGAEVKKPGESL | EIVLTQSPGTLSLSPGERA | na | na |
| (Format: Whole | KISCKGSGYIFTNYWIAWV | TLSCRASQSVSSSFFAWY | | |
| mAb) | RQMPGKGLESMGIIYPGD | QQKPGQAPRLLIYGASSR | | |
| | SDIRYSPSFQGQVTISADKS | ATGIPDRLSGSGSGTDFT | | |
| | ITTAYLQWSSLKASDTAM | LTITRLEPEDFAVYYCQQY | | |
| | YYCARHDIEGFDYWGRGT | DSSAITFGQGTRLEIK | | |
| | LVTVSS (SEQ ID NO: 51) | (SEQ ID NO: 52) | | |
| Anrukinzumab | EVQLVESGGGLVQPGGSL | DIQMTQSPSSLSASVGD | na | na |
| (Format: Whole | RLSCAASGFTFISYAMSWV | RVTITCKASESVDNYGKS | | |
| mAb) | RQAPGKGLEWVASISSGG | LMHWYQQKPGKAPKLLI | | |
| | NTYYPDSVKGRFTISRDNA | YRASNLESGVPSRFSGSG | | |
| | KNSLYLQMNSLRAEDTAV | SGTDFTLTISSLQPEDFAT | | |
| | YYCARLDGYYFGFAYWGQ | YYCQQSNEDPWTFGGG | | |
| | GTLVTVSS (SEQ ID NO: | TKVEIK | | |
| | 53) | (SEQ ID NO: 54) | | |
| Apamistamab | EVKLLESGGGLVQPGGSLK | DIALTQSPASLAVSLGQR | na | na |
| (Format: Whole | LSCAASGFDFSRYWMSW | ATISCRASKSVSTSGYSYL | | |
| mAb | VRQAPGKGLEWIGEINPT | HWYQQKPGQPPKLLIYL | | |
| Radiolabelled) | SSTINFTPSLKDKVFISRDN | ASNLESGVPARFSGSGSG | | |
| | AKNTLYLQMSKVRSEDTA | TDFTLNIHPVEEEDAATY | | |
| | LYYCARGNYYRYGDAMDY | YCQHSRELPFTFGSGTKL | | |
| | WGQGTSVTVSS (SEQ ID | EIK (SEQ ID NO: 56) | | |
| | NO: 55) | | | |
| Aprutumab | EVQLLESGGGLVQPGGSL | QSVLTQPPSASGTPGQR | na | na |
| (Format: Whole | RLSCAASGFTFSSYAMSW | VTISCSGSSSNIGNNYVS | | |
| mAb ADC) | VRQAPGKGLEWVSAISGS | WYQQLPGTAPKLLIYENY | | |
| | GTSTYYADSVKGRFTISRD | NRPAGVPDRFSGSKSGT | | |
| | NSKNTLYLQMNSLRAEDT | SASLAISGLRSEDEADYYC | | |
| | AVYYCARVRYNWNHGD | SSWDDSLNYWVFGGGT | | |
| | WFDPWGQGTLVTVSS | KLTVL | | |
| | (SEQ ID NO: 57) | (SEQ ID NO: 58) | | |
| Ascrinvacumab | QVQLQESGPGLVKPSQTL | EIVLTQSPGTLSLSPGERA | na | na |
| (Format: Whole | SLTCTVSGGSISSGEYYWN | TLSCRASQSVSSSYLAWY | | |
| mAb) | WIRQHPGKGLEWIGYIYYS | QQKPGQAPRLLIYGTSSR | | |
| | GSTYYNPSLKSRVTISVDTS | ATGIPDRFSGSGSGTDFT | | |
| | KNQFSLKLSSVTAADTAVY | LTISRLEPEDFAVYYCQQY | | |
| | YCARESVAGFDYWGQGTL | GSSPITFGQGTRLEIK | | |
| | VTVSS (SEQ ID NO: 59) | (SEQ ID NO: 60) | | |
| Atezolizumab | EVQLVESGGGLVQPGGSL | DIQMTQSPSSLSASVGD | na | na |
| (Format: Whole | RLSCAASGFTFSDSWIHW | RVTITCRASQDVSTAVA | | |
| mAb) | VRQAPGKGLEWVAWISP | WYQQKPGKAPKLLIYSAS | | |
| | YGGSTYYADSVKGRFTISA | FLYSGVPSRFSGSGSGTD | | |
| | DTSKNTAYLQMNSLRAED | FTLTISSLQPEDFATYYCQ | | |
| | TAVYYCARRHWPGGFDY | QYLYHPATFGQGTKVEIK | | |
| | WGQGTLVTVSS (SEQ ID | (SEQ ID NO: 62) | | |
| | NO: 61) | | | |
| Atidortoxumab | EVQLQESGPGLVRPSETLS | DIQMTQSPSSVSASVGD | na | na |
| (Format: Whole | LTCAVSGYSISSGMGWG | RVTITCRASQGISRWLA | | |
| mAb) | WIRQPPGKGLEWIGSIDQ | WYQQKPGKAPKLLIYAA | | |
| | RGSTYYNPSLKSRVTISVDT | SSLQSGVPSRFSGSGSGT | | |
| | SKNQFSLKLSSVTAADTAV | DFTLTISSLQPEDFATYYC | | |
| | YYCARDAGHAVDMDVW | QQGYVFPLTFGGGTKVEI | | |
| | GKGTTVTVSS (SEQ ID | K (SEQ ID NO: 64) | | |
| | NO: 63) | | | |
| Atinumab | EVQLVESGGGLVQPGGSL | EIVLTQSPATLSLSPGERA | na | na |
| (Format: Whole | RLSCAASGFTFSNYWMS | TLSCRASQSVSSYLAWYQ | | |
| mAb) | WVRQAPGKGLEWVATIK | QKPGQAPRLLIYDASNRA | | |
| | QDGSQKNYVDSVKGRFTI | TGIPARFSGSGSGTDFTLT | | |
| | SRDNAKNSLYLRLNSLRAE | ISSLEPEDFAVYYCQQRS | | |
| | DTAVYYCATELFDLWGRG | NWPITFGQGTKLEIK | | |
| | SLVTVSS (SEQ ID NO: 65) | (SEQ ID NO: 66) | | |
| Atoltivimab | QVQLVESGGGVVQPGRSL | DIQMTQSPSSLSASVGD | na | na |
| (Format: Whole | RLSCAASGFTFNNYGMH | RITITCRASQSISTYLHWY | | |
| mAb) | WVRQAPGMGLEWVAVI | QQKPGKAPKLLIYAASTL | | |
| | WHDGSDKYYADSVKGRF | QSGVPSRFSGSGSGTDFT | | |
| | TISRDNSKNTLYLQMNSLR | LTISSLQPEDFATYYCQQS | | |
| | AEDTAVYYCARNWNLFDY | FSTPPINFGQGTKLEIK | | |
| | WGQGTLVTVSS (SEQ ID | (SEQ ID NO: 68) | | |
| | NO: 67) | | | |
| Avelumab | EVQLLESGGGLVQPGGSL | QSALTQPASVSGSPGQSI | na | na |
| (Bintrafusp) | RLSCAASGFTFSSYIMMW | TISCTGTSSDVGGYNYVS | | |
| (Format: Whole | VRQAPGKGLEWVSSIYPS | WYQQHPGKAPKLMIYD | | |
| mAb) | GGITFYADTVKGRFTISRD | VSNRPSGVSNRFSGSKSG | | |
| | NSKNTLYLQMNSLRAEDT | NTASLTISGLQAEDEADY | | |
| | AVYYCARIKLGTVTTVDY | YCSSYTSSSTRVFGTGTKV | | |
| | WGQGTLVTVSS (SEQ ID | TVL (SEQ ID NO: 70) | | |
| | NO: 69) | | | |
| Azintuxizumab | EVQLVESGGGLVQPGGSL | DVVMTQTPLSLSVTPGQ | na | na |
| (Format: Whole | RLSCAASGFTFSDYYMAW | PASISCRSSQSLVHSNGN | | |
| mAb ADC) | VRQAPGKGLEWVASINYD | TYLHWYLQKPGQSPQLLI | | |
| | GSSTYYVDSVKGRFTISRD | YKVSNRFSGVPDRFSGSG | | |
| | NAKNSLYLQMNSLRAEDT | SGTDFTLKISRVEAEDVG | | |
| | AVYYCARDRGYYFDYWG | VYFCSQSTHVPPFTFGGG | | |
| | QGTTVTVSS (SEQ ID NO: | TKVEIK (SEQ ID NO: 72) | | |
| | 71) | | | |
| Balstilimab | QVQLVESGGGVVQPGRSL | EIVMTQSPATLSVSPGER | na | na |
| (Format: Whole | RLSCAASGFTFSSYGMHW | ATLSCRASQSVSSNLAWY | | |
| mAb) | VRQAPGKGLEWVAVIWY | QQKPGQAPRLLIYGASTR | | |
| | DGSNKYYADSVKGRFTISR | ATGIPARFSGSGSGTEFTL | | |
| | DNSKNTLYLQMNSLRAED | TISSLQSEDFAVYYCQQY | | |
| | TAVYYCASNGDHWGQGT | NNWPRTFGQGTKVEIK | | |
| | LVTVSS (SEQ ID NO: 73) | (SEQ ID NO: 74) | | |
| Bapineuzumab | EVQLLESGGGLVQPGGSL | DVVMTQSPLSLPVTPGE | na | na |
| (Format: Whole | RLSCAASGFTFSNYGMSW | PASISCKSSQSLLDSDGKT | | |
| mAb) | VRQAPGKGLEWVASIRSG | YLNWLLQKPGQSPQRLIY | | |
| | GGRTYYSDNVKGRFTISRD | LVSKLDSGVPDRFSGSGS | | |
| | NSKNTLYLQMNSLRAEDT | GTDFTLKISRVEAEDVGV | | |
| | AVYYCVRYDHYSGSSDYW | YYCWQGTHFPRTFGQGT | | |
| | GQGTLVTVSS (SEQ ID | KVEIK | | |
| | NO: 75) | (SEQ ID NO: 76) | | |
| Basiliximab | QVQLQQSGTVLARPGASV | QIVSTQSPAIMSASPGEK | na | na |
| (Format: Whole | KMSCKASGYSFTRYWMH | VTMTCSASSSRSYMQW | | |
| mAb) | WIKQRPGQGLEWIGAIYP | YQQKPGTSPKRWIYDTS | | |
| | GNSDTSYNQKFEGKAKLT | KLASGVPARFSGSGSGTS | | |
| | AVTSASTAYMELSSLTHED | YSLTISSMEAEDAATYYC | | |
| | SAVYYCSRDYGYYFDFWG | HQRSSYTFGGGTKLEIK | | |
| | QGTTLTVSS (SEQ ID NO: | (SEQ ID NO: 78) | | |
| | 77) | | | |
| Bavituximab | EVQLQQSGPELEKPGASV | DIQMTQSPSSLSASLGER | na | na |
| (Format: Whole | KLSCKASGYSFTGYNMNW | VSLTCRASQDIGSSLNWL | | |
| mAb) | VKQSHGKSLEWIGHIDPYY | QQGPDGTIKRLIYATSSL | | |
| | GDTSYNQKFRGKATLTVD | DSGVPKRFSGSRSGSDYS | | |
| | KSSSTAYMQLKSLTSEDSA | LTISSLESEDFVDYYCLQY | | |
| | VYYCVKGGYYGHWYFDV | VSSPPTFGAGTKLELK | | |
| | WGAGTTVTVSS (SEQ ID | (SEQ ID NO: 80) | | |
| | NO: 79) | | | |
| Bedinvetmab | EVQLVESGGDLVKPGGSL | QSVLTQPTSVSGSLGWR | na | na |
| (Format: Canine | RLSCVASGFTFSSHGMHW | VTISCSGSTNNIGILGAS | | |
| Whole mAb) | VRQSPGKGLQWVAVINS | WYQLFPGKAPKLLVYGN | | |
| | GGSSTYYTDAVKGRFTISR | GNRPSGVPDRFSGADSG | | |
| | DNAKNTVYLQMNSLRAE | DSVTLTITGLQAEDEADY | | |
| | DTAMYYCAKESVGGWEQ | YCQSFDTTLGAHVFGGG | | |
| | LVGPHFDYWGQGTLVIVS | THLTVL | | |
| | S (SEQ ID NO: 81) | (SEQ ID NO: 82) | | |
| Begelomab | QVQLQQSGAELVKPGASV | QIVLTQSPAIMSASPGEK | na | na |
| (Format: Whole | KLSCKASGYTFRSYDINWV | VTITCSASSSVSYMNWF | | |
| mAb) | RQRPEQGLEWIGWIFPGD | QQKPGTSPKLWIYSTSNL | | |
| | GSTKYNEKFKGKATLTTDK | ASGVPARFSGSGSGTSYS | | |
| | SSSTAYMQLSRLTSEDSAV | LTISRMEAEDAATYYCQQ | | |
| | YFCARWTVVGPGYFDVW | RSSYPNTFGGGTKLEIK | | |
| | GAGTTVTVSS (SEQ ID | (SEQ ID NO: 84) | | |
| | NO: 83) | | | |
| Belantamab | QVQLVQSGAEVKKPGSSV | DIQMTQSPSSLSASVGD | na | na |
| (Format: Whole | KVSCKASGGTFSNYWMH | RVTITCSASQDISNYLNW | | |
| mAb ADC) | WVRQAPGQGLEWMGAT | YQQKPGKAPKLLIYYTSNL | | |
| | YRGHSDTYYNQKFKGRVTI | HSGVPSRFSGSGSGTDFT | | |
| | TADKSTSTAYMELSSLRSE | LTISSLQPEDFATYYCQQY | | |
| | DTAVYYCARGAIYDGYDVL | RKLPWTFGQGTKLEIK | | |
| | DNWGQGTLVTVSS (SEQ | (SEQ ID NO: 86) | | |
| | ID NO: 85) | | | |
| Belimumab | QVQLQQSGAEVKKPGSSV | SSELTQDPAVSVALGQTV | na | na |
| (Format: Whole | RVSCKASGGTFNNNAIN | RVTCQGDSLRSYYASWY | | |
| mAb) | WVRQAPGQGLEWMGGII | QQKPGQAPVLVIYGKNN | | |
| | PMFGTAKYSQNFQGRVAI | RPSGIPDRFSGSSSGNTA | | |
| | TADESTGTASMELSSLRSE | SLTITGAQAEDEADYYCS | | |
| | DTAVYYCARSRDLLLFPHH | SRDSSGNHWVFGGGTEL | | |
| | ALSPWGRGTMVTVSS | TVL (SEQ ID NO: 88) | | |
| | (SEQ ID NO: 87) | | | |
| Bemarituzumab | QVQLVQSGAEVKKPGSSV | DIQMTQSPSSLSASVGD | na | na |
| (Format: Whole | KVSCKASGYIFTTYNVHW | RVTITCKASQGVSNDVA | | |
| mAb) | VRQAPGQGLEWIGSIYPD | WYQQKPGKAPKLLIYSAS | | |
| | NGDTSYNQNFKGRATITA | YRYTGVPSRFSGSGSGTD | | |
| | DKSTSTAYMELSSLRSEDT | FTFTISSLQPEDIATYYCQ | | |
| | AVYYCARGDFAYWGQGT | QHSTTPYTFGQGTKLEIK | | |
| | LVTVSS (SEQ ID NO: 89) | (SEQ ID NO: 90) | | |
| Benralizumab | EVQLVQSGAEVKKPGASV | DIQMTQSPSSLSASVGD | na | na |
| (Format: Whole | KVSCKASGYTFTSYVIHWV | RVTITCGTSEDIINYLNWY | | |
| mAb) | RQRPGQGLAWMGYINPY | QQKPGKAPKLLIYHTSRL | | |
| | NDGTKYNERFKGKVTITSD | QSGVPSRFSGSGSGTDFT | | |
| | RSTSTVYMELSSLRSEDTA | LTISSLQPEDFATYYCQQ | | |
| | VYLCGREGIRYYGLLGDYW | GYTLPYTFGQGTKVEIK | | |
| | GQGTLVTVSS (SEQ ID | (SEQ ID NO: 92) | | |
| | NO: 91) | | | |
| Berlimatoxumab | ELQLQESGPGLVKPSETLSL | DIQMTQSPSSLSASVGD | na | na |
| (Format: Whole | TCTVSGGSISSGSYYWDWI | RVTITCRASQSINSYLNW | | |
| mAb) | RQPPGKGLEWIGNIYKSGS | YQQKPGKAPKLLIYAASSL | | |
| | TYYNPSLKSRVTISVDTSKN | QSGVPSRFSGSGSGTDFT | | |
| | QFSLKLSSVTAADTAVYYC | LTISSLQPEDFATYYCQQ | | |
| | ARERGMHYMDVWGKGT | QFDPPFTFGGGTKVEIK | | |
| | TVTVSS (SEQ ID NO: 93) | (SEQ ID NO: 94) | | |
| Bermekimab | QVQLVESGGGVVQPGRSL | DIQMTQSPSSVSASVGD | na | na |
| (Format: Whole | RLSCTASGFTFSMFGVHW | RVTITCRASQGISSWLAW | | |
| mAb) | VRQAPGKGLEWVAAVSY | YQQKPGKAPKLLIYEASN | | |
| | DGSNKYYAESVKGRFTISR | LETGVPSRFSGSGSGSDF | | |
| | DNSKNILFLQMDSLRLEDT | TLTISSLQPEDFATYYCQQ | | |
| | AVYYCARGRPKVVIPAPLA | TSSFLLSFGGGTKVEHK | | |
| | HWGQGTLVTFSS (SEQ ID | (SEQ ID NO: 96) | | |
| | NO: 95) | | | |
| Bersanlimab | EVQLLESGGGLVQPGGSL | QSVLTQPPSASGTPGQR | na | na |
| (Format: Whole | RLSCAASGFTFSNAWMS | VTISCTGSSSNIGAGYDV | | |
| mAb) | WVRQAPGKGLEWVAFIW | HWYQQLPGTAPKLLIYD | | |
| | YDGSNKYYADSVKGRFTIS | NNNRPSGVPDRFSGSKS | | |
| | RDNSKNTLYLQMNSLRAE | GTSASLAISGLRSEDEADY | | |
| | DTAVYYCARYSGWYFDY | YCQSYDSSLSAWLFGGG | | |
| | WGQGTLVTVSS (SEQ ID | TKLTVL (SEQ ID NO: 98) | | |
| | NO: 97) | | | |
| Bevacizumab | EVQLVESGGGLVQPGGSL | DIQMTQSPSSLSASVGD | na | na |
| (Format: Whole | RLSCAASGYTFTNYGMN | RVTITCSASQDISNYLNW | | |
| mAb) | WVRQAPGKGLEWVGWI | YQQKPGKAPKVLIYFTSSL | | |
| | NTYTGEPTYAADFKRRFTF | HSGVPSRFSGSGSGTDFT | | |
| | SLDTSKSTAYLQMNSLRAE | LTISSLQPEDFATYYCQQY | | |
| | DTAVYYCAKYPHYYGSSH | STVPWTFGQGTKVEIK | | |
| | WYFDVWGQGTLVTVSS | (SEQ ID NO: 100) | | |
| | (SEQ ID NO: 99) | | | |
| Bezlotoxumab | EVQLVQSGAEVKKSGESLK | EIVLTQSPGTLSLSPGERA | na | na |
| (Format: Whole | ISCKGSGYSFTSYWIGWVR | TLSCRASQSVSSSYLAWY | | |
| mAb) | QMPGKGLEWMGIFYPGD | QQKPGQAPRLLIYGASSR | | |
| | SSTRYSPSFQGQVTISADK | ATGIPDRFSGSGSGTDFT | | |
| | SVNTAYLQWSSLKASDTA | LTISRLEPEDFAVYYCQQY | | |
| | MYYCARRRNWGNAFDI | GSSTWTFGQGTKVEIK | | |
| | WGQGTMVTVSS (SEQ ID | (SEQ ID NO: 102) | | |
| | NO: 101) | | | |
| Blmagrumab | QVQLVQSGAEVKKPGASV | QSALTQPASVSGSPGQSI | na | na |
| (Format: Whole | KVSCKASGYTFTSSYINWV | TISCTGTSSDVGSYNYVN | | |
| mAb) | RQAPGQGLEWMGTINPV | WYQQHPGKAPKLMIYG | | |
| | SGSTSYAQKFQGRVTMTR | VSKRPSGVSNRFSGSKSG | | |
| | DTSISTAYMELSRLRSDDT | NTASLTISGLQAEDEADY | | |
| | AVYYCARGGWFDYWGQ | YCGTFAGGSYYGVFGGG | | |
| | GTLVTVSS (SEQ ID NO: | TKLTVL (SEQ ID NO: | | |
| | 103) | 104) | | |
| Bimekizumab | EVQLVESGGGLVQPGGSL | AIQLTQSPSSLSASVGDR | na | na |
| (Format: Whole | RLSCAASGFTFSDYNMAW | VTITCRADESVRTLMHW | | |
| mAb) | VRQAPGKGLEWVATITYE | YQQKPGKAPKLLIYLVSN | | |
| | GRNTYYRDSVKGRFTISRD | SEIGVPDRFSGSGSGTDF | | |
| | NAKNSLYLQMNSLRAEDT | RLTISSLQPEDFATYYCQQ | | |
| | AVYYCASPPQYYEGSIYRL | TWSDPWTFGQGTKVEIK | | |
| | WFAHWGQGTLVTVSS | (SEQ ID NO: 106) | | |
| | (SEQ ID NO: 105) | | | |
| Birtamimab | EVQLVESGGGLVQPGGSL | DVVMTQSPLSLPVTPGE | na | na |
| (Format: Whole | RLSCAASGFTFNTYAMYW | PASISCRSSQSLVHSTGNT | | |
| mAb) | IRQAPGKGLEWVARIRSKS | YLHWYLQKPGQSPQLLIY | | |
| | NNYAIYYADSVKDRFTISR | KVSNRFSGVPDRFSGSGS | | |
| | DDSKNSLYLQMNSLKTED | GTDFTLKISRVEAEDVGV | | |
| | TAVYYCARPYSDSFAYWG | YYCSQSTHVPFTFGGGTK | | |
| | QGTLVTVSS (SEQ ID NO: | VEIK (SEQ ID NO: 108) | | |
| | 107) | | | |
| Bleselumab | QLQLQESGPGLLKPSETLS | AIQLTQSPSSLSASVGDR | na | na |
| (Format: Whole | LTCTVSGGSISSPGYYGGW | VTITCRASQGISSALAWY | | |
| mAb) | IRQPPGKGLEWIGSIYKSG | QQKPGKAPKLLIYDASNL | | |
| | STYHNPSLKSRVTISVDTSK | ESGVPSRFSGSGSGTDFT | | |
| | NQFSLKLSSVTAADTAVYY | LTISSLQPEDFATYYCQQF | | |
| | CTRPVVRYFGWFDPWGQ | NSYPTFGQGTKVEIK | | |
| | GTLVTVSS (SEQ ID NO: | (SEQ ID NO: 110) | | |
| | 109) | | | |
| Blinatumomab | QVQLQQSGAELVRPGSSV | DIQLTQSPASLAVSLGQR | DIKLQSGAELARPG | DIQLTQSPAIMSA |
| (Format: | KISCKASGYAFSSYWMNW | ATISCKASQSVDYDGDSY | ASVKMSCKTSGYT | SPGEKVTMTCRA |
| Bispecific | VKQRPGQGLEWIGQIWP | LNWYQQIPGQPPKLLIYD | FTRYTMHWVKQR | SSSVSYMNWYQ |
| T-Cell | GDGDTNYNGKFKGKATLT | ASNLVSGIPPRFSGSGSG | PGQGLEWIGYINP | QKSGTSPKRWIY |
| Engager) | ADESSSTAYMQLSSLASED | TDFTLNIHPVEKVDAATY | SRGYTNYNQKFKD | DTSKVASGVPYRF |
| | SAVYFCARRETTTVGRYYY | HCQQSTEDPWTFGGGT | KATLTTDKSSSTAY | SGSGSGTSYSLTIS |
| | AMDYWGQGTTVTVSS | KLEIK | MQLSSLTSEDSAVY | SMEAEDAATYYC |
| | (SEQ ID NO: 111) | (SEQ ID NO: 112) | YCARYYDDHYCLD | QQWSSNPLTFGA |
| | | | YWGQGTTLTVSS | GTKLELK (SEQ ID |
| | | | (SEQ ID NO: 113) | NO: 114) |
| Blontuvetmab | QVQLQQSRAELVRPGASV | DVVMSQSPSSLAVSVGE | na | na |
| (Format: Canine | TLSCKPSGYTFTDYEVHW | KVTMSCKSSQSLLYSGN | | |
| Whole mAb) | VKQTPVHGLEWIGAIDPE | QKNYLAWYQQKPGQSP | | |
| | TGGTADNQKFKGKAILTA | RLLIYWASTRESGVPDRF | | |
| | DKSSSTAYMELRSLTSEDS | TGSGSGTDFTLTISSVKAE | | |
| | AVYYCTNFVDVWGTGTT | DLAVFYCQQYYNYPLTFG | | |
| | VTVSS (SEQ ID NO: 115) | GGTHL (SEQ ID NO: | | |
| | | 116) | | |
| Blosozumab | QVQLVQSGAEVKKPGASV | DIQMTQSPSSLSASVGD | na | na |
| (Format: Whole | KVSCKVSGFPIKDTFQHW | RVTITCKASQDVHTAVA | | |
| mAb) | VRQAPGKGLEWMGWSD | WYQQKPGKAPKLLIYWA | | |
| | PEIGDTEYASKFQGRVTM | STRWTGVPSRFSGSGSG | | |
| | TEDTSTDTAYMELSSLRSE | TDFTLTISSLQPEDFATYY | | |
| | DTAVYYCATGDTTYKFDF | CQQYSDYPWTFGGGTK | | |
| | WGQGTTVTVSS (SEQ ID | VEIK (SEQ ID NO: 118) | | |
| | NO: 117) | | | |
| Bococizumab | QVQLVQSGAEVKKPGASV | DIQMTQSPSSLSASVGD | na | na |
| (Format: Whole | KVSCKASGYTFTSYYMHW | RVTITCRASQGISSALAW | | |
| mAb) | VRQAPGQGLEWMGEISP | YQQKPGKAPKLLIYSASY | | |
| | FGGRTNYNEKFKSRVTMT | RYTGVPSRFSGSGSGTDF | | |
| | RDTSTSTVYMELSSLRSED | TFTISSLQPEDIATYYCQQ | | |
| | TAVYYCARERPLYASDLW | RYSLWRTFGQGTKLEIK | | |
| | GQGTTVTVSS (SEQ ID | (SEQ ID NO: 120) | | |
| | NO: 119) | | | |
| Brazikumab | QVQLVESGGGVVQPGRSL | QSVLTQPPSVSGAPGQR | na | na |
| (Format: Whole | RLSCAASGFTFSSYGMHW | VTISCTGSSSNTGAGYDV | | |
| mAb) | VRQAPGKGLEWVAVIWY | HWYQQVPGTAPKLLIYG | | |
| | DGSNEYYADSVKGRFTISR | SGNRPSGVPDRFSGSKS | | |
| | DNSKNTLYLQMNSLRAED | GTSASLAITGLQAEDEAD | | |
| | TAVYYCARDRGYTSSWYP | YYCQSYDSSLSGWVFGG | | |
| | DAFDIWGQGTMVTVSS | GTRLTVL (SEQ ID NO: | | |
| | (SEQ ID NO: 121) | 122) | | |
| Brentuximab | QIQLQQSGPEVVKPGASV | DIVLTQSPASLAVSLGQR | na | na |
| (Format: Whole | KISCKASGYTFTDYYITWV | ATISCKASQSVDFDGDSY | | |
| mAb ADC) | KQKPGQGLEWIGWIYPGS | MNWYQQKPGQPPKVLI | | |
| | GNTKYNEKFKGKATLTVD | YAASNLESGIPARFSGSG | | |
| | TSSSTAFMQLSSLTSEDTA | SGTDFTLNIHPVEEEDAA | | |
| | VYFCANYGNYWFAYWGQ | TYYCQQSNEDPWTFGG | | |
| | GTQVTVSA (SEQ ID NO: | GTKLEIK (SEQ ID NO: | | |
| | 123) | 124) | | |
| Briakinumab | QVQLVESGGGVVQPGRSL | QSVLTQPPSVSGAPGQR | na | na |
| (Format: Whole | RLSCAASGFTFSSYGMHW | VTISCSGSRSNIGSNTVK | | |
| mAb) | VRQAPGKGLEWVAFIRYD | WYQQLPGTAPKLLIYYN | | |
| | GSNKYYADSVKGRFTISRD | DQRPSGVPDRFSGSKSG | | |
| | NSKNTLYLQMNSLRAEDT | TSASLAITGLQAEDEADY | | |
| | AVYYCKTHGSHDNWGQG | YCQSYDRYTHPALLFGTG | | |
| | TMVTVSS (SEQ ID NO: | TKVTVL (SEQ ID NO: | | |
| | 125) | 126) | | |
| Brodalumab | QVQLVQSGAEVKKPGASV | EIVMTQSPATLSVSPGER | na | na |
| (Format: Whole | KVSCKASGYTFTRYGISWV | ATLSCRASQSVSSNLAWF | | |
| mAb) | RQAPGQGLEWMGWISTY | QQKPGQAPRPLIYDASTR | | |
| | SGNTNYAQKLQGRVTMT | ATGVPARFSGSGSGTDFT | | |
| | TDTSTSTAYMELRSLRSDD | LTISSLQSEDFAVYYCQQY | | |
| | TAVYYCARRQLYFDYWGQ | DNWPLTFGGGTKVEIK | | |
| | GTLVTVSS (SEQ ID NO: | (SEQ ID NO: 128) | | |
| | 127) | | | |
| Brolucizumab | EVQLVESGGGLVQPGGSL | MEIVMTQSPSTLSASVG | na | na |
| (Format: scFv) | RLSCTASGFSLTDYYYMT | DRVIITCQASEIIHSWLA | | |
| | WVRQAPGKGLEWVGFID | WYQQKPGKAPKLLIYLAS | | |
| | PDDDPYYATWAKGRFTIS | TLASGVPSRFSGSGSGAE | | |
| | RDNSKNTLYLQMNSLRAE | FTLTISSLQPDDFATYYCQ | | |
| | DTAVYYCAGGDHNSGWG | NVYLASTNGANFGQGTK | | |
| | LDIWGQGTLVTVSS (SEQ | LTVLG (SEQ ID NO: 130) | | |
| | ID NO: 129) | | | |
| Brontictuzumab | QVQLVQSGAEVKKPGASV | QAVVTQEPSLTVSPGGT | na | na |
| (Format: Whole | KISCKVSGYTLRGYWIEWV | VTLTCRSSTGAVTTSNYA | | |
| mAb) | RQAPGKGLEWIGQILPGT | NWFQQKPGQAPRTLIG | | |
| | GRTNYNEKFKGRVTMTA | GTNNRAPGVPARFSGSL | | |
| | DTSTDTAYMELSSLRSEDT | LGGKAALTLSGAQPEDEA | | |
| | AVYYCARFDGNYGYYAM | EYYCALWYSNHWVFGG | | |
| | DYWGQGTTVTVSS (SEQ | GTKLTVL (SEQ ID NO: | | |
| | ID NO: 131) | 132) | | |
| Budigalimab | EIQLVQSGAEVKKPGSSVK | DVVMTQSPLSLPVTPGE | na | na |
| (Format: Whole | VSCKASGYTFTHYGMNW | PASISCRSSQSIVHSHGDT | | |
| mAb) | VRQAPGQGLEWVGWVN | YLEWYLQKPGQSPQLLIY | | |
| | TYTGEPTYADDFKGRLTFT | KVSNRFSGVPDRFSGSGS | | |
| | LDTSTSTAYMELSSLRSED | GTDFTLKISRVEAEDVGV | | |
| | TAVYYCTREGEGLGFGDW | YYCFQGSHIPVTFGQGTK | | |
| | GQGTTVTVSS (SEQ ID | LEIK (SEQ ID NO: 134) | | |
| | NO: 133) | | | |
| Burosumab | QVQLVQSGAEVKKPGASV | AIQLTQSPSSLSASVGDR | na | na |
| (Format: Whole | KVSCKASGYTFTNHYMH | VTITCRASQGISSALVWY | | |
| mAb) | WVRQAPGQGLEWMGIIN | QQKPGKAPKLLIYDASSL | | |
| | PISGSTSNAQKFQGRVTM | ESGVPSRFSGSGSGTDFT | | |
| | TRDTSTSTVYMELSSLRSE | LTISSLQPEDFATYYCQQF | | |
| | DTAVYYCARDIVDAFDFW | NDYFTFGPGTKVDIK | | |
| | GQGTMVTVSS (SEQ ID | (SEQ ID NO: 136) | | |
| | NO: 135) | | | |
| Cabiralizumab | QVQLVQSGAEVKKPGSSV | EIVLTQSPATLSLSPGERA | na | na |
| (Format: Whole | KVSCKASGYTFTDNYMIW | TLSCKASQSVDYDGDNY | | |
| mAb) | VRQAPGQGLEWMGDINP | MNWYQQKPGQAPRLLI | | |
| | YNGGTTFNQKFKGRVTIT | YAASNLESGIPARFSGSG | | |
| | ADKSTSTAYMELSSLRSED | SGTDFTLTISSLEPEDFAV | | |
| | TAVYYCARESPYFSNLYVM | YYCHLSNEDLSTFGGGTK | | |
| | DYWGQGTLVTVSS (SEQ | VEIK (SEQ ID NO: 138) | | |
| | ID NO: 137) | | | |
| Camidanlumab | QVQLVQSGAEVKKPGSSV | EIVLTQSPGTLSLSPGERA | na | na |
| (Format: Whole | KVSCKASGGTFSRYIINWV | TLSCRASQSVSSYLAWYQ | | |
| mAb ADC) | RQAPGQGLEWMGRIIPIL | QKPGQAPRLLIYGASSRA | | |
| | GVENYAQKFQGRVTITAD | TGIPDRFSGSGSGTDFTL | | |
| | KSTSTAYMELSSLRSEDTA | TISRLEPEDFAVYYCQQY | | |
| | VYYCARKDWFDYWGQGT | GSSPLTFGGGTKVEIK | | |
| | LVTVSS (SEQ ID NO: 139) | (SEQ ID NO: 140) | | |
| Camrelizumab | EVQLVESGGGLVQPGGSL | DIQMTQSPSSLSASVGD | na | na |
| (Format: Whole | RLSCAASGFTFSSYMMSW | RVTITCLASQTIGTWLTW | | |
| mAb) | VRQAPGKGLEWVATISGG | YQQKPGKAPKLLIYTATSL | | |
| | GANTYYPDSVKGRFTISRD | ADGVPSRFSGSGSGTDFT | | |
| | NAKNSLYLQMNSLRAEDT | LTISSLQPEDFATYYCQQ | | |
| | AVYYCARQLYYFDYWGQ | VYSIPWTFGGGTKVEIK | | |
| | GTTVTVSS (SEQ ID NO: | (SEQ ID NO: 142) | | |
| | 141) | | | |
| Canakinumab | QVQLVESGGGVVQPGRSL | EIVLTQSPDFQSVTPKEK | na | na |
| (Format: Whole | RLSCAASGFTFSVYGMNW | VTITCRASQSIGSSLHWY | | |
| mAb) | VRQAPGKGLEWVAIIWYD | QQKPDQSPKLLIKYASQS | | |
| | GDNQYYADSVKGRFTISR | FSGVPSRFSGSGSGTDFT | | |
| | DNSKNTLYLQMNGLRAED | LTINSLEAEDAAAYYCHQ | | |
| | TAVYYCARDLRTGPFDYW | SSSLPFTFGPGTKVDIK | | |
| | GQGTLVTVSS (SEQ ID | (SEQ ID NO: 144) | | |
| | NO: 143) | | | |
| Cantuzumab | QVQLVQSGAEVKKPGETV | DIVMTQSPLSVPVTPGEP | na | na |
| (Format: Whole | KISCKASDYTFTYYGMNW | VSISCRSSKSLLHSNGNTY | | |
| mAb ADC) | VKQAPGQGLKWMGWID | LYWFLQRPGQSPQLLIYR | | |
| | TTTGEPTYAQKFQGRIAFS | MSNLVSGVPDRFSGSGS | | |
| | LETSASTAYLQIKSLKSEDT | GTAFTLRISRVEAEDVGV | | |
| | ATYFCARRGPYNWYFDV | YYCLQHLEYPFTFGPGTK | | |
| | WGQGTTVTVSS (SEQ ID | LELK (SEQ ID NO: 146) | | |
| | NO: 145) | | | |
| Caplacizumab | EVQLVESGGGLVQPGGSL | na | na | na |
| (Format: | RLSCAASGRTFSYNPMG | | | |
| Nanobody) | WFRQAPGKGRELVAAISR | | | |
| | TGGSTYYPDSVEGRFTISR | | | |
| | DNAKRMVYLQMNSLRAE | | | |
| | DTAVYYCAAAGVRAEDGR | | | |
| | VRTLPSEYTFWGQGTQVT | | | |
| | VSS (SEQ ID NO: 147) | | | |
| Carlumab | QVQLVQSGAEVKKPGSSV | EIVLTQSPATLSLSPGERA | na | na |
| (Format: Whole | KVSCKASGGTFSSYGISWV | TLSCRASQSVSDAYLAWY | | |
| mAb) | RQAPGQGLEWMGGIIPIF | QQKPGQAPRLLIYDASSR | | |
| | GTANYAQKFQGRVTITAD | ATGVPARFSGSGSGTDFT | | |
| | ESTSTAYMELSSLRSEDTA | LTISSLEPEDFAVYYCHQY | | |
| | VYYCARYDGIYGELDFWG | IQLHSFTFGQGTKVEIK | | |
| | QGTLVTVSS (SEQ ID NO: | (SEQ ID NO: 149) | | |
| | 148) | | | |
| Carotuximab | EVKLEESGGGLVQPGGSM | QIVLSQSPAILSASPGEKV | na | na |
| (Format: Whole | KLSCAASGFTFSDAWMD | TMTCRASSSVSYMHWY | | |
| mAb) | WVRQSPEKGLEWVAEIRS | QQKPGSSPKPWIYATSNL | | |
| | KASNHATYYAESVKGRFTI | ASGVPVRFSGSGSGTSYS | | |
| | SRDDSKSSVYLQMNSLRA | LTISRVEAEDAATYYCQQ | | |
| | EDTGIYYCTRWRRFFDSW | WSSNPLTFGAGTKLELK | | |
| | GQGTTLTVSS (SEQ ID | (SEQ ID NO: 151) | | |
| | NO: 150) | | | |
| Cemiplimab | EVQLLESGGVLVQPGGSL | DIQMTQSPSSLSASVGDS | na | na |
| (Format: Whole | RLSCAASGFTFSNFGMTW | ITITCRASLSINTFLNWYQ | | |
| mAb) | VRQAPGKGLEWVSGISGG | QKPGKAPNLLIYAASSLH | | |
| | GRDTYFADSVKGRFTISRD | GGVPSRFSGSGSGTDFTL | | |
| | NSKNTLYLQMNSLKGEDT | TIRTLQPEDFATYYCQQS | | |
| | AVYYCVKWGNIYFDYWG | SNTPFTFGPGTVVDFR | | |
| | QGTLVTVSS (SEQ ID NO: | (SEQ ID NO: 153) | | |
| | 152) | | | |
| Cendakimab | EVTLRESGPGLVKPTQTLT | DIQMTQSPSSLSASVGD | na | na |
| (Format: Whole | LTCTLYGFSLSTSDMGVD | RVTISCRASQDIRNYLNW | | |
| mAb) | WIRQPPGKGLEWLAHIW | YQQKPGKAPKLLIFYTSKL | | |
| | WDDVKRYNPALKSRLTISK | HSGVPSRFSGSGSGTDYT | | |
| | DTSKNQVVLKLTSVDPVD | LTISSLQPEDIATYYCQQG | | |
| | TATYYCARTVSSGYIYYAM | NTLPLTFGGGTKVEIK | | |
| | DYWGQGTLVTVSS (SEQ | (SEQ ID NO: 155) | | |
| | ID NO: 154) | | | |
| Cergutuzumab | QVQLVQSGAEVKKPGASV | DIQMTQSPSSLSASVGD | na | na |
| (Format: Whole | KVSCKASGYTFTEFGMNW | RVTITCKASAAVGTYVA | | |
| mAb Fusion) | VRQAPGQGLEWMGWIN | WYQQKPGKAPKLLIYSAS | | |
| | TKTGEATYVEEFKGRVTFT | YRKRGVPSRFSGSGSGTD | | |
| | TDTSTSTAYMELRSLRSDD | FTLTISSLQPEDFATYYCH | | |
| | TAVYYCARWDFAYYVEA | QYYTYPLFTFGQGTKLEIK | | |
| | MDYWGQGTTVTVSS | (SEQ ID NO: 157) | | |
| | (SEQ ID NO: 156) | | | |
| Certolizumab | EVQLVESGGGLVQPGGSL | DIQMTQSPSSLSASVGD | na | na |
| (Format: Fab) | RLSCAASGYVFTDYGMN | RVTITCKASQNVGTNVA | | |
| | WVRQAPGKGLEWMGWI | WYQQKPGKAPKALIYSA | | |
| | NTYIGEPIYADSVKGRFTFS | SFLYSGVPYRFSGSGSGT | | |
| | LDTSKSTAYLQMNSLRAE | DFTLTISSLQPEDFATYYC | | |
| | DTAVYYCARGYRSYAMDY | QQYNIYPLTFGQGTKVEI | | |
| | WGQGTLVTVSS (SEQ ID | K (SEQ ID NO: 159) | | |
| | NO: 158) | | | |
| Cetrelimab | QVQLVQSGAEVKKPGSSV | EIVLTQSPATLSLSPGERA | na | na |
| (Format: Whole | KVSCKASGGTFSSYAISWV | TLSCRASQSVRSYLAWYQ | | |
| mAb) | RQAPGQGLEWMGGIIPIF | QKPGQAPRLLIYDASNRA | | |
| | DTANYAQKFQGRVTITAD | TGIPARFSGSGSGTDFTLT | | |
| | ESTSTAYMELSSLRSEDTA | ISSLEPEDFAVYYCQQRN | | |
| | VYYCARPGLAAAYDTGSL | YWPLTFGQGTKVEIK | | |
| | DYWGQGTLVTVSS (SEQ | (SEQ ID NO: 161) | | |
| | ID NO: 160) | | | |
| Cetuximab | QVQLKQSGPGLVQPSQSL | DILLTQSPVILSVSPGERV | na | na |
| (Format: Whole | SITCTVSGFSLTNYGVHWV | SFSCRASQSIGTNIHWYQ | | |
| mAb) | RQSPGKGLEWLGVIWSG | QRTNGSPRLLIKYASESIS | | |
| | GNTDYNTPFTSRLSINKDN | GIPSRFSGSGSGTDFTLSI | | |
| | SKSQVFFKMNSLQSNDTA | NSVESEDIADYYCQQNN | | |
| | IYYCARALTYYDYEFAYWG | NWPTTFGAGTKLELK | | |
| | QGTLVTVSA (SEQ ID NO: | (SEQ ID NO: 163) | | |
| | 162) | | | |
| Cibisatamab | QVQLVQSGAEVKKPGASV | DIQMTQSPSSLSASVGD | QVQLVQSGAEVKK | QAVVTQEPSLTVS |
| (Format: | KVSCKASGYTFTEFGMNW | RVTITCKASAAVGTYVA | PGASVKVSCKASG | PGGTVTLTCGSST |
| Bispecific mAb | VRQAPGQGLEWMGWIN | WYQQKPGKAPKLLIYSAS | YTFTEFGMNWVR | GAVTTSNYANW |
| with Domain | TKTGEATYVEEFKGRVTFT | YRKRGVPSRFSGSGSGTD | QAPGQGLEWMG | VQEKPGQAFRGLI |
| Crossover) | TDTSTSTAYMELRSLRSDD | FTLTISSLQPEDFATYYCH | WINTKTGEATYVE | GGTNKRAPGTPA |
| | TAVYYCARWDFAYYVEA | QYYTYPLFTFGQGTKLEIK | EFKGRVTFTTDTST | RFSGSLLGGKAAL |
| | MDYWGQGTTVTVSS | (SEQ ID NO: 165) | STAYMELRSLRSDD | TLSGAQPEDEAEY |
| | (SEQ ID NO: 164) | | TAVYYCARWDFAY | YCALWYSNLWVF |
| | | | YVEAMDYWGQGT | GGGTKLTVL (SEQ |
| | | | TVTVSS (SEQ ID | ID NO: 167) |
| | | | NO: 166) | |
| Cinpanemab | EVQLVESGGGLVEPGGSL | SYELTQPPSVSVSPGQTA | na | na |
| (Format: Whole | RLSCAVSGFDFEKAWMS | RITCSGEALPMQFAHWY | | |
| mAb) | WVRQAPGQGLQWVARIK | QQRPGKAPVIVVYKDSER | | |
| | STADGGTTSYAAPVEGRFI | PSGVPERFSGSSSGTTAT | | |
| | ISRDDSRNMLYLQMNSLK | LTITGVQAEDEADYYCQS | | |
| | TEDTAVYYCTSAHWGQG | PDSTNTYEVFGGGTKLTV | | |
| | TLVTVSS (SEQ ID NO: | L (SEQ ID NO: 169) | | |
| | 168) | | | |
| Citatuzumab | EVQLVQSGPGLVQPGGSV | DIQMTQSPSSLSASVGD | na | na |
| (Format: Fab | RISCAASGYTFTNYGMNW | RVTITCRSTKSLLHSNGIT | | |
| Fusion) | VKQAPGKGLEWMGWIN | YLYWYQQKPGKAPKLLIY | | |
| | TYTGESTYADSFKGRFTFSL | QMSNLASGVPSRFSSSG | | |
| | DTSASAAYLQINSLRAEDT | SGTDFTLTISSLQPEDFAT | | |
| | AVYYCARFAIKGDYWGQG | YYCAQNLEIPRTFGQGTK | | |
| | TLLTVSS (SEQ ID NO: | VELK (SEQ ID NO: 171) | | |
| | 170) | | | |
| Cixutumumab | EVQLVQSGAEVKKPGSSV | SSELTQDPAVSVALGQTV | na | na |
| (Format: Whole | KVSCKASGGTFSSYAISWV | RITCQGDSLRSYYATWYQ | | |
| mAb) | RQAPGQGLEWMGGIIPIF | QKPGQAPILVIYGENKRP | | |
| | GTANYAQKFQGRVTITAD | SGIPDRFSGSSSGNTASLT | | |
| | KSTSTAYMELSSLRSEDTA | ITGAQAEDEADYYCKSRD | | |
| | VYYCARAPLRFLEWSTQD | GSGQHLVFGGGTKLTVL | | |
| | HYYYYYMDVWGKGTTVT | (SEQ ID NO: 173) | | |
| | VSS (SEQ ID NO: 172) | | | |
| Clazakizumab | EVQLVESGGGLVQPGGSL | AIQMTQSPSSLSASVGDR | na | na |
| (Format: Whole | RLSCAASGFSLSNYYVTWV | VTITCQASQSINNELSWY | | |
| mAb) | RQAPGKGLEWVGIIYGSD | QQKPGKAPKLLIYRASTL | | |
| | ETAYATSAIGRFTISRDNSK | ASGVPSRFSGSGSGTDFT | | |
| | NTLYLQMNSLRAEDTAVY | LTISSLQPDDFATYYCQQ | | |
| | YCARDDSSDWDAKFNLW | GYSLRNIDNAFGGGTKVE | | |
| | GQGTLVTVSS (SEQ ID | IK (SEQ ID NO: 175) | | |
| | NO: 174) | | | |
| Clervonafusp | EVQLQESGGGVVQPGGSL | DIQMTQSPSSLSASVGD | na | na |
| (Format: di-Fab | RLSCAASGFTFSNYGMH | RVTISCRASKSVSTSSYSY | | |
| Fusion) | WIRQAPGKGLEWVSYISS | MHWYQQKPEKALIKYAS | | |
| | GSSTIYYADSVKGRFTISRD | YLQSGVPSRFSGSGSGTD | | |
| | NSKNTLYLQMNSLRSEDT | FTLTISSLQPEDVATYYCQ | | |
| | AVYYCARRGLLLDYWGQG | HSRETFGAGTKLELK | | |
| | TLVTVSS (SEQ ID NO: | (SEQ ID NO: 177) | | |
| | 176) | | | |
| Clivatuzumab | QVQLQQSGAEVKKPGAS | DIQLTQSPSSLSASVGDR | na | na |
| (Format: Whole | VKVSCEASGYTFPSYVLH | VTMTCSASSSVSSSYLYW | | |
| mAb | WVKQAPGQGLEWIGYIN | YQQKPGKAPKLWIYSTS | | |
| Radiolabelled) | PYNDGTQYNEKFKGKATL | NLASGVPARFSGSGSGT | | |
| | TRDTSINTAYMELSRLRSD | DFTLTISSLQPEDSASYFC | | |
| | DTAVYYCARGFGGSYGFA | HQWNRYPYTFGGGTRLE | | |
| | YWGQGTLVTVSS (SEQ ID | IK (SEQ ID NO: 179) | | |
| | NO: 178) | | | |
| Cobolimab | EVQLLESGGGLVQPGGSL | DIQMTQSPSSLSASVGD | na | na |
| (Format: Whole | RLSCAAASGFTFSSYDMS | RVTITCRASQSIRRYLNW | | |
| mAb) | WVRQAPGKGLDWVSTIS | YHQKPGKAPKLLIYGAST | | |
| | GGGTYTYYQDSVKGRFTIS | LQSGVPSRFSGSGSGTDF | | |
| | RDNSKNTLYLQMNSLRAE | TLTISSLQPEDFAVYYCQ | | |
| | DTAVYYCASMDYWGQGT | QSHSAPLTFGGGTKVEIK | | |
| | TVTVSS (SEQ ID NO: 180) | (SEQ ID NO: 181) | | |
| Codrituzumab | QVQLVQSGAEVKKPGASV | DVVMTQSPLSLPVTPGE | na | na |
| (Format: Whole | KVSCKASGYTFTDYEMHW | PASISCRSSQSLVHSNRN | | |
| mAb) | VRQAPGQGLEWMGALD | TYLHWYLQKPGQSPQLLI | | |
| | PKTGDTAYSQKFKGRVTLT | YKVSNRFSGVPDRFSGSG | | |
| | ADKSTSTAYMELSSLTSED | SGTDFTLKISRVEAEDVG | | |
| | TAVYYCTRFYSYTYWGQG | VYYCSQNTHVPPTFGQG | | |
| | TLVTVSS (SEQ ID NO: | TKLEIK (SEQ ID NO: | | |
| | 182) | 183) | | |
| Cofetuzumab | QVQLVQSGPEVKKPGASV | EIVLTQSPATLSLSPGERA | na | na |
| (Format: Whole | KVSCKASGYTFTDYAVHW | TLSCRASESVDSYGKSFM | | |
| mAb ADC) | VRQAPGKRLEWIGVISTYN | HWYQQKPGQAPRLLIYR | | |
| | DYTYNNQDFKGRVTMTR | ASNLESGIPARFSGSGSG | | |
| | DTSASTAYMELSRLRSEDT | TDFTLTISSLEPEDFAVYY | | |
| | AVYYCARGNSYFYALDYW | CQQSNEDPWTFGGGTK | | |
| | GQGTSVTVSS (SEQ ID | LEIK (SEQ ID NO: 185) | | |
| | NO: 184) | | | |
| Coltuximab | QVQLVQPGAEVVKPGAS | EIVLTQSPAIMSASPGER | na | na |
| (Format: Whole | VKLSCKTSGYTFTSNWMH | VTMTCSASSGVNYMHW | | |
| mAb ADC) | WVKQAPGQGLEWIGEID | YQQKPGTSPRRWIYDTS | | |
| | PSDSYTNYNQNFQGKAKL | KLASGVPARFSGSGSGTD | | |
| | TVDKSTSTAYMEVSSLRSD | YSLTISSMEPEDAATYYC | | |
| | DTAVYYCARGSNPYYYAM | HQRGSYTFGGGTKLEIK | | |
| | DYWGQGTSVTVSS (SEQ | (SEQ ID NO: 187) | | |
| | ID NO: 186) | | | |
| Conatumumab | QVQLQESGPGLVKPSQTL | EIVLTQSPGTLSLSPGERA | na | na |
| (Format: Whole | SLTCTVSGGSISSGDYFWS | TLSCRASQGISRSYLAWY | | |
| mAb) | WIRQLPGKGLEWIGHIHN | QQKPGQAPSLLIYGASSR | | |
| | SGTTYYNPSLKSRVTISVDT | ATGIPDRFSGSGSGTDFT | | |
| | SKKQFSLRLSSVTAADTAV | LTISRLEPEDFAVYYCQQF | | |
| | YYCARDRGGDYYYGMDV | GSSPWTFGQGTKVEIK | | |
| | WGQGTTVTVSS (SEQ ID | (SEQ ID NO: 189) | | |
| | NO: 188) | | | |
| Concizumab | EVQLVESGGGLVKPGGSL | DIVMTQTPLSLSVTPGQP | na | na |
| (Format: Whole | RLSCAASGFTFSNYAMSW | ASISCKSSQSLLESDGKTY | | |
| mAb) | VRQTPEKRLEWVATISRSG | LNWYLQKPGQSPQLLIYL | | |
| | SYSYFPDSVQGRFTISRDN | VSILDSGVPDRFSGSGSG | | |
| | AKNSLYLQMNSLRAEDTA | TDFTLKISRVEAEDVGVY | | |
| | VYYCARLGGYDEGDAMD | YCLQATHFPQTFGGGTK | | |
| | SWGQGTTVTVSS (SEQ ID | VEIK (SEQ ID NO: 191) | | |
| | NO: 190) | | | |
| Cosfroviximab | DVKLLESGGGLVQPGGSL | DIVMTQSPLSLSTSVGDR | na | na |
| (Format: Whole | KLSCAASGFSLSTSGVGVG | VSLTCKASQNVGTAVAW | | |
| mAb) | WFRQPSGKGLEWLALIW | YQQKPGQSPKLLIYSASN | | |
| | WDDDKYYNPSLKSQLSISK | RYTGVPDRFTGSGSGTDF | | |
| | DFSRNQVFLKISNVDIADT | TLTISNMQSEDLADYFCQ | | |
| | ATYYCARRDPFGYDNAM | QYSSYPLTFGAGTKLELR | | |
| | GYWGQGTSVTVSS (SEQ | (SEQ ID NO: 193) | | |
| | ID NO: 192) | | | |
| Crenezumab | EVQLVESGGGLVQPGGSL | DIVMTQSPLSLPVTPGEP | na | na |
| (Format: Whole | RLSCAASGFTFSSYGMSW | ASISCRSSQSLVYSNGDTY | | |
| mAb) | VRQAPGKGLELVASINSN | LHWYLQKPGQSPQLLIYK | | |
| | GGSTYYPDSVKGRFTISRD | VSNRFSGVPDRFSGSGS | | |
| | NAKNSLYLQMNSLRAEDT | GTDFTLKISRVEAEDVGV | | |
| | AVYYCASGDYWGQGTTV | YYCSQSTHVPWTFGQGT | | |
| | TVSS (SEQ ID NO: 194) | KVEIK | | |
| | | (SEQ ID NO: 195) | | |
| Crizanlizumab | QVQLVQSGAEVKKPGASV | DIQMTQSPSSLSASVGD | na | na |
| (Format: Whole | KVSCKVSGYTFTSYDINWV | RVTITCKASQSVDYDGHS | | |
| mAb) | RQAPGKGLEWMGWIYPG | YMNWYQQKPGKAPKLLI | | |
| | DGSIKYNEKFKGRVTMTV | YAASNLESGVPSRFSGSG | | |
| | DKSTDTAYMELSSLRSEDT | SGTDFTLTISSLQPEDFAT | | |
| | AVYYCARRGEYGNYEGA | YYCQQSDENPLTFGGGT | | |
| | MDYWGQGTLVTVSS | KVEIK | | |
| | (SEQ ID NO: 196) | (SEQ ID NO: 197) | | |
| Crotedumab | EVQLVESGGGLVQPGGSL | DIQMTQSPSSLSASVGD | na | na |
| (Format: Whole | RLSCAASGFTFSNYLMNW | RVTITCRASQGIRNDLG | | |
| mAb) | VRQAPGKGLEWLANIQED | WYQQKPGKAPKRLIYAA | | |
| | GIEKYYVDSVKGRFTISRD | SSLQSGVPSRFSGSGSGT | | |
| | NAKNSLYLQMNSLRAEDT | EFILTVSSLQPEDFATYYC | | |
| | AVYYCAREPSHYDILTGYD | LQYNSNPFTFGPGTKVDI | | |
| | YYYGMDVWGQGTTVTVS | K (SEQ ID NO: 199) | | |
| | S (SEQ ID NO: 198) | | | |
| Crovalimab | QVQLVESGGGLVQPGRSL | DIQMTQSPSSLSASVGD | na | na |
| (Format: Whole | RLSCAASGFTVHSSYYMA | RVTITCRASQGISSSLAW | | |
| mAb) | WVRQAPGKGLEWVGAIF | YQQKPGKAPKLLIYGASE | | |
| | TGSGAEYKAEWAKGRVTI | TESGVPSRFSGSGSGTDF | | |
| | SKDTSKNQVVLTMTNMD | TLTISSLQPEDFATYYCQN | | |
| | PVDTATYYCASDAGYDYP | TKVGSSYGNTFGGGTKV | | |
| | THAMHYWGQGTLVTVSS | EIK (SEQ ID NO: 201) | | |
| | (SEQ ID NO: 200) | | | |
| Cusatuzumab | EVQLVESGGGLVQPGGSL | QAVVTQEPSLTVSPGGT | na | na |
| (Format: Whole | RLSCAASGFTFSVYYMNW | VTLTCGLKSGSVTSDNFP | | |
| mAb) | VRQAPGKGLEWVSDINNE | TWYQQTPGQAPRLLIYN | | |
| | GGTTYYADSVKGRFTISRD | TNTRHSGVPDRFSGSILG | | |
| | NSKNSLYLQMNSLRAEDT | NKAALTITGAQADDEAEY | | |
| | AVYYCARDAGYSNHVPIF | FCALFISNPSVEFGGGTQ | | |
| | DSWGQGTLVTVSS (SEQ | LTVL (SEQ ID NO: 203) | | |
| | ID NO: 202) | | | |
| Dacetuzumab | EVQLVESGGGLVQPGGSL | DIQMTQSPSSLSASVGD | na | na |
| (Format: Whole | RLSCAASGYSFTGYYIHWV | RVTITCRSSQSLVHSNGN | | |
| mAb) | RQAPGKGLEWVARVIPNA | TFLHWYQQKPGKAPKLLI | | |
| | GGTSYNQKFKGRFTLSVD | YTVSNRFSGVPSRFSGSG | | |
| | NSKNTAYLQMNSLRAEDT | SGTDFTLTISSLQPEDFAT | | |
| | AVYYCAREGIYWWGQGT | YFCSQTTHVPWTFGQGT | | |
| | LVTVSS | KVEI | | |
| | (SEQ ID NO: 204) | (SEQ ID NO: 205) | | |
| Daclizumab | QVQLVQSGAEVKKPGSSV | DIQMTQSPSTLSASVGD | na | na |
| (Format: Whole | KVSCKASGYTFTSYRMHW | RVTITCSASSSISYMHWY | | |
| mAb) | VRQAPGQGLEWIGYINPS | QQKPGKAPKLLIYTTSNL | | |
| | TGYTEYNQKFKDKATITAD | ASGVPARFSGSGSGTEFT | | |
| | ESTNTAYMELSSLRSEDTA | LTISSLQPDDFATYYCHQ | | |
| | VYYCARGGGVFDYWGQG | RSTYPLTFGQGTKVEVK | | |
| | TLVTVSS (SEQ ID NO: | (SEQ ID NO: 207) | | |
| | 206) | | | |
| Dalotuzumab | QVQLQESGPGLVKPSETLS | DIVMTQSPLSLPVTPGEP | na | na |
| (Format: Whole | LTCTVSGYSITGGYLWNWI | ASISCRSSQSIVHSNGNTY | | |
| mAb) | RQPPGKGLEWIGYISYDGT | LQWYLQKPGQSPQLLIYK | | |
| | NNYKPSLKDRVTISRDTSK | VSNRLYGVPDRFSGSGS | | |
| | NQFSLKLSSVTAADTAVYY | GTDFTLKISRVEAEDVGV | | |
| | CARYGRVFFDYWGQGTL | YYCFQGSHVPWTFGQGT | | |
| | VTVSS | KVEIK | | |
| | (SEQ ID NO: 208) | (SEQ ID NO: 209) | | |
| Dapirolizumab | EVQLVESGGGLVQPGGSL | DIQMTQSPSSLSASVGD | na | na |
| (Format: Fab) | RLSCAVSGFSSTNYHVHW | RVTITCRASEDLYYNLAW | | |
| | VRQAPGKGLEWMGVIW | YQRKPGKAPKLLIYDTYRL | | |
| | GDGDTSYNSVLKSRFTISR | ADGVPSRFSGSGSGTDYT | | |
| | DTSKNTVYLQMNSLRAED | LTISSLQPEDFASYYCQQY | | |
| | TAVYYCARQLTHYYVLAA | YKFPFTFGQGTKVEIK | | |
| | WGQGTLVTVSS (SEQ ID | (SEQ ID | | |
| | NO: 210) | NO: 211) | | |
| Daratumumab | EVQLLESGGGLVQPGGSL | EIVLTQSPATLSLSPGERA | na | na |
| (Format: Whole | RLSCAVSGFTFNSFAMSW | TLSCRASQSVSSYLAWYQ | | |
| mAb) | VRQAPGKGLEWVSAISGS | QKPGQAPRLLIYDASNRA | | |
| | GGGTYYADSVKGRFTISRD | TGIPARFSGSGSGTDFTLT | | |
| | NSKNTLYLQMNSLRAEDT | ISSLEPEDFAVYYCQQRS | | |
| | AVYFCAKDKILWFGEPVFD | NWPPTFGQGTKVEIK | | |
| | YWGQGTLVTVSS (SEQ ID | (SEQ ID NO: 213) | | |
| | NO: 212) | | | |
| Dectrekumab | EVQLVESGGGVVQPGRSL | EIVLTQSPATLSLSPGERAI | na | na |
| (Format: Whole | RLSCAASGFTFSSYGMHW | LSCRAGQSVSSYLVWYQ | | |
| mAb) | VRQAPGKGLEWVAIIWYD | QKPGQAPRLLIYDASNRA | | |
| | GSNKYYADSVKGRFTISRD | TGIPARFSGSGSGTDFTLT | | |
| | NSKNTLYLQMNSLRAEDT | ISSLEPEDFAVYYCQQRSS | | |
| | AVYYCARLWFGDLDAFDI | WPPVYTFGQGTKLEIK | | |
| | WGQGTMVTVSS (SEQ ID | (SEQ ID NO: 215) | | |
| | NO: 214) | | | |
| Demcizumab | QVQLVQSGAEVKKPGASV | DIVMTQSPDSLAVSLGER | na | na |
| (Format: Whole | KISCKASGYSFTAYYIHWV | ATISCRASESVDNYGISF | | |
| mAb) | KQAPGQGLEWIGYISSYN | MKWFQQKPGQPPKLLIY | | |
| | GATNYNQKFKGRVTFTTD | AASNQGSGVPDRFSGSG | | |
| | TSTSTAYMELRSLRSDDTA | SGTDFTLTISSLQAEDVAV | | |
| | VYYCARDYDYDVGMDYW | YYCQQSKEVPWTFGGGT | | |
| | GQGTLVTVSS (SEQ ID | KVEIK (SEQ ID | | |
| | NO: 216) | NO: 217) | | |
| Denintuzumab | QVQLQESGPGLVKPSQTL | EIVLTQSPATLSLSPGERA | na | na |
| (Format: Whole | SLTCTVSGGSISTSGMGVG | TLSCSASSSVSYMHWYQ | | |
| mAb ADC) | WIRQHPGKGLEWIGHIW | QKPGQAPRLLIYDTSKLA | | |
| | WDDDKRYNPALKSRVTIS | SGIPARFSGSGSGTDFTLT | | |
| | VDTSKNQFSLKLSSVTAAD | ISSLEPEDVAVYYCFQGSV | | |
| | TAVYYCARMELWSYYFDY | YPFTFGQGTKLEIK (SEQ | | |
| | WGQGTLVTVSS (SEQ ID | ID | | |
| | NO: 218) | NO: 219) | | |
| Denosumab | EVQLLESGGGLVQPGGSL | EIVLTQSPGTLSLSPGERA | na | na |
| (Format: Whole | RLSCAASGFTFSSYAMSW | TLSCRASQSVRGRYLAW | | |
| mAb) | VRQAPGKGLEWVSGITGS | YQQKPGQAPRLLIYGASS | | |
| | GGSTYYADSVKGRFTISRD | RATGIPDRFSGSGSGTDF | | |
| | NSKNTLYLQMNSLRAEDT | TLTISRLEPEDFAVFYCQQ | | |
| | AVYYCAKDPGTTVIMSWF | YGSSPRTFGQGTKVEIK | | |
| | DPWGQGTLVTVSS (SEQ | (SEQ ID NO: 221) | | |
| | ID NO: 220) | | | |
| Depatuxizumab | QVQLQESGPGLVKPSQTL | DIQMTQSPSSMSVSVGD | na | na |
| (Format: Whole | SLTCTVSGYSISSDFAWN | RVTITCHSSQDINSNIGW | | |
| mAb ADC) | WIRQPPGKGLEWMGYISY | LQQKPGKSFKGLIYHGTN | | |
| | SGNTRYQPSLKSRITISRDT | LDDGVPSRFSGSGSGTDY | | |
| | SKNQFFLKLNSVTAADTAT | TLTISSLQPEDFATYYCVQ | | |
| | YYCVTAGRGFPYWGQGTL | YAQFPWTFGGGTKLEIK | | |
| | VTVSS (SEQ ID NO: 222) | (SEQ ID NO: 223) | | |
| Derlotuximab | QVQLKESGPGLVAPSQSLS | ENVLTQSPAIMSASPGEK | na | na |
| (Format: Whole | ITCTVSGFSLTDYGVRWIR | VTMTCRASSSVSSSYLH | | |
| mAb | QPPGKGLEWLGVIWGGG | WYQQKSGASPKLWIYST | | |
| Radiolabelled) | STYYNSALKSRLSISKDNSK | SNLASGVPARFSGSGSGT | | |
| | SQVFLKMNSLQTDDTAM | SYSLTISSVEAEDAATYYC | | |
| | YYCAKEKRRGYYYAMDY | QQYSGYPLTFGGGTKLEI | | |
| | WGQGTSVTVSS (SEQ ID | K (SEQ ID NO: 225) | | |
| | NO: 224) | | | |
| Dezamizumab | QVQLVQSGAEVKKPGSSV | DIQMTQSPSSLSASVGD | na | na |
| (Format: Whole | KVSCKASGFTFATYNMH | RVTITCRASENIYSYLAWY | | |
| mAb) | WVRQAPGQGLEWMGYI | QQKPGKAPKLLIHNAKTL | | |
| | YPGDGNANYNQQFKGRV | AEGVPSRFSGSGSGTDFT | | |
| | TITADKSTSTAYMELSSLRS | LTISSLQPEDFATYYCQH | | |
| | EDTAVYYCARGDFDYDGG | HYGAPLTFGQGTKLEIK | | |
| | YYFDSWGQGTLVTVSS | (SEQ ID NO: 227) | | |
| | (SEQ ID NO: 226) | | | |
| Dilpacimab | EVQLVESGGGLVQPGGSL | DIQMTQSPSSLSASVGD | EVQLVESGGGLVQ | DIQMTQSPSSLSA |
| (Format: | RLSCAASGFTFSNFPMAW | RVTITCRASEDIYSNLAW | PGGSLRLSCAASGY | SVGDRVTITCSAS |
| Bispecific | VRQAPGKGLEWVATISSS | YQQKPGKAPKLLIYDTNN | TFTNYGMNWVRQ | QDISNYLNWYQQ |
| mAb) | DGTTYYRDSVKGRFTISRD | LADGVPSRFSGSGSGTDF | APGKGLEWVGWI | KPGKAPKVLIYFTS |
| | NAKNSLYLQMNSLRAEDT | TLTISSLQPEDFATYYCQQ | NTYTGEPTYAADFK | SLHSGVPSRFSGS |
| | AVYYCARGYYNSPFAYWG | YNNYPPTFGQGTKLEIK | RRFTFSLDTSKSTAY | GSGTDFTLTISSLQ |
| | QGTLVTVSS (SEQ ID | (SEQ ID NO: 229) | LQMNSLRAEDTAV | PEDFATYYCQQYS |
| | NO: 228) | | YYCAKYPHYYGSSH | TVPWTFGQGTKV |
| | | | WYFDVWGQGTLV | EIK (SEQ ID NO: |
| | | | TVSS (SEQ ID NO: | 231) |
| | | | 230) | |
| Dinutuximab | EVQLLQSGPELEKPGASV | EIVMTQSPATLSVSPGER | na | na |
| (Format: Whole | MISCKASGSSFTGYNMN | ATLSCRSSQSLVHRNGNT | | |
| mAb) | WVRQNIGKSLEWIGAIDP | YLHWYLQKPGQSPKLLIH | | |
| | YYGGTSYNQKFKGRATLT | KVSNRFSGVPDRFSGSGS | | |
| | VDKSSSTAYMHLKSLTSED | GTDFTLKISRVEAEDLGVY | | |
| | SAVYYCVSGMEYWGQGT | FCSQSTHVPPLTFGAGTK | | |
| | SVTVSS | LELK | | |
| | (SEQ ID NO: 232) | (SEQ ID NO: 233) | | |
| Diridavumab | EVQLVESGAEVKKPGSSVK | QSVLTQPPSVSAAPGQK | na | na |
| (Format: Whole | VSCKASGGPFRSYAISWVR | VTISCSGSSSNIGNDYVS | | |
| mAb) | QAPGQGPEWMGGIIPIFG | WYQQLPGTAPKLLIYDN | | |
| | TTKYAPKFQGRVTITADDF | NKRPSGIPDRFSGSKSGT | | |
| | AGTVYMELSSLRSEDTAM | SATLGITGLQTGDEANYY | | |
| | YYCAKHMGYQVRETMDV | CATWDRRPTAYVVFGG | | |
| | WGKGTTVTVSS (SEQ ID | GTKLTVL (SEQ ID NO: | | |
| | NO: 234) | 235) | | |
| Disitamab | EVQLVQSGAEVKKPGATV | DIQMTQSPSSVSASVGD | na | na |
| (Format: Whole | KISCKVSGYTFTDYYIHWV | RVTITCKASQDVGTAVA | | |
| mAb ADC) | QQAPGKGLEWMGRVNP | WYQQKPGKAPKLLIYWA | | |
| | DHGDSYYNQKFKDKATIT | SIRHTGVPSRFSGSGSGT | | |
| | ADKSTDTAYMELSSLRSED | DFTLTISSLQPEDFATYYC | | |
| | TAVYFCARNYLFDHWGQ | HQFATYTFGGGTKVEIK | | |
| | GTLVTVSS (SEQ ID NO: | (SEQ ID NO: 237) | | |
| | 236) | | | |
| Domagrozumab | EVQLLESGGGLVQPGGSL | DIQMTQSPSSLSASVGD | na | na |
| (Format: Whole | RLSCAASGFTFSSYAMSW | RVTITCKASQDVSTAVA | | |
| mAb) | VRQAPGKGLEWVSTISSG | WYQQKPGKAPKLLIYSAS | | |
| | GSYTSYPDSVKGRFTISRD | YRYTGVPSRFSGSGSGTD | | |
| | NSKNTLYLQMNSLRAEDT | FTLTISSLQPEDFATYYCQ | | |
| | AVYYCAKQDYAMNYWG | QHYSTPWTFGGGTKVEI | | |
| | QGTLVTVSS (SEQ ID NO: | K (SEQ ID NO: 239) | | |
| | 238) | | | |
| Donanemab | QVQLVQSGAEVKKPGSSV | DIVMTQTPLSLSVTPGQP | na | na |
| (Format: Whole | KVSCKASGYDFTRYYINW | ASISCKSSQSLLYSRGKTY | | |
| mAb) | VRQAPGQGLEWMGWIN | LNWLLQKPGQSPQLLIYA | | |
| | PGSGNTKYNEKFKGRVTIT | VSKLDSGVPDRFSGSGSG | | |
| | ADESTSTAYMELSSLRSED | TDFTLKISRVEAEDVGVY | | |
| | TAVYYCAREGITVYWGQG | YCVQGTHYPFTFGQGTK | | |
| | TTVTVSS (SEQ ID NO: | LEIK (SEQ ID NO: 241) | | |
| | 240) | | | |
| Dostarlimab | EVQLLESGGGLVQPGGSL | DIQLTQSPSFLSAYVGDR | na | na |
| (Format: Whole | RLSCAASGFTFSSYDMSW | VTITCKASQDVGTAVAW | | |
| mAb) | VRQAPGKGLEWVSTISGG | YQQKPGKAPKLLIYWAST | | |
| | GSYTYYQDSVKGRFTISRD | LHTGVPSRFSGSGSGTEF | | |
| | NSKNTLYLQMNSLRAEDT | TLTISSLQPEDFATYYCQH | | |
| | AVYYCASPYYAMDYWGQ | YSSYPWTFGQGTKLEIK | | |
| | GTTVTVSS (SEQ ID NO: | (SEQ ID NO: 243) | | |
| | 242) | | | |
| Drozitumab | EVQLVQSGGGVERPGGSL | SELTQDPAVSVALGQTV | na | na |
| (Format: Whole | RLSCAASGFTFDDYAMSW | RITCSGDSLRSYYASWYQ | | |
| mAb) | VRQAPGKGLEWVSGINW | QKPGQAPVLVIYGANNR | | |
| | QGGSTGYADSVKGRVTIS | PSGIPDRFSGSSSGNTASL | | |
| | RDNAKNSLYLQMNSLRAE | TITGAQAEDEADYYCNSA | | |
| | DTAVYYCAKILGAGRGWY | DSSGNHVVFGGGTKLTV | | |
| | FDYWGKGTTVTVSS (SEQ | L (SEQ ID NO: 245) | | |
| | ID NO: 244) | | | |
| Duligotuzumab | EVQLVESGGGLVQPGGSL | DIQMTQSPSSLSASVGD | na | na |
| (Format: Whole | RLSCAASGFTLSGDWIHW | RVTITCRASQNIATDVA | | |
| mAb) | VRQAPGKGLEWVGEISAA | WYQQKPGKAPKLLIYSAS | | |
| | GGYTDYADSVKGRFTISAD | FLYSGVPSRFSGSGSGTD | | |
| | TSKNTAYLQMNSLRAEDT | FTLTISSLQPEDFATYYCQ | | |
| | AVYYCARESRVSFEAAMD | QSEPEPYTFGQGTKVEIK | | |
| | YWGQGTLVTVSS (SEQ ID | (SEQ ID NO: 247) | | |
| | NO: 246) | | | |
| Dupilumab | EVQLVESGGGLEQPGGSL | DIVMTQSPLSLPVTPGEP | na | na |
| (Format: Whole | RLSCAGSGFTFRDYAMTW | ASISCRSSQSLLYSIGYNYL | | |
| mAb) | VRQAPGKGLEWVSSISGS | DWYLQKSGQSPQLLIYLG | | |
| | GGNTYYADSVKGRFTISRD | SNRASGVPDRFSGSGSG | | |
| | NSKNTLYLQMNSLRAEDT | TDFTLKISRVEAEDVGFYY | | |
| | AVYYCAKDRLSITIRPRYYG | CMQALQTPYTFGQGTKL | | |
| | LDVWGQGTTVTVSS (SEQ | EIK (SEQ ID NO: 249) | | |
| | ID NO: 248) | | | |
| Durvalumab | EVQLVESGGGLVQPGGSL | EIVLTQSPGTLSLSPGERA | na | na |
| (Format: Whole | RLSCAASGFTFSRYWMS | TLSCRASQRVSSSYLAWY | | |
| mAb) | WVRQAPGKGLEWVANIK | QQKPGQAPRLLIYDASSR | | |
| | QDGSEKYYVDSVKGRFTIS | ATGIPDRFSGSGSGTDFT | | |
| | RDNAKNSLYLQMNSLRAE | LTISRLEPEDFAVYYCQQY | | |
| | DTAVYYCAREGGWFGELA | GSLPWTFGQGTKVEIK | | |
| | FDYWGQGTLVTVSS (SEQ | (SEQ ID NO: 251) | | |
| | ID NO: 250) | | | |
| Dusigitumab | QVQLVQSGAEVKKPGASV | QSVLTQPPSVSAAPGQK | na | na |
| (Format: Whole | KVSCKASGYTFTSYDINWV | VTISCSGSSSNIENNHVS | | |
| mAb) | RQATGQGLEWMGWMN | WYQQLPGTAPKLLIYDN | | |
| | PNSGNTGYAQKFQGRVT | NKRPSGIPDRFSGSKSGT | | |
| | MTRNTSISTAYMELSSLRS | SATLGITGLQTGDEADYY | | |
| | EDTAVYYCARDPYYYYYG | CETWDTSLSAGRVFGGG | | |
| | MDVWGQGTTVTVSS | TKLTVL (SEQ ID NO: | | |
| | (SEQ ID NO: 252) | 253) | | |
| Duvortuxizumab | QVTLRESGPALVKPTQTLT | ENVLTQSPATLSVTPGEK | EVQLVESGGGLVQ | QAVVTQEPSLTVS |
| (Format: | LTCTFSGFSLSTSGMGVG | ATITCRASQSVSYMHWY | PGGSLRLSCAASGF | PGGTVTLTCRSST |
| Bispecific scFv | WIRQPPGKALEWLAHIW | QQKPGQAPRLLIYDASN | TFSTYAMNWVRQ | GAVTTSNYANW |
| with Crossover) | WDDDKRYNPALKSRLTISK | RASGVPSRFSGSGSGTD | APGKGLEWVGRIR | VQQKPGQAPRGL |
| | DTSKNQVFLTMTNMDPV | HTLTISSLEAEDAATYYCF | SKYNNYATYYADS | IGGTNKRAPWTP |
| | DTATYYCARMELWSYYFD | QGSVYPFTFGQGTKLEIK | VKGRFTISRDDSKN | ARFSGSLLGGKAA |
| | YWGQGTTVTVSS (SEQ ID | (SEQ ID NO: 255) | SLYLQMNSLKTEDT | LTITGAQAEDEAD |
| | NO: 254) | | AVYYCVRHGNFGN | YYCALWYSNLWV |
| | | | SYVSWFAYWGQG | FGGGTKLTVL |
| | | | TLVTVSS (SEQ ID | (SEQ ID NO: 257) |
| | | | NO: 256) | |
| Eculizumab | QVQLVQSGAEVKKPGASV | DIQMTQSPSSLSASVGD | na | na |
| (Format: Whole | KVSCKASGYIFSNYWIOW | RVTITCGASENIYGALNW | | |
| mAb) | VRQAPGQGLEWMGEILP | YQQKPGKAPKLLIYGATN | | |
| | GSGSTEYTENFKDRVTMT | LADGVPSRFSGSGSGTDF | | |
| | RDTSTSTVYMELSSLRSED | TLTISSLQPEDFATYYCQN | | |
| | TAVYYCARYFFGSSPNWY | VLNTPLTFGQGTKVEIK | | |
| | FDVWGQGTLVTVSS (SEQ | (SEQ ID NO: 259) | | |
| | ID NO: 258) | | | |
| Edrecolomab | QVQLQQSGAELVRPGTSV | NIVMTQSPKSMSMSVG | na | na |
| (Format: Whole | KVSCKASGYAFTNYLIEWV | ERVTLTCKASENVVTYVS | | |
| mAb) | KQRPGQGLEWIGVINPGS | WYQQKPEQSPKLLIYGAS | | |
| | GGTNYNEKFKGKATLTAD | NRYTGVPDRFTGSGSAT | | |
| | KSSSTAYMQLSSLTSDDSA | DFTLTISSVQAEDLADYH | | |
| | VYFCARDGPWFAYWGQ | CGQGYSYPYTFGGGTKLE | | |
| | GTLVTVSA (SEQ ID NO: | IK (SEQ ID NO: 261) | | |
| | 260) | | | |
| Efalizumab | EVQLVESGGGLVQPGGSL | DIQMTQSPSSLSASVGD | na | na |
| (Format: Whole | RLSCAASGYSFTGHWMN | RVTITCRASKTISKYLAWY | | |
| mAb) | WVRQAPGKGLEWVGMI | QQKPGKAPKLLIYSGSTL | | |
| | HPSDSETRYNQKFKDRFTI | QSGVPSRFSGSGSGTDFT | | |
| | SVDKSKNTLYLQMNSLRA | LTISSLQPEDFATYYCQQ | | |
| | EDTAVYYCARG1YFYGTTY | HNEYPLTFGQGTKVEIK | | |
| | FDYWGQGTLVTVSS (SEQ | (SEQ ID NO: 263) | | |
| | ID NO: 262) | | | |
| Efungumab | MAEVQLVESGAEVKKPGE | DVVMTQSPSFLSAFVGD | na | na |
| (Format: scFv) | SLRISCKGSGCIISSYWISW | RITITCRASSGISRYLAWY | | |
| | VRQMPGKGLEWMGKIDP | QQAPGKAPKLLIYAASTL | | |
| | GDSYINYSPSFQGHVTISA | QTGVPSRFSGSGSGTEFT | | |
| | DKSINTAYLQWNSLKASD | LTINSLQPEDFATYYCQH | | |
| | TAMYYCARGGRDFGDSF | LNSYPLTFGGGTKVDIK | | |
| | DYWGQGTLVTVSS (SEQ | (SEQ ID NO: 265) | | |
| | ID NO: 264) | | | |
| Eldelumab | QMQLVESGGGVVQPGRS | EIVLTQSPGTLSLSPGERA | na | na |
| (Format: Whole | LRLSCTASGFTFSNNGMH | TLSCRASQSVSSSYLAWY | | |
| mAb) | WVRQAPGKGLEWVAVI | QQKPGQAPRLLIYGASSR | | |
| | WFDGMNKFYVDSVKGRF | ATGIPDRFSGSGSGTDFT | | |
| | TISRDNSKNTLYLEMNSLR | LTISRLEPEDFAVYYCQQY | | |
| | AEDTAIYYCAREGDGSGIY | GSSPIFTFGPGTKVDIK | | |
| | YYYGMDVWGQGTTVTVS | (SEQ ID NO: 267) | | |
| | S (SEQ ID NO: 266) | | | |
| Elezanumab | EVQLVQSGAEVKKPGASV | QSALTQPRSVSGSPGQS | na | na |
| (Format: Whole | KVSCKASGYTFTSHGISWV | VTISCTGTSSSVGDSIYVS | | |
| mAb) | RQAPGQGLDWMGWISP | WYQQHPGKAPKLMLYD | | |
| | YSGNTNYAQKLQGRVTM | VTKRPSGVPDRFSGSKSG | | |
| | TTDTSTSTAYMELSSLRSE | NTASLTISGLQAEDEADY | | |
| | DTAVYYCARVGSGPYYYM | YCYSYAGTDTLFGGGTKV | | |
| | DVWGQGTLVTVSS (SEQ | TVL (SEQ ID NO: 269) | | |
| | ID NO: 268) | | | |
| Elgemtumab | EVQLLESGGGLVQPGGSL | DIQMTQSPSSLSASVGD | na | na |
| (Format: Whole | RLSCAASGFTFSSYAMSW | RVTITCRASQGISNWLA | | |
| mAb) | VRQAPGKGLEWVSAINSQ | WYQQKPGKAPKLLIYGA | | |
| | GKSTYYADSVKGRFTISRD | SSLQSGVPSRFSGSGSGT | | |
| | NSKNTLYLQMNSLRAEDT | DFTLTISSLQPEDFATYYC | | |
| | AVYYCARWGDEGFDIWG | QQYSSFPTTFGQGTKVEI | | |
| | QGTLVTVSS (SEQ ID NO: | K (SEQ ID NO: 271) | | |
| | 270) | | | |
| Elipovimab | QMQLQESGPGLVKPSETL | SDISVAPGETARISCGEKS | na | na |
| (Format: Whole | SLTCSVSGASISDSYWSWI | LGSRAVQWYQHRAGQA | | |
| mAb) | RRSPGKGLEWIGYVHKSG | PSLIIYNNQDRPSGIPERF | | |
| | DTNYNPSLKSRVHLSLDTS | SGSPDSRPGTTATLTITSV | | |
| | KNQVSLSLTGVTAADSGK | EAGDEADYYCHIWDSRV | | |
| | YYCARTLHGRRIYGIVAFN | PTKWVFGGGTTLTVL | | |
| | EWFTYFYMDVWGTGTOV | (SEQ ID NO: 273) | | |
| | TVSS (SEQ ID NO: 272) | | | |
| Elotuzumab | EVQLVESGGGLVQPGGSL | DIQMTQSPSSLSASVGD | na | na |
| (Format: Whole | RLSCAASGFDFSRYWMS | RVTITCKASQDVGIAVA | | |
| mAb) | WVRQAPGKGLEWIGEINP | WYQQKPGKVPKLLIYWA | | |
| | DSSTINYAPSLKDKFIISRD | STRHTGVPDRFSGSGSGT | | |
| | NAKNSLYLQMNSLRAEDT | DFTLTISSLQPEDVATYYC | | |
| | AVYYCARPDGNYWYFDV | QQYSSYPYTFGQGTKVEI | | |
| | WGQGTLVTVSS (SEQ ID | K (SEQ ID NO: 275) | | |
| | NO: 274) | | | |
| Emactuzumab | QVQLVQSGAEVKKPGASV | DIQMTQSPSSLSASVGD | na | na |
| (Format: Whole | KVSCKASGYTFTSYDISWV | RVTITCRASEDVNTYVSW | | |
| mAb) | RQAPGQGLEWMGVIWT | YQQKPGKAPKLLIYAASN | | |
| | DGGTNYAQKLQGRVTMT | RYTGVPSRFSGSGSGTDF | | |
| | TDTSTSTAYMELRSLRSDD | TLTISSLQPEDFATYYCQQ | | |
| | TAVYYCARDQRLYFDVWG | SFSYPTFGQGTKLEIK | | |
| | QGTTVTVSS (SEQ ID NO: | (SEQ ID NO: 277) | | |
| | 276) | | | |
| Emapalumab | EVQLLESGGGLVQPGGSL | NFMLTQPHSVSESPGKT | na | na |
| (Format: Whole | RLSCAASGFTFSSYAMSW | VTISCTRSSGSIASNYVQ | | |
| mAb) | VRQAPGKGLEWVSAISGS | WYQQRPGSSPTTVIYED | | |
| | GGSTYYADSVKGRFTISRD | NQRPSGVPDRFSGSIDSS | | |
| | NSKNTLYLQMNSLRAEDT | SNSASLTISGLKTEDEADY | | |
| | AVYYCAKDGSSGWYVPH | YCQSYDGSNRWMFGGG | | |
| | WFDPWGQGTLVTVSS | TKLTVL (SEQ ID NO: | | |
| | (SEQ ID NO: 278) | 279) | | |
| Emicizumab | QVQLVQSGSELKKPGASV | DIQMTQSPSSLSASVGD | QVQLVESGGGLVQ | DIQMTQSPSSLSA |
| (Format: | KVSCKASGYTFTDNNMD | RVTITCKASRNIERQLAW | PGGSLRLSCAASGF | SVGDRVTITCKAS |
| Bispecific mAb) | WVRQAPGQGLEWMGDI | YQQKPGQAPELLIYQASR | TFSYYDIQWVRQA | RNIERQLAWYQQ |
| | NTRSGGSIYNEEFQDRVI | KESGVPDRFSGSRYGTDF | PGKGLEWVSSISPS | KPGQAPELLIYQA |
| | MTVDKSTDTAYMELSSLR | TLTISSLQPEDIATYYCQQ | GQSTYYRREVKGR | SRKESGVPDRFSG |
| | SEDTATYHCARRKSYGYYL | YSDPPLTFGGGTKVEIK | FTISRDNSKNTLYL | SRYGTDFTLTISSL |
| | DEWGEGTLVTVSS (SEQ | (SEQ ID NO: 281) | QMNSLRAEDTAVY | QPEDIATYYCQQY |
| | ID NO: 280) | | YCARRTGREYGGG | SDPPLTFGGGTKV |
| | | | WYFDYWGQGTLV | EIK (SEQ ID |
| | | | TVSS | NO: 283) |
| | | | (SEQ ID NO: 282) | |
| Emibetuzumab | QVQLVQSGAEVKKPGASV | DIQMTQSPSSLSASVGD | na | na |
| (Format: Whole | KVSCKASGYTFTDYYMHW | RVTITCSVSSSVSSIYLHW | | |
| mAb) | VRQAPGQGLEWMGRVN | YQQKPGKAPKLLIYSTSNL | | |
| | PNRRGTTYNQKFEGRVT | ASGVPSRFSGSGSGTDFT | | |
| | MTTDTSTSTAYMELRSLRS | LTISSLQPEDFATYYCQVY | | |
| | DDTAVYYCARANWLDYW | SGYPLTFGGGTKVEIK | | |
| | GQGTTVTVSS (SEQ ID | (SEQ ID NO: 285) | | |
| | NO: 284) | | | |
| Enapotamab | EVQLLESGGGLVQPGGSL | EIVLTQSPGTLSLSPGERA | na | na |
| (Format: Whole | RLSCAASGFTFSSYAMNW | TLSCRASQSVSSSYLAWY | | |
| mAb ADC) | VRQAPGKGLEWVSTTSGS | QQKPGQAPRLLIYGASSR | | |
| | GASTYYADSVKGRFTISRD | ATGIPDRFSGSGSGTDFT | | |
| | NSKNTLYLQMNSLRAEDT | LTISRLEPEDFAVYYCQQY | | |
| | AVYYCAKIWIAFDIWGQG | GSSPYTFGQGTKLEIK | | |
| | TMVTVSS (SEQ ID NO: | (SEQ ID NO: 287) | | |
| | 286) | | | |
| Enavatuzumab | EVQLVESGGGLVQPGGSL | DIQMTQSPSSLSASVGD | na | na |
| (Format: Whole | RLSCAASGFTFSSYWMSW | RVTITCRASQSVSTSSYSY | | |
| mAb) | VRQAPGKGLEWVAEIRLK | MHWYQQKPGKAPKLLIK | | |
| | SDNYATHYAESVKGRFTIS | YASNLESGVPSRFSGSGS | | |
| | RDDSKNSLYLQMNSLRAE | GTDFTLTISSLQPEDFATY | | |
| | DTAVYYCTGYYADAMDY | YCQHSWEIPYTFGGGTK | | |
| | WGQGTLVTVSS (SEQ ID | VEIK (SEQ ID NO: 289) | | |
| | NO: 288) | | | |
| Enfortumab | EVQLVESGGGLVQPGGSL | DIQMTQSPSSVSASVGD | na | na |
| (Format: Whole | RLSCAASGFTFSSYNMNW | RVTITCRASQGISGWLA | | |
| mAb ADC) | VRQAPGKGLEWVSYISSSS | WYQQKPGKAPKFLIYAA | | |
| | STIYYADSVKGRFTISRDNA | STLQSGVPSRFSGSGSGT | | |
| | KNSLSLQMNSLRDEDTAV | DFTLTISSLQPEDFATYYC | | |
| | YYCARAYYYGMDVWGQG | QQANSFPPTFGGGTKVEI | | |
| | TTVTVSS (SEQ ID NO: | K (SEQ ID NO: 291) | | |
| | 290) | | | |
| (Format: Whole | EVQLVESGGGLVQPGGSL | DIQLTQSPSFLSASVGDR | na | na |
| mAb) | RLSCAASGFTFSSFGMHW | VTITCKASQNVDTNVAW | | |
| | VRQAPGKGLEWVAYISSD | YQQKPGKAPKALIYSASY | | |
| | SSAIYYADTVKGRFTISRDN | RYSGVPSRFSGSGSGTDF | | |
| | AKNSLYLQMNSLRDEDTA | TLTISSLQPEDFATYYCQQ | | |
| | VYYCGRGRENIYYGSRLDY | YNNYPFTFGQGTKLEIK | | |
| | WGQGTTVTVSS (SEQ ID | (SEQ ID NO: 293) | | |
| | NO: 292) | | | |
| Enokizumab | QVQLVQSGAEVKKPGSSV | DIQMTQSPSSLSASVGD | na | na |
| (Format: Whole | KVSCKASGGTFSYYWIEW | RVTITCKASQHVITHVTW | | |
| mAb) | VRQAPGQGLEWMGEILP | YQQKPGKAPKLLIYGTSY | | |
| | GSGTTNPNEKFKGRVTITA | SYSGVPSRFSGSGSGTDF | | |
| | DESTSTAYMELSSLRSEDT | TLTISSLQPEDFATYYCQQ | | |
| | AVYYCARADYYGSDYVKF | FYEYPLTFGGGTKVEIK | | |
| | DYWGQGTLVTVSS (SEQ | (SEQ ID NO: 295) | | |
| | ID NO: 294) | | | |
| Enoticumab | QVQLVESGGGVVQPGRSL | EIVLTQSPATLSLSPGERA | na | na |
| (Format: Whole | RLSCAASGFTFSSYGMHW | TLSCRASQSVSSYLAWYQ | | |
| mAb) | VRQAPGKGLEWVSFLWY | QKPGQAPRLLIYDASNRA | | |
| | DGTNKNYVESVKGRFTISR | TGIPARFSGSGSGTDFTLT | | |
| | DNSKNMLYLEMNSLRAED | ISSLEPEDFAVYYCQHRS | | |
| | TAVYYCARDHDFRSGYEG | NWPPTFGGGTKVEIK | | |
| | WFDPWGQGTLVTVSS | (SEQ ID NO: 297) | | |
| | (SEQ ID NO: 296) | | | |
| Ensituximab | QVQLKESGPDLVAPSQSLS | QVVLTQSPVIMSASPGEK | na | na |
| (Format: Whole | ITCTVSGFSLSKFGVNWVR | VTMTCSASSSISYMYWY | | |
| mAb) | QPPGKGLEWLGVIWGDG | QQKPGTSPKRWIYDTSKL | | |
| | STSYNSGLISRLSISKENSKS | ASGVPARFSGSGSGTSYS | | |
| | QVFLKLNSLQADDTATYYC | LTISNMEAGDAATYYCH | | |
| | VKPGGDYWGHGTSVTVS | QRDSYPWTFGGGTNLEI | | |
| | S (SEQ ID NO: 298) | K (SEQ ID NO: 299) | | |
| Envafolimab | QVQLVESGGGLVQPGGSL | na | na | na |
| (Format: Single | RLSCAASGKMSSRRCMA | | | |
| Domain Variable | WFRQAPGKERERVAKLLT | | | |
| Fragment; H) | TSGSTYLADSVKGRFTISRD | | | |
| | NSKNTVYLQMNSLRAEDT | | | |
| | AVYYCAADSFEDPTCTLVT | | | |
| | SS (SEQ ID NO: 300) | | | |
| Epratuzumab | QVQLVQSGAEVKKPGSSV | DIQLTQSPSSLSASVGDR | na | na |
| (Format: Whole | KVSCKASGYTFTSYWLHW | VTMSCKSSQSVLYSANH | | |
| mAb) | VRQAPGQGLEWIGYINPR | KNYLAWYQQKPGKAPKL | | |
| | NDYTEYNQNFKDKATITA | LIYWASTRESGVPSRFSG | | |
| | DESTNTAYMELSSLRSEDT | SGSGTDFTFTISSLQPEDI | | |
| | AFYFCARRDITTFYWGQG | ATYYCHQYLSSWTFGGG | | |
| | TTVTVSS (SEQ ID NO: | TKLEIK (SEQ ID NO: | | |
| | 301) | 302) | | |
| Eptinezumab | EVQLVESGGGLVQPGGSL | QVLTQSPSSLSASVGDRV | na | na |
| (Format: Whole | RLSCAVSGIDLSGYYMNW | TINCQASQSVYHNTYLA | | |
| mAb) | VRQAPGKGLEWVGVIGIN | WYQQKPGKVPKQLIYDA | | |
| | GATYYASWAKGRFTISRD | STLASGVPSRFSGSGSGT | | |
| | NSKTTVYLQMNSLRAEDT | DFTLTISSLQPEDVATYYC | | |
| | AVYFCARGDIWGQGTLVT | LGSYDCTNGDCFVFGGG | | |
| | VSS (SEQ ID NO: 303) | TKVEIK (SEQ ID NO: | | |
| | | 304) | | |
| Erenumab | QVQLVESGGGVVQPGRSL | QSVLTQPPSVSAAPGQK | na | na |
| (Format: Whole | RLSCAASGFTFSSFGMHW | VTISCSGSSSNIGNNYVS | | |
| mAb) | VRQAPGKGLEWVAVISFD | WYQQLPGTAPKLLIYDN | | |
| | GSIKYSVDSVKGRFTISRD | NKRPSGIPDRFSGSKSGT | | |
| | NSKNTLFLQMNSLRAEDT | STTLGITGLQTGDEADYY | | |
| | AVYYCARDRLNYYDSSGYY | CGTWDSRLSAVVFGGGT | | |
| | HYKYYGMAVWGQGTTVT | KLTVL (SEQ ID NO: 306) | | |
| | VSS (SEQ ID NO: 305) | | | |
| Etaracizumab | QVQLVESGGGVVQPGRSL | EIVLTQSPATLSLSPGERA | na | na |
| (Format: Whole | RLSCAASGFTFSSYDMSW | TLSCQASQSISNFLHWYQ | | |
| mAb) | VRQAPGKGLEWVAKVSS | QRPGQAPRLLIRYRSQSIS | | |
| | GGGSTYYLDTVQGRFTISR | GIPARFSGSGSGTDFTLTI | | |
| | DNSKNTLYLQMNSLRAED | SSLEPEDFAVYYCQQSGS | | |
| | TAVYYCARHLHGSFASWG | WPLTFGGGTKVEIK (SEQ | | |
| | QGTTVTVSS (SEQ ID NO: | ID NO: 308) | | |
| | 307) | | | |
| Etigilimab | QVQLQESGPGLVKPSETLS | DIQMTQSPSSLSASVGD | na | na |
| (Format: Whole | LTCAVSGYSITSDYAWNW | RVTITCKASQDVSTAVA | | |
| mAb) | IRQPPGKGLEWIGYISYSG | WYQQKPGKAPKLLIYSAS | | |
| | STSYNPSLRSRVTISRDTSK | YRYTGVPSRFSGSGSGTD | | |
| | NQFFLKLSSVTAADTAVYY | FTFTISSLQPEDIATYYCQ | | |
| | CARRQVGLGFAYWGQGT | QHYSTPWTFGQGTKVEI | | |
| | LVTVSS (SEQ ID NO: 309) | K (SEQ ID NO: 310) | | |
| Etokimab | QVQLMQSGAEVKKPGAS | DIQLTQSPSFLSASVGDR | na | na |
| (Format: Whole | VKVSCKASGYTFTSYWMH | VTITCKASQDVGTAVAW | | |
| mAb) | WVRQAPGQGLEWMGTI | YQQKPGKAPKLLIYWAST | | |
| | YPRNSNTDYNQKFKARVT | RHTGVPSRFSGSGSGTEF | | |
| | MTRDTSTSTVYMELSSLRS | TLTISSLQPEDFATYYCQQ | | |
| | EDTAVYYCARPLYYYLTSPP | AKTYPFTFGSGTKLEIK | | |
| | TLFWGQGTLVTVSS (SEQ | (SEQ ID NO: 312) | | |
| | ID NO: 311) | | | |
| Etrolizumab | EVQLVESGGGLVQPGGSL | DIQMTQSPSSLSASVGD | na | na |
| (Format: Whole | RLSCAASGFFITNNYWGW | RVTITCRASESVDDLLHW | | |
| mAb) | VRQAPGKGLEWVGYISYS | YQQKPGKAPKLLIKYASQ | | |
| | GSTSYNPSLKSRFTISRDTS | SISGVPSRFSGSGSGTDFT | | |
| | KNTFYLQMNSLRAEDTAV | LTISSLQPEDFATYYCQQ | | |
| | YYCARTGSSGYFDFWGQG | GNSLPNTFGQGTKVEIK | | |
| | TLVTVSS (SEQ ID NO: | (SEQ ID NO: 314) | | |
| | 313) | | | |
| Evinacumab | EVQLVESGGGVIQPGGSL | DIQMTQSPSTLSASVGD | na | na |
| (Format: Whole | RLSCAASGFTFDDYAMN | RVTITCRASQSIRSWLAW | | |
| mAb) | WVRQGPGKGLEWVSAIS | YQQKPGKAPKLLIYKASSL | | |
| | GDGGSTYYADSVKGRFTIS | ESGVPSRFSGSGSGTEFT | | |
| | RDNSKNSLYLQMNSLRAE | LTISSLQPDDFATYYCQQ | | |
| | DTAFFYCAKDLRNTIFGVVI | YNSYSYTFGQGTKLEIK | | |
| | PDAFDIWGQGTMVTVSS | (SEQ ID NO: 316) | | |
| | (SEQ ID NO: 315) | | | |
| Evolocumab | EVQLVQSGAEVKKPGASV | ESALTQPASVSGSPGQSI | na | na |
| (Format: Whole | KVSCKASGYTLTSYGISWV | TISCTGTSSDVGGYNSVS | | |
| mAb) | RQAPGQGLEWMGWVSF | WYQQHPGKAPKLMIYEV | | |
| | YNGNTNYAQKLQGRGTM | SNRPSGVSNRFSGSKSG | | |
| | TTDPSTSTAYMELRSLRSD | NTASLTISGLQAEDEADY | | |
| | DTAVYYCARGYGMDVW | YCNSYTSTSMVFGGGTKL | | |
| | GQGTTVTVSS (SEQ ID | TVL (SEQ ID NO: 318) | | |
| | NO: 317) | | | |
| Faricimab | EVQLVESGGGLVQPGGSL | DIQLTQSPSSLSASVGDR | QVQLVQSGAEVKK | SYVLTQPPSVSVA |
| (Format: | RLSCAASGYDFTHYGMN | VTITCSASQDISNYLNWY | PGASVKVSCKASG | PGQTARITCGGN |
| Bispecific mAb) | WVRQAPGKGLEWVGWI | QQKPGKAPKVLIYFTSSL | YTFTGYYMHWVR | NIGSKSVHWYQQ |
| | NTYTGEPTYAADFKRRFTF | HSGVPSRFSGSGSGTDFT | QAPGQGLEWMG | KPGQAPVLVVYD |
| | SLDTSKSTAYLQMNSLRAE | LTISSLQPEDFATYYCQQY | WINPNSGGTNYA | DSDRPSGIPERFS |
| | DTAVYYCAKYPYYYGTSH | STVPWTFGQGTKVEIK | QKFQGRVTMTRD | GSNSGNTATLTIS |
| | WYFDVWGQGTLVTVSS | (SEQ ID NO: 320) | TSISTAYMELSRLRS | RVEAGDEADYYC |
| | (SEQ ID NO: 319) | | DDTAVYYCARSPN | QVWDSSSDHWV |
| | | | PYYYDSSGYYYPGA | FGGGTKLTVL |
| | | | FDIWGQGTMVTV | (SEQ ID NO: 322) |
| | | | SS (SEQ ID NO: | |
| | | | 321) | |
| Farletuzumab | EVQLVESGGGVVQPGRSL | DIQLTQSPSSLSASVGDR | na | na |
| (Format: Whole | RLSCSASGFTFSGYGLSWV | VTITCSVSSSISSNNLHWY | | |
| mAb) | RQAPGKGLEWVAMISSG | QQKPGKAPKPWIYGTSN | | |
| | GSYTYYADSVKGRFAISRD | LASGVPSRFSGSGSGTDY | | |
| | NAKNTLFLQMDSLRPEDT | TFTISSLQPEDIATYYCQQ | | |
| | GVYFCARHGDDPAWFAY | WSSYPYMYTFGQGTKVE | | |
| | WGQGTPVTVSS (SEQ ID | IK (SEQ ID NO: 324) | | |
| | NO: 323) | | | |
| Fasinumab | QVQLVQSGAEVKKPGASV | DIQMTQSPSSLSASAGD | na | na |
| (Format: Whole | KVSCKVSGFTLTELSIHWV | RVTITCRASQAIRNDLGW | | |
| mAb) | RQAPGKGLEWMGGFDPE | YQQKPGKAPKRLIYAAFN | | |
| | DGETIYAQKFQGRVTMTE | LQSGVPSRFSGSGSGTEF | | |
| | DTSTDTAYMELTSLRSEDT | TLTISSLQPEDLASYYCQQ | | |
| | AVYYCSTIFGVVTNFDNW | YNRYPWTFGQGTKVEIK | | |
| | GQGTLVTVSS (SEQ ID | (SEQ ID NO: 326) | | |
| | NO: 325) | | | |
| Fezakinumab | QVQLVQSGAEVKKPGASV | QAVLTQPPSVSGAPGQR | na | na |
| (Format: Whole | KVSCKASGYTFTNYYMH | VTISCTGSSSNIGAGYGV | | |
| mAb) | WVRQAPGQGLEWVGWI | HWYQQLPGTAPKLLIYG | | |
| | NPYTGSAFYAQKFRGRVT | DSNRPSGVPDRFSGSKS | | |
| | MTRDTSISTAYMELSRLRS | GTSASLAITGLQAEDEAD | | |
| | DDTAVYYCAREPEKFDSD | YYCQSYDNSLSGYVFGG | | |
| | DSDVWGRGTLVTVSS | GTQLTVL (SEQ ID NO: | | |
| | (SEQ ID NO: 327) | 328) | | |
| Ficlatuzumab | QVQLVQPGAEVKKPGTSV | DIVMTQSPDSLAMSLGE | na | na |
| (Format: Whole | KLSCKASGYTFTTYWMH | RVTLNCKASENVVSYVS | | |
| mAb) | WVRQAPGQGLEWIGEIN | WYQQKPGQSPKLLIYGA | | |
| | PTNGHTNYNQKFQGRATL | SNRESGVPDRFSGSGSAT | | |
| | TVDKSTSTAYMELSSLRSE | DFTLTISSVQAEDVADYH | | |
| | DTAVYYCARNYVGSIFDY | CGQSYNYPYTFGQGTKLE | | |
| | WGQGTLLTVSS (SEQ ID | IK (SEQ ID NO: 330) | | |
| | NO: 329) | | | |
| Figitumumab | EVQLLESGGGLVQPGGSL | DIQMTQFPSSLSASVGD | na | na |
| (Format: Whole | RLSCTASGFTFSSYAMNW | RVTITCRASQGIRNDLG | | |
| mAb) | VRQAPGKGLEWVSAISGS | WYQQKPGKAPKRLIYAA | | |
| | GGTTFYADSVKGRFTISRD | SRLHRGVPSRFSGSGSGT | | |
| | NSRTTLYLQMNSLRAEDT | EFTLTISSLQPEDFATYYCL | | |
| | AVYYCAKDLGWSDSYYYY | QHNSYPCSFGQGTKLEIK | | |
| | YGMDVWGQGTTVTVSS | (SEQ ID NO: 332) | | |
| | (SEQ ID NO: 331) | | | |
| Firivumab | QVQLVQSGAEVKMPGSS | EIVLTQSPATLSLSPGERA | na | na |
| (Format: Whole | VKVSCKTSGVFFSSHAISW | TLSCRASENIWNNLAWY | | |
| mAb) | VRQAPGQGLEWMGGISP | QQKPGQAPRLLISGASTG | | |
| | MFGTTHYAQKFQGRVTIT | ATGVPSRFRGSGSRTEFT | | |
| | ADQSTTTAYMELTSLTSED | LTISSLQSEDFAIYFCQQY | | |
| | TAVYYCARDGAGSYYPLN | NSWPRTFGPGTKVEIK | | |
| | WFDPWGQGTLVTVSS | (SEQ ID NO: 334) | | |
| | (SEQ ID NO: 333) | | | |
| Flanvotumab | QVQLVQSGSELKKPGASV | EIVLTQSPATLSLSPGERA | na | na |
| (Format: Whole | KISCKASGYTFTSYAMNW | TLSCRASQSVSSYLAWYQ | | |
| mAb) | VRQAPGQGLESMGWINT | QKPGQAPRLLIYDASNRA | | |
| | NTGNPTYAQGFTGRFVFS | TGIPARFSGSGSGTDFTLT | | |
| | MDTSVSTAYLQISSLKAED | ISSLEPEDFAVYYCQQRS | | |
| | TAIYYCAPRYSSSWYLDYW | NWLMYTFGQGTKLEIK | | |
| | GQGTLVTVSS (SEQ ID | (SEQ ID NO: 336) | | |
| | NO: 335) | | | |
| Fletikumab | QVQLVQSGAEVKRPGASV | AIQLTQSPSSLSASVGDR | na | na |
| (Format: Whole | KVSCKASGYTFTNDIIHWV | VTITCRASQGISSALAWY | | |
| mAb) | RQAPGQRLEWMGWINA | QQKPGKAPKLLIYDASSL | | |
| | GYGNTQYSQNFQDRVSIT | ESGVPSRFSGSGSGTDFT | | |
| | RDTSASTAYMELISLRSED | LTISSLQPEDFATYYCQQF | | |
| | TAVYYCAREPLWFGESSP | NSYPLTFGGGTKVEIK | | |
| | HDYYGMDVWGQGTTVT | (SEQ ID NO: 338) | | |
| | VSS (SEQ ID NO: 337) | | | |
| Flotetuzumab | EVQLVQSGAELKKPGASV | DFVMTQSPDSLAVSLGE | EVQLVESGGGLVQ | QAVVTQEPSLTVS |
| (Format: | KVSCKASGYTFTDYYMKW | RVTMSCKSSQSLLNSGN | PGGSLRLSCAASGF | PGGTVTLTCRSST |
| Bispecific scFv | VRQAPGQGLEWIGDIIPS | QKNYLTWYQQKPGQPP | TFSTYAMNWVRQ | GAVTTSNYANW |
| with Crossover) | NGATFYNQKFKGRVTITV | KLLIYWASTRESGVPDRF | APGKGLEWVGRIR | VQQKPGQAPRGL |
| | DKSTSTAYMELSSLRSEDT | SGSGSGTDFTLTISSLQAE | SKYNNYATYYADS | IGGTNKRAPWTP |
| | AVYYCARSHLLRASWFAY | DVAVYYCQNDYSYPYTF | VKDRFTISRDDSKN | ARFSGSLLGGKAA |
| | WGQGTLVTVSS (SEQ ID | GQGTKLEIK (SEQ ID NO: | SLYLQMNSLKTEDT | LTITGAQAEDEAD |
| | NO: 339) | 340) | AVYYCVRHGNFGN | YYCALWYSNLWV |
| | | | SYVSWFAYWGQG | FGGGTKLTVL |
| | | | TLVTVSS (SEQ ID | (SEQ ID NO: 342) |
| | | | NO: 341) | |
| Fontolizumab | QVQLVQSGAELKKPGSSV | DIQMTQSPSTLSASVGD | na | na |
| (Format: Whole | KVSCKASGYIFTSSWINWV | RVTITCKASENVDTYVSW | | |
| mAb) | KQAPGQGLEWIGRIDPSD | YQQKPGKAPKLLIYGASN | | |
| | GEVHYNQDFKDKATLTVD | RYTGVPSRFSGSGSGTDF | | |
| | KSTNTAYMELSSLRSEDTA | TLTISSLQPDDFATYYCG | | |
| | VYYCARGFLPWFADWGQ | QSYNYPFTFGQGTKVEV | | |
| | GTLVTVSS (SEQ ID NO: | K (SEQ ID NO: 344) | | |
| | 343) | | | |
| Foralumab | QVQLVESGGGVVQPGRSL | EIVLTQSPATLSLSPGERA | na | na |
| (Format: Whole | RLSCAASGFKFSGYGMH | TLSCRASQSVSSYLAWYQ | | |
| mAb) | WVRQAPGKGLEWVAVI | QKPGQAPRLLIYDASNRA | | |
| | WYDGSKKYYVDSVKGRFT | TGIPARFSGSGSGTDFTLT | | |
| | ISRDNSKNTLYLQMNSLRA | ISSLEPEDFAVYYCQQRS | | |
| | EDTAVYYCARQMGYWHF | NWPPLTFGGGTKVEIK | | |
| | DLWGRGTLVTVSS (SEQ | (SEQ ID NO: 346) | | |
| | ID NO: 345) | | | |
| Foravirumab | QVQLVESGGGAVQPGRSL | DIQMTQSPSSLSASVGD | na | na |
| (Format: Whole | RLSCAASGFTFSSYGMHW | RVTITCRASQGIRNDLG | | |
| mAb) | VRQAPGKGLEWVAVILYD | WYQQKPGKAPKLLIYAA | | |
| | GSDKFYADSVKGRFTISRD | SSLQSGVPSRFSGSGSGT | | |
| | NSKNTLYLQMNSLRAEDT | DFTLTISSLQPEDFATYYC | | |
| | AVYYCAKVAVAGTHFDY | QQLNSYPPTFGGGTKVEI | | |
| | WGQGTLVTVSS (SEQ ID | K (SEQ ID NO: 348) | | |
| | NO: 347) | | | |
| Fremanezumab | EVQLVESGGGLVQPGGSL | EIVLTQSPATLSLSPGERA | na | na |
| (Format: Whole | RLSCAASGFTFSNYWISW | TLSCKASKRVTTYVSWYQ | | |
| mAb) | VRQAPGKGLEWVAEIRSE | QKPGQAPRLLIYGASNRY | | |
| | SDASATHYAEAVKGRFTIS | LGIPARFSGSGSGTDFTLT | | |
| | RDNAKNSLYLQMNSLRAE | ISSLEPEDFAVYYCSQSYN | | |
| | DTAVYYCLAYFDYGLAIQN | YPYTFGQGTKLEIK (SEQ | | |
| | YWGQGTLVTVSS (SEQ ID | ID NO: 350) | | |
| | NO: 349) | | | |
| Fresolimumab | QVQLVQSGAEVKKPGSSV | ETVLTQSPGTLSLSPGER | na | na |
| (Format: Whole | KVSCKASGYTFSSNVISWV | ATLSCRASQSLGSSYLAW | | |
| mAb) | RQAPGQGLEWMGGVIPI | YQQKPGQAPRLLIYGASS | | |
| | VDIANYAQRFKGRVTITAD | RAPGIPDRFSGSGSGTDF | | |
| | ESTSTTYMELSSLRSEDTA | TLTISRLEPEDFAVYYCQQ | | |
| | VYYCASTLGLVLDAMDYW | YADSPITFGQGTRLEIK | | |
| | GQGTLVTVSS (SEQ ID | (SEQ ID NO: 352) | | |
| | NO: 351) | | | |
| Frovocimab | EVQLVESGGGLVKPGGSL | DIVMTQSPLSLPVTPGEP | na | na |
| (Format: Whole | RLSCAASGFPFSKLGMVW | ASISCRSSKSLLHRNGITYS | | |
| mAb) | VRQAPGKGLEWVSTISSG | YWYLQKPGQSPQLLIYQL | | |
| | GGYTYYPDSVKGRFTISRD | SNLASGVPDRFSGSGSGT | | |
| | NAKNSLYLQMNSLRAEDT | DFTLKISRVEAEDVGVYY | | |
| | AVYYCAREGISFQGGTYTY | CYQNLELPLTFGQGTKVE | | |
| | VMDYWGQGTLVTVSS | IK (SEQ ID NO: 354) | | |
| | (SEQ ID NO: 353) | | | |
| Frunevetmab | QVQLVESGAELVQPGESL | DIEMTQSPLSLSVTPGES | na | na |
| (Format: Feline | RLTCAASGFSLTNNNVNW | VSISCRASEDIYNALAWYL | | |
| Whole mAb) | VRQAPGKGLEWMGGVW | QKPGRSPRLLIYNTDTLH | | |
| | AGGATDYNSALKSRLTITR | TGVPDRFSGSGSGTDFTL | | |
| | DTSKNTVFLQMHSLQSED | KISRVQTEDVGVYFCQHY | | |
| | TATYYCARDGGYSSSTLYA | FHYPRTFGQGTKLELK | | |
| | MDAWGQGTTVTVSA | (SEQ ID NO: 356) | | |
| | (SEQ ID NO: 355) | | | |
| Fulranumab | EVQLVESGGGLVQPGGSL | AIQLTQSPSSLSASVGDR | na | na |
| (Format: Whole | RLSCAASGFTLRSYSMNW | VTITCRASQGISSALAWY | | |
| mAb) | VRQAPGKGLEWVSYISRSS | QQKPGKAPKLLIYDASSL | | |
| | HTIFYADSVKGRFTISRDN | ESGVPSRFSGSGSGTDFT | | |
| | AKNSLYLQMDSLRDEDTA | LTISSLQPEDFATYYCQQF | | |
| | MYYCARVYSSGWHVSDY | NSYPLTFGGGTKVEIK | | |
| | FDYWGQGILVTVSS (SEQ | (SEQ ID NO: 358) | | |
| | ID NO: 357) | | | |
| Futuximab | EVQLQQPGSELVRPGASV | DIQMTQTTSSLSASLGDR | na | na |
| (Zatuximab) | KLSCKASGYTFTSYWMH | VTISCRTSQDIGNYLNWY | | |
| (Format: Whole | WVKQRPGQGLEWIGNIY | QQKPDGTVKLLIYYTSRL | | |
| mAb) | PGSRSTNYDEKFKSKATLT | HSGVPSRFSGSGSGTDFS | | |
| | VDTSSSTAYMQLSSLTSED | LTINNVEQEDVATYFCQH | | |
| | SAVYYCTRNGDYYVSSGD | YNTVPPTFGGGTKLEIK | | |
| | AMDYWGQGTSVTVSS | (SEQ ID NO: 360) | | |
| | (SEQ ID NO: 359) | | | |
| Galcanezumab | QVQLVQSGAEVKKPGSSV | DIQMTQSPSSLSASVGD | na | na |
| (Format: Whole | KVSCKASGYTFGNYWMQ | RVTITCRASKDISKYLNW | | |
| mAb) | WVRQAPGQGLEWMGAI | YQQKPGKAPKLLIYYTSG | | |
| | YEGTGKTVYIQKFADRVTI | YHSGVPSRFSGSGSGTDF | | |
| | TADKSTSTAYMELSSLRSE | TLTISSLQPEDFATYYCQQ | | |
| | DTAVYYCARLSDYVSGFGY | GDALPPTFGGGTKVEIK | | |
| | WGQGTTVTVSS (SEQ ID | (SEQ ID NO: 362) | | |
| | NO: 361) | | | |
| Gallximab | QVQLQESGPGLVKPSETLS | ESALTQPPSVSGAPGQK | na | na |
| (Format: Whole | LTCAVSGGSISGGYGWG | VTISCTGSTSNIGGYDLH | | |
| mAb) | WIRQPPGKGLEWIGSFYS | WYQQLPGTAPKLLIYDIN | | |
| | SSGNTYYNPSLKSQVTIST | KRPSGISDRFSGSKSGTA | | |
| | DTSKNQFSLKLNSMTAAD | ASLAITGLQTEDEADYYC | | |
| | TAVYYCVRDRLFSVVGMV | QSYDSSLNAQVFGGGTR | | |
| | YNNWFDVWGPGVLVTVS | LTVL (SEQ ID NO: 364) | | |
| | S (SEQ ID NO: 363) | | | |
| Gancotamab | QVQLVESGGGLVQPGGSL | QSVLTQPPSVSGAPGQR | na | na |
| (Format: scFv) | RLSCAASGFTFRSYAMSW | VTISCTGSSSNIGAGYGV | | |
| | VRQAPGKGLEWVSAISGR | HWYQQLPGTAPKLLIYG | | |
| | GDNTYYADSVKGRFTISRD | NTNRPSGVPDRFSGFKS | | |
| | NSKNTLYLQMNSLRAEDT | GTSASLAITGLQAEDEAD | | |
| | AVYYCAKMTSNAFAFDY | YYCQSYDSSLSGWVFGG | | |
| | WGQGTLVTVSS (SEQ ID | GTKLTVL (SEQ ID NO: | | |
| | NO: 365) | 366) | | |
| Ganitumab | QVQLQESGPGLVKPSGTL | DVVMTQSPLSLPVTPGE | na | na |
| (Format: Whole | SLTCAVSGGSISSSNWWS | PASISCRSSQSLLHSNGY | | |
| mAb) | WVRQPPGKGLEWIGEIYH | NYLDWYLQKPGQSPQLL | | |
| | SGSTNYNPSLKSRVTISVD | IYLGSNRASGVPDRFSGS | | |
| | KSKNQFSLKLSSVTAADTA | GSGTDFTLKISRVEAEDV | | |
| | VYYCARWTGRTDAFDIW | GVYYCMQGTHWPLTFG | | |
| | GQGTMVTVSS (SEQ ID | QGTKVEIK (SEQ ID NO: | | |
| | NO: 367) | 368) | | |
| Gantenerumab | QVELVESGGGLVQPGGSL | DIVLTQSPATLSLSPGERA | na | na |
| (Format: Whole | RLSCAASGFTFSSYAMSW | TLSCRASQSVSSSYLAWY | | |
| mAb) | VRQAPGKGLEWVSAINAS | QQKPGQAPRLLIYGASSR | | |
| | GTRTYYADSVKGRFTISRD | ATGVPARFSGSGSGTDFT | | |
| | NSKNTLYLQMNSLRAEDT | LTISSLEPEDFATYYCLQIY | | |
| | AVYYCARGKGNTHKPYGY | NMPITFGQGTKVEIK | | |
| | VRYFDVWGQGTLVTVSS | (SEQ ID NO: 370) | | |
| | (SEQ ID NO: 369) | | | |
| Garadacimab | EVQLLESGGGLVQPGGSL | QSVLTQPPSASGTPGQR | na | na |
| (Format: Whole | RLSCAASGFTFSKYIMQW | VTISCSGSSSNIGRNYVY | | |
| mAb) | VRQAPGKGLEWVSGIDIP | WYQQLPGTAPKLLIYSN | | |
| | TKGTVYADSVKGRFTISRD | NQRPSGVPDRFSGSKSG | | |
| | NSKNTLYLQMNSLRAEDT | TSASLAISGLRSEDEADYY | | |
| | AVYYCARALPRSGYLISPH | CAAWDASLRGVFGGGT | | |
| | YYYYALDVWGQGTTVTVS | KLTVL (SEQ ID NO: 372) | | |
| | S (SEQ ID NO: 371) | | | |
| Garetosmab | QVQLQESGPGLVKPSETLS | EIVLTQSPGTLSLSPGERA | na | na |
| (Format: Whole | LTCTVSGGSFSSHFWSWI | TLSCRASQSVSSSYLAWY | | |
| mAb) | RQPPGKGLEWIGYILYTGG | QQKPGQAPRLLIYGASSR | | |
| | TSFNPSLKSRVSMSVGTSK | ATGIPDRFSGSGSGTDFT | | |
| | NQFSLKLSSVTAADTAVYY | LTISRLEPEDFAVYYCQQY | | |
| | CARARSGITFTGIIVPGSFD | GSSPWTFGQGTKVEIK | | |
| | IWGQGTMVTVSS (SEQ | (SEQ ID NO: 374) | | |
| | ID NO: 373) | | | |
| Gatipotuzumab | EVQLVESGGGLVQPGGS | DIVMTQSPLSNPVTPGEP | na | na |
| (Format: Whole | MRLSCVASGFPFSNYWM | ASISCRSSKSLLHSNGITYF | | |
| mAb) | NWVRQAPGKGLEWVGEI | FWYLQKPGQSPQLLIYQ | | |
| | RLKSNNYTTHYAESVKGRF | MSNLASGVPDRFSGSGS | | |
| | TISRDDSKNSLYLQMNSLK | GTDFTLRISRVEAEDVGV | | |
| | TEDTAVYYCTRHYYFDYW | YYCAQNLELPPTFGQGTK | | |
| | GQGTLVTVSS (SEQ ID | VEIK (SEQ ID NO: 376) | | |
| | NO: 375) | | | |
| Gedivumab | EVQLVESGGGVVQPGKSL | EIVLTQSPATLSVSPGERA | na | na |
| (Format: Whole | RLSCAASGLTFSSYAVHW | TLSCRASQVISHNLAWY | | |
| mAb) | VRQAPGKGLEWVTLISYD | QQKPGQAPRLLIYGASTR | | |
| | GANQYYADSVKGRFTISR | ASGIPARFSGSGSGTDYT | | |
| | DNSKNTVYLQMNSLRPED | LTITSLQSEDFAVYYCQHY | | |
| | TAVYYCAVPGPVFGIFPP | SNWPPRLTFGGGTKVEIK | | |
| | WSYFDNWGQGILVTVSS | (SEQ ID NO: 378) | | |
| | (SEQ ID NO: 377) | | | |
| Gemtuzumab | EVQLVQSGAEVKKPGSSV | DIQLTQSPSTLSASVGDR | na | na |
| (Format: Whole | KVSCKASGYTITDSNIHWV | VTITCRASESLDNYGIRFL | | |
| mAb ADC) | RQAPGQSLEWIGYIYPYN | TWFQQKPGKAPKLLMY | | |
| | GGTDYNQKFKNRATLTVD | AASNQGSGVPSRFSGSG | | |
| | NPTNTAYMELSSLRSEDTA | SGTEFTLTISSLQPDDFAT | | |
| | FYYCVNGNPWLAYWGQ | YYCQQTKEVPWSFGQGT | | |
| | GTLVTVSS (SEQ ID NO: | KVEVK (SEQ ID NO: | | |
| | 379) | 380) | | |
| Gevokizumab | QVQLQESGPGLVKPSQTL | DIQMTQSTSSLSASVGDR | na | na |
| (Format: Whole | SLTCSFSGFSLSTSGMGVG | VTITCRASQDISNYLSWY | | |
| mAb) | WIRQPSGKGLEWLAHIW | QQKPGKAVKLLIYYTSKL | | |
| | WDGDESYNPSLKSRLTISK | HSGVPSRFSGSGSGTDYT | | |
| | DTSKNQVSLKITSVTAADT | LTISSLQQEDFATYFCLQG | | |
| | AVYFCARNRYDPPWFVD | KMLPWTFGQGTKLEIK | | |
| | WGQGTLVTVSS (SEQ ID | (SEQ ID NO: 382) | | |
| | NO: 381) | | | |
| Gilvetmab | EVQLVQSGGDLVKPGGSV | DIVMTQTPLSLSVSLGEP | na | na |
| (Format: Canine | RLSCVASGFNIKNTYMHW | ASISCHASQNINVWLSW | | |
| Whole mAb) | VRQAPGKGLQWIGRIAPA | YRQKPGQIPQLLIYKASHL | | |
| | NVDTKYAPKFQGKATISA | HTGVPDRFSGSGSGTDF | | |
| | DTAKNTAYMQLNSLRAED | TLRISRVEADDAGVYYCQ | | |
| | TAVYYCVLIYYDYDGDIDV | QGQSWPLTFGQGTKVEI | | |
| | WGQGTLVTVSS (SEQ ID | K (SEQ ID NO: 384) | | |
| | NO: 383) | | | |
| Gimsilumab | EVQLVESGGGLVQPGGSL | EIVLTQSPVTLSVSPGERV | na | na |
| (Format: Whole | RLSCAASGFTFSRHWMH | TLSCRASQSVSTNLAWY | | |
| mAb) | WLRQVPGKGPVWVSRIN | QQKLGQGPRLLIYGASTR | | |
| | GAGTSITYADSVRGRFTISR | ATDIPARFSGSGSETEFTL | | |
| | DNANNTLFLQMNSLRAD | TISSLQSEDFAVYYCQQY | | |
| | DTALYFCARANSVWFRGL | DKWPDTFGQGTKLEIK | | |
| | FDYWGQGTPVTVSS (SEQ | (SEQ ID NO: 386) | | |
| | ID NO: 385) | | | |
| Girentuximab | DVKLVESGGGLVKLGGSLK | DIVMTQSQRFMSTTVGD | na | na |
| (Format: Whole | LSCAASGFTFSNYYMSWV | RVSITCKASQNVVSAVA | | |
| mAb | RQTPEKRLELVAAINSDGG | WYQQKPGQSPKLLIYSAS | | |
| Radiolabelled) | ITYYLDTVKGRFTISRDNAK | NRYTGVPDRFTGSGSGT | | |
| | NTLYLQMSSLKSEDTALFY | DFTLTISNMQSEDLADFF | | |
| | CARHRSGYFSMDYWGQG | CQQYSNYPWTFGGGTKL | | |
| | TSVTVSS (SEQ ID NO: | EIK (SEQ ID NO: 388) | | |
| | 387) | | | |
| Glembatumumab | QVQLQESGPGLVKPSQTL | EIVMTQSPATLSVSPGER | na | na |
| (Format: | SLTCTVSGGSISSFNYYWS | ATLSCRASQSVDNNLVW | | |
| Whole mAb ADC) | WIRHHPGKGLEWIGYIYYS | YQQKPGQAPRLLIYGAST | | |
| | GSTYSNPSLKSRVTISVDTS | RATGIPARFSGSGSGTEF | | |
| | KNQFSLTLSSVTAADTAVY | TLTISSLQSEDFAVYYCQQ | | |
| | YCARGYNWNYFDYWGQ | YNNWPPWTFGQGTKVE | | |
| | GTLVTVSS (SEQ ID NO: | IK (SEQ ID NO: 390) | | |
| | 389) | | | |
| Glenzocimab | QVQLVQSGAEVKKPGASV | DIQMTQSPSSLSASVGD | na | na |
| (Format: Fab) | KVSCKASGYTFTSYNMHW | RVTITCRSSQSLENSNGN | | |
| | VRQAPGQGLEWMGGIYP | TYLNWYQQKPGKAPKLLI | | |
| | GNGDTSYNQKFQGRVTM | YRVSNRFSGVPSRFSGSG | | |
| | TRDTSTSTVYMELSSLRSE | SGRDFTFTISSLQPEDIAT | | |
| | DTAVYYCARGTVVGDWY | YYCLQLTHVPWTFGQGT | | |
| | FDVWGQGTLVTVSS (SEQ | KVEIT (SEQ ID NO: 392) | | |
| | ID NO: 391) | | | |
| Golimumab | QVQLVESGGGVVQPGRSL | EIVLTQSPATLSLSPGERA | na | na |
| (Format: Whole | RLSCAASGFIFSSYAMHW | TLSCRASQSVYSYLAWYQ | | |
| mAb) | VRQAPGNGLEWVAFMSY | QKPGQAPRLLIYDASNRA | | |
| | DGSNKKYADSVKGRFTISR | TGIPARFSGSGSGTDFTLT | | |
| | DNSKNTLYLQMNSLRAED | ISSLEPEDFAVYYCQQRS | | |
| | TAVYYCARDRGIAAGGNY | NWPPFTFGPGTKVDIK | | |
| | YYYGMDVWGQGTTVTVS | (SEQ ID NO: 394) | | |
| | S (SEQ ID NO: 393) | | | |
| Gosuranemab | EVHLVESGGALVKPGGSLR | DVVMTQSPLSLPVTLGQ | na | na |
| (Format: Whole | LSCAASGFSFSKYGMSWV | PASISCKSSQSIVHSNGNT | | |
| mAb) | RQAPGKGLEWVATISSSG | YLEWYLQKPGQSPQLLV | | |
| | SRTYYPDSVKGRFTISRDN | YKVSNRFSGVPDRFSGSG | | |
| | AKNTLYLQMNSLRAEDTA | SGTDFTLKISRVEAEDVG | | |
| | MYYCSISWDGAMDYWG | TYYCFQGSLVPWAFGGG | | |
| | QGTTVTVSS (SEQ ID NO: | TKVEIK (SEQ ID NO: | | |
| | 395) | 396) | | |
| Guselkumab | EVQLVQSGAEVKKPGESL | QSVLTQPPSVSGAPGQR | na | na |
| (Format: Whole | KISCKGSGYSFSNYWIGW | VTISCTGSSSNIGSGYDVH | | |
| mAb) | VRQMPGKGLEWMGIIDP | WYQQLPGTAPKLLIYGNS | | |
| | SNSYTRYSPSFQGQVTISA | KRPSGVPDRFSGSKSGTS | | |
| | DKSISTAYLQWSSLKASDT | ASLAITGLQSEDEADYYC | | |
| | AMYYCARWYYKPFDVWG | ASWTDGLSLVVFGGGTK | | |
| | QGTLVTVSS (SEQ ID NO: | LTVL (SEQ ID NO: 398) | | |
| | 397) | | | |
| Ianalumab | QVQLQQSGPGLVKPSQTL | DIVLTQSPATLSLSPGERA | na | na |
| (Format: Whole | SLTCAISGDSVSSNSAAWG | TLSCRASQFILPEYLSWYQ | | |
| mAb) | WIRQSPGRGLEWLGRIYY | QKPGQAPRLLIYGSSSRA | | |
| | RSKWYNSYAVSVKSRITIN | TGVPARFSGSGSGTDFTL | | |
| | PDTSKNQFSLQLNSVTPED | TISSLEPEDFAVYYCQQFY | | |
| | TAVYYCARYQWVPKIGVF | SSPLTFGQGTKVEIK | | |
| | DSWGQGTLVTVSS (SEQ | (SEQ ID NO: 400) | | |
| | ID NO: 399) | | | |
| Ibalizumab | QVQLQQSGPEVVKPGAS | DIVMTQSPDSLAVSLGER | na | na |
| (Format: Whole | VKMSCKASGYTFTSYVIH | VTMNCKSSQSLLYSTNQ | | |
| mAb) | WVRQKPGQGLDWIGYIN | KNYLAWYQQKPGQSPKL | | |
| | PYNDGTDYDEKFKGKATL | LIYWASTRESGVPDRFSG | | |
| | TSDTSTSTAYMELSSLRSE | SGSGTDFTLTISSVQAED | | |
| | DTAVYYCAREKDNYATGA | VAVYYCQQYYSYRTFGG | | |
| | WFAYWGQGTLVTVSS | GTKLEIK (SEQ ID NO: | | |
| | (SEQ ID NO: 401) | 402) | | |
| Icrucumab | QAQVVESGGGVVQSGRS | EIVLTQSPGTLSLSPGERA | na | na |
| (Format: Whole | LRLSCAASGFAFSSYGMH | TLSCRASQSVSSSYLAWY | | |
| mAb) | WVRQAPGKGLEWVAVI | QQKPGQAPRLLIYGASSR | | |
| | WYDGSNKYYADSVRGRFT | ATGIPDRFSGSGSGTDFT | | |
| | ISRDNSENTLYLQMNSLRA | LTISRLEPEDFAVYYCQQY | | |
| | EDTAVYYCARDHYGSGVH | GSSPLTFGGGTKVEIK | | |
| | HYFYYGLDVWGQGTTVT | (SEQ ID NO: 404) | | |
| | VSS (SEQ ID NO: 403) | | | |
| Idarucizumab | QVQLQESGPGLVKPSETLS | DVVMTQSPLSLPVTLGQ | na | na |
| (Format: Fab) | LTCTVSGFSLTSYIVDWIRQ | PASISCKSSQSLLYTDGKT | | |
| | PPGKGLEWIGVIWAGGST | YLYWFLQRPGQSPRRLIY | | |
| | GYNSALRSRVSITKDTSKN | LVSKLDSGVPDRFSGSGS | | |
| | QFSLKLSSVTAADTAVYYC | GTDFTLKISRVEAEDVGV | | |
| | ASAAYYSYYNYDGFAYWG | YYCLQSTHFPHTFGGGTK | | |
| | QGTLVTVSS (SEQ ID NO: | VEIK (SEQ ID NO: 406) | | |
| | 405) | | | |
| leramilimab | QVQLVQSGAEVKKPGASV | DIQMTQSPSSLSASVGD | na | na |
| (Format: Whole | KVSCKASGFTLTNYGMN | RVTITCSSSQDISNYLNW | | |
| mAb) | WVRQARGQRLEWIGWIN | YLQKPGQSPQLLIYYTSTL | | |
| | TDTGEPTYADDFKGRFVFS | HLGVPSRFSGSGSGTEFT | | |
| | LDTSVSTAYLQISSLKAEDT | LTISSLQPDDFATYYCQQ | | |
| | AVYYCARNPPYYYGTNNA | YYNLPWTFGQGTKVEIK | | |
| | EAMDYWGQGTTVTVSS | (SEQ ID NO: 408) | | |
| | (SEQ ID NO: 407) | | | |
| Ifabotuzumab | QVQLVQSGAEVKKPGASV | DIQMTQSPSFLSASVGD | na | na |
| (Format: Whole | KVSCKASGYTFTGYWMN | RVTITCRASQGIISYLAWY | | |
| mAb) | WVRQAPGQGLEWMGDI | QQKPEKAPKRLIYAASSL | | |
| | YPGSGNTNYDEKFQGRVT | QSGVPSRFSGSGSGTEFT | | |
| | MTRDTSISTAYMELSRLRS | LTISSLQPEDFATYYCGQY | | |
| | DDTAVYYCARGGYYEDFD | ANYPYTFGQGTKLEIK | | |
| | SWGQGTTVTVSS (SEQ ID | (SEQ ID NO: 410) | | |
| | NO: 409) | | | |
| lladatuzumab | EVQLVESGGGLVQPGGSL | DIQLTQSPSSLSASVGDR | na | na |
| (Format: Whole | RLSCAASGYTFSSYWIEWV | VTITCKASQSVDYEGDSF | | |
| mAb ADC) | RQAPGKGLEWIGEILPGG | LNWYQQKPGKAPKLLIY | | |
| | GDTNYNEIFKGRATFSADT | AASNLESGVPSRFSGSGS | | |
| | SKNTAYLQMNSLRAEDTA | GTDFTLTISSLQPEDFATY | | |
| | VYYCTRRVPIRLDYWGQG | YCQQSNEDPLTFGQGTK | | |
| | TLVTVSS (SEQ ID NO: | VEIK (SEQ ID NO: 412) | | |
| | 411) | | | |
| Imalumab | EVQLLESGGGLVQPGGSL | DIQMTQSPSSLSASVGD | na | na |
| (Format: Whole | RLSCAASGFTFSIYSMNW | RVTITCRSSQRIMTYLNW | | |
| mAb) | VRQAPGKGLEWVSSIGSS | YQQKPGKAPKLLIFVASH | | |
| | GGTTYYADSVKGRFTISRD | SQSGVPSRFRGSGSETDF | | |
| | NSKNTLYLQMNSLRAEDT | TLTISGLQPEDSATYYCQ | | |
| | AVYYCAGSQWLYGMDV | QSFWTPLTFGGGTKVEIK | | |
| | WGQGTTVTVSS (SEQ ID | (SEQ ID NO: 414) | | |
| | NO: 413) | | | |
| Imaprelimab | QVTLKESGPVLVKPTETLTL | DIQMTQSPSSLSASVGD | na | na |
| (Format: Whole | TCTVSGFSLTSNAVSWVR | RVTINCKASQNIYNSLAW | | |
| mAb) | QPPGKALEWIAAISSGGTT | YQQKPGKAPKVLIFNANS | | |
| | YYNSAFKSRLTISRDTSKSQ | LQTGIPSRFSGSGSGTDF | | |
| | VVLTMTNMDPVDTATYY | TLTISSLQPEDFATYYCQQ | | |
| | CARRYGYGWYFDFWGQG | FYSGYTFGQGTKLEIK | | |
| | TLVTVSS (SEQ ID NO: | (SEQ ID NO: 416) | | |
| | 415) | | | |
| Imgatuzumab | QVQLVQSGAEVKKPGSSV | DIQMTQSPSSLSASVGD | na | na |
| (Format: Whole | KVSCKASGFTFTDYKIHWV | RVTITCRASQGINNYLN | | |
| mAb) | RQAPGQGLEWMGYFNP | WYQQKPGKAPKRLIYNT | | |
| | NSGYSTYAQKFQGRVTITA | NNLQTGVPSRFSGSGSG | | |
| | DKSTSTAYMELSSLRSEDT | TEFTLTISSLQPEDFATYY | | |
| | AVYYCARLSPGGYYVMDA | CLQHNSFPTFGQGTKLEI | | |
| | WGQGTTVTVSS (SEQ ID | K (SEQ ID NO: 418) | | |
| | NO: 417) | | | |
| Inclacumab | EVQLVESGGGLVRPGGSL | EIVLTQSPATLSLSPGERA | na | na |
| (Format: Whole | RLSCAASGFTFSNYDMH | TLSCRASQSVSSYLAWYQ | | |
| mAb) | WVRQATGKGLEWVSAIT | QKPGQAPRLLIYDASNRA | | |
| | AAGDIYYPGSVKGRFTISR | TGIPARFSGSGSGTDFTLT | | |
| | ENAKNSLYLQMNSLRAGD | ISSLEPEDFAVYYCQQRS | | |
| | TAVYYCARGRYSGSGSYY | NWPLTFGGGTKVEIK | | |
| | NDWFDPWGQGTLVTVSS | (SEQ ID NO: 420) | | |
| | (SEQ ID NO: 419) | | | |
| Indatuximab | QVQLQQSGSELMMPGAS | DIQMTQSTSSLSASLGDR | na | na |
| (Format: Whole | VKISCKATGYTFSNYWIEW | VTISCSASQGINNYLNWY | | |
| mAb ADC) | VKQRPGHGLEWIGEILPG | QQKPDGTVELLIYYTSTL | | |
| | TGRTIYNEKFKGKATFTADI | QSGVPSRFSGSGSGTDYS | | |
| | SSNTVQMQLSSLTSEDSA | LTISNLEPEDIGTYYCQQY | | |
| | VYYCARRDYYGNFYYAMD | SKLPRTFGGGTKLEIK | | |
| | YWGQGTSVTVSS (SEQ ID | (SEQ ID NO: 422) | | |
| | NO: 421) | | | |
| Indusatumab | QVQLQQWGAGLLKPSETL | EIVMTQSPATLSVSPGER | na | na |
| (Format: Whole | SLTCAVFGGSFSGYYWSW | ATLSCRASQSVSRNLAW | | |
| mAb ADC) | IRQPPGKGLEWIGEINHRG | YQQKPGQAPRLLIYGAST | | |
| | NTNDNPSLKSRVTISVDTS | RATGIPARFSGSGSGTEF | | |
| | KNQFALKLSSVTAADTAVY | TLTIGSLQSEDFAVYYCQ | | |
| | YCARERGYTYGNFDHWG | QYKTWPRTFGQGTNVEI | | |
| | QGTLVTVSS (SEQ ID NO: | K (SEQ ID NO: 424) | | |
| | 423) | | | |
| Inebilizumab | EVQLVESGGGLVQPGGSL | EIVLTQSPDFQSVTPKEK | na | na |
| (Format: Whole | RLSCAASGFTFSSSWMN | VTITCRASESVDTFGISFM | | |
| mAb) | WVRQAPGKGLEWVGRIY | NWFQQKPDQSPKLLIHE | | |
| | PGDGDTNYNVKFKGRFTI | ASNQGSGVPSRFSGSGS | | |
| | SRDDSKNSLYLQMNSLKT | GTDFTLTINSLEAEDAATY | | |
| | EDTAVYYCARSGFITTVRD | YCQQSKEVPFTFGGGTK | | |
| | FDYWGQGTLVTVSS (SEQ | VEIK (SEQ ID NO: 426) | | |
| | ID NO: 425) | | | |
| Infliximab | EVKLEESGGGLVQPGGSM | DILLTQSPAILSVSPGERV | na | na |
| (Format: Whole | KLSCVASGFIFSNHWMN | SFSCRASQFVGSSIHWYQ | | |
| mAb) | WVRQSPEKGLEWVAEIRS | QRTNGSPRLLIKYASESM | | |
| | KSINSATHYAESVKGRFTIS | SGIPSRFSGSGSGTDFTLS | | |
| | RDDSKSAVYLQMTDLRTE | INTVESEDIADYYCQQSH | | |
| | DTGVYYCSRNYYGSTYDY | SWPFTFGSGTNLEVK | | |
| | WGQGTTLTVSS (SEQ ID | (SEQ ID NO: 428) | | |
| | NO: 427) | | | |
| Inotuzumab | EVQLVQSGAEVKKPGASV | DVQVTQSPSSLSASVGD | na | na |
| (Format: Whole | KVSCKASGYRFTNYWIHW | RVTITCRSSQSLANSYGN | | |
| mAb ADC) | VRQAPGQGLEWIGGINPG | TFLSWYLHKPGKAPQLLI | | |
| | NNYATYRRKFQGRVTMT | YGISNRFSGVPDRFSGSG | | |
| | ADTSTSTVYMELSSLRSED | SGTDFTLTISSLQPEDFAT | | |
| | TAVYYCTREGYGNYGAWF | YYCLQGTHQPYTFGQGT | | |
| | AYWGQGTLVTVSS (SEQ | KVEIK (SEQ ID NO: 430) | | |
| | ID NO: 429) | | | |
| Intetumumab | QVQLVESGGGVVQPGRS | EIVLTQSPATLSLSPGERA | na | na |
| (Format: Whole | RRLSCAASGFTFSRYTMH | TLSCRASQSVSSYLAWYQ | | |
| mAb) | WVRQAPGKGLEWVAVIS | QKPGQAPRLLIYDASNRA | | |
| | FDGSNKYYVDSVKGRFTIS | TGIPARFSGSGSGTDFTLT | | |
| | RDNSENTLYLQVNILRAED | ISSLEPEDFAVYYCQQRS | | |
| | TAVYYCAREARGSYAFDI | NWPPFTFGPGTKVDIK | | |
| | WGQGTMVTVSS (SEQ ID | (SEQ ID NO: 432) | | |
| | NO: 431) | | | |
| Ipilimumab | QVQLVESGGGVVQPGRSL | EIVLTQSPGTLSLSPGERA | na | na |
| (Format: Whole | RLSCAASGFTFSSYTMHW | TLSCRASQSVGSSYLAWY | | |
| mAb) | VRQAPGKGLEWVTFISYD | QQKPGQAPRLLIYGAFSR | | |
| | GNNKYYADSVKGRFTISRD | ATGIPDRFSGSGSGTDFT | | |
| | NSKNTLYLQMNSLRAEDT | LTISRLEPEDFAVYYCQQY | | |
| | AIYYCARTGWLGPFDYWG | GSSPWTFGQGTKVEIK | | |
| | QGTLVTVSS (SEQ ID NO: | (SEQ ID NO: 434) | | |
| | 433) | | | |
| Iratumumab | QVQLQQWGAGLLKPSETL | DIQMTQSPTSLSASVGD | na | na |
| (Format: Whole | SLTCAVYGGSFSAYYWSW | RVTITCRASQGISSWLTW | | |
| mAb) | IRQPPGKGLEWIGDINHG | YQQKPEKAPKSLIYAASSL | | |
| | GGTNYNPSLKSRVTISVDT | QSGVPSRFSGSGSGTDFT | | |
| | SKNQFSLKLNSVTAADTAV | LTISSLQPEDFATYYCQQY | | |
| | YYCASLTAYWGQGSLVTV | DSYPITFGQGTRLEIK | | |
| | SS (SEQ ID NO: 435) | (SEQ ID NO: 436) | | |
| Isatuximab | QVQLVQSGAEVAKPGTSV | DIVMTQSHLSMSTSLGD | na | na |
| (Format: Whole | KLSCKASGYTFTDYWMQ | PVSITCKASQDVSTVVA | | |
| mAb) | WVKQRPGQGLEWIGTIYP | WYQQKPGQSPRRLIYSA | | |
| | GDGDTGYAQKFQGKATLT | SYRYIGVPDRFTGSGAGT | | |
| | ADKSSKTVYMHLSSLASED | DFTFTISSVQAEDLAVYYC | | |
| | SAVYYCARGDYYGSNSLD | QQHYSPPYTFGGGTKLEI | | |
| | YWGQGTSVTVSS (SEQ ID | K (SEQ ID NO: 438) | | |
| | NO: 437) | | | |
| Iscalimab | QVQLVESGGGVVQPGRSL | DIVMTQSPLSLTVTPGEP | na | na |
| (Format: Whole | RLSCAASGFTFSSYGMHW | ASISCRSSQSLLYSNGYNY | | |
| mAb) | VRQAPGKGLEWVAVISYE | LDWYLQKPGQSPQVLISL | | |
| | ESNRYHADSVKGRFTISRD | GSNRASGVPDRFSGSGS | | |
| | NSKITLYLQMNSLRTEDTA | GTDFTLKISRVEAEDVGV | | |
| | VYYCARDGGIAAPGPDYW | YYCMQARQTPFTFGPGT | | |
| | GQGTLVTVSS (SEQ ID | KVDIR (SEQ ID NO: 440) | | |
| | NO: 439) | | | |
| Istiratumab | EVQLLQSGGGLVQPGGSL | DIQMTQSPSSLSASLGDR | QVQLVQSGGGLV | SYELTQDPAVSVA |
| (Format: | RLSCAASGFMFSRYPMH | VTITCRASQGISSYLAWY | QPGGSLRLSCAAS | LGQTVRITCQGDS |
| Bispecific Mixed | WVRQAPGKGLEWVGSIS | QQKPGKAPKLLIYAKSTL | GFTFDDYAMHWV | LRSYYASWYQQK |
| mAb and scFv) | GSGGATPYADSVKGRFTIS | QSGVPSRFSGSGSGTDFT | RQAPGKGLEWVA | PGQAPVLVIYGKN |
| | RDNSKNTLYLQMNSLRAE | LTISSLQPEDSATYYCQQY | GISWDSGSTGYAD | NRPSGIPDRFSGS |
| | DTAVYYCAKDFYQILTGNA | WTFPLTFGGGTKVEIK | SVKGRFTISRDNAK | TSGNSASLTITGA |
| | FDYWGQGTTVTVSS (SEQ | (SEQ ID NO: 442) | NSLYLQMNSLRAE | QAEDEADYYCNS |
| | ID NO: 441) | | DTALYYCARDLGAY | RDSPGNQWVFG |
| | | | QWVEGFDYWGQ | GGTKVTVL (SEQ |
| | | | GTLVTVSS | ID NO: 444) |
| | | | (SEQ ID NO: 443) | |
| Itolizumab | EVQLVESGGGLVKPGGSL | DIQMTQSPSSLSASVGD | na | na |
| (Format: Whole | KLSCAASGFKFSRYAMSW | RVTITCKASRDIRSYLTWY | | |
| mAb) | VRQAPGKRLEWVATISSG | QQKPGKAPKTLIYYATSL | | |
| | GSYIYYPDSVKGRFTISRDN | ADGVPSRFSGSGSGQDY | | |
| | VKNTLYLQMSSLRSEDTA | SLTISSLESDDTATYYCLQ | | |
| | MYYCARRDYDLDYFDSW | HGESPFTLGSGTKLEIK | | |
| | GQGTLVTVSS (SEQ ID | (SEQ ID NO: 446) | | |
| | NO: 445) | | | |
| Ixekizumab | QVQLVQSGAEVKKPGSSV | DIVMTQTPLSLSVTPGQP | na | na |
| (Format: Whole | KVSCKASGYSFTDYHIHW | ASISCRSSRSLVHSRGNTY | | |
| mAb) | VRQAPGQGLEWMGVINP | LHWYLQKPGQSPQLLIYK | | |
| | MYGTTDYNQRFKGRVTIT | VSNRFIGVPDRFSGSGSG | | |
| | ADESTSTAYMELSSLRSED | TDFTLKISRVEAEDVGVY | | |
| | TAVYYCARYDYFTGTGVY | YCSQSTHLPFTFGQGTKL | | |
| | WGQGTLVTVSS (SEQ ID | EIK (SEQ ID NO: 448) | | |
| | NO: 447) | | | |
| Labetuzumab | EVQLVESGGGVVQPGRSL | DIQLTQSPSSLSASVGDR | na | na |
| (Format: Whole | RLSCSASGFDFTTYWMS | VTITCKASQDVGTSVAW | | |
| mAb ADC) | WVRQAPGKGLEWIGEIHP | YQQKPGKAPKLLIYWTST | | |
| | DSSTINYAPSLKDRFTISRD | RHTGVPSRFSGSGSGTDF | | |
| | NAKNTLFLQMDSLRPEDT | TFTISSLQPEDIATYYCQQ | | |
| | GVYFCASLYFGFPWFAYW | YSLYRSFGQGTKVEIK | | |
| | GQGTPVTVSS (SEQ ID | (SEQ ID NO: 450) | | |
| | NO: 449) | | | |
| Lacnotuzumab | QVQLQESGPGLVKPSQTL | DIVLTQSPAFLSVTPGEKV | na | na |
| (Format: Whole | SLTCTVSDYSITSDYAWN | TFTCQASQSIGTSIHWYQ | | |
| mAb) | WIRQFPGKGLEWMGYISY | QKTDQAPKLLIKYASESIS | | |
| | SGSTSYNPSLKSRITISRDTS | GIPSRFSGSGSGTDFTLTI | | |
| | KNQFSLQLNSVTAADTAV | SSVEAEDAADYYCQQINS | | |
| | YYCASFDYAHAMDYWGQ | WPTTFGGGTKLEIK (SEQ | | |
| | GTTVTVSS (SEQ ID NO: | ID NO: 452) | | |
| | 451) | | | |
| Lacutamab | QIQLVQSGSELKKPGASVK | DIQMTQSPSFLSASVGD | na | na |
| (Format: Whole | VSCKASGYTFTTAGMQW | RVTITCKASQDVSTAVA | | |
| mAb) | VRQAPGQGLEWIGWINS | WYQQKPGQPPKLLIYWT | | |
| | HSGVPKYAEDFKGRFVFSL | STRHTGVPDRFSGSGSGT | | |
| | DTSVSTAYLQISSLKAEDTA | DYTLTISSLQAEDVAVYYC | | |
| | VYFCARGGDEGVMDYW | QQHYSTPWTFGGGTKVE | | |
| | GQGTTVTVSS (SEQ ID | IK (SEQ ID NO: 454) | | |
| | NO: 453) | | | |
| Ladiratuzumab | QVQLVQSGAEVKKPGASV | DVVMTQSPLSLPVTLGQ | na | na |
| (Format: Whole | KVSCKASGLTIEDYYMHW | PASISCRSSQSLLHSSGNT | | |
| mAb ADC) | VRQAPGQGLEWMGWID | YLEWYQQRPGQSPRPLIY | | |
| | PENGDTEYGPKFQGRVT | KISTRFSGVPDRFSGSGS | | |
| | MTRDTSINTAYMELSRLRS | GTDFTLKISRVEAEDVGV | | |
| | DDTAVYYCAVHNAHYGT | YYCFQGSHVPYTFGGGT | | |
| | WFAYWGQGTLVTVSS | KVEIK (SEQ ID NO: 456) | | |
| | (SEQ ID NO: 455) | | | |
| Lampalizumab | EVQLVQSGPELKKPGASV | DIQVTQSPSSLSASVGDR | na | na |
| (Format: Fab) | KVSCKASGYTFTNYGMN | VTITCITSTDIDDDMNWY | | |
| | WVRQAPGQGLEWMGWI | QQKPGKVPKLLISGGNTL | | |
| | NTYTGETTYADDFKGRFVF | RPGVPSRFSGSGSGTDFT | | |
| | SLDTSVSTAYLQISSLKAED | LTISSLQPEDVATYYCLQS | | |
| | TAVYYCEREGGVNNWGQ | DSLPYTFGQGTKVEIK | | |
| | GTLVTVSS (SEQ ID NO: | (SEQ ID NO: 458) | | |
| | 457) | | | |
| Lanadelumab | EVQLLESGGGLVQPGGSL | DIQMTQSPSTLSASVGD | na | na |
| (Format: Whole | RLSCAASGFTFSHYIMMW | RVTITCRASQSISSWLAW | | |
| mAb) | VRQAPGKGLEWVSGIYSS | YQQKPGKAPKLLIYKASTL | | |
| | GGITVYADSVKGRFTISRD | ESGVPSRFSGSGSGTEFT | | |
| | NSKNTLYLQMNSLRAEDT | LTISSLQPDDFATYYCQQ | | |
| | AVYYCAYRRIGVPRRDEFD | YNTYWTFGQGTKVEIK | | |
| | IWGQGTMVTVSS (SEQ | (SEQ ID NO: 460) | | |
| | ID NO: 459) | | | |
| Landogrozumab | EVQLVESGGGLVQPGGSL | EIVLTQSPGTLSLSPGERA | na | na |
| (Format: Whole | RLSCAASGLTFSRYPMSW | TLSCRASSSVSSSYLHWY | | |
| mAb) | VRQAPGKGLVWVSAITSS | QQKPGQAPRLLIYSTSNL | | |
| | GGSTYYSDTVKGRFTISRD | VAGIPDRFSGSGSGTDFT | | |
| | NAKNTLYLQMNSLRAEDT | LTISRLEPEDFAVYYCQH | | |
| | AVYYCARLPDYWGQGTLV | HSGYHFTFGGGTKVEIK | | |
| | TVSS (SEQ ID NO: 461) | (SEQ ID NO: 462) | | |
| Laprituximab | QVQLVQSGAEVAKPGASV | DIQMTQSPSSLSASVGD | na | na |
| (Format: Whole | KLSCKASGYTFTSYWMQ | RVTITCRASQDINNYLAW | | |
| mAb ADC) | WVKQRPGQGLECIGTIYP | YQHKPGKGPKLLIHYTSTL | | |
| | GDGDTTYTQKFQGKATLT | HPGIPSRFSGSGSGRDYS | | |
| | ADKSSSTAYMQLSSLRSED | FSISSLEPEDIATYYCLQYD | | |
| | SAVYYCARYDAPGYAMDY | NLLYTFGQGTKLEIK (SEQ | | |
| | WGQGTLVTVSS (SEQ ID | ID NO: 464) | | |
| | NO: 463) | | | |
| Larcaviximab | EVQLQESGPELEMPGASV | DIQMTQSPASLSASVGET | na | na |
| (Format: Whole | KISCKASGSSFTGFSMNW | VTITCRASENIYSYLAWY | | |
| mAb) | VKQSNGKSLEWIGNIDTYY | QQKQGKSPQLLVYNAKT | | |
| | GGTTYNQKFKGKATLTVD | LIEGVPSRFSGSGSGTQFS | | |
| | KSSSTAYMQLKSLTSEDSA | LKINSLQPEDFGSYFCQH | | |
| | VYYCARSAYYGSTFAYWG | HFGTPFTFGSGTELEIK | | |
| | QGTLVTVSA (SEQ ID NO: | (SEQ ID NO: 466) | | |
| | 465) | | | |
| Lebrikizumab | QVTLRESGPALVKPTQTLT | DIVMTQSPDSLSVSLGER | na | na |
| (Format: Whole | LTCTVSGFSLSAYSVNWIR | ATINCRASKSVDSYGNSF | | |
| mAb) | QPPGKALEWLAMIWGDG | MHWYQQKPGQPPKLLIY | | |
| | KIVYNSALKSRLTISKDTSK | LASNLESGVPDRFSGSGS | | |
| | NQVVLTMTNMDPVDTAT | GTDFTLTISSLQAEDVAV | | |
| | YYCAGDGYYPYAMDNWG | YYCQQNNEDPRTFGGGT | | |
| | QGSLVTVSS (SEQ ID NO: | KVEIK (SEQ ID NO: 468) | | |
| | 467) | | | |
| Lenvervimab | EVQLVESGGGLVKPGGSL | DIVVTQSPSSLSASVGDR | na | na |
| (Format: Whole | RLSCSASGFSLTKYKMTW | VTITCRASQGIYNSIAWY | | |
| mAb) | VRQAPGKGLEWVSSISSTS | QQKPGKAPKLLLYSTSTLL | | |
| | RDIDYADSVKGRFTISRDN | SGVPSRFSGSGSGTDYTL | | |
| | AKNSLFLQMSSLRVDDTA | TITNLQPEDFATYYCQQY | | |
| | VYYCTRDGWLWGWDVR | FVTPETFGQGTKLEIK | | |
| | SNYYYNALDVWGQGTTV | (SEQ ID NO: 470) | | |
| | TVSS (SEQ ID NO: 469) | | | |
| Lenzilumab | QVQLVQSGAEVKKPGASV | EIVLTQSPATLSVSPGERA | na | na |
| (Format: Whole | KVSCKASGYSFTNYYIHWV | TLSCRASQSVGTNVAWY | | |
| mAb) | RQAPGQRLEWMGWINA | QQKPGQAPRVLIYSTSSR | | |
| | GNGNTKYSQKFQGRVTIT | ATGITDRFSGSGSGTDFT | | |
| | RDTSASTAYMELSSLRSED | LTISRLEPEDFAVYYCQQF | | |
| | TAVYYCVRRQRFPYYFDY | NKSPLTFGGGTKVEIK | | |
| | WGQGTLVTVSS (SEQ ID | (SEQ ID NO: 472) | | |
| | NO: 471) | | | |
| Leronlimab | EVQLVESGGGLVKPGGSL | DIVMTQSPLSLPVTPGEP | na | na |
| (Format: Whole | RLSCAASGYTFSNYWIGW | ASISCRSSQRLLSSYGHTY | | |
| mAb) | VRQAPGKGLEWIGDIYPG | LHWYLQKPGQSPQLLIYE | | |
| | GNYIRNNEKFKDKTTLSAD | VSNRFSGVPDRFSGSGS | | |
| | TSKNTAYLQMNSLKTEDT | GTDFTLKISRVEAEDVGV | | |
| | AVYYCGSSFGSNYVFAWF | YYCSQSTHVPLTFGQGTK | | |
| | TYWGQGTLVTVSS (SEQ | VEIK (SEQ ID NO: 474) | | |
| | ID NO: 473) | | | |
| Lesofavumab | EVQLVQSGAEVKKPGESL | DIVMTQSPLSLPVTPGEP | na | na |
| (Format: Whole | KISCKVSGYSFTSQWIGW | ASISCRSSQSLLRSNGYNY | | |
| mAb) | VRQMPGKGLEWIGMMY | LDWYLQKPGQSPQLLIYL | | |
| | PGESETIYSPSFQGQVTISA | GSNRASGVPDRFSGSGS | | |
| | DNSISTAYLQWSSLKASDT | GTDFTLKISRVEAEDVGV | | |
| | AIYYCASGPGYSGYHYGW | YYCMQALQTPYTFGQGT | | |
| | FDTWGQGTLVTVSS | KLEIK | | |
| | (SEQ ID NO: 475) | (SEQ ID NO: 476) | | |
| Letolizumab | EVQLLESGGGLVQPGGSL | na | na | na |
| (Format: Single | RLSCAASGFTFNWELMG | | | |
| Domain Variable | WARQAPGKGLEWVSGIE | | | |
| Fragment; H) | GPGDVTYYADSVKGRFTIS | | | |
| | RDNSKNTLYLQMNSLRAE | | | |
| | DTAVYYCVKVGKDAKSDY | | | |
| | RGQGTLVTVSS (SEQ ID | | | |
| | NO: 477) | | | |
| Levilimab | QVQLVQSGGGLVQPGGS | DIQLTQSPSSVSVSVGER | na | na |
| (Format: Whole | LRLSCAASGFTFSSYYMSW | VTIDCKSSQSVLSASNTYL | | |
| mAb) | VRQAPGKGLEWVSGIYSD | NWYQQKPGQAPQLLIYY | | |
| | GTTHYGDSVKGRFTISRDN | ASTRESGVPDRFSGSGSG | | |
| | AKNTVYLQLNSLRAEDTA | TDFTLTISSLQAEDAAVYY | | |
| | MYYCAKGAGPTWWYAL | CQQAYRAPVTFGQGTKL | | |
| | DAWGQGTLVTVSS (SEQ | EIK (SEQ ID NO: 479) | | |
| | ID NO: 478) | | | |
| Lexatumumab | EVQLVQSGGGVERPGGSL | SSELTQDPAVSVALGQTV | na | na |
| (Format: Whole | RLSCAASGFTFDDYGMSW | RITCQGDSLRSYYASWYQ | | |
| mAb) | VRQAPGKGLEWVSGINW | QKPGQAPVLVIYGKNNR | | |
| | NGGSTGYADSVKGRVTIS | PSGIPDRFSGSSSGNTASL | | |
| | RDNAKNSLYLQMNSLRAE | TITGAQAEDEADYYCNSR | | |
| | DTAVYYCAKILGAGRGWY | DSSGNHVVFGGGTKLTV | | |
| | FDLWGKGTTVTVSS (SEQ | L (SEQ ID NO: 481) | | |
| | ID NO: 480) | | | |
| Lifastuzumab | EVQLVESGGGLVQPGGSL | DIQMTQSPSSLSASVGD | na | na |
| (Format: Whole | RLSCAASGFSFSDFAMSW | RVTITCRSSETLVHSSGNT | | |
| mAb ADC) | VRQAPGKGLEWVATIGRV | YLEWYQQKPGKAPKLLIY | | |
| | AFHTYYPDSMKGRFTISRD | RVSNRFSGVPSRFSGSGS | | |
| | NSKNTLYLQMNSLRAEDT | GTDFTLTISSLQPEDFATY | | |
| | AVYYCARHRGFDVGHFDF | YCFQGSFNPLTFGQGTK | | |
| | WGQGTLVTVSS (SEQ ID | VEIK (SEQ ID NO: 483) | | |
| | NO: 482) | | | |
| Ligelizumab | QVQLVQSGAEVMKPGSS | EIVMTQSPATLSVSPGER | na | na |
| (Format: Whole | VKVSCKASGYTFSWYWLE | ATLSCRASQSIGTNIHWY | | |
| mAb) | WVRQAPGHGLEWMGEI | QQKPGQAPRLLIYYASESI | | |
| | DPGTFTTNYNEKFKARVTF | SGIPARFSGSGSGTEFTLT | | |
| | TADTSTSTAYMELSSLRSE | ISSLQSEDFAVYYCQQSW | | |
| | DTAVYYCARFSHFSGSNY | SWPTTFGGGTKVEIK | | |
| | DYFDYWGQGTLVTVSS | (SEQ ID NO: 485) | | |
| | (SEQ ID NO: 484) | | | |
| Lilotomab | EIQLQQSGPELVKPGASVK | DIVMTQSHKLLSTSVGDR | na | na |
| (Format: Whole | VSCKASGYSFTDYNMYW | VSITCKASQDVSTAVDW | | |
| mAb ADC) | VKQSHGKSLEWIGYIDPYN | YQQKPGQSPKLLINWAS | | |
| | GDTTYNQKFKGKATLTVD | TRHTGVPDRFTGSGSGT | | |
| | KSSSTAFIHLNSLTSEDSAV | DYTLTISSMQAEDLALYY | | |
| | YYCARSPYGHYAMDYWG | CRQHYSTPFTFGSGTKLEI | | |
| | QGTSVTVSS (SEQ ID NO: | K (SEQ ID NO: 487) | | |
| | 486) | | | |
| Lintuzumab | QVQLVQSGAEVKKPGSSV | DIQMTQSPSSLSASVGD | na | na |
| (Format: Whole | KVSCKASGYTFTDYNMH | RVTITCRASESVDNYGISF | | |
| mAb | WVRQAPGQGLEWIGYIYP | MNWFQQKPGKAPKLLIY | | |
| Radiolabelled) | YNGGTGYNQKFKSKATIT | AASNQGSGVPSRFSGSG | | |
| | ADESTNTAYMELSSLRSED | SGTDFTLTISSLQPDDFAT | | |
| | TAVYYCARGRPAMDYWG | YYCQQSKEVPWTFGQGT | | |
| | QGTLVTVSS (SEQ ID NO: | KVEIK (SEQ ID NO: 489) | | |
| | 488) | | | |
| Lirilumab | QVQLVQSGAEVKKPGSSV | EIVLTQSPVTLSLSPGERA | na | na |
| (Format: Whole | KVSCKASGGTFSFYAISWV | TLSCRASQSVSSYLAWYQ | | |
| mAb) | RQAPGQGLEWMGGFIPIF | QKPGQAPRLLIYDASNRA | | |
| | GAANYAQKFQGRVTITAD | TGIPARFSGSGSGTDFTLT | | |
| | ESTSTAYMELSSLRSDDTA | ISSLEPEDFAVYYCQQRS | | |
| | VYYCARIPSGSYYYDYDMD | NWMYTFGQGTKLEIK | | |
| | VWGQGTTVTVSS (SEQ ID | (SEQ ID NO: 491) | | |
| | NO: 490) | | | |
| Lodelcizumab | QVQLVQSGAEVKKPGASV | QIVLTQSPATLSVSPGER | na | na |
| (Format: Whole | KVSCKASGYTFSTMYMS | ATLSCRASQSVSYMHWY | | |
| mAb) | WVRQAPGQGLEWMGRI | QQKPGQAPRLLIYGVFRR | | |
| | DPANEHTNYAQKFQGRV | ATGIPDRFSGSGSGTDFT | | |
| | TMTRDTSISTAYMELSRLT | LTIGRLEPEDFAVYYCLQ | | |
| | SDDTAVYYCARSYYYYNM | WSSDPPTFGQGTKLEIK | | |
| | DYWGQGTLVTVSS (SEQ | (SEQ ID NO: 493) | | |
| | ID NO: 492) | | | |
| Lokivetmab | EVQLVESGGDLVKPGGSL | EIVMTQSPASLSLSQEEK | na | na |
| (Format: Canine | RLSCVASGFTFSNYGMSW | VTITCKASQSVSFAGTGL | | |
| Whole mAb) | VRQAPGKGLQWVATISYG | MHWYQQKPGQAPKLLI | | |
| | GSYTYYPDNIKGRFTISRD | YRASNLEAGVPSRFSGSG | | |
| | NAKNTLYLQMNSLRAEDT | SGTDFSFTISSLEPEDVAV | | |
| | AMYYCVRGYGYDTMDY | YYCQQSREYPWTFGQGT | | |
| | WGQGTLVTVSS (SEQ ID | KLEIK | | |
| | NO: 494) | (SEQ ID NO: 495) | | |
| Loncastuximab | QVQLVQPGAEVVKPGAS | EIVLTQSPAIMSASPGER | na | na |
| (Format: Whole | VKLSCKTSGYTFTSNWMH | VTMTCSASSGVNYMHW | | |
| mAb ADC) | WVKQAPGQGLEWIGEID | YQQKPGTSPRRWIYDTS | | |
| | PSDSYTNYNQNFQGKAKL | KLASGVPARFSGSGSGTS | | |
| | TVDKSTSTAYMEVSSLRSD | YSLTISSMEPEDAATYYC | | |
| | DTAVYYCARGSNPYYYAM | HQRGSYTFGGGTKLEIK | | |
| | DYWGQGTSVTVSS | (SEQ ID NO: 497) | | |
| | (SEQ ID NO: 496) | | | |
| Lorukafusp | EVQLVQSGAEVEKPGASV | DVVMTQTPLSLPVTPGE | na | na |
| (Format: Whole | KISCKASGSSFTGYNMNW | PASISCRSSQSLVHRNGN | | |
| mAb Fusion) | VRQNIGKSLEWIGAIDPYY | TYLHWYLQKPGQSPKLLI | | |
| | GGTSYNQKFKGRATLTVD | HKVSNRFSGVPDRFSGS | | |
| | KSTSTAYMHLKSLRSEDTA | GSGTDFTLKISRVEAEDL | | |
| | VYYCVSGMEYWGQGTSV | GVYFCSQSTHVPPLTFGA | | |
| | TVSS (SEQ ID NO: 498) | GTKLELK | | |
| | | (SEQ ID NO: 499) | | |
| Lorvotuzumab | QVQLVESGGGVVQPGRSL | DVVMTQSPLSLPVTLGQ | na | na |
| (Format: Whole | RLSCAASGFTFSSFGMHW | PASISCRSSQIIIHSDGNTY | | |
| mAb ADC) | VRQAPGKGLEWVAYISSG | LEWFQQRPGQSPRRLIYK | | |
| | SFTIYYADSVKGRFTISRDN | VSNRFSGVPDRFSGSGS | | |
| | SKNTLYLQMNSLRAEDTA | GTDFTLKISRVEAEDVGV | | |
| | VYYCARMRKGYAMDYW | YYCFQGSHVPHTFGQGT | | |
| | GQGTLVTVSS | KVEIK | | |
| | (SEQ ID NO: 500) | (SEQ ID NO: 501) | | |
| Losatuxizumab | EVQLQESGPGLVKPSQTLS | DIQMTQSPSSMSVSVGD | na | na |
| (Serclutamab) | LTCTVSGYSISRDFAWNWI | RVTITCHSSQDINSNIGW | | |
| (Format: Whole | RQPPGKGLEWMGYISYN | LQQKPGKSFKGLIYHGTN | | |
| mAb ADC) | GNTRYQPSLKSRITISRDTS | LDDGVPSRFSGSGSGTDY | | |
| | KNQFFLKLNSVTAADTATY | TLTISSLQPEDFATYYCVQ | | |
| | YCVTASRGFPYWGQGTLV | YAQFPWTFGGGTKLEIK | | |
| | TVSS (SEQ ID NO: 502) | (SEQ ID NO: 503) | | |
| Lucatumumab | QVQLVESGGGVVQPGRSL | DIVMTQSPLSLTVTPGEP | na | na |
| (Format: Whole | RLSCAASGFTFSSYGMHW | ASISCRSSQSLLYSNGYNY | | |
| mAb) | VRQAPGKGLEWVAVISYE | LDWYLQKPGQSPQVLISL | | |
| | ESNRYHADSVKGRFTISRD | GSNRASGVPDRFSGSGS | | |
| | NSKITLYLQMNSLRTEDTA | GTDFTLKISRVEAEDVGV | | |
| | VYYCARDGGIAAPGPDYW | YYCMQARQTPFTFGPGT | | |
| | GQGTLVTVSS (SEQ ID | KVDIR (SEQ ID NO: 505) | | |
| | NO: 504) | | | |
| Lulizumab | na | DIQMTQSPSSLSASVGD | na | na |
| (Format: Single | | RVTITCRASRPIWPFLEW | | |
| Domain Variable | | YQQKPGKAPKLLIYFTSRL | | |
| Fragment; L) | | RHGVPSRFSGSGSGTCFT | | |
| | | LTISSLQPEDFATYYCLQN | | |
| | | VANPATFSQGTKVEIK | | |
| | | (SEQ ID NO: 506) | | |
| Lumiliximab | EVQLVESGGGLAKPGGSL | DIQMTQSPSSLSASVGD | na | na |
| (Format: Whole | RLSCAASGFRFTFNNYYM | RVTITCRASQDIRYYLNW | | |
| mAb) | DWVRQAPGQGLEWVSRI | YQQKPGKAPKLLIYVASSL | | |
| | SSSGDPTWYADSVKGRFTI | QSGVPSRFSGSGSGTEFT | | |
| | SRENANNTLFLQMNSLRA | LTVSSLQPEDFATYYCLQ | | |
| | EDTAVYYCASLTTGSDSW | VYSTPRTFGQGTKVEIK | | |
| | GQGVLVTVSS (SEQ ID | (SEQ ID NO: 508) | | |
| | NO: 507) | | | |
| Lumretuzumab | QVQLVQSGAEVKKPGASV | DIVMTQSPDSLAVSLGER | na | na |
| (Format: Whole | KVSCKASGYTFRSSYISWV | ATINCKSSQSVLNSGNQK | | |
| mAb) | RQAPGQGLEWMGWIYA | NYLTWYQQKPGQPPKLL | | |
| | GTGSPSYNQKLQGRVTM | IYWASTRESGVPDRFSGS | | |
| | TTDTSTSTAYMELRSLRSD | GSGTDFTLTISSLQAEDV | | |
| | DTAVYYCARHRDYYSNSLT | AVYYCQSDYSYPYTFGQ | | |
| | YWGQGTLVTVSS (SEQ ID | GTKLEIK (SEQ ID NO: | | |
| | NO: 509) | 510) | | |
| Lupartumab | EVQLLESGGGLVQPGGSL | ESVLTQPPSVSGAPGQR | na | na |
| (Format: Whole | RLSCAASGFTFSNAWMS | VTISCTGSSSNIGAGYVV | | |
| mAb ADC) | WVRQAPGKGLEWVSYISS | HWYQQLPGTAPKLLIYD | | |
| | SGSTIYYADSVKGRFTISRD | NNKRPSGVPDRFSGSKS | | |
| | NSKNTLYLQMNSLRAEDT | GTSASLAISGLRSEDEADY | | |
| | AVYYCAREGLWAFDYWG | YCAAWDDRLNGPVFGG | | |
| | QGTLVTVSS (SEQ ID NO: | GTKLTVL (SEQ ID NO: | | |
| | 511) | 512) | | |
| Lutikizumab | EVQLVESGGGVVQPGRSL | DIQMTQSPSSLSASVGD | QVQLVESGGGVV | DIQMTQSPSSVS |
| (Format: | RLSCSASGFIFSRYDMSWV | RVTITCRASGNIHNYLTW | QPGRSLRLSCTASG | ASVGDRVTITCRA |
| Bispecific Dual | RQAPGKGLEWVAYISHGG | YQQTPGKAPKLLIYNAKT | FTFSMFGVHWVR | SQGISSWLAWYQ |
| Variable Domain | AGTYYPDSVKGRFTISRDN | LADGVPSRFSGSGSGTDY | QAPGKGLEWVAA | QKPGKAPKLLIYE |
| IG) | SKNTLFLQMDSLRPEDTG | TFTISSLQPEDIATYYCQH | VSYDGSNKYYAESV | ASNLETGVPSRFS |
| | VYFCARGGVTKGYFDVW | FWSIPYTFGQGTKLQIT | KGRFTISRDNSKNIL | GSGSGSDFTLTISS |
| | GQGTPVTVSS (SEQ ID | (SEQ ID NO: 514) | FLQMDSLRLEDTA | LQPEDFATYYCQ |
| | NO: 513) | | VYYCARGRPKVVIP | QTSSFLLSFGGGT |
| | | | APLAHWGQGTLV | KVEHK (SEQ ID |
| | | | TFSS (SEQ ID NO: | NO: 516) |
| | | | 515) | |
| Maftivimab | EVQLVESGGGLVQPGGSL | DIQMTQSPSSLSASVGD | na | na |
| (Format: Whole | RLSCAASGFTSSSYAMNW | RVTITCRASQSISSFLNWY | | |
| mAb) | VRQAPGKGLEWVSTISGM | QQKPGKAPKLLIYAASSL | | |
| | GGSTYYADSVKGRFTISRD | QSGVPSRFSGSGSGTDFT | | |
| | NSKNTLYLQMNSLRAEDT | LTISSLQPEDFATYYCQQS | | |
| | AVYYCAKRGYPHSFDIWG | YSTLTFGQGTRLEIK (SEQ | | |
| | QGTMVTVSS (SEQ ID | ID NO: 518) | | |
| | NO: 517) | | | |
| Magrolimab | QVQLVQSGAEVKKPGASV | DIVMTQSPLSLPVTPGEP | na | na |
| (Format: Whole | KVSCKASGYTFTNYNMH | ASISCRSSQSIVYSNGNTY | | |
| mAb) | WVRQAPGQRLEWMGTIY | LGWYLQKPGQSPQLLIYK | | |
| | PGNDDTSYNQKFKDRVTI | VSNRFSGVPDRFSGSGS | | |
| | TADTSASTAYMELSSLRSE | GTDFTLKISRVEAEDVGV | | |
| | DTAVYYCARGGYRAMDY | YYCFQGSHVPYTFGQGT | | |
| | WGQGTLVTVSS (SEQ ID | KLEIK (SEQ ID NO: 520) | | |
| | NO: 519) | | | |
| Margetuximab | QVQLQQSGPELVKPGASL | DIVMTQSHKFMSTSVGD | na | na |
| (Format: Whole | KLSCTASGFNIKDTYIHWV | RVSITCKASQDVNTAVA | | |
| mAb) | KQRPEQGLEWIGRIYPTN | WYQQKPGHSPKLLIYSAS | | |
| | GYTRYDPKFQDKATITADT | FRYTGVPDRFTGSRSGTD | | |
| | SSNTAYLQVSRLTSEDTAV | FTFTISSVQAEDLAVYYC | | |
| | YYCSRWGGDGFYAMDY | QQHYTTPPTFGGGTKVEI | | |
| | WGQGASVTVSS (SEQ ID | K (SEQ ID NO: 522) | | |
| | NO: 521) | | | |
| Marstacimab | EVQLLESGGGLVQPGGSL | QSVLTQPPSVSGAPGQR | na | na |
| (Format: Whole | RLSCAASGFTFSSYAMSW | VTISCTGSSSNIGAGYDV | | |
| mAb) | VRQAPGKGLEWVSAISGS | HWYQQLPGTAPKLLIYG | | |
| | GGSTYYADSVKGRFTISRD | NSNRPSGVPDRFSGSKS | | |
| | NSKNTLYLQMNSLRAEDT | GTSASLAITGLQAEDEAD | | |
| | AVYYCAILGATSLSAFDIW | YYCQSYDSSLSGSGVFGG | | |
| | GQGTMVTVSS (SEQ ID | GTKLTVL (SEQ ID NO: | | |
| | NO: 523) | 524) | | |
| Matuzumab | QVQLVQSGAEVKKPGASV | DIQMTQSPSSLSASVGD | na | na |
| (Format: Whole | KVSCKASGYTFTSHWMH | RVTITCSASSSVTYMYWY | | |
| mAb) | WVRQAPGQGLEWIGEFN | QQKPGKAPKLLIYDTSNL | | |
| | PSNGRTNYNEKFKSKATM | ASGVPSRFSGSGSGTDYT | | |
| | TVDTSTNTAYMELSSLRSE | FTISSLQPEDIATYYCQQ | | |
| | DTAVYYCASRDYDYDGRY | WSSHIFTFGQGTKVEIK | | |
| | FDYWGQGTLVTVSS (SEQ | (SEQ ID NO: 526) | | |
| | ID NO: 525) | | | |
| Mavrilimumab | QVQLVQSGAEVKKPGASV | QSVLTQPPSVSGAPGQR | na | na |
| (Format: Whole | KVSCKVSGYTLTELSIHWV | VTISCTGSGSNIGAPYDV | | |
| mAb) | RQAPGKGLEWMGGFDPE | SWYQQLPGTAPKLLIYHN | | |
| | ENEIVYAQRFQGRVTMTE | NKRPSGVPDRFSGSKSGT | | |
| | DTSTDTAYMELSSLRSEDT | SASLAITGLQAEDEADYY | | |
| | AVYYCAIVGSFSPLTLGLW | CATVEAGLSGSVFGGGT | | |
| | GQGTMVTVSS | KLTVL | | |
| | (SEQ ID NO: 527) | (SEQ ID NO: 528) | | |
| Mepolizumab | QVTLRESGPALVKPTQTLT | DIVMTQSPDSLAVSLGER | na | na |
| (Format: Whole | LTCTVSGFSLTSYSVHWVR | ATINCKSSQSLLNSGNQK | | |
| mAb) | QPPGKGLEWLGVIWASG | NYLAWYQQKPGQPPKLL | | |
| | GTDYNSALMSRLSISKDTS | IYGASTRESGVPDRFSGS | | |
| | RNQVVLTMTNMDPVDTA | GSGTDFTLTISSLQAEDV | | |
| | TYYCARDPPSSLLRLDYWG | AVYYCQNVHSFPFTFGG | | |
| | RGTPVTVSS (SEQ ID NO: | GTKLEIK (SEQ ID NO: | | |
| | 529) | 530) | | |
| Milatuzumab | QVQLQQSGSELKKPGASV | DIQLTQSPLSLPVTLGQP | na | na |
| (Format: Whole | KVSCKASGYTFTNYGVNW | ASISCRSSQSLVHRNGNT | | |
| mAb) | IKQAPGQGLQWMGWIN | YLHWFQQRPGQSPRLLIY | | |
| | PNTGEPTFDDDFKGRFAF | TVSNRFSGVPDRFSGSGS | | |
| | SLDTSVSTAYLQISSLKADD | GTDFTLKISRVEAEDVGV | | |
| | TAVYFCSRSRGKNEAWFA | YFCSQSSHVPPTFGAGTR | | |
| | YWGQGTLVTVSS (SEQ ID | LEIK (SEQ ID NO: 532) | | |
| | NO: 531) | | | |
| Mirikizumab | QVQLVQSGAEVKKPGSSV | DIQMTQSPSSLSASVGD | na | na |
| (Format: Whole | KVSCKASGYKFTRYVMH | RVTITCKASDHILKFLTWY | | |
| mAb) | WVRQAPGQGLEWMGYI | QQKPGKAPKLLIYGATSL | | |
| | NPYNDGTNYNEKFKGRVT | ETGVPSRFSGSGSGTDFT | | |
| | ITADKSTSTAYMELSSLRSE | LTISSLQPEDFATYYCQM | | |
| | DTAVYYCARNWDTGLWG | YWSTPFTFGGGTKVEIK | | |
| | QGTTVTVSS (SEQ ID NO: | (SEQ ID NO: 534) | | |
| | 533) | | | |
| Mirvetuximab | QVQLVQSGAEVVKPGASV | DIVLTQSPLSLAVSLGQPA | na | na |
| (Format: Whole | KISCKASGYTFTGYFMNW | IISCKASQSVSFAGTSLM | | |
| mAb ADC) | VKQSPGQSLEWIGRIHPY | HWYHQKPGQQPRLLIYR | | |
| | DGDTFYNQKFQGKATLTV | ASNLEAGVPDRFSGSGSK | | |
| | DKSSNTAHMELLSLTSEDF | TDFTLTISPVEAEDAATYY | | |
| | AVYYCTRYDGSRAMDYW | CQQSREYPYTFGGGTKLE | | |
| | GQGTTVTVSS (SEQ ID | IK (SEQ ID NO: 536) | | |
| | NO: 535) | | | |
| Mitazalimab | EVQLLESGGGLVQPGGSL | QSVLTQPPSASGTPGQR | na | na |
| (Vanalimab) | RLSCAASGFTFSTYGMHW | VTISCTGSSSNIGAGYNV | | |
| (Format: Whole | VRQAPGKGLEWLSYISGG | YWYQQLPGTAPKLLIYG | | |
| mAb) | SSYIFYADSVRGRFTISRDN | NINRPSGVPDRFSGSKSG | | |
| | SENALYLQMNSLRAEDTA | TSASLAISGLRSEDEADYY | | |
| | VYYCARILRGGSGMDLW | CAAWDKSISGLVFGGGT | | |
| | GQGTLVTVSS | KLTVL (SEQ ID NO: 538) | | |
| | (SEQ ID NO: 537) | | | |
| Modotuximab | QVQLQQPGAELVEPGGS | DIVMTQAAFSNPVTLGT | na | na |
| (Format: Whole | VKLSCKASGYTFTSHWMH | SASISCRSSKSLLHSNGITY | | |
| mAb) | WVKQRPGQGLEWIGEIN | LYWYLQKPGQSPQLLIYQ | | |
| | PSSGRNNYNEKFKSKATLT | MSNLASGVPDRFSSSGS | | |
| | VDKSSSTAYMQFSSLTSED | GTDFTLRISRVEAEDVGV | | |
| | SAVYYCVRYYGYDEAMDY | YYCAQNLELPYTFGGGTK | | |
| | WGQGTSVTVSS (SEQ ID | LEIK (SEQ ID NO: 540) | | |
| | NO: 539) | | | |
| Mogamulizumab | EVQLVESGGDLVQPGRSL | DVLMTQSPLSLPVTPGEP | na | na |
| (Format: Whole | RLSCAASGFIFSNYGMSW | ASISCRSSRNIVHINGDTY | | |
| mAb) | VRQAPGKGLEWVATISSA | LEWYLQKPGQSPQLLIYK | | |
| | STYSYYPDSVKGRFTISRD | VSNRFSGVPDRFSGSGS | | |
| | NAKNSLYLQMNSLRVEDT | GTDFTLKISRVEAEDVGV | | |
| | ALYYCGRHSDGNFAFGYW | YYCFQGSLLPWTFGQGT | | |
| | GQGTLVTVSS | KVEIK (SEQ ID NO: 542) | | |
| | (SEQ ID NO: 541) | | | |
| Monalizumab | QVQLVQSGAEVKKPGASV | DIQMTQSPSSLSASVGD | na | na |
| (Format: Whole | KVSCKASGYTFTSYWMN | RVTITCRASENIYSYLAWY | | |
| mAb) | WVRQAPGQGLEWMGRI | QQKPGKAPKLLIYNAKTL | | |
| | DPYDSETHYAQKLQGRVT | AEGVPSRFSGSGSGTDFT | | |
| | MTTDTSTSTAYMELRSLRS | LTISSLQPEDFATYYCQH | | |
| | DDTAVYYCARGGYDFDVG | HYGTPRTFGGGTKVEIK | | |
| | TLYWFFDVWGQGTTVTV | (SEQ ID NO: 544) | | |
| | SS (SEQ ID NO: 543) | | | |
| Mosunetuzumab | EVQLVQSGAEVKKPGASV | DIVMTQSPDSLAVSLGER | EVQLVESGGGLVQ | DIQMTQSPSSLSA |
| (Format: | KVSCKASGYTFTNYYIHWV | ATINCKSSQSLLNSRTRK | PGGSLRLSCAASGY | SVGDRVTITCRAS |
| Bispecific mAb) | RQAPGQGLEWIGWIYPG | NYLAWYQQKPGQPPKLL | TFTSYNMHWVRQ | SSVSYMHWYQQ |
| | DGNTKYNEKFKGRATLTA | IYWASTRESGVPDRFSGS | APGKGLEWVGAIY | KPGKAPKPLIYAP |
| | DTSTSTAYLELSSLRSEDTA | GSGTDFTLTISSLQAEDV | PGNGDTSYNQKFK | SNLASGVPSRFSG |
| | VYYCARDSYSNYYFDYWG | AVYYCTQSFILRTFGQGT | GRFTISVDKSKNTL | SGSGTDFTLTISSL |
| | QGTLVTVSS | KVEIK (SEQ ID NO: 546) | YLQMNSLRAEDTA | QPEDFATYYCQQ |
| | (SEQ ID NO: 545) | | VYYCARVVYYSNSY | WSFNPPTFGQGT |
| | | | WYFDVWGQGTLV | KVEIK (SEQ ID |
| | | | TVSS (SEQ ID NO: | NO: 548) |
| | | | 547) | |
| Motavizumab | QVTLRESGPALVKPTQTLT | DIQMTQSPSTLSASVGD | na | na |
| (Format: Whole | LTCTFSGFSLSTAGMSVG | RVTITCSASSRVGYMHW | | |
| mAb) | WIRQPPGKALEWLADIW | YQQKPGKAPKLLIYDTSKL | | |
| | WDDKKHYNPSLKDRLTISK | ASGVPSRFSGSGSGTEFT | | |
| | DTSKNQVVLKVTNMDPA | LTISSLQPDDFATYYCFQG | | |
| | DTATYYCARDMIFNFYFD | SGYPFTFGGGTKVEIK | | |
| | VWGQGTTVTVSS (SEQ ID | (SEQ ID NO: 550) | | |
| | NO: 549) | | | |
| Moxetumomab | EVQLVESGGGLVKPGGSL | DIQMTQTTSSLSASLGDR | na | na |
| (Format: Fv | KLSCAASGFAFSIYDMSW | VTISCRASQDISNYLNWY | | |
| Fusion) | VRQTPEKCLEWVAYISSG | QQKPDGTVKLLIYYTSILH | | |
| | GGTTYYPDTVKGRFTISRD | SGVPSRFSGSGSGTDYSL | | |
| | NAKNTLYLQMSSLKSEDT | TISNLEQEDFATYFCQQG | | |
| | AMYYCARHSGYGTHWGV | NTLPWTFGCGTKLEI | | |
| | LFAYWGQGTLVTVSA | (SEQ ID NO: 552) | | |
| | (SEQ ID NO: 551) | | | |
| Murlentamab | QVRLVQSGAEVKKPGASV | DIQMTQSPSTLSASVGD | na | na |
| (Format: Whole | KVSCKASGYTFTSYHIHWV | RVTITCRASSSVRYIAWY | | |
| mAb) | RQAPGQRLEWMGWIYP | QQKPGKAPKLLTYPTSSL | | |
| | GDDSTKYSQKFQGRVTITR | KSGVPSRFSGSGSGTEFT | | |
| | DTSASTAYMELSSLRSEDT | LTISSLQPDDFATYYCLQ | | |
| | AVYYCTRGDRFAYWGQG | WSSYPWTFGGGTKVEIK | | |
| | TLVTVSS (SEQ ID NO: | (SEQ ID NO: 554) | | |
| | 553) | | | |
| Muromonab | QVQLQQSGAELARPGASV | QIVLTQSPAIMSASPGEK | na | na |
| (Zolimomab) | KMSCKASGYTFTRYTMH | VTMTCSASSSVSYMNW | | |
| (Format: Whole | WVKQRPGQGLEWIGYIN | YQQKSGTSPKRWIYDTSK | | |
| mAb) | PSRGYTNYNQKFKDKATL | LASGVPAHFRGSGSGTSY | | |
| | TTDKSSSTAYMQLSSLTSE | SLTISGMEAEDAATYYCQ | | |
| | DSAVYYCARYYDDHYCLD | QWSSNPFTFGSGTKLEIN | | |
| | YWGQGTTLTVSS (SEQ ID | (SEQ ID NO: 556) | | |
| | NO: 555) | | | |
| Namilumab | QVQLVQSGAEVKKPGASV | DIQMTQSPSSVSASVGD | na | na |
| (Format: Whole | KVSCKAFGYPFTDYLLHW | RVTIACRASQNIRNILNW | | |
| mAb) | VRQAPGQGLEWVGWLN | YQQRPGKAPQLLIYAASN | | |
| | PYSGDTNYAQKFQGRVT | LQSGVPSRFSGSGSGTDF | | |
| | MTRDTSISTAYMELSRLRS | TLTINSLQPEDFATYYCQ | | |
| | DDTAVYYCTRTTLISVYFDY | QSYSMPRTFGGGTKLEIK | | |
| | WGQGTMVTVSS (SEQ ID | (SEQ ID NO: 558) | | |
| | NO: 557) | | | |
| Naptumomab | EVQLQQSGPDLVKPGASV | SIVMTQTPTSLLVSAGDR | na | na |
| (Format: Fab | KISCKASGYSFTGYYMHW | VTITCKASQSVSNDVAW | | |
| Fusion) | VKQSPGKGLEWIGRINPN | YQQKPGQSPKLLISYTSSR | | |
| | NGVTLYNQKFKDKATLTV | YAGVPDRFSGSGYGTDF | | |
| | DKSSTTAYMELRSLTSEDS | TLTISSVQAEDAAVYFCQ | | |
| | AVYYCARSTMITNYVMDY | QDYNSPPTFGGGTKLEIK | | |
| | WGQGTSVTVSS (SEQ ID | (SEQ ID NO: 560) | | |
| | NO: 559) | | | |
| Naratuximab | QVQVQESGPGLVAPSQTL | DIQMTQSPSSLSVSVGER | na | na |
| (Format: Whole | SITCTVSGFSLTTSGVSWV | VTITCRASENIRSNLAWY | | |
| mAb ADC) | RQPPGKGLEWLGVIWGD | QQKPGKSPKLLVNVATN | | |
| | GSTNYHPSLKSRLSIKKDHS | LADGVPSRFSGSGSGTDY | | |
| | KSQVFLKLNSLTAADTATY | SLKINSLQPEDFGTYYCQ | | |
| | YCAKGGYSLAHWGQGTL | HYWGTTWTFGQGTKLEI | | |
| | VTVSS (SEQ ID NO: 561) | K (SEQ ID NO: 562) | | |
| Narnatumab | EVQLVESGGGLVQPGGSL | EIVLTQSPATLSLSPGERA | na | na |
| (Format: Whole | RLSCAASGFTFSSYLMTW | TLSCRASQSVSRYLAWYQ | | |
| mAb) | VRQAPGKGLEWVANIKQ | QKPGQAPRLLIYDASNRA | | |
| | DGSEKYYVDSVKGRFTISR | TGIPARFSGSGSGTDFTLT | | |
| | DNAKNSLNLQMNSLRAE | ISSLEPEDFAVYYCQQRS | | |
| | DTAVYYCTRDGYSSGRHY | NWPRTFGQGTKVEIK | | |
| | GMDVWGQGTTVIVSS | (SEQ ID NO: 564) | | |
| | (SEQ ID NO: 563) | | | |
| Natalizumab | QVQLVQSGAEVKKPGASV | DIQMTQSPSSLSASVGD | na | na |
| (Format: Whole | KVSCKASGFNIKDTYIHWV | RVTITCKTSQDINKYMA | | |
| mAb) | RQAPGQRLEWMGRIDPA | WYQQTPGKAPRLLIHYTS | | |
| | NGYTKYDPKFQGRVTITA | ALQPGIPSRFSGSGSGRD | | |
| | DTSASTAYMELSSLRSEDT | YTFTISSLQPEDIATYYCL | | |
| | AVYYCAREGYYGNYGVYA | QYDNLWTFGQGTKVEIK | | |
| | MDYWGQGTLVTVSS | (SEQ ID NO: 566) | | |
| | (SEQ ID NO: 565) | | | |
| Navivumab | QVQLVQSGAEVKKPGASV | EVVLTQSPGTLALPPGER | na | na |
| (Format: Whole | KVSCKTSGYSFSTYGVSWV | ATLSCRASHRVGSTYIAW | | |
| mAb) | RQAPGQGPEWVGWISAY | YQQKSGQAPRRLIYGAS | | |
| | TGITDYAQKFQGRVTLTTD | NRATDIPDRFSGSGSGTD | | |
| | ATTATAFLDLRSLRPDDTA | FTLTIRRLEPEDSAVYYCQ | | |
| | TYFCARDKVQGRVEVGSG | QFSVSPWTFGQGTRVEI | | |
| | GRHDYWGQGTLVIVSS | K (SEQ ID NO: 568) | | |
| | (SEQ ID NO: 567) | | | |
| Navicixizumab | QVQLVQSGAEVKKPGASV | DIVMTQSPDSLAVSLGER | QVQLVQSGAEVKK | DIVMTQSPDSLA |
| (Format: | KISCKASGYSFTAYYIHWV | ATISCRASESVDNYGISF | PGASVKVSCKASG | VSLGERATISCRAS |
| Bispecific mAb) | KQAPGQGLEWIGYISNYN | MKWFQQKPGQPPKLLIY | YTFTNYWMHWVR | ESVDNYGISFMK |
| | RATNYNQKFKGRVTFTTD | AASNQGSGVPDRFSGSG | QAPGQGLEWMG | WFQQKPGQPPKL |
| | TSTSTAYMELRSLRSDDTA | SGTDFTLTISSLQAEDVAV | DINPSNGRTSYKEK | LIYAASNQGSGVP |
| | VYYCARDYDYDVGMDYW | YYCQQSKEVPWTFGGGT | FKRRVTLSVDKSSS | DRFSGSGSGTDFT |
| | GQGTLVTVSS (SEQ ID | KVEIK (SEQ ID NO: 570) | TAYMELSSLRSEDT | LTISSLQAEDVAV |
| | NO: 569) | | AVYFCTIHYDDKYY | YYCQQSKEVPWT |
| | | | PLMDYWGQGTLV | FGGGTKVEIK |
| | | | TVSS (SEQ ID NO: | (SEQ ID NO: 572) |
| | | | 571) | |
| Naxitamab | QVQLVESGPGVVQPGRSL | EIVMTQTPATLSVSAGER | na | na |
| (Format: Whole | RISCAVSGFSVTNYGVHW | VTITCKASQSVSNDVTW | | |
| mAb) | VRQPPGKGLEWLGVIWA | YQQKPGQAPRLLIYSASN | | |
| | GGITNYNSAFMSRLTISKD | RYSGVPARFSGSGYGTEF | | |
| | NSKNTVYLQMNSLRAEDT | TFTISSVQSEDFAVYFCQ | | |
| | AMYYCASRGGHYGYALDY | QDYSSFGQGTKLEIK | | |
| | WGQGTLVTVSS (SEQ ID | (SEQ ID NO: 574) | | |
| | NO: 573) | | | |
| Necitumumab | QVQLQESGPGLVKPSQTL | EIVMTQSPATLSLSPGER | na | na |
| (Format: Whole | SLTCTVSGGSISSGDYYWS | ATLSCRASQSVSSYLAWY | | |
| mAb) | WIRQPPGKGLEWIGYIYYS | QQKPGQAPRLLIYDASN | | |
| | GSTDYNPSLKSRVTMSVD | RATGIPARFSGSGSGTDF | | |
| | TSKNQFSLKVNSVTAADT | TLTISSLEPEDFAVYYCHQ | | |
| | AVYYCARVSIFGVGTFDY | YGSTPLTFGGGTKAEIK | | |
| | WGQGTLVTVSS (SEQ ID | (SEQ ID NO: 576) | | |
| | NO: 575) | | | |
| Nemolizumab | QVQLVQSGAEVKKPGASV | DIQMTQSPSSLSASVGD | na | na |
| (Format: Whole | KVSCKASGYTFTGYIMNW | RVTITCQASEDIYSFVAW | | |
| mAb) | VRQAPGQGLEWMGLINP | YQQKPGKAPKLLIYNAQT | | |
| | YNGGTDYNPQFQDRVTIT | EAQGVPSRFSGSGSGTD | | |
| | ADKSTSTAYMELSSLRSED | FTLTISSLQPEDFATYYCQ | | |
| | TAVYYCARDGYDDGPYTL | HHYDSPLTFGGGTKVEIK | | |
| | ETWGQGTLVTVSS (SEQ | (SEQ ID NO: 578) | | |
| | ID NO: 577) | | | |
| Nesvacumab | EVQLVESGGGLVQPGGSL | EIVLTQSPGTLSLSPGERA | na | na |
| (Format: Whole | RLSCAASGFTFSSYDIHWV | TLSCRASQSVSSTYLAWY | | |
| mAb) | RQATGKGLEWVSAIGPAG | QQKPGQAPRLLIYGASSR | | |
| | DTYYPGSVKGRFTISRENA | ATGIPDRFSGSGSGTDFT | | |
| | KNSLYLQMNSLRAGDTAV | LTISRLEPEDFAVYYCQHY | | |
| | YYCARGLITFGGLIAPFDY | DNSQTFGQGTKVEIK | | |
| | WGQGTLVTVSS (SEQ ID | (SEQ ID NO: 580) | | |
| | NO: 579) | | | |
| Netakimab | QVQLVQSGGGLVQAGGS | EIVLTQSPGTLSLSPGERA | na | na |
| (Format: Whole | LRLSCAASGGTFATSPMG | TLSCRASQSVSSSYLAWY | | |
| mAb) | WLRQAPGKGTEFVAAISP | QQKPGQAPRLLIYDASSR | | |
| | SGGDRIYADSVKGRFTISR | ATGIPDRFSGSGSGTDFT | | |
| | DNAGYFIYLQMNSLKPED | LTISRLEPEDFAVYYCQQY | | |
| | TAVYYCAVRRRFDGTSYYT | SYSPVTFGQGTKVEIK | | |
| | GDYDSWGQGTLVTVSS | (SEQ ID NO: 582) | | |
| | (SEQ ID NO: 581) | | | |
| Nidanilimab | QVQLVQSGAEVKKPGSSV | DIQMTQSPSSLSASVGD | na | na |
| (Format: Whole | KVSCKASGYAFTSSWMN | RVTITCQASQGINNYLN | | |
| mAb) | WVRQAPGQGLEWMGRI | WYQQKPGKAPKLLIHYTS | | |
| | YPGDGNTHYAQKFQGRV | GLHAGVPSRFSGSGSGT | | |
| | TLTADKSTSTAYMELSSLR | DYTLTISSLEPEDVATYYC | | |
| | SEDTAVYYCGEGYLDPMD | QQYSILPWTFGGGTKVEI | | |
| | YWGQGTLVTVSS (SEQ ID | K (SEQ ID NO: 584) | | |
| | NO: 583) | | | |
| Nimacimab | QVQLVQSGAEVKKPGSSV | EIVLTQSPATLSLSPGERA | na | na |
| (Format: Whole | KVSCKASGYEFSYYWMN | TLSCRASQSVSSSYLHWY | | |
| mAb) | WVRQAPGQGLEWMGQI | QQKPGQAPRLLIYSTSNL | | |
| | YPGDGETKYAQKFQGRVT | ASGIPARFSGSGSGTDFT | | |
| | ITADKSTSTAYMELSSLRSE | LTISRLEPEDFAVYYCHQY | | |
| | DTAVYYCARSHGNYLPYW | HRSPPTFGQGTKVEIK | | |
| | GQGTLVTVSS (SEQ ID | (SEQ ID NO: 586) | | |
| | NO: 585) | | | |
| Nimotuzumab | QVQLQQSGAEVKKPGSSV | DIQMTQSPSSLSASVGD | na | na |
| (Format: Whole | KVSCKASGYTFTNYYIYWV | RVTITCRSSQNIVHSNGN | | |
| mAb) | RQAPGQGLEWIGGINPTS | TYLDWYQQTPGKAPKLLI | | |
| | GGSNFNEKFKTRVTITADE | YKVSNRFSGVPSRFSGSG | | |
| | SSTTAYMELSSLRSEDTAF | SGTDFTFTISSLQPEDIAT | | |
| | YFCTRQGLWFDSDGRGFD | YYCFQYSHVPWTFGQGT | | |
| | FWGQGTTVTVSS (SEQ ID | KLQIT (SEQ ID NO: 588) | | |
| | NO: 587) | | | |
| Nirsevimab | QVQLVQSGAEVKKPGSSV | DIQMTQSPSSLSAAVGD | na | na |
| (Format: Whole | MVSCQASGGLLEDYIINW | RVTITCQASQDIVNYLN | | |
| mAb) | VRQAPGQGPEWMGGIIP | WYQQKPGKAPKLLIYVAS | | |
| | VLGTVHYGPKFQGRVTITA | NLETGVPSRFSGSGSGTD | | |
| | DESTDTAYMELSSLRSEDT | FSLTISSLQPEDVATYYCQ | | |
| | AMYYCATETALVVSETYLP | QYDNLPLTFGGGTKVEIK | | |
| | HYFDNWGQGTLVTVSS | (SEQ ID NO: 590) | | |
| | (SEQ ID NO: 589) | | | |
| Nivolumab | QVQLVESGGGVVQPGRSL | EIVLTQSPATLSLSPGERA | na | na |
| (Format: Whole | RLDCKASGITFSNSGMHW | TLSCRASQSVSSYLAWYQ | | |
| mAb) | VRQAPGKGLEWVAVIWY | QKPGQAPRLLIYDASNRA | | |
| | DGSKRYYADSVKGRFTISR | TGIPARFSGSGSGTDFTLT | | |
| | DNSKNTLFLQMNSLRAED | ISSLEPEDFAVYYCQQSS | | |
| | TAVYYCATNDDYWGQGT | NWPRTFGQGTKVEIK | | |
| | LVTVSS (SEQ ID NO: 591) | (SEQ ID NO: 592) | | |
| Obexelimab | EVQLVESGGGLVKPGGSL | DIVMTQSPATLSLSPGER | na | na |
| (Format: Whole | KLSCAASGYTFTSYVMHW | ATLSCRSSKSLQNVNGNT | | |
| mAb) | VRQAPGKGLEWIGYINPY | YLYWFQQKPGQSPQLLIY | | |
| | NDGTKYNEKFQGRVTISS | RMSNLNSGVPDRFSGSG | | |
| | DKSISTAYMELSSLRSEDTA | SGTEFTLTISSLEPEDFAVY | | |
| | MYYCARGTYYYGTRVFDY | YCMQHLEYPITFGAGTKL | | |
| | WGQGTLVTVSS (SEQ ID | EIK (SEQ ID NO: 594) | | |
| | NO: 593) | | | |
| Obiltoxaximab | QVQLQQSGPELKKPGASV | DIQMTQSPSSLSASVGD | na | na |
| (Format: Whole | KVSCKDSGYAFSSSWMN | RVTITCRASQDIRNYLNW | | |
| mAb) | WVRQAPGQGLEWIGRIY | YQQKPGKAVKLLIYYTSRL | | |
| | PGDGDTNYNGKFQGRVTI | LPGVPSRFSGSGSGTDYS | | |
| | TADKSSSTAYMELSSLRSE | LTISSQEQEDIGTYFCQQ | | |
| | DTAVYFCARSGLLRYAMD | GNTLPWTFGQGTKVEIR | | |
| | YWGQGTLVTVSS (SEQ ID | (SEQ ID NO: 596) | | |
| | NO: 595) | | | |
| Obinutuzumab | QVQLVQSGAEVKKPGSSV | DIVMTQTPLSLPVTPGEP | na | na |
| (Afutuzumab) | KVSCKASGYAFSYSWINW | ASISCRSSKSLLHSNGITYL | | |
| (Format: Whole | VRQAPGQGLEWMGRIFP | YWYLQKPGQSPQLLIYQ | | |
| mAb) | GDGDTDYNGKFKGRVTIT | MSNLVSGVPDRFSGSGS | | |
| | ADKSTSTAYMELSSLRSED | GTDFTLKISRVEAEDVGV | | |
| | TAVYYCARNVFDGYWLVY | YYCAQNLELPYTFGGGTK | | |
| | WGQGTLVTVSS (SEQ ID | VEIK (SEQ ID NO: 598) | | |
| | NO: 597) | | | |
| Ocaratuzumab | EVQLVQSGAEVKKPGESL | EIVLTQSPGTLSLSPGERA | na | na |
| (Format: Whole | KISCKGSGRTFTSYNMHW | TLSCRASSSVPYIHWYQQ | | |
| mAb) | VRQMPGKGLEWMGAIYP | KPGQAPRLLIYATSALAS | | |
| | LTGDTSYNQKSKLQVTISA | GIPDRFSGSGSGTDFTLTI | | |
| | DKSISTAYLQWSSLKASDT | SRLEPEDFAVYYCQQWL | | |
| | AMYYCARSTYVGGDWQF | SNPPTFGQGTKLEIK | | |
| | DVWGKGTTVTVSS (SEQ | (SEQ ID NO: 600) | | |
| | ID NO: 599) | | | |
| Ocrelizumab | EVQLVESGGGLVQPGGSL | DIQMTQSPSSLSASVGD | na | na |
| (Format: Whole | RLSCAASGYTFTSYNMHW | RVTITCRASSSVSYMHW | | |
| mAb) | VRQAPGKGLEWVGAIYPG | YQQKPGKAPKPLIYAPSN | | |
| | NGDTSYNQKFKGRFTISVD | LASGVPSRFSGSGSGTDF | | |
| | KSKNTLYLQMNSLRAEDT | TLTISSLQPEDFATYYCQQ | | |
| | AVYYCARVVYYSNSYWYF | WSFNPPTFGQGTKVEIK | | |
| | DVWGQGTLVTVSS (SEQ | (SEQ ID NO: 602) | | |
| | ID NO: 601) | | | |
| Ofatumumab | EVQLVESGGGLVQPGRSL | EIVLTQSPATLSLSPGERA | na | na |
| (Format: Whole | RLSCAASGFTFNDYAMH | TLSCRASQSVSSYLAWYQ | | |
| mAb) | WVRQAPGKGLEWVSTIS | QKPGQAPRLLIYDASNRA | | |
| | WNSGSIGYADSVKGRFTIS | TGIPARFSGSGSGTDFTLT | | |
| | RDNAKKSLYLQMNSLRAE | ISSLEPEDFAVYYCQQRS | | |
| | DTALYYCAKDIQYGNYYYG | NWPITFGQGTRLEIK | | |
| | MDVWGQGTTVTVSS | (SEQ ID NO: 604) | | |
| | (SEQ ID NO: 603) | | | |
| Olaratumab | QLQLQESGPGLVKPSETLS | EIVLTQSPATLSLSPGERA | na | na |
| (Format: Whole | LTCTVSGGSINSSSYYWG | TLSCRASQSVSSYLAWYQ | | |
| mAb) | WLRQSPGKGLEWIGSFFY | QKPGQAPRLLIYDASNRA | | |
| | TGSTYYNPSLRSRLTISVDT | TGIPARFSGSGSGTDFTLT | | |
| | SKNQFSLMLSSVTAADTA | ISSLEPEDFAVYYCQQRS | | |
| | VYYCARQSTYYYGSGNYY | NWPPAFGQGTKVEIK | | |
| | GWFDRWDQGTLVTVSS | (SEQ ID NO: 606) | | |
| | (SEQ ID NO: 605) | | | |
| Oleclumab | EVQLLESGGGLVQPGGSL | QSVLTQPPSASGTPGQR | na | na |
| (Format: Whole | RLSCAASGFTFSSYAYSWV | VTISCSGSLSNIGRNPVN | | |
| mAb) | RQAPGKGLEWVSAISGSG | WYQQLPGTAPKLLIYLDN | | |
| | GRTYYADSVKGRFTISRDN | LRLSGVPDRFSGSKSGTS | | |
| | SKNTLYLQMNSLRAEDTA | ASLAISGLQSEDEADYYC | | |
| | VYYCARLGYGRVDEWGR | ATWDDSHPGWTFGGGT | | |
| | GTLVTVSS | KLTVL (SEQ ID NO: 608) | | |
| | (SEQ ID NO: 607) | | | |
| Olendalizumab | QVQLVQSGAEVKKPGASV | DIQMTQSPSSLSASVGD | na | na |
| (Format: Whole | KVSCKASGYTFTDYSMDW | RVTITCRASESVDSYGNS | | |
| mAb) | VRQAPGQGLEWMGAIHL | FMHWYQQKPGKAPKLLI | | |
| | NTGYTNYNQKFKGRVTM | YRASNLESGVPSRFSGSG | | |
| | TRDTSTSTVYMELSSLRSE | SGTDFTLTISSLQPEDFAT | | |
| | DTAVYYCARGFYDGYSPM | YYCQQSNEDPYTFGGGT | | |
| | DYWGQGTTVTVSS (SEQ | KVEIK | | |
| | ID NO: 609) | (SEQ ID NO: 610) | | |
| Olinvacimab | QMQLVQSGAEVKKPGAS | NFMLTQPPSVSVSPGKT | na | na |
| (Tanibirumab) | VKLSCKASGYTFSSYWMH | ARITCRGDNLGDVNVH | | |
| (Format: Whole | WVRQAPGQRLEWMGEI | WYQQRPGQAPVLVMYY | | |
| mAb) | NPGNGHTNYNEKFKSRVT | DADRPSGIPERFSGSNSG | | |
| | ITVDKSASTAYMELSSLRSE | NTATLTISGVEAGDEADY | | |
| | DTAVYYCAKIWGPSLTSPF | YCQVWDRTSEYVFGTGT | | |
| | DYWGQGTLVTVSS (SEQ | KVTVL | | |
| | ID NO: 611) | (SEQ ID NO: 612) | | |
| Olokizumab | EVQLVESGGGLVQPGGSL | DIQMTQSPSSLSASVGD | na | na |
| (Format: Whole | RLSCAASGFNFNDYFMN | RVTITCQASQDIGISLSW | | |
| mAb) | WVRQAPGKGLEWVAQM | YQQKPGKAPKLLIYNAN | | |
| | RNKNYQYGTYYAESLEGRF | NLADGVPSRFSGSGSGT | | |
| | TISRDDSKNSLYLQMNSLK | DFTLTISSLQPEDFATYYC | | |
| | TEDTAVYYCARESYYGFTS | LQHNSAPYTFGQGTKLEI | | |
| | YWGQGTLVTVSS (SEQ ID | K (SEQ ID NO: 614) | | |
| | NO: 613) | | | |
| Omalizumab | EVQLVESGGGLVQPGGSL | DIQLTQSPSSLSASVGDR | na | na |
| (Format: Whole | RLSCAVSGYSITSGYSWN | VTITCRASQSVDYDGDSY | | |
| mAb) | WIRQAPGKGLEWVASITY | MNWYQQKPGKAPKLLIY | | |
| | DGSTNYNPSVKGRITISRD | AASYLESGVPSRFSGSGS | | |
| | DSKNTFYLQMNSLRAEDT | GTDFTLTISSLQPEDFATY | | |
| | AVYYCARGSHYFGHWHF | YCQQSHEDPYTFGQGTK | | |
| | AVWGQGTLVTVSS (SEQ | VEIK (SEQ ID NO: 616) | | |
| | ID NO: 615) | | | |
| Omburtamab | QVQLQQSGAELVKPGASV | DIVMTQSPATLSVTPGD | na | na |
| (Format: Whole | KLSCKASGYTFTNYDINWV | RVSLSCRASQSISDYLHW | | |
| mAb | RQRPEQGLEWIGWIFPGD | YQQKSHESPRLLIKYASQS | | |
| Radiolabelled) | GSTQYNEKFKGKATLTTDT | ISGIPSRFSGSGSGSDFTL | | |
| | SSSTAYMQLSRLTSEDSAV | SINSVEPEDVGVYYCQNG | | |
| | YFCARQTTATWFAYWGQ | HSFPLTFGAGTKLELK | | |
| | GTLVTVSA (SEQ ID NO: | (SEQ ID NO: 618) | | |
| | 617) | | | |
| Onartuzumab | EVQLVESGGGLVQPGGSL | DIQMTQSPSSLSASVGD | na | na |
| (Format: Fab + | RLSCAASGYTFTSYWLHW | RVTITCKSSQSLLYTSSQK | | |
| di-Fc) | VRQAPGKGLEWVGMIDP | NYLAWYQQKPGKAPKLL | | |
| | SNSDTRFNPNFKDRFTISA | IYWASTRESGVPSRFSGS | | |
| | DTSKNTAYLQMNSLRAED | GSGTDFTLTISSLQPEDFA | | |
| | TAVYYCATYRSYVTPLDYW | TYYCQQYYAYPWTFGQG | | |
| | GQGTLVTVSS (SEQ ID | TKVEIK (SEQ ID NO: | | |
| | NO: 619) | 620) | | |
| Ontamalimab | QVQLVQSGAEVKKPGASV | DIVMTQTPLSLSVTPGQP | na | na |
| (Format: Whole | KVSCKASGYTFTSYG1NWV | ASISCKSSQSLLHTDGTTY | | |
| mAb) | RQAPGQGLEWMGWISV | LYWYLQKPGQPPQLLIYE | | |
| | YSGNTNYAQKVQGRVTM | VSNRFSGVPDRFSGSGS | | |
| | TADTSTSTAYMDLRSLRSD | GTDFTLKISRVEAEDVGIY | | |
| | DTAVYYCAREGSSSSGDYY | YCMQNIQLPWTFGQGT | | |
| | YGMDVWGQGTTVTVSS | KVEIK (SEQ ID NO: 622) | | |
| | (SEQ ID NO: 621) | | | |
| Ontuxizumab | QVQLQESGPGLVRPSQTL | DIQMTQSPSSLSASVGD | na | na |
| (Format: Whole | SLTCTASGYTFTDYVIHWV | RVTITCRASQNVGTAVA | | |
| mAb) | KQPPGRGLEWIGYINPYD | WLQQTPGKAPKLLIYSAS | | |
| | DDTTYNQKFKGRVTMLV | NRYTGVPSRFSGSGSGTD | | |
| | DTSSNTAYLRLSSVTAEDT | YTFTISSLQPEDIATYYCQ | | |
| | AVYYCARRGNSYDGYFDY | QYTNYPMYTFGQGTKV | | |
| | SMDYWGSGTPVTVSS | QIK (SEQ ID NO: 624) | | |
| | (SEQ ID NO: 623) | | | |
| Onvatilimab | QVQLVQSGAEVKKPGSSV | DIQMTQSPSSLSASVGD | na | na |
| (Format: Whole | KVSCKASGGTFSSYAISWV | RVTITCRASQSIDTRLNW | | |
| mAb) | RQAPGQGLEWMGGIIPIF | YQQKPGKAPKLLIYSASSL | | |
| | GTANYAQKFQGRVTITAD | QSGVPSRFSGSGSGTDFT | | |
| | ESTSTAYMELSSLRSEDTA | LTISSLQPEDFATYYCQQS | | |
| | VYYCARSSYGWSYEFDYW | AYNPITFGQGTKVEIK | | |
| | GQGTLVTVSS (SEQ ID | (SEQ ID NO: 626) | | |
| | NO: 625) | | | |
| Opicinumab | EVQLLESGGGLVQPGGSL | DIQMTQSPATLSLSPGER | na | na |
| (Format: Whole | RLSCAASGFTFSAYEMKW | ATLSCRASQSVSSYLAWY | | |
| mAb) | VRQAPGKGLEWVSVIGPS | QQKPGQAPRLLIYDASN | | |
| | GGFTFYADSVKGRFTISRD | RATGIPARFSGSGSGTDF | | |
| | NSKNTLYLQMNSLRAEDT | TLTISSLEPEDFAVYYCQQ | | |
| | AVYYCATEGDNDAFDIWG | RSNWPMYTFGQGTKLEI | | |
| | QGTTVTVSS (SEQ ID NO: | K (SEQ ID NO: 628) | | |
| | 627) | | | |
| Oportuzumab | EVQLVQSGPGLVQPGGSV | DIQMTQSPSSLSASVGD | na | na |
| (Format: scFv) | RISCAASGYTFTNYGMNW | RVTITCRSTKSLLHSNGIT | | |
| | VKQAPGKGLEWMGWIN | YLYWYQQKPGKAPKLLIY | | |
| | TYTGESTYADSFKGRFTFSL | QMSNLASGVPSRFSSSG | | |
| | DTSASAAYLQINSLRAEDT | SGTDFTLTISSLQPEDFAT | | |
| | AVYYCARFAIKGDYWGQG | YYCAQNLEIPRTFGQGTK | | |
| | TLLTVSS (SEQ ID NO: | VELK (SEQ ID NO: 630) | | |
| | 629) | | | |
| Orilanolimab | QVQLVQSGAELKKPGASV | DIQMTQSPSSLSASVGD | na | na |
| (Format: Whole | KLSCKASGYTFTSYGISWV | RVTITCKASDHINNWLA | | |
| mAb) | KQATGQGLEWIGEIYPRS | WYQQKPGQAPRLLISGA | | |
| | GNTYYNEKFKGRATLTAD | TSLETGVPSRFSGSGTGK | | |
| | KSTSTAYMELRSLRSEDSA | DYTLTISSLQPEDFATYYC | | |
| | VYFCARSTTVRPPGIWGT | QQYWSTPYTFGGGTKVE | | |
| | GTTVTVSS (SEQ ID NO: | IK (SEQ ID NO: 632) | | |
| | 631) | | | |
| Orticumab | EVQLLESGGGLVQPGGSL | QSVLTQPPSASGTPGQR | na | na |
| (Format: Whole | RLSCAASGFTFSNAWMS | VTISCSGSNTNIGKNYVS | | |
| mAb) | WVRQAPGKGLEWVSSISV | WYQQLPGTAPKLLIYANS | | |
| | GGHRTYYADSVKGRSTISR | NRPSGVPDRFSGSKSGTS | | |
| | DNSKNTLYLQMNSLRAED | ASLAISGLRSEDEADYYCA | | |
| | TAVYYCARIRVGPSGGAFD | SWDASLNGWVFGGGTK | | |
| | YWGQGTLVTVSS (SEQ ID | LTVL (SEQ ID NO: 634) | | |
| | NO: 633) | | | |
| Osocimab | EVQLLESGGGLVQPGGSL | DIQMTQSPSSLSASVGD | na | na |
| (Format: Whole | RLSCAASGFTFSQYGMD | RVTITCQASQDISNYLNW | | |
| mAb) | WVRQAPGKGLEWVSGIG | YQQKPGKAPKLLIYDASN | | |
| | PSGGSTVYADSVKGRFTIS | LETGVPSRFSGSGSGTDF | | |
| | RDNSKNTLYLQMNSLRAE | TFTISSLQPEDIATYYCQQ | | |
| | DTAVYYCTRGGPYYYYGM | ADSFPVTFGGGTKVEIK | | |
| | DVWGQGTTVTVSS (SEQ | (SEQ ID NO: 636) | | |
| | ID NO: 635) | | | |
| Otelixizumab | EVQLLESGGGLVQPGGSL | DIQLTQPNSVSTSLGSTV | na | na |
| (Format: Whole | RLSCAASGFTFSSFPMAW | KLSCTLSSGNIENNYVHW | | |
| mAb) | VRQAPGKGLEWVSTISTS | YQLYEGRSPTTMIYDDDK | | |
| | GGRTYYRDSVKGRFTISRD | RPDGVPDRFSGSIDRSSN | | |
| | NSKNTLYLQMNSLRAEDT | SAFLTIHNVAIEDEAIYFC | | |
| | AVYYCAKFRQYSGGFDYW | HSYVSSFNVFGGGTKLTV | | |
| | GQGTLVTVSS (SEQ ID | L (SEQ ID NO: 638) | | |
| | NO: 637) | | | |
| Otilimab | QVQLVESGGGLVQPGGSL | DIELTQPPSVSVAPGQTA | na | na |
| (Format: Whole | RLSCAASGFTFSSYWMN | RISCSGDSIGKKYAYWYQ | | |
| mAb) | WVRQAPGKGLEWVSGIE | QKPGQAPVLVIYKKRPSG | | |
| | NKYAGGATYYAASVKGRF | IPERFSGSNSGNTATLTIS | | |
| | TISRDNSKNTLYLQMNSLR | GTQAEDEADYYCSAWG | | |
| | AEDTAVYYCARGFGTDFW | DKGMVFGGGTKLTVL | | |
| | GQGTLVTVSS (SEQ ID | (SEQ ID NO: 640) | | |
| | NO: 639) | | | |
| Otlertuzumab | EVQLVQSGAEVKKPGESL | EIVLTQSPATLSLSPGERA | na | na |
| (Format: di- | KISCKGSGYSFTGYNMNW | TLSCRASENVYSYLAWYQ | | |
| scFv+Fc) | VRQMPGKGLEWMGNID | QKPGQAPRLLIYFAKTLA | | |
| | PYYGGTTYNRKFKGQVTIS | EGIPARFSGSGSGTDFTLT | | |
| | ADKSISTAYLQWSSLKASD | ISSLEPEDFAVYYCQHHS | | |
| | TAMYYCARSVGPFDSWG | DNPWTFGQGTKVEIK | | |
| | QGTLVTVSS (SEQ ID NO: | (SEQ ID NO: 642) | | |
| | 641) | | | |
| Oxelumab | EVQLLESGGGLVQPGGSL | DIQMTQSPSSLSASVGD | na | na |
| (Format: Whole | RLSCAASGFTFNSYAMSW | RVTITCRASQGISSWLAW | | |
| mAb) | VRQAPGKGLEWVSIISGSG | YQQKPEKAPKSLIYAASSL | | |
| | GFTYYADSVKGRFTISRDN | QSGVPSRFSGSGSGTDFT | | |
| | SRTTLYLQMNSLRAEDTA | LTISSLQPEDFATYYCQQY | | |
| | VYYCAKDRLVAPGTFDYW | NSYPYTFGQGTKLEIK | | |
| | GQGALVTVSS (SEQ ID | (SEQ ID NO: 644) | | |
| | NO: 643) | | | |
| Ozanezumab | QVQLVQSGAEVKKPGASV | DIVMTQSPLSNPVTLGQ | na | na |
| (Format: Whole | KVSCKASGYTFTSYWMH | PVSISCRSSKSLLYKDGKT | | |
| mAb) | WVRQAPGQGLEWIGNIN | YLNWFLQRPGQSPQLLIY | | |
| | PSNGGTNYNEKFKSKATM | LMSTRASGVPDRFSGGG | | |
| | TRDTSTSTAYMELSSLRSE | SGTDFTLKISRVEAEDVG | | |
| | DTAVYYCELMQGYWGQG | VYYCQQLVEYPLTFGQGT | | |
| | TLVTVSS (SEQ ID NO: | KLEIK | | |
| | 645) | (SEQ ID NO: 646) | | |
| Ozoralizumab | EVQLVESGGGLVQPGGSL | na | EVQLVESGGGLVQ | na |
| (Format: | RLSCAASGFTFSDYWMY | | PGNSLRLSCAASGF | |
| Bispecific | WVRQAPGKGLEWVSEIN | | TFSSFGMSWVRQ | |
| Single | TNGLITKYPDSVKGRFTISR | | APGKGLEWVSSIS | |
| Domains (VH- | DNAKNTLYLQMNSLRPED | | GSGSDTLYADSVK | |
| VH′-VH) | TAVYYCARSPSGFNRGQG | | GRFTISRDNAKTTL | |
| | TLVTVSS (SEQ ID NO: | | YLQMNSLRPEDTA | |
| | 647) | | VYYCTIGGSLSRSS | |
| | | | QGTLVTVSS (SEQ | |
| | | | ID NO: 648) | |
| Pabinafusp | EVQLVQSGAEVKKPGESL | DIVMTQTPLSLSVTPGQP | na | na |
| (Format: Whole | KISCKGSGYSFTNYWLGW | ASISCRSSQSLVHSNGNT | | |
| mAb Fusion) | VRQMPGKGLEWMGDIYP | YLHWYLQKPGQSPQLLIY | | |
| | GGDYPTYSEKFKVQVTISA | KVSNRFSGVPDRFSGSGS | | |
| | DKSISTAYLQWSSLKASDT | GTDFTLKISRVEAEDVGV | | |
| | AMYYCARSGNYDEVAYW | YYCSQSTHVPWTFGQGT | | |
| | GQGTLVTVSS (SEQ ID | KVEIK | | |
| | NO: 649) | (SEQ ID NO: 650) | | |
| Palivizumab | QVTLRESGPALVKPTQTLT | DIQMTQSPSTLSASVGD | na | na |
| (Format: Whole | LTCTFSGFSLSTSGMSVG | RVTITCKCQLSVGYMHW | | |
| mAb) | WIRQPPGKALEWLADIW | YQQKPGKAPKLLIYDTSKL | | |
| | WDDKKDYNPSLKSRLTISK | ASGVPSRFSGSGSGTEFT | | |
| | DTSKNQVVLKVTNMDPA | LTISSLQPDDFATYYCFQG | | |
| | DTATYYCARSMITNWYFD | SGYPFTFGGGTKLEIK | | |
| | VWGAGTTVTVSS (SEQ ID | | | |
| | NO: 651) | (SEQ ID NO: 652) | | |
| Pamrevlumab | EGQLVQSGGGLVHPGGSL | DIQMTQSPSSLSASVGD | na | na |
| (Format: Whole | RLSCAGSGFTFSSYGMHW | RVTITCRASQGISSWLAW | | |
| mAb) | VRQAPGKGLEWVSGIGTG | YQQKPEKAPKSLIYAASSL | | |
| | GGTYSTDSVKGRFTISRDN | QSGVPSRFSGSGSGTDFT | | |
| | AKNSLYLQMNSLRAEDM | LTISSLQPEDFATYYCQQY | | |
| | AVYYCARGDYYGSGSFFD | NSYPPTFGQGTKLEIK | | |
| | CWGQGTLVTVSS (SEQ ID | (SEQ ID NO: 654) | | |
| | NO: 653) | | | |
| Panitumumab | QVQLQESGPGLVKPSETLS | DIQMTQSPSSLSASVGD | na | na |
| (Format: Whole | LTCTVSGGSVSSGDYYWT | RVTITCQASQDISNYLNW | | |
| mAb) | WIRQSPGKGLEWIGHIYYS | YQQKPGKAPKLLIYDASN | | |
| | GNTNYNPSLKSRLTISIDTS | LETGVPSRFSGSGSGTDF | | |
| | KTQFSLKLSSVTAADTAIYY | TFTISSLQPEDIATYFCQH | | |
| | CVRDRVTGAFDIWGQGT | FDHLPLAFGGGTKVEIK | | |
| | MVTVSS (SEQ ID NO: | | | |
| | 655) | (SEQ ID NO: 656) | | |
| Panobacumab | EEQVVESGGGFVQPGGSL | DVVMTQSPLSLPVTLGQ | na | na |
| (Format: Whole | RLSCAASGFTFSPYWMH | PASISCRSSQSLVYSDGNT | | |
| mAb) | WVRQAPGKGLVWVSRIN | YLNWFQQRPGQSPRRLI | | |
| | SDGSTYYADSVKGRFTISR | YKVSNRDSGVPDRFSGS | | |
| | DNARNTLYLQMNSLRAED | GSGTDFTLKISRVEAEDV | | |
| | TAVYYCARDRYYGPEMW | GVYYCMQGTHWPLTFG | | |
| | GQGTMVTVSS (SEQ ID | GGTKVEIK (SEQ ID NO: | | |
| | NO: 657) | 658) | | |
| Parsatuzumab | EVQLVESGGGLVQPGGSL | DIQMTQSPSSLSASVGD | na | na |
| (Format: Whole | RLSCAASGYTFIDYYMNW | RVTITCRTSQSLVHINAIT | | |
| mAb) | VRQAPGKGLEWVGDINL | YLHWYQQKPGKAPKLLIY | | |
| | DNSGTHYNQKFKGRFTISR | RVSNRFSGVPSRFSGSGS | | |
| | DKSKNTAYLQMNSLRAED | GTDFTLTISSLQPEDFATY | | |
| | TAVYYCAREGVYHDYDDY | YCGQSTHVPLTFGQGTK | | |
| | AMDYWGQGTLVTVSS | VEIK | | |
| | (SEQ ID NO: 659) | (SEQ ID NO: 660) | | |
| Pasotuxizumab | QVQLVESGGGLVKPGESL | DIQMTQSPSSLSASVGD | EVQLVESGGGLVQ | QTVVTQEPSLTVS |
| (Format: | RLSCAASGFTFSDYYMYW | RVTITCKASQNVDTNVA | PGGSLKLSCAASGF | PGGTVTLTCGSST |
| Bispecific scFv) | VRQAPGKGLEWVAIISDG | WYQQKPGQAPKSLIYSA | TFNKYAMNWVRQ | GAVTSGNYPNW |
| | GYYTYYSDIIKGRFTISRDN | SYRYSDVPSRFSGSASGT | APGKGLEWVARIR | VQQKPGQAPRGL |
| | AKNSLYLQMNSLKAEDTA | DFTLTISSVQSEDFATYYC | SKYNNYATYYADS | IGGTKFLAPGTPA |
| | VYYCARGFPLLRHGAMDY | QQYDSYPYTFGGGTKLEI | VKDRFTISRDDSKN | RFSGSLLGGKAAL |
| | WGQGTLVTVSS (SEQ ID | K (SEQ ID NO: 662) | TAYLQMNNLKTED | TLSGVQPEDEAEY |
| | NO: 661) | | TAVYYCVRHGNFG | YCVLWYSNRWVF |
| | | | NSYISYWAYWGQ | GGGTKLTVL (SEQ |
| | | | GTLVTVSS (SEQ ID | ID NO: 664) |
| | | | NO: 663) | |
| Pateclizumab | EVQLVESGGGLVQPGGSL | DIQMTQSPSSLSASVGD | na | na |
| (Format: Whole | RLSCAASGYTFTSYVIHWV | RVTITCRASQAVSSAVA | | |
| mAb) | RQAPGKGLEWVGYNNPY | WYQQKPGKAPKLLIYSAS | | |
| | NAGTNYNEKFKGRFTISSD | HRYTGVPSRFSGSGSGTD | | |
| | KSKNTAYLQMNSLRAEDT | FTLTISSLQPEDFATYYCQ | | |
| | AVYYCSRPTMLPWFAYW | ESYSTPWTFGQGTKVEIK | | |
| | GQGTLVTVSS (SEQ ID | (SEQ ID NO: 666) | | |
| | NO: 665) | | | |
| Patritumab | QVQLQQWGAGLLKPSETL | DIEMTQSPDSLAVSLGER | na | na |
| (Format: Whole | SLTCAVYGGSFSGYYWSW | ATINCRSSQSVLYSSSNR | | |
| mAb) | IRQPPGKGLEWIGEINHSG | NYLAWYQQNPGQPPKLL | | |
| | STNYNPSLKSRVTISVETSK | IYWASTRESGVPDRFSGS | | |
| | NQFSLKLSSVTAADTAVYY | GSGTDFTLTISSLQAEDV | | |
| | CARDKWTWYFDLWGRG | AVYYCQQYYSTPRTFGQ | | |
| | TLVTVSS (SEQ ID NO: | GTKVEIK (SEQ ID NO: | | |
| | 667) | 668) | | |
| Pembrolizumab | QVQLVQSGVEVKKPGASV | EIVLTQSPATLSLSPGERA | na | na |
| (Lambrolizumab) | KVSCKASGYTFTNYYMYW | TLSCRASKGVSTSGYSYLH | | |
| (Format: Whole | VRQAPGQGLEWMGGINP | WYQQKPGQAPRLLIYLA | | |
| mAb) | SNGGTNFNEKFKNRVTLT | SYLESGVPARFSGSGSGT | | |
| | TDSSTTTAYMELKSLQFDD | DFTLTISSLEPEDFAVYYC | | |
| | TAVYYCARRDYRFDMGFD | QHSRDLPLTFGGGTKVEI | | |
| | YWGQGTTVTVSS (SEQ ID | K (SEQ ID NO: 670) | | |
| | NO: 669) | | | |
| Pepinemab | QVQLVQSGAEVKKPGSSV | DIVMTQSPDSLAVSLGER | na | na |
| (Format: Whole | KVSCKASGYSFSDYYMHW | ATINCKASQSVDYDGDSY | | |
| mAb) | VRQAPGQGLEWMGQIN | MNWYQQKPGQPPKLLIY | | |
| | PTTGGASYNQKFKGKATIT | AASNLESGVPDRFSGSGS | | |
| | VDKSTSTAYMELSSLRSED | GTDFTLTISSLQAEDVAV | | |
| | TAVYYCARYYYGRHFDVW | YYCQQSNEDPYTFGQGT | | |
| | GQGTTVTVSS (SEQ ID | KLEIK | | |
| | NO: 671) | (SEQ ID NO: 672) | | |
| Perakizumab | EVQLVESGGGLVQPGGSL | DIQMTQSPSSLSASVGD | na | na |
| (Format: Whole | RLSCAASGFTFSDYTMLW | RVTITCRASQDINSYLSW | | |
| mAb) | VRQAPGKGLEWVAIIKSG | FQQKPGKAPKSLIVRANR | | |
| | GSYSYYPDSVKGRFTISRD | LVDGVPSRFSGSGSGQD | | |
| | NAKNSLYLQMNSLRAEDT | YSLTISSLQPEDFATYYCL | | |
| | AVYYCARDGDYGSSYGA | QYDAFPPYTFGQGTKLEI | | |
| | MDYWGQGTLVTVSS | K (SEQ ID NO: 674) | | |
| | (SEQ ID NO: 673) | | | |
| Pertuzumab | EVQLVESGGGLVQPGGSL | DIQMTQSPSSLSASVGD | na | na |
| (Format: Whole | RLSCAASGFTFTDYTMDW | RVTITCKASQDVSIGVAW | | |
| mAb) | VRQAPGKGLEWVADVNP | YQQKPGKAPKLLIYSASY | | |
| | NSGGSIYNQRFKGRFTLSV | RYTGVPSRFSGSGSGTDF | | |
| | DRSKNTLYLQMNSLRAED | TLTISSLQPEDFATYYCQQ | | |
| | TAVYYCARNLGPSFYFDY | YYIYPYTFGQGTKVEIK | | |
| | WGQGTLVTVSS (SEQ ID | (SEQ ID NO: 676) | | |
| | NO: 675) | | | |
| Pldilizumab | QVQLVQSGSELKKPGASV | EIVLTQSPSSLSASVGDRV | na | na |
| (Format: Whole | KISCKASGYTFTNYGMNW | TITCSARSSVSYMHWFQ | | |
| mAb) | VRQAPGQGLQWMGWIN | QKPGKAPKLWIYRTSNLA | | |
| | TDSGESTYAEEFKGRFVFS | SGVPSRFSGSGSGTSYCL | | |
| | LDTSVNTAYLQITSLTAEDT | TINSLQPEDFATYYCQQR | | |
| | GMYFCVRVGYDALDYWG | SSFPLTFGGGTKLEIK | | |
| | QGTLVTVSS (SEQ ID NO: | (SEQ ID NO: 678) | | |
| | 677) | | | |
| Pinatuzumab | EVQLVESGGGLVQPGGSL | DIQMTQSPSSLSASVGD | na | na |
| (Format: Whole | RLSCAASGYEFSRSWMN | RVTITCRSSQSIVHSVGNT | | |
| mAb ADC) | WVRQAPGKGLEWVGRIY | FLEWYQQKPGKAPKLLIY | | |
| | PGDGDTNYSGKFKGRFTIS | KVSNRFSGVPSRFSGSGS | | |
| | ADTSKNTAYLQMNSLRAE | GTDFTLTISSLQPEDFATY | | |
| | DTAVYYCARDGSSWDWY | YCFQGSQFPYTFGQGTK | | |
| | FDVWGQGTLVTVSS (SEQ | VEIK (SEQ ID NO: 680) | | |
| | ID NO: 679) | | | |
| Placulumab | na | DIQMTQSPSSLSASVGD | na | na |
| (Format: Single | | RVTITCRASQAIDSYLHW | | |
| Domain Variable | | YQQKPGKAPKLLIYSASN | | |
| Fragment + Fc) | | LETGVPSRFSGSGSGTDF | | |
| | | TLTISSLLPEDFATYYCQQ | | |
| | | VVWRPFTFGQGTKVEIK | | |
| | | (SEQ ID NO: 681) | | |
| Plamotamab | QVQLVQSGAEVKKPGASV | QIVLTQSPSSLSASVGDR | EVQLVESGGGLVQ | QAVVTQEPSLTVS |
| (Format: | KVSCKASGYTFTSYNMHW | VTITCRASSSVSYIHWFQ | PGGSLRLSCAASGF | PGGTVTLTCGSST |
| Bispecific Mixed | VRQAPGQGLEWMGAIYP | QKPGKSPKPLIYATSNLAS | TFSTYAMNWVRQ | GAVTTSNYANW |
| mAb and scFV) | GNGDTSYNQKFQGRVTIT | GVPVRFSGSGSGTDYTLT | APGKGLEWVGRIR | VQQKPGKSPRGLI |
| | ADKSISTAYMELSSLRSEDT | ISSLQPEDFATYYCQQWT | SKYNNYATYYADS | GGTNKRAPGVPA |
| | AVYYCARSTYYGGDWYFN | SNPPTFGGGTKVEIK | VKGRFTISRDDSKN | RFSGSLLGGKAAL |
| | VWGAGTLVTVSS (SEQ ID | (SEQ ID NO: 683) | TLYLQMNSLRAED | TISGAQPEDEADY |
| | NO: 682) | | TAVYYCVRHGNFG | YCALWYSNHWV |
| | | | DSYVSWFAYWGQ | FGGGTKLTVLEPK |
| | | | GTLVTVSS (SEQ ID | (SEQ ID NO: 685) |
| | | | NO: 684) | |
| Plozalizumab | EVQLVESGGGLVKPGGSL | DVVMTQSPLSLPVTLGQ | na | na |
| (Format: Whole | RLSCAASGFTFSAYAMNW | PASISCKSSQSLLDSDGKT | | |
| mAb) | VRQAPGKGLEWVGRIRTK | FLNWFQQRPGQSPRRLI | | |
| | NNNYATYYADSVKDRFTIS | YLVSKLDSGVPDRFSGSG | | |
| | RDDSKNTLYLQMNSLKTE | SGTDFTLKISRVEAEDVG | | |
| | DTAVYYCTTFYGNGVWG | VYYCWQGTHFPYTFGQ | | |
| | QGTLVTVSS (SEQ ID NO: | GTRLEIK (SEQ ID NO: | | |
| | 686) | 687) | | |
| Polatuzumab | EVQLVESGGGLVQPGGSL | DIQLTQSPSSLSASVGDR | na | na |
| (Format: Whole | RLSCAASGYTFSSYWIEWV | VTITCKASQSVDYEGDSF | | |
| mAb ADC) | RQAPGKGLEWIGEILPGG | LNWYQQKPGKAPKLLIY | | |
| | GDTNYNEIFKGRATFSADT | AASNLESGVPSRFSGSGS | | |
| | SKNTAYLQMNSLRAEDTA | GTDFTLTISSLQPEDFATY | | |
| | VYYCTRRVPIRLDYWGQG | YCQQSNEDPLTFGQGTK | | |
| | TLVTVSS (SEQ ID NO: | VEIK (SEQ ID NO: 689) | | |
| | 688) | | | |
| Ponezumab | QVQLVQSGAEVKKPGASV | DVVMTQSPLSLPVTLGQ | na | na |
| (Format: Whole | KVSCKASGYYTEAYYIHWV | PASISCKSSQSLLYSDAKT | | |
| mAb) | RQAPGQGLEWMGRIDPA | YLNWFQQRPGQSPRRLI | | |
| | TGNTKYAPRLQDRVTMTR | YQISRLDPGVPDRFSGSG | | |
| | DTSTSTVYMELSSLRSEDT | SGTDFTLKISRVEAEDVG | | |
| | AVYYCASLYSLPVYWGQG | VYYCLQGTHYPVLFGQG | | |
| | TTVTVSS (SEQ ID NO: | TRLEIK (SEQ ID NO: | | |
| | 690) | 691) | | |
| Porgaviximab | EVQLQESGGGLMQPGGS | DIQMTQSPASLSVSVGET | na | na |
| (Format: Whole | MKLSCVASGFTFSNYWM | VSITCRASENIYSSLAWY | | |
| mAb) | NWVRQSPEKGLEWVAEIR | QQKQGKSPQLLVYSATIL | | |
| | LKSNNYATHYAESVKGRFT | ADGVPSRFSGSGSGTQY | | |
| | ISRDDSKRSVYLQMNTLRA | SLKINSLQSEDFGTYYCQ | | |
| | EDTGIYYCTRGNGNYRAM | HFWGTPYTFGGGTKLEIK | | |
| | DYWGQGTSVTVSS (SEQ | (SEQ ID NO: 693) | | |
| | ID NO: 692) | | | |
| Prasinezumab | EVQLVESGGGLVQPGGSL | DIQMTQSPSSLSASVGD | na | na |
| (Format: Whole | RLSCAASGFTFSNYGMSW | RVTITCKSIQTLLYSSNQK | | |
| mAb) | VRQAPGKGLEWVASISSG | NYLAWFQQKPGKAPKLL | | |
| | GGSTYYPDNVKGRFTISRD | IYWASIRKSGVPSRFSGS | | |
| | DAKNSLYLQMNSLRAEDT | GSGTDFTLTISSLQPEDLA | | |
| | AVYYCARGGAGIDYWGQ | TYYCQQYYSYPLTFGGGT | | |
| | GTLVTVSS (SEQ ID NO: | KLEIK (SEQ ID NO: | | |
| | 694) | 695) | | |
| Prezalumab | EVQLVESGGGLVQPGGSL | DIQMTQSPSSLSASVGD | na | na |
| (Format: Whole | RLSCAASGFTFSSYWMSW | RVTITCRASQGISNWLA | | |
| mAb) | VRQAPGKGLEWVAYIKQD | WYQQKPEKAPKSLIYAAS | | |
| | GNEKYYVDSVKGRFTISRD | SLQSGVPSRFSGSGSGTD | | |
| | NAKNSLYLQMNSLRAEDT | FTLTISSLQPEDFATYYCQ | | |
| | AVYYCAREGILWFGDLPTF | QYDSYPRTFGQGTKVEIK | | |
| | WGQGTLVTVSS (SEQ ID | (SEQ ID NO: 697) | | |
| | NO: 696) | | | |
| Pritoxaximab | QVQLQESGAELVRSGASV | DIVMSQSHKFMSTSVGD | na | na |
| (Format: Whole | RMSCKASGYTFTSYNMH | RVSITCKASQDVGTAVA | | |
| mAb) | WVKQTPGQGLEWIGYIYP | WYQQNPGQSPKFLIYW | | |
| | GNGGTNYIQKFKGKAILTA | ASTRHTGVPDRFTGSGS | | |
| | DTSSSTAYMQISSLTSEDS | GTDFTLTITNVQSEDLAD | | |
| | AVYFCTRSPSHYSSDPYFD | YFCQQYSSYPLTFGAGTS | | |
| | YWGQGTTLTVSS (SEQ ID | LELK (SEQ ID NO: 699) | | |
| | NO: 698) | | | |
| Prolgolimab | QVQLVQSGGGLVQPGGS | QPVLTQPLSVSVALGQTA | na | na |
| (Format: Whole | LRLSCAASGFTFSSYWMY | RITCGGNNIGSKNVHWY | | |
| mAb) | WVRQVPGKGLEWVSAID | QQKPGQAPVLVIYRDSN | | |
| | TGGGRTYYADSVKGRFAIS | RPSGIPERFSGSNSGNTA | | |
| | RVNAKNTMYLQMNSLRA | TLTISRAQAGDEADYYCQ | | |
| | EDTAVYYCARDEGGGTG | VWDSSTAVFGTGTKLTV | | |
| | WGVLKDWPYGLDAWGQ | L (SEQ ID NO: 701) | | |
| | GTLVTVSS (SEQ ID NO: | | | |
| | 700) | | | |
| Quetmolimab | QVQLVQSGAEVKKPGASV | DIQMTQSPSSLSASVGD | na | na |
| (Format: Whole | KVSCKASGYTFTNYYIHWV | RVTITCRASGNIHNFLAW | | |
| mAb) | KQAPGQGLEWIGWIYPG | YQQKPGKAPKLLIYNEKT | | |
| | DGSPKFNERFKGRTTLTAD | LADGVPSRFSGSGSGTDY | | |
| | KSTNTAYMLLSSLRSEDTA | TLTISSLQPEDFATYFCQQ | | |
| | VYFCATGPTDGDYFDYW | FWSTPYTFGGGTKVEIK | | |
| | GQGTTVTVSS (SEQ ID | (SEQ ID NO: 703) | | |
| | NO: 702) | | | |
| Quilizumab | EVQLVESGGGLVQPGGSL | DIQMTQSPSSLSASVGD | na | na |
| (Format: Whole | RLSCAASGFTFSDYGIAWV | RVTITCRSSQSLVHNNAN | | |
| mAb) | RQAPGKGLEWVAFISDLA | TYLHWYQQKPGKAPKLLI | | |
| | YTIYYADTVTGRFTISRDNS | YKVSNRFSGVPSRFSGSG | | |
| | KNTLYLQMNSLRAEDTAV | SGTDFTLTISSLQPEDFAT | | |
| | YYCARDNWDAMDYWGQ | YYCSQNTLVPWTFGQGT | | |
| | GTLVTVSS (SEQ ID NO: | KVEIK (SEQ ID NO: | | |
| | 704) | 705) | | |
| Racotumomab | QVQLQQSGAELVKPGASV | DIQMTQTTSSLSASLGDR | na | na |
| (Format: Whole | KLSCKASGYTFTSYDINWV | VTISCRASQDISNYLNWY | | |
| mAb) | RQRPEQGLEWIGWIFPGD | QQKPDGTVKLLIYYTSRL | | |
| | GSTKYNEKFKGKATLTTDK | HSGVPSRFSGSGSGTDYS | | |
| | SSSTAYMQLSRLTSEDSAV | LTISNLEQEDIATYFCQQ | | |
| | YFCAREDYYDNSYYFDYW | GNTLPWTFGGGTKLEIK | | |
| | GQGTTLTVSS (SEQ ID | (SEQ ID NO: 707) | | |
| | NO: 706) | | | |
| Radretumab | EVQLLESGGGLVQPGGSL | EIVLTQSPGTLSLSPGERA | na | na |
| (Bifikafusp/ | RLSCAASGFTFSSFSMSW | TLSCRASQSVSSSFLAWY | | |
| Onfekafusp) | VRQAPGKGLEWVSSISGS | QQKPGQAPRLLIYYASSR | | |
| (Format: | SGTTYYADSVKGRFTISRD | ATGIPDRFSGSGSGTDFT | | |
| di-scFv + CH4) | NSKNTLYLQMNSLRAEDT | LTISRLEPEDFAVYYCQQT | | |
| | AVYYCAKPFPYFDYWGQG | GRIPPTFGQGTKVEIK | | |
| | TLVTVSS (SEQ ID NO: | (SEQ ID NO: 709) | | |
| | 708) | | | |
| Rafivirumab | QVQLVQSGAEVKKPGSSV | QSALTQPRSVSGSPGQS | na | na |
| (Format: Whole | KVSCKASGGTFNRYTVNW | VTISCTGTSSDIGGYNFVS | | |
| mAb) | VRQAPGQGLEWMGGIIPI | WYQQHPGKAPKLMIYD | | |
| | FGTANYAQRFQGRLTITA | ATKRPSGVPDRFSGSKSG | | |
| | DESTSTAYMELSSLRSDDT | NTASLTISGLQAEDEADY | | |
| | AVYFCARENLDNSGTYYYF | YCCSYAGDYTPGVVFGG | | |
| | SGWFDPWGQGTLVTVSS | GTKLTVL (SEQ ID NO: | | |
| | (SEQ ID NO: 710) | 711) | | |
| Ralpancizumab | QVQLVQSGAEVKKPGASV | DIQMTQSPSSLSASVGD | na | na |
| (Format: Whole | KVSCKASGYTFTSYYMHW | RVTITCKASQDVHTAVA | | |
| mAb) | VRQAPGQGLEWMGEIHP | WYQQKPGKAPKLLIYHA | | |
| | SGGRTNYNEKFKSRVTMT | SYRYTGVPSRFSGSGSGT | | |
| | RDTSTSTVYMELSSLRSED | DFTFTISSLQPEDIATYYC | | |
| | TAVYYCARERPLYASDLW | QQRYSLWRTFGQGTKLE | | |
| | GQGTTVTVSS (SEQ ID | IK (SEQ ID NO: 713) | | |
| | NO: 712) | | | |
| Ramucirumab | EVQLVQSGGGLVKPGGSL | DIQMTQSPSSVSASIGDR | na | na |
| (Format: Whole | RLSCAASGFTFSSYSMNW | VTITCRASQGIDNWLGW | | |
| mAb) | VRQAPGKGLEWVSSISSSS | YQQKPGKAPKLLIYDASN | | |
| | SYIYYADSVKGRFTISRDNA | LDTGVPSRFSGSGSGTYF | | |
| | KNSLYLQMNSLRAEDTAV | TLTISSLQAEDFAVYFCQ | | |
| | YYCARVTDAFDIWGQGT | QAKAFPPTFGGGTKVDIK | | |
| | MVTVSS (SEQ ID NO: | (SEQ ID NO: 715) | | |
| | 714) | | | |
| Ranevetmab | EVQLVESGGGLVQPGGSL | DIVMTQSPASLSLSQGET | na | na |
| (Format: Canine | RLSCVASGFSLTNNNVNW | VTITCRASEDIYNALAWY | | |
| Whole mAb) | VRQAPGKGLEWVGGVW | QQKPGQAPKLLIYNTDTL | | |
| | AGGATDYNSALKSRFTISR | HTGVPSRFSGSGSGTDFS | | |
| | DNAKNTVFLQMHSLRSED | LTISSLEPEDVAVYYCQHY | | |
| | TAVYYCARDGGYSSSTLYA | FHYPRTFGQGTKVELK | | |
| | MDAWGQGTSVTVSS | (SEQ ID NO: 717) | | |
| | (SEQ ID NO: 716) | | | |
| Ranibizumab | EVQLVESGGGLVQPGGSL | DIQLTQSPSSLSASVGDR | na | na |
| (Format: Fab) | RLSCAASGYDFTHYGMN | VTITCSASQDISNYLNWY | | |
| | WVRQAPGKGLEWVGWI | QQKPGKAPKVLIYFTSSL | | |
| | NTYTGEPTYAADFKRRFTF | HSGVPSRFSGSGSGTDFT | | |
| | SLDTSKSTAYLQMNSLRAE | LTISSLQPEDFATYYCQQY | | |
| | DTAVYYCAKYPYYYGTSH | STVPWTFGQGTKVEIK | | |
| | WYFDVWGQGTLVTVSS | (SEQ ID NO: 719) | | |
| | (SEQ ID NO: 718) | | | |
| Ravagalimab | EVQLVESGGGLVKPGGSL | DIVMTQSPDSLAVSLGER | na | na |
| (Format: Whole | RLSCAASGFTFSDYGMN | ATINCKSSQSLLNRGNQK | | |
| mAb) | WVRQAPGKGLEWIAYISS | NYLTWFQQKPGQPPKLL | | |
| | GRGNIYYADTVKGRFTISR | IYWASTRESGVPDRFSGS | | |
| | DNAKNSLYLQMNSLRAED | GSGTDFTLTISSLQAEDV | | |
| | TAVYYCARSWGYFDVWG | AVYYCQNDYTYPLTFGQ | | |
| | QGTTVTVSS (SEQ ID NO: | GTKLEIK (SEQ ID NO: | | |
| | 720) | 721) | | |
| Ravulizumab | QVQLVQSGAEVKKPGASV | DIQMTQSPSSLSASVGD | na | na |
| (Format: Whole | KVSCKASGHIFSNYWIQW | RVTITCGASENIYGALNW | | |
| mAb) | VRQAPGQGLEWMGEILP | YQQKPGKAPKLLIYGATN | | |
| | GSGHTEYTENFKDRVTMT | LADGVPSRFSGSGSGTDF | | |
| | RDTSTSTVYMELSSLRSED | TLTISSLQPEDFATYYCQN | | |
| | TAVYYCARYFFGSSPNWY | VLNTPLTFGQGTKVEIK | | |
| | FDVWGQGTLVTVSS (SEQ | (SEQ ID NO: 723) | | |
| | ID NO: 722) | | | |
| Refanezumab | QVQLVQSGSELKKPGASV | DIVMTQSPDSLAVSLGER | na | na |
| (Format: Whole | KVSCKASGYTFTNYGMN | ATINCKSSHSVLYSSNQK | | |
| mAb) | WVRQAPGQGLEWMGWI | NYLAWYQQKPGQPPKLL | | |
| | NTYTGEPTYADDFTGRFVF | IYWASTRESGVPDRFSGS | | |
| | SLDTSVSTAYLQISSLKAED | GSGTDFTLTISSLQAEDV | | |
| | TAVYYCARNPINYYGINYE | AVYYCHQYLSSLTFGQGT | | |
| | GYVMDYWGQGTLVTVSS | KLEIK (SEQ ID NO: | | |
| | (SEQ ID NO: 724) | 725) | | |
| Relatlimab | QVQLQQWGAGLLKPSETL | EIVLTQSPATLSLSPGERA | na | na |
| (Format: Whole | SLTCAVYGGSFSDYYWN | TLSCRASQSISSYLAWYQ | | |
| mAb) | WIRQPPGKGLEWIGEINH | QKPGQAPRLLIYDASNRA | | |
| | RGSTNSNPSLKSRVTLSLD | TGIPARFSGSGSGTDFTLT | | |
| | TSKNQFSLKLRSVTAADTA | ISSLEPEDFAVYYCQQRS | | |
| | VYYCAFGYSDYEYNWFDP | NWPLTFGQGTNLEIK | | |
| | WGQGTLVTVSS (SEQ ID | (SEQ ID NO: 727) | | |
| | NO: 726) | | | |
| Relfovetmab | DVQLVESGGDLVKPGGSL | EIQMTQSPSSLSASPGDR | na | na |
| (Format: Feline | RLTCVASGFTYSNYWMH | VTITCRASENIYSFLAWY | | |
| Whole mAb) | WVRQAPGKGLQWVARID | QQKPGKVPKLLIYNANTL | | |
| | PYGGGTKHNEKFKRRFTIS | AEGVPSRFSGSGSGTDFT | | |
| | RDNAKNTLYLQMNSLKTE | LTISSLEPEDAATYYCQHH | | |
| | DTATYYCVRSGYDYYFDV | FGTPFTFGSGTKLEIK | | |
| | WGQGTLVTVSS (SEQ ID | (SEQ ID NO: 729) | | |
| | NO: 728) | | | |
| Remtolumab | EVQLVESGGGLVQPGRSL | DIQMTQSPSSLSASVGD | EVQLVQSGAEVKK | EIVLTQSPDFQSV |
| (Format: | RLSCAASGFTFDDYAMH | RVTITCRASQGIRNYLAW | PGSSVKVSCKASG | TPKEKVTITCRAS |
| Bispecific Dual | WVRQAPGKGLEWVSAIT | YQQKPGKAPKLLIYAAST | GSFGGYGIGWVR | QDIGSELHWYQQ |
| Variable Domain | WNSGHIDYADSVEGRFTIS | LQSGVPSRFSGSGSGTDF | QAPGQGLEWMG | KPDQPPKLLIKYAS |
| IG) | RDNAKNSLYLQMNSLRAE | TLTISSLQPEDVATYYCQR | GITPFFGFADYAQK | HSTSGVPSRFSGS |
| | DTAVYYCAKVSYLSTASSL | YNRAPYTFGQGTKVEIK | FQGRVTITADESTT | GSGTDFTLTINGL |
| | DYWGQGTLVTVSS (SEQ | (SEQ ID NO: 731) | TAYMELSGLTSDD | EAEDAGTYYCHQ |
| | ID NO: 730) | | TAVYYCARDPNEF | TDSLPYTFGPGTK |
| | | | WNGYYSTHDFDS | VDIK (SEQ ID NO: |
| | | | WGQGTTVTVSS | 733) |
| | | | (SEQ ID NO: 732) | |
| Reslizumab | EVQLVESGGGLVQPGGSL | DIQMTQSPSSLSASVGD | na | na |
| (Format: Whole | RLSCAVSGLSLTSNSVNWI | RVTITCLASEGISSYLAWY | | |
| mAb) | RQAPGKGLEWVGLIWSN | QQKPGKAPKLLIYGANSL | | |
| | GDTDYNSAIKSRFTISRDTS | QTGVPSRFSGSGSATDYT | | |
| | KSTVYLQMNSLRAEDTAV | LTISSLQPEDFATYYCQQS | | |
| | YYCAREYYGYFDYWGQGT | YKFPNTFGQGTKVEVK | | |
| | LVTVSS (SEQ ID NO: 734) | (SEQ ID NO: 735) | | |
| Rilotumumab | QVQLQESGPGLVKPSETLS | EIVMTQSPATLSVSPGER | na | na |
| (Format: Whole | LTCTVSGGSISIYYWSWIR | ATLSCRASQSVDSNLAW | | |
| mAb) | QPPGKGLEWIGYVYYSGS | YRQKPGQAPRLLIYGAST | | |
| | TNYNPSLKSRVTISVDTSK | RATGIPARFSGSGSGTEF | | |
| | NQFSLKLNSVTAADTAVYY | TLTISSLQSEDFAVYYCQQ | | |
| | CARGGYDFWSGYFDYWG | YINWPPITFGQGTRLEIK | | |
| | QGTLVTVSS (SEQ ID NO: | (SEQ ID NO: 737) | | |
| | 736) | | | |
| Rinucumab | QLQLQESGPGLVKPSETLS | EIVLTQSPDTISLSPGERA | na | na |
| (Format: Whole | LTCTVSGGSITSSSYYWG | TLSCRASQSISSIYLAWYQ | | |
| mAb) | WIRQPPGKGLEWIGSIYYR | QKPGQAPRLLIYGASSRV | | |
| | GSTNYNPSLKSRVTISVDSS | TGIPDRFSVSGSGTDFTLT | | |
| | KNQFYLKVSSVTAVDTAVY | ISRLEPEDFAVYYCQHYGI | | |
| | YCARQNGAARPSWFDPW | SPFTFGPGTKVDIR (SEQ | | |
| | GQGTLVTVSS (SEQ ID | ID NO: 739) | | |
| | NO: 738) | | | |
| Risankizumab | QVQLVQSGAEVKKPGSSV | DIQMTQSPSSLSASVGD | na | na |
| (Format: Whole | KVSCKASGYTFTDQTIHW | RVTITCKASRDVAIAVAW | | |
| mAb) | MRQAPGQGLEWIGYIYPR | YQQKPGKVPKLLIYWAST | | |
| | DDSPKYNENFKGKVTITAD | RHTGVPSRFSGSGSRTDF | | |
| | KSTSTAYMELSSLRSEDTA | TLTISSLQPEDVADYFCH | | |
| | VYYCAIPDRSGYAWFIYW | QYSSYPFTFGSGTKLEIK | | |
| | GQGTLVTVSS (SEQ ID | (SEQ ID NO: 741) | | |
| | NO: 740) | | | |
| Rituximab | QVQLQQPGAELVKPGASV | QIVLSQSPAILSASPGEKV | na | na |
| (Format: Whole | KMSCKASGYTFTSYNMH | TMTCRASSSVSYIHWFQ | | |
| mAb) | WVKQTPGRGLEWIGAIYP | QKPGSSPKPWIYATSNLA | | |
| | GNGDTSYNQKFKGKATLT | SGVPVRFSGSGSGTSYSL | | |
| | ADKSSSTAYMQLSSLTSED | TISRVEAEDAATYYCQQ | | |
| | SAVYYCARSTYYGGDWYF | WTSNPPTFGGGTKLEIK | | |
| | NVWGAGTTVTVSA (SEQ | (SEQ ID NO: 743) | | |
| | ID NO: 742) | | | |
| Rivabazumab | EVQLVESGGGVVQPGRSL | DIQLTQSPSTLSASVGDS | na | na |
| (Format: Fab) | RLSCAASGFTFSNYPMHW | VTITCRASEGVDRWLAW | | |
| | VRQAPGKGLEWVAVISYD | YQQKPGRAPKLLIYDAST | | |
| | GSEKWYADSVKGRFTISR | LQSGVPSRFSGSGSGTEF | | |
| | DNSKNTLYLEMNSLRPED | SLTISSLQPDDVATYYCQ | | |
| | TAVYYCARNRGDIYYDFTY | HFWGTPYTFGQGTKLEIK | | |
| | AMDIWGQGTTVTVSS | (SEQ ID NO: 745) | | |
| | (SEQ ID NO: 744) | | | |
| Robatumumab | EVQLVQSGGGLVKPGGSL | EIVLTQSPGTLSVSPGERA | na | na |
| (Format: Whole | RLSCAASGFTFSSFAMHW | TLSCRASQSIGSSLHWYQ | | |
| mAb) | VRQAPGKGLEWISVIDTR | QKPGQAPRLLIKYASQSL | | |
| | GATYYADSVKGRFTISRDN | SGIPDRFSGSGSGTDFTLT | | |
| | AKNSLYLQMNSLRAEDTA | ISRLEPEDFAVYYCHQSSR | | |
| | VYYCARLGNFYYGMDVW | LPHTFGQGTKVEIK (SEQ | | |
| | GQGTTVTVSS (SEQ ID | ID NO: 747) | | |
| | NO: 746) | | | |
| Roledumab | QVQLVESGGGVVQPGRSL | AIRMTQSPSSFSASTGDR | na | na |
| (Format: Whole | RLSCTASGFTFKNYAMHW | VTITCRASQDIRNYVAWY | | |
| mAb) | VRQAPAKGLEWVATISYD | QQKSGKAPKFLIYAASTL | | |
| | GRNIQYADSVKGRFTFSRD | QSGVPSRFSGSGSGTDFT | | |
| | NSQDTLYLQLNSLRPEDTA | LTINSLQSEDFATYYCQQ | | |
| | VYYCARPVRSRWLQLGLE | YYNSPPTFGQGTRVEIT | | |
| | DAFHIWGQGTMVTVSS | (SEQ ID NO: 749) | | |
| | (SEQ ID NO: 748) | | | |
| Rolinsatamab | EVQLVQSGAEVKKPGSSV | DIQMTQSPSSVSASVGD | na | na |
| (Format: Whole | KVSCKASGYTFTTYWMH | RVTITCKASQYVGTAVA | | |
| mAb ADC) | WVRQAPGQGLEWIGEID | WYQQKPGKSPKLLIYSAS | | |
| | PSDSYSNYNQKFKDRATLT | NRYTGVPSRFSDSGSGTD | | |
| | VDKSTSTAYMELSSLRSED | FTLTISSLQPEDFATYFCQ | | |
| | TAVYYCARNGGLGPAWFS | QYSSYPWTFGGGTKVEIK | | |
| | YWGQGTLVTVSS (SEQ ID | (SEQ ID NO: 751) | | |
| | NO: 750) | | | |
| Romilkimab | EVQLKESGPGLVAPGGSLS | DIVLTQSPASLAVSLGQR | QVQLQQSGPELVK | DIQMTQSPASLSV |
| (Format: | ITCTVSGFSLTDSSINWVR | ATISCRASESVDSYGQSY | PGASVKISCKASGY | SVGDTITLTCHAS |
| Bispecific Dual | QPPGKGLEWLGMIWGD | MHWYQQKAGQPPKLLI | SFTSYWIHWIKQR | QNIDVWLSWFQ |
| Variable Domain | GRIDYADALKSRLSISKDSS | YLASNLESGVPARFSGSG | PGQGLEWIGMIDP | QKPGNIPKLLIYKA |
| IG) | KSQVFLEMTSLRTDDTATY | SRTDFTLTIDPVQAEDAA | SDGETRLNQRFQG | SNLHTGVPSRFSG |
| | YCARDGYFPYAMDFWGQ | TYYCQQNAEDSRTFGGG | RATLTVDESTSTAY | SGSGTGFTLTISSL |
| | GTSVTVSS (SEQ ID NO: | TKLEIK (SEQ ID NO: | MQLRSPTSEDSAV | QPEDIATYYCQQ |
| | 752) | 753) | YYCTRLKEYGNYDS | AHSYPFTFGGGTK |
| | | | FYFDVWGAGTLVT | LEIK (SEQ ID NO: |
| | | | VSS (SEQ ID NO: | 755) |
| | | | 754) | |
| Romosozumab | EVQLVQSGAEVKKPGASV | DIQMTQSPSSLSASVGD | na | na |
| (Format: Whole | KVSCKASGYTFTDYNMH | RVTITCRASQDISNYLNW | | |
| mAb) | WVRQAPGQGLEWMGEI | YQQKPGKAPKLLIYYTSRL | | |
| | NPNSGGAGYNQKFKGRV | LSGVPSRFSGSGSGTDFT | | |
| | TMTTDTSTSTAYMELRSLR | LTISSLQPEDFATYYCQQ | | |
| | SDDTAVYYCARLGYDDIYD | GDTLPYTFGGGTKVEIK | | |
| | DWYFDVWGQGTTVTVSS | (SEQ ID NO: 757) | | |
| | (SEQ ID NO: 756) | | | |
| Rontalizumab | EVQLVESGGGLVQPGGSL | DIQMTQSPSSLSASVGD | na | na |
| (Format: Whole | RLSCATSGYTFTEYIIHWVR | RVTITCRASQSVSTSSYSY | | |
| mAb) | QAPGKGLEWVASINPDYD | MHWYQQKPGKAPKVLIS | | |
| | ITNYNQRFKGRFTISLDKSK | YASNLESGVPSRFSGSGS | | |
| | RTAYLQMNSLRAEDTAVY | GTDFTLTISSLQPEDFATY | | |
| | YCASWISDFFDYWGQGTL | YCQHSWGIPRTFGQGTK | | |
| | VTVSS (SEQ ID NO: 758) | VEIK (SEQ ID NO: 759) | | |
| Rosmantuzumab | QVQLVQSGAEVKKPGASV | DIQMTQSPSSLSASVGD | na | na |
| (Format: Whole | KVSCKASGYTFTDYSIHWV | RVTITCKASQSVDYDGDS | | |
| mAb) | RQAPGQGLEWIGYIYPSN | YMNWYQQKPGKAPKLLI | | |
| | GDSGYNQKFKNRVTMTR | YAASNLESGVPSRFSGSG | | |
| | DTSTSTAYMELSRLRSEDT | SGTDFTLTISPVQAEDFA | | |
| | AVYYCATYFANNFDYWG | TYYCQQSNEDPLTFGAG | | |
| | QGTTLTVSS (SEQ ID NO: | TKLELK (SEQ ID NO: | | |
| | 760) | 761) | | |
| Rovalpituzumab | QVQLVQSGAEVKKPGASV | EIVMTQSPATLSVSPGER | na | na |
| (Format: Whole | KVSCKASGYTFTNYGMN | ATLSCKASQSVSNDVVW | | |
| mAb ADC) | WVRQAPGQGLEWMGWI | YQQKPGQAPRLLIYYASN | | |
| | NTYTGEPTYADDFKGRVT | RYTGIPARFSGSGSGTEFT | | |
| | MTTDTSTSTAYMELRSLRS | LTISSLQSEDFAVYYCQQ | | |
| | DDTAVYYCARIGDSSPSDY | DYTSPWTFGQGTKLEIK | | |
| | WGQGTLVTVSS (SEQ ID | (SEQ ID NO: 763) | | |
| | NO: 762) | | | |
| Rozanolixizumab | EVPLVESGGGLVQPGGSL | DIQMTQSPSSLSASVGD | na | na |
| (Format: Whole | RLSCAVSGFTFSNYGMVW | RVTITCKSSQSLVGASGK | | |
| mAb) | VRQAPGKGLEWVAYIDSD | TYLYWLFQKPGKAPKRLI | | |
| | GDNTYYRDSVKGRFTISRD | YLVSTLDSGIPSRFSGSGS | | |
| | NAKSSLYLQMNSLRAEDT | GTEFTLTISSLQPEDFATY | | |
| | AVYYCTTGIVRPFLYWGQ | YCLQGTHFPHTFGQGTK | | |
| | GTLVTVSS (SEQ ID NO: | LEIK (SEQ ID NO: 765) | | |
| | 764) | | | |
| Rozipafusp | EVQLVESGGGLVQPGGSL | DIQMTQSPSSLSASVGD | na | na |
| (Format: Whole | RLSCAASGFTFSSYWMSW | RVTITCRASQGISNWLA | | |
| mAb Fusion) | VRQAPGKGLEWVAYIKQD | WYQQKPEKAPKSLIYAAS | | |
| | GNEKYYVDSVKGRFTISRD | SLQSGVPSRFSGSGSGTD | | |
| | NAKNSLYLQMNSLRAEDT | FTLTISSLQPEDFATYYCQ | | |
| | AVYYCAREGILWFGDLPTF | QYDSYPRTFGQGTKVEIK | | |
| | WGQGTLVTVSS (SEQ ID | (SEQ ID NO: 767) | | |
| | NO: 766) | | | |
| Ruplizumab | QVQLVQSGAEVVKPGASV | DIVLTQSPATLSVSPGER | na | na |
| (Format: Whole | KLSCKASGYIFTSYYMYWV | ATISCRASQRVSSSTYSY | | |
| mAb) | KQAPGQGLEWIGEINPSN | MHWYQQKPGQPPKLLIK | | |
| | GDTNFNEKFKSKATLTVDK | YASNLESGVPARFSGSGS | | |
| | SASTAYMELSSLRSEDTAV | GTDFTLTISSVEPEDFATY | | |
| | YYCTRSDGRNDMDSWG | YCQHSWEIPPTFGGGTKL | | |
| | QGTLVTVSS (SEQ ID NO: | EIK (SEQ ID NO: 769) | | |
| | 768) | | | |
| Sacituzumab | QVQLQQSGSELKKPGASV | DIQLTQSPSSLSASVGDR | na | na |
| (Format: Whole | KVSCKASGYTFTNYGMN | VSITCKASQDVSIAVAWY | | |
| mAb ADC) | WVKQAPGQGLKWMGWI | QQKPGKAPKLLIYSASYR | | |
| | NTYTGEPTYTDDFKGRFAF | YTGVPDRFSGSGSGTDFT | | |
| | SLDTSVSTAYLQISSLKADD | LTISSLQPEDFAVYYCQQ | | |
| | TAVYFCARGGFGSSYWYF | HYITPLTFGAGTKVEIK | | |
| | DVWGQGSLVTVSS (SEQ | (SEQ ID NO: 771) | | |
| | ID NO: 770) | | | |
| Samalizumab | QVQLQQSGSELKKPGASV | DIQMTQSPSSLSASIGDR | na | na |
| (Format: Whole | KISCKASGYSFTDYIILWVR | VTITCKASQDINSYLSWF | | |
| mAb) | QNPGKGLEWIGHIDPYYG | QQKPGKAPKLLIYRANRL | | |
| | SSNYNLKFKGRVTITADQS | VDGVPSRFSGSGSGTDYT | | |
| | TTTAYMELSSLRSEDTAVY | LTISSLQPEDFAVYYCLQY | | |
| | YCGRSKRDYFDYWGQGT | DEFPYTFGGGTKLEIK | | |
| | TLTVSS (SEQ ID NO: 772) | (SEQ ID NO: 773) | | |
| Samrotamab | EVQLVQSGAEVKKPGASV | DIQMTQSPSSLSASVGD | na | na |
| (Format: Whole | KVSCKASGYKFSSYWIEW | RVTITCRASQDISNYLNW | | |
| mAb ADC) | VKQAPGQGLEWIGEILPG | YQQKPGGAVKFLIYYTSR | | |
| | SDTTNYNEKFKDRATFTSD | LHSGVPSRFSGSGSGTDY | | |
| | TSINTAYMELSRLRSDDTA | TLTISSLQPEDFATYFCQQ | | |
| | VYYCARDRGNYRAWFGY | GEALPWTFGGGTKVEIK | | |
| | WGQGTLVTVSS (SEQ ID | (SEQ ID NO: 775) | | |
| | NO: 774) | | | |
| Sarilumab | EVQLVESGGGLVQPGRSL | DIQMTQSPSSVSASVGD | na | na |
| (Format: Whole | RLSCAASRFTFDDYAMH | RVTITCRASQGISSWLAW | | |
| mAb) | WVRQAPGKGLEWVSGIS | YQQKPGKAPKLLIYGASS | | |
| | WNSGRIGYADSVKGRFTIS | LESGVPSRFSGSGSGTDF | | |
| | RDNAENSLFLQMNGLRAE | TLTISSLQPEDFASYYCQQ | | |
| | DTALYYCAKGRDSFDIWG | ANSFPYTFGQGTKLEIK | | |
| | QGTMVTVSS (SEQ ID | (SEQ ID NO: 777) | | |
| | NO: 776) | | | |
| Satralizumab | QVQLQESGPGLVKPSETLS | DIQMTQSPSSLSASVGDS | na | na |
| (Sapelizumab) | LTCAVSGHSISHDHAWSW | VTITCQASTDISSHLNWY | | |
| (Format: Whole | VRQPPGEGLEWIGFISYSG | QQKPGKAPELLIYYGSHL | | |
| mAb) | ITNYNPSLQGRVTISRDNS | LSGVPSRFSGSGSGTDFT | | |
| | KNTLYLQMNSLRAEDTAV | FTISSLEAEDAATYYCGQ | | |
| | YYCARSLARTTAMDYWGE | GNRLPYTFGQGTKVEIE | | |
| | GTLVTVSS (SEQ ID NO: | (SEQ ID NO: 779) | | |
| | 778) | | | |
| Secukinumab | EVQLVESGGGLVQPGGSL | EIVLTQSPGTLSLSPGERA | na | na |
| (Format: Whole | RLSCAASGFTFSNYWMN | TLSCRASQSVSSSYLAWY | | |
| mAb) | WVRQAPGKGLEWVAAIN | QQKPGQAPRLLIYGASSR | | |
| | QDGSEKYYVGSVKGRFTIS | ATGIPDRFSGSGSGTDFT | | |
| | RDNAKNSLYLQMNSLRVE | LTISRLEPEDFAVYYCQQY | | |
| | DTAVYYCVRDYYDILTDYYI | GSSPCTFGQGTRLEIK | | |
| | HYWYFDLWGRGTLVTVSS | (SEQ ID NO: 781) | | |
| | (SEQ ID NO: 780) | | | |
| Selicrelumab | QVQLVQSGAEVKKPGASV | DIQMTQSPSSVSASVGD | na | na |
| (Format: Whole | KVSCKASGYTFTGYYMHW | RVTITCRASQGIYSWLA | | |
| mAb) | VRQAPGQGLEWMGWIN | WYQQKPGKAPNLLIYTA | | |
| | PDSGGTNYAQKFQGRVT | STLQSGVPSRFSGSGSGT | | |
| | MTRDTSISTAYMELNRLRS | DFTLTISSLQPEDFATYYC | | |
| | DDTAVYYCARDQPLGYCT | QQANIFPLTFGGGTKVEI | | |
| | NGVCSYFDYWGQGTLVT | K (SEQ ID NO: 783) | | |
| | VSS (SEQ ID NO: 782) | | | |
| Semorinemab | EVQLVESGGGLVQPGGSL | DDVLTQTPLSLPVTPGQP | na | na |
| (Format: Whole | RLSCAASGLIFRSYGMSW | ASISCRSSQSIVHSNGNTY | | |
| mAb) | VRQAPGKGLEWVATINSG | LEWYLQKPGQSPQLLIYK | | |
| | GTYTYYPDSVKGRFTISRD | VSNRFSGVPDRFSGSGS | | |
| | NSKNTLYLQMNSLRAEDT | GTDFTLKISRVEAEDVGV | | |
| | AVYYCANSYSGAMDYWG | YYCFQGSLVPWTFGQGT | | |
| | QGTLVTVSS (SEQ ID NO: | KVEIK (SEQ ID NO: 785) | | |
| | 784) | | | |
| Seribantumab | EVQLLESGGGLVQPGGSL | QSALTQPASVSGSPGQSI | na | na |
| (Format: Whole | RLSCAASGFTFSHYVMAW | TISCTGTSSDVGSYNVVS | | |
| mAb) | VRQAPGKGLEWVSSISSS | WYQQHPGKAPKLIIYEVS | | |
| | GGWTLYADSVKGRFTISR | QRPSGVSNRFSGSKSGN | | |
| | DNSKNTLYLQMNSLRAED | TASLTISGLQTEDEADYYC | | |
| | TAVYYCTRGLKMATIFDY | CSYAGSSIFVIFGGGTKVT | | |
| | WGQGTLVTVSS (SEQ ID | VL (SEQ ID NO: 787) | | |
| | NO: 786) | | | |
| Setoxaximab | EVQLQQPGPELEKPGASV | DIVLSQSPSSLVVSVGEKV | na | na |
| (Format: Whole | KLSCKASGYSFTDYNMNW | TMSCKSSQSLLYSRNQKN | | |
| mAb) | VKQNNGESLEWIGKIDPY | YLAWYQQKPGQSPKVLI | | |
| | YGGPSYNQKFKDKATLTV | YWASTRESGVPDRLTGS | | |
| | DKSSSTAYMQLKSLTSEDS | GSGTDFTLTISSVKAEDLA | | |
| | AVYYCTRGGNRDWYFDV | VYYCQQYYSYPLTFGAGT | | |
| | WGAGTTLTVSA (SEQ ID | KLELK | | |
| | NO: 788) | (SEQ ID NO: 789) | | |
| Setrusumab | QVQLVESGGGLVQPGGSL | DIALTQPASVSGSPGQSIT | na | na |
| (Format: Whole | RLSCAASGFTFRSHWLSW | ISCTGTSSDVGDINDVSW | | |
| mAb) | VRQAPGKGLEWVSNINYD | YQQHPGKAPKLMIYDVN | | |
| | GSSTYYADSVKGRFTISRD | NRPSGVSNRFSGSKSGN | | |
| | NSKNTLYLQMNSLRAEDT | TASLTISGLQAEDEADYY | | |
| | AVYYCARDTYLHFDYWGQ | CQSYAGSYLSEVFGGGTK | | |
| | GTLVTVSS (SEQ ID NO: | LTVL (SEQ ID NO: 791) | | |
| | 790) | | | |
| Sifalimumab | QVQLVQSGAEVKKPGASV | EIVLTQSPGTLSLSPGERA | na | na |
| (Format: Whole | KVSCKASGYTFTSYSISWV | TLSCRASQSVSSTYLAWY | | |
| mAb) | RQAPGQGLEWMGWISV | QQKPGQAPRLLIYGASSR | | |
| | YNGNTNYAQKFQGRVTM | ATGIPDRFSGSGSGTDFT | | |
| | TTDTSTSTAYLELRSLRSDD | LTISRLEPEDFAVYYCQQY | | |
| | TAVYYCARDPIAAGYWGQ | GSSPRTFGQGTKVEIK | | |
| | GTLVTVSS (SEQ ID NO: | (SEQ ID NO: 793) | | |
| | 792) | | | |
| Siltuximab | EVQLVESGGKLLKPGGSLK | QIVLIQSPAIMSASPGEKV | na | na |
| (Format: Whole | LSCAASGFTFSSFAMSWF | TMTCSASSSVSYMYWYQ | | |
| mAb) | RQSPEKRLEWVAEISSGGS | QKPGSSPRLLIYDTSNLAS | | |
| | YTYYPDTVTGRFTISRDNA | GVPVRFSGSGSGTSYSLTI | | |
| | KNTLYLEMSSLRSEDTAM | SRMEAEDAATYYCQQW | | |
| | YYCARGLWGYYALDYWG | SGYPYTFGGGTKLEIK | | |
| | QGTSVTVSS (SEQ ID NO: | (SEQ ID NO: 795) | | |
| | 794) | | | |
| Simtuzumab | QVQLVQSGAEVKKPGASV | DIVMTQTPLSLSVTPGQP | na | na |
| (Format: Whole | KVSCKASGYAFTYYLIEWV | ASISCRSSKSLLHSNGNTY | | |
| mAb) | RQAPGQGLEWIGVINPGS | LYWFLQKPGQSPQFLIYR | | |
| | GGTNYNEKFKGRATITAD | MSNLASGVPDRFSGSGS | | |
| | KSTSTAYMELSSLRSEDTA | GTDFTLKISRVEAEDVGV | | |
| | VYFCARNWMNFDYWGQ | YYCMQHLEYPYTFGGGT | | |
| | GTTVTVSS (SEQ ID NO: | KVEIK (SEQ ID | | |
| | 796) | NO: 797) | | |
| Sintilimab | QVQLVQSGAEVKKPGSSV | DIQMTQSPSSVSASVGD | na | na |
| (Format: Whole | KVSCKASGGTFSSYAISWV | RVTITCRASQGISSWLAW | | |
| mAb) | RQAPGQGLEWMGLIIPM | YQQKPGKAPKLLISAASSL | | |
| | FDTAGYAQKFQGRVAITV | QSGVPSRFSGSGSGTDFT | | |
| | DESTSTAYMELSSLRSEDT | LTISSLQPEDFATYYCQQ | | |
| | AVYYCARAEHSSTGTFDY | ANHLPFTFGGGTKVEIK | | |
| | WGQGTLVTVSS (SEQ ID | (SEQ ID NO: 799) | | |
| | NO: 798) | | | |
| Sirtratumab | QVQLVESGGGVVQPGRSL | DIVMTQSPLSLPVTPGEP | na | na |
| (Format: Whole | RLSCAASGFTFSSYGMHW | ASISCRSSQSLLLSHGFNY | | |
| mAb ADC) | VRQAPGKGLEWVAVIWY | LDWYLQKPGQSPQLLIYL | | |
| | DGSNQYYADSVKGRFTISR | GSSRASGVPDRFSGSGS | | |
| | DNSKNTLFLQMHSLRAED | GTDFTLKISRVEAEDVGLY | | |
| | TAVYYCARGLTSGRYGMD | YCMQPLQIPWTFGQGTK | | |
| | VWGQGTTVTVSS (SEQ ID | VEIK (SEQ ID NO: 801) | | |
| | NO: 800) | | | |
| Sirukumab | EVQLVESGGGLVQPGGSL | EIVLTQSPATLSLSPGERA | na | na |
| (Format: Whole | RLSCAASGFTFSPFAMSW | TLSCSASISVSYMYWYQQ | | |
| mAb) | VRQAPGKGLEWVAKISPG | KPGQAPRLLIYDMSNLAS | | |
| | GSWTYYSDTVTGRFTISRD | GIPARFSGSGSGTDFTLTI | | |
| | NAKNSLYLQMNSLRAEDT | SSLEPEDFAVYYCMQWS | | |
| | AVYYCARQLWGYYALDIW | GYPYTFGGGTKVEIK | | |
| | GQGTTVTVSS (SEQ ID | (SEQ ID NO: 803) | | |
| | NO: 802) | | | |
| Sofituzumab | EVQLVESGGGLVQPGGSL | DIQMTQSPSSLSASVGD | na | na |
| (Format: Whole | RLSCAASGYSITNDYAWN | RVTITCKASDLIHNWLA | | |
| mAb ADC) | WVRQAPGKGLEWVGYIS | WYQQKPGKAPKLLIYGA | | |
| | YSGYTTYNPSLKSRFTISRD | TSLETGVPSRFSGSGSGT | | |
| | TSKNTLYLQMNSLRAEDT | DFTLTISSLQPEDFATYYC | | |
| | AVYYCARWTSGLDYWGQ | QQYWTTPFTFGQGTKVE | | |
| | GTLVTVSS (SEQ ID NO: | IK (SEQ ID NO: 805) | | |
| | 804) | | | |
| Solanezumab | EVQLVESGGGLVQPGGSL | DVVMTQSPLSLPVTLGQ | na | na |
| (Format: Whole | RLSCAASGFTFSRYSMSW | PASISCRSSQSLIYSDGNA | | |
| mAb) | VRQAPGKGLELVAQINSV | YLHWFLQKPGQSPRLLIY | | |
| | GNSTYYPDTVKGRFTISRD | KVSNRFSGVPDRFSGSGS | | |
| | NAKNTLYLQMNSLRAEDT | GTDFTLKISRVEAEDVGV | | |
| | AVYYCASGDYWGQGTLV | YYCSQSTHVPWTFGQGT | | |
| | TVSS (SEQ ID NO: 806) | KVEIK | | |
| | | (SEQ ID NO: 807) | | |
| Solitomab | EVQLLEQSGAELVRPGTSV | ELVMTQSPSSLTVTAGEK | DVQLVQSGAEVKK | DIVLTQSPATLSLS |
| (Format: | KISCKASGYAFTNYWLGW | VTMSCKSSQSLLNSGNQ | PGASVKVSCKASG | PGERATLSCRASQ |
| Bispecific scFV) | VKQRPGHGLEWIGDIFPG | KNYLTWYQQKPGQPPKL | YTFTRYTMHWVR | SVSYMNWYQQK |
| | SGNIHYNEKFKGKATLTAD | LIYWASTRESGVPDRFTG | QAPGQGLEWIGYI | PGKAPKRWIYDTS |
| | KSSSTAYMQLSSLTFEDSA | SGSGTDFTLTISSVQAEDL | NPSRGYTNYADSV | KVASGVPARFSGS |
| | VYFCARLRNWDEPMDYW | AVYYCQNDYSYPLTFGA | KGRFTITTDKSTST | GSGTDYSLTINSLE |
| | GQGTTVTVSS (SEQ ID | GTKLEIK (SEQ ID NO: | AYMELSSLRSEDTA | AEDAATYYCQQ |
| | NO: 808) | 809) | TYYCARYYDDHYCL | WSSNPLTFGGGT |
| | | | DYWGQGTTVTVSS | KVEIK (SEQ ID |
| | | | (SEQ ID NO: 810) | NO: 811) |
| Spartalizumab | EVQLVQSGAEVKKPGESL | EIVLTQSPATLSLSPGERA | na | na |
| (Format: Whole | RISCKGSGYTFTTYWMH | TLSCKSSQSLLDSGNQKN | | |
| mAb) | WVRQATGQGLEWMGNI | FLTWYQQKPGQAPRLLIY | | |
| | YPGTGGSNFDEKFKNRVTI | WASTRESGVPSRFSGSG | | |
| | TADKSTSTAYMELSSLRSE | SGTDFTFTISSLEAEDAAT | | |
| | DTAVYYCTRWTTGTGAY | YYCQNDYSYPYTFGQGT | | |
| | WGQGTTVTVSS (SEQ ID | KVEIK | | |
| | NO: 812) | (SEQ ID NO: 813) | | |
| Spesolimab | QVQLVQSGAEVKKPGASV | QIVLTQSPGTLSLSPGERA | na | na |
| (Format: Whole | KVSCKASGYSFTSSWIHW | TMTCTASSSVSSSYFHWY | | |
| mAb) | VKQAPGQGLEWMGEINP | QQKPGQAPRLWIYRTSR | | |
| | GNVRTNYNENFRNKVTM | LASGVPDRFSGSGSGTDF | | |
| | TVDTSISTAYMELSRLRSD | TLTISRLEPEDAATYYCHQ | | |
| | DTAVYYCTVVFYGEPYFPY | FHRSPLTFGAGTKLEIK | | |
| | WGQGTLVTVSS (SEQ ID | (SEQ ID NO: 815) | | |
| | NO: 814) | | | |
| Suptavumab | EVQLVESGGDLVQPGRSL | EIVMTQSPATLSVSPGER | na | na |
| (Format: Whole | RLSCVASGFTFDDYAMH | ATLSCRASQTILSNLAWY | | |
| mAb) | WVRQAPGKGLEWVSGVS | LQKPGQAPRLLIYGASTR | | |
| | WSGSTVGYADSVKGRFTV | ATGLPARFSGSGSGTEFT | | |
| | SRDNAQKSLYLQMNSLRA | LTISSLQSEDFAVYYCQQY | | |
| | EDTALYYCVKDAYKFNYYY | NNWPLTFGGGTKVEIK | | |
| | YGLDVWGQGTTVTVSS | (SEQ ID NO: 817) | | |
| | (SEQ ID NO: 816) | | | |
| Sutimlimab | EVQLVESGGGLVKPGGSL | QIVLTQSPATLSLSPGERA | na | na |
| (Format: Whole | RLSCAASGFTFSNYAMSW | TMSCTASSSVSSSYLHWY | | |
| mAb) | VRQAPGKGLEWVATISSG | QQKPGKAPKLWIYSTSNL | | |
| | GSHTYYLDSVKGRFTISRD | ASGVPSRFSGSGSGTDYT | | |
| | NSKNTLYLQMNSLRAEDT | LTISSLQPEDFATYYCHQY | | |
| | ALYYCARLFTGYAMDYW | YRLPPITFGQGTKLEIK | | |
| | GQGTLVTVSS (SEQ ID | (SEQ ID NO: 819) | | |
| | NO: 818) | | | |
| Suvizumab | QVQLVQSGAEVKKPGASV | DIQMTQRPDSLSASVGD | na | na |
| (Format: Whole | KVSCKASGYTFTNSWIGW | RVTMSCKSSQSLLNSGD | | |
| mAb) | FRQAPGQGLEWIGDIYPG | QKNYLTWYQQKPGQPP | | |
| | GGYTNYNEIFKGKATMTA | KLLIYWASTGESGVPDRF | | |
| | DTSTNTAYMELSSLRSEDT | SGSGSGTDFTFTISSLQPE | | |
| | AVYYCSRGIPGYAMDYW | DIATYYCQNDYSYPWTF | | |
| | GQGTLVTVSS (SEQ ID | GQGTKVEIK (SEQ ID | | |
| | NO: 820) | NO: 821) | | |
| Suvratoxumab | EVQLVESGGGLVQPGGSL | DIQMTQSPSTLSASVGD | na | na |
| (Format: Whole | RLSCAASGFTFSSHDMHW | RVTITCRASQSISSWLAW | | |
| mAb) | VRQATGKGLEWVSGIGTA | YQQKPGKAPKLLIYKASSL | | |
| | GDTYYPDSVKGRFTISREN | ESGVPSRFSGSGSGTEFT | | |
| | AKNSLYLQMNSLRAGDTA | LTISSLQPDDFATYYCKQY | | |
| | VYYCARDRYSPTGHYYGM | ADYWTFGQGTKVEIK | | |
| | DVWGQGTTVTVSS (SEQ | (SEQ ID NO: 823) | | |
| | ID NO: 822) | | | |
| Tabalumab | QVQLQQWGAGLLKPSETL | EIVLTQSPATLSLSPGERA | na | na |
| (Format: Whole | SLTCAVYGGSFSGYYWSW | TLSCRASQSVSRYLAWYQ | | |
| mAb) | IRQPPGKGLEWIGEINHSG | QKPGQAPRLLIYDASNRA | | |
| | STNYNPSLKSRVTISVDTSK | TGIPARFSGSGSGTDSTLT | | |
| | NQFSLKLSSVTAADTAVYY | ISSLEPEDFAVYYCQQRS | | |
| | CARGYYDILTGYYYYFDYW | NWPRTFGQGTKVEIK | | |
| | GQGTLVTVSS (SEQ ID | (SEQ ID NO: 825) | | |
| | NO: 824) | | | |
| Tabituximab | EVQLQQSGAELVKPGASV | DIQMTQSPASLSVSVGET | na | na |
| (Format: Whole | KLSCTASGFNINDTYMHW | VTITCRASENIYSNLAWY | | |
| mAb ADC) | VKQRPEQGLEWIGRIDPA | QQKQGKSPQLLVYVATN | | |
| | NGNTKYDPKFQGKATITA | LADGVPSRFSGSGSGTQY | | |
| | DTSSNTAYLQLSSLTSEDT | SLKINSLQSEDFGSYYCQ | | |
| | AVYYCARGARGSRFAYW | HFWGTPYTFGGGTKLEIK | | |
| | GQGTLVTVSA (SEQ ID | (SEQ ID NO: 827) | | |
| | NO: 826) | | | |
| Tadocizumab | QVQLVQSGAEVKKPGSSV | DIQMTQTPSTLSASVGD | na | na |
| (Format: Fab) | KVSCKASGYAFTNYLIEWV | RVTISCRASQDINNYLNW | | |
| | RQAPGQGLEWIGVIYPGS | YQQKPGKAPKLLIYYTSTL | | |
| | GGTNYNEKFKGRVTLTVD | HSGVPSRFSGSGSGTDYT | | |
| | ESTNTAYMELSSLRSEDTA | LTISSLQPDDFATYFCQQ | | |
| | VYFCARRDGNYGWFAYW | GNTLPWTFGQGTKVEVK | | |
| | GQGTLVTVSS (SEQ ID | (SEQ ID NO: 829) | | |
| | NO: 828) | | | |
| Tafasitamab | EVQLVESGGGLVKPGGSL | DIVMTQSPATLSLSPGER | na | na |
| (Format: Whole | KLSCAASGYTFTSYVMHW | ATLSCRSSKSLQNVNGNT | | |
| mAb) | VRQAPGKGLEWIGYINPY | YLYWFQQKPGQSPQLLIY | | |
| | NDGTKYNEKFQGRVTISS | RMSNLNSGVPDRFSGSG | | |
| | DKSISTAYMELSSLRSEDTA | SGTEFTLTISSLEPEDFAVY | | |
| | MYYCARGTYYYGTRVFDY | YCMQHLEYPITFGAGTKL | | |
| | WGQGTLVTVSS (SEQ ID | EIK (SEQ ID NO: 831) | | |
| | NO: 830) | | | |
| Talacotuzumab | EVQLVQSGAEVKKPGESL | DIVMTQSPDSLAVSLGER | na | na |
| (Format: Whole | KISCKGSGYSFTDYYMKW | ATINCESSQSLLNSGNQK | | |
| mAb) | ARQMPGKGLEWMGDIIP | NYLTWYQQKPGQPPKPL | | |
| | SNGATFYNQKFKGQVTIS | IYWASTRESGVPDRFSGS | | |
| | ADKSISTTYLQWSSLKASD | GSGTDFTLTISSLQAEDV | | |
| | TAMYYCARSHLLRASWFA | AVYYCQNDYSYPYTFGQ | | |
| | YWGQGTMVTVSS (SEQ | GTKLEIK (SEQ ID NO: | | |
| | ID NO: 832) | 833) | | |
| Tamrintamab | QVQLVQSGAEVKKPGSSV | EIVLTQSPATLSLSPGERA | na | na |
| (Format: Whole | KVSCKASGGTFSSYWIEW | TLSCTASSSVNSFYLHWY | | |
| mAb ADC) | VRQAPGQGLEWMGEILP | QQKPGLAPRLLIYSTSNLA | | |
| | GSGNTYYNERFKDRVTITA | SGIPDRFSGSGSGTDFTLT | | |
| | DESTSTAYMELSSLRSEDT | ISRLEPEDFAVYYCHQYH | | |
| | AVYYCARRAAAYYSNPEW | RSPYTFGQGTKLEIK | | |
| | FAYWGQGTLVTVSS (SEQ | (SEQ ID NO: 835) | | |
| | ID NO: 834) | | | |
| Tamtuvetmab | EVKLLESGGGLVQPGGSM | DIKMTQSPSFLSASVGDR | na | na |
| (Format: Canine | RLSCAGSGFTFTDFYMNW | VTLNCKASQNIDKYLNW | | |
| Whole mAb) | IRQPAGKAPEWLGFIRDK | YQQKLGESPKLLIYNTNN | | |
| | AKGYTTEYNPSVKGRFTIS | LQTGIPSRFSGSGSGTDF | | |
| | RDNTQNMLYLQMNTLRA | TLTISSLQPEDVATYFCLQ | | |
| | EDTATYYCAREGHTAAPF | HISRPRTFGGGTHLTVL | | |
| | DYWGQGTLVTVSS (SEQ | (SEQ ID NO: 837) | | |
| | ID NO: 836) | | | |
| Tanezumab | QVQLQESGPGLVKPSETLS | DIQMTQSPSSLSASVGD | na | na |
| (Format: Whole | LTCTVSGFSLIGYDLNWIR | RVTITCRASQSISNNLNW | | |
| mAb) | QPPGKGLEWIGIIWGDGT | YQQKPGKAPKLLIYYTSRF | | |
| | TDYNSAVKSRVTISKDTSK | HSGVPSRFSGSGSGTDFT | | |
| | NQFSLKLSSVTAADTAVYY | FTISSLQPEDIATYYCQQE | | |
| | CARGGYWYATSYYFDYW | HTLPYTFGQGTKLEIK | | |
| | GQGTLVTVSS (SEQ ID | (SEQ ID NO: 839) | | |
| | NO: 838) | | | |
| Tarextumab | EVQLVESGGGLVQPGGSL | DIVLTQSPATLSLSPGERA | na | na |
| (Format: Whole | RLSCAASGFTFSSSGMSW | TLSCRASQSVRSNYLAWY | | |
| mAb) | VRQAPGKGLEWVSVIASS | QQKPGQAPRLLIYGASSR | | |
| | GSNTYYADSVKGRFTISRD | ATGVPARFSGSGSGTDFT | | |
| | NSKNTLYLQMNSLRAEDT | LTISSLEPEDFAVYYCQQY | | |
| | AVYYCARSIFYTTWGQGTL | SNFPITFGQGTKVEIK | | |
| | VTVSS (SEQ ID NO: 840) | (SEQ ID NO: 841) | | |
| Tavolimab | QVQLQESGPGLVKPSQTL | DIQMTQSPSSLSASVGD | na | na |
| (Tavolixizumab) | SLTCAVYGGSFSSGYWN | RVTITCRASQDISNYLNW | | |
| (Format: Whole | WIRKHPGKGLEYIGYISYN | YQQKPGKAPKLLIYYTSKL | | |
| mAb) | GITYHNPSLKSRITINRDTS | HSGVPSRFSGSGSGTDYT | | |
| | KNQYSLQLNSVTPEDTAV | LTISSLQPEDFATYYCQQ | | |
| | YYCARYKYDYDGGHAMD | GSALPWTFGQGTKVEIK | | |
| | YWGQGTLVTVSS (SEQ ID | (SEQ ID NO: 843) | | |
| | NO: 842) | | | |
| Tebentafusp | EVQLVESGGGLVQPGGSL | AIQMTQSPSSLSASVGDR | na | na |
| (Format: scFv | RLSCAASGYSFTGYTMNW | VTITCRASQDIRNYLNWY | | |
| Fusion) | VRQAPGKGLEWVALINPY | QQKPGKAPKLLIYYTSRLE | | |
| | KGVSTYNQKFKDRFTISVD | SGVPSRFSGSGSGTDYTL | | |
| | KSKNTAYLQMNSLRAEDT | TISSLQPEDFATYYCQQG | | |
| | AVYYCARSGYYGDSDWYF | NTLPWTFGQGTKVEIK | | |
| | DVWGQGTLVTVSS (SEQ | (SEQ ID NO: 845) | | |
| | ID NO: 844) | | | |
| Teclistamab | QLQLQESGPGLVKPSETLS | SYVLTQPPSVSVAPGQTA | EVQLVESGGGLVQ | QTVVTQEPSLTVS |
| (Format: | LTCTVSGGSISSGSYFWG | RITCGGNNIGSKSVHWY | PGGSLRLSCAASGF | PGGTVTLTCRSST |
| Bispecific mAb) | WIRQPPGKGLEWIGSIYYS | QQPPGQAPVVVVYDDS | TFNTYAMNWVRQ | GAVTTSNYANW |
| | GITYYNPSLKSRVTISVDTS | DRPSGIPERFSGSNSGNT | APGKGLEWVARIR | VQQKPGQAPRGL |
| | KNQFSLKLSSVTAADTAVY | ATLTISRVEAGDEAVYYC | SKYNNYATYYAASV | IGGTNKRAPGTPA |
| | YCARHDGAVAGLFDYWG | QVWDSSSDHVVFGGGT | KGRFTISRDDSKNS | RFSGSLLGGKAAL |
| | QGTLVTVSS (SEQ ID NO: | KLTVL | LYLQMNSLKTEDT | TLSGVQPEDEAEY |
| | 846) | (SEQ ID NO: 847) | AVYYCARHGNFGN | YCALWYSNLWVF |
| | | | SYVSWFAYWGQG | GGGTKLTVL (SEQ |
| | | | TLVTVSS (SEQ ID | ID NO: 849) |
| | | | NO: 848) | |
| Telisotuzumab | QVQLVQSGAEVKKPGASV | DIVMTQSPDSLAVSLGER | na | na |
| (Format: Whole | KVSCKASGYIFTAYTMHW | ATINCKSSESVDSYANSFL | | |
| mAb ADC) | VRQAPGQGLEWMGWIK | HWYQQKPGQPPKLLIYR | | |
| | PNNGLANYAQKFQGRVT | ASTRESGVPDRFSGSGSG | | |
| | MTRDTSISTAYMELSRLRS | TDFTLTISSLQAEDVAVYY | | |
| | DDTAVYYCARSEITTEFDY | CQQSKEDPLTFGGGTKV | | |
| | WGQGTLVTVSS (SEQ ID | EIK (SEQ ID NO: 851) | | |
| | NO: 850) | | | |
| Temelimab | QVQLVQSGAEVKKPGSSV | QIQLTQSPSSLSASVGDR | na | na |
| (Format: Whole | KVSCKASGYTFTDYEMHW | VTITCSASSSVSYMYWYQ | | |
| mAb) | VRQAPGQGLEWIGAVAP | QKPGKAPKAWIYRTSNL | | |
| | ETGGTAYNQKFKGRATITA | ASGVPSRFSGSGSGTDYT | | |
| | DKSTSTAYMELSSLRSEDT | LTISSLQPEDFATYYCQQY | | |
| | AVYYCTSTVVPFAYWGQG | QSLPLTFGGGTKVEIK | | |
| | TLVTVSS (SEQ ID NO: | (SEQ ID NO: 853) | | |
| | 852) | | | |
| Tenatumomab | EIQLQQSGPELVKPGASVK | DIVMTQAAPSVPVTPGE | na | na |
| (Format: Whole | VSCKASGYAFTSYNMYWV | SVSISCRSSKSLLHSNGNT | | |
| mAb | KQSHGKSLEWIGYIDPYN | YLYWFLQRPGQSPQLLIY | | |
| Radiolabelled) | GVTSYNQKFKGKATLTVD | RMSNLASGVPDRFSGSG | | |
| | KSSSTAYMHLNSLTSEDSA | SGTAFTLRISRVEAEDVG | | |
| | VYYCARGGGSIYYAMDY | VYYCMQHLEYPLTFGAG | | |
| | WGQGTSVTVSS (SEQ ID | TKLELK (SEQ ID NO: | | |
| | NO: 854) | 855) | | |
| Teplizumab | QVQLVQSGGGVVQPGRS | DIQMTQSPSSLSASVGD | na | na |
| (Format: Whole | LRLSCKASGYTFTRYTMH | RVTITCSASSSVSYMNWY | | |
| mAb) | WVRQAPGKGLEWIGYINP | QQTPGKAPKRWIYDTSK | | |
| | SRGYTNYNQKVKDRFTISR | LASGVPSRFSGSGSGTDY | | |
| | DNSKNTAFLQMDSLRPED | TFTISSLQPEDIATYYCQQ | | |
| | TGVYFCARYYDDHYCLDY | WSSNPFTFGQGTKLQIT | | |
| | WGQGTPVTVSS (SEQ ID | (SEQ ID NO: 857) | | |
| | NO: 856) | | | |
| Tepoditamab | QVQLVQSGAEVKKPGASV | DIQMTQSPSSLSASVGD | QVQLVQSGGGVV | DIQMTQSPSSLSA |
| (Format: | KVSCKASGYTFTSYYMHW | RVTITCRASQSISSYLNWY | QPGRSLRLSCVASG | SVGDRVTITCRAS |
| Bispecific mAb) | VRQAPGQGLEWMGIINP | QQKPGKAPKLLIYAASSL | FTFSSYGMHWVR | QSISSYLNWYQQ |
| | SGGSTSYAQKFQGRVTMT | QSGVPSRFSGSGSGTDFT | QAPGKGLEWVAAI | KPGKAPKLLIYAAS |
| | RDTSTSTVYMELSSLRSED | LTISSLQPEDFATYYCQQS | WYNARKQDYADS | SLQSGVPSRFSGS |
| | TAVYYCAKGTTGDWFDY | YSTPPTFGQGTKVEIK | VKGRFTISRDNSKN | GSGTDFTLTISSLQ |
| | WGQGTLVTVSS (SEQ ID | (SEQ ID NO: 859) | TLYLQMNSLRAED | PEDFATYYCQQSY |
| | NO: 858) | | TAVYYCTRGTGYN | STPPTFGQGTKVE |
| | | | WFDPWGQGTLVT | IK (SEQ ID NO: |
| | | | VSS (SEQ ID NO: | 861) |
| | | | 860) | |
| Teprotumumab | QVELVESGGGVVQPGRS | EIVLTQSPATLSLSPGERA | na | na |
| (Format: Whole | QRLSCAASGFTFSSYGMH | TLSCRASQSVSSYLAWYQ | | |
| mAb) | WVRQAPGKGLEWVAIIW | QKPGQAPRLLIYDASKRA | | |
| | FDGSSTYYADSVRGRFTIS | TGIPARFSGSGSGTDFTLT | | |
| | RDNSKNTLYLQMNSLRAE | ISSLEPEDFAVYYCQQRSK | | |
| | DTAVYFCARELGRRYFDL | WPPWTFGQGTKVESK | | |
| | WGRGTLVSVSS (SEQ ID | (SEQ ID NO: 863) | | |
| | NO: 862) | | | |
| Tesidolumab | EVQLVQSGAEVKKPGSSV | SYELTQPLSVSVALGQTA | na | na |
| (Format: Whole | KVSCKASGGTFSSYAISWV | RITCSGDSIPNYYVYWYQ | | |
| mAb) | RQAPGQGLEWMGGIGPF | QKPGQAPVLVIYDDSNR | | |
| | FGTANYAQKFQGRVTITA | PSGIPERFSGSNSGNTAT | | |
| | DESTSTAYMELSSLRSEDT | LTISRAQAGDEADYYCQS | | |
| | AVYYCARDTPYFDYWGQ | FDSSLNAEVFGGGTKLTV | | |
| | GTLVTVSS (SEQ ID NO: | L (SEQ ID NO: 865) | | |
| | 864) | | | |
| Tezepelumab | QMQLVESGGGVVQPGRS | SYVLTQPPSVSVAPGQTA | na | na |
| (Format: Whole | LRLSCAASGFTFRTYGMH | RITCGGNNLGSKSVHWY | | |
| mAb) | WVRQAPGKGLEWVAVI | QQKPGQAPVLVVYDDSD | | |
| | WYDGSNKHYADSVKGRF | RPSWIPERFSGSNSGNTA | | |
| | TITRDNSKNTLNLQMNSL | TLTISRGEAGDEADYYCQ | | |
| | RAEDTAVYYCARAPQWEL | VWDSSSDHVVFGGGTKL | | |
| | VHEAFDIWGQGTMVTVS | TVL (SEQ ID NO: 867) | | |
| | S (SEQ ID NO: 866) | | | |
| Tibulizumab | QVQLQQWGAGLLKPSETL | EIVLTQSPATLSLSPGERA | QVQLVQSGAEVKK | DIVMTQTPLSLSV |
| (Format: | SLTCAVYGGSFSGYYWSW | TLSCRASQSVSRYLAWYQ | PGSSVKVSCKASGY | TPGQPASISCRSS |
| Bispecific Mixed | IRQPPGKGLEWIGEINHSG | QKPGQAPRLLIYDASNRA | KFTDYHIHWVRQA | RSLVHSRGETYLH |
| mAb and scFV) | STNYNPSLKSRVTISVDTSK | TGIPARFSGSGSGTDSTLT | PGQCLEWMGVIN | WYLQKPGQSPQL |
| | NQFSLKLSSVTAADTAVYY | ISSLEPEDFAVYYCQQRS | PTYGTTDYNQRFK | LIYKVSNRFIGVPD |
| | CARGYYDILTGYYYYFDYW | NWPRTFGQGTKVEIK | GRVTITADESTSTA | RFSGSGSGTDFTL |
| | GQGTLVTVSS (SEQ ID | (SEQ ID NO: 869) | YMELSSLRSEDTAV | KISRVEAEDVGVY |
| | NO: 868) | | YYCARYDYFTGTG | YCSQSTHLPFTFG |
| | | | VYWGQGTLVTVSS | CGTKLEIK (SEQ |
| | | | (SEQ ID NO: 870) | ID NO: 871) |
| Tidutamab | EVQLVESGGGLVQPGGSL | DIVMTQSPDSLAVSLGER | EVQLVESGGGLVQ | QAVVTQEPSLTVS |
| (Format: | RLSCAASGFTFSDYGMAW | ATINCKSSQSLLNSRNRK | PGGSLRLSCAASGF | PGGTVTLTCGSST |
| Bispecific Mixed | FRQAPGKGLEWVSFISNL | NYLAWYQQKPDQSPKLL | TFSTYAMNWVRQ | GAVTTSNYANW |
| mAb and scFV) | GYSIYYADSVKGRFTISRD | IYWASTRESGVPDRFSGS | APGKGLEWVGRIR | VQQKPGKSPRGLI |
| | NAKNSLYLQMNSLRAEDT | GSGTDFTLTISSLQAEDV | SKYNNYATYYADS | GGTNKRAPGVPA |
| | AVYYCARAPYDYDSFDPM | AVYYCKQSYYLWTFGGG | VKGRFTISRDDSKN | RFSGSLLGGKAAL |
| | DYWGQGTLVTVSS (SEQ | TKVEIK (SEQ ID NO: | TLYLQMNSLRAED | TISGAQPEDEADY |
| | ID NO: 872) | 873) | TAVYYCVRHGNFG | YCALWYSNHWV |
| | | | DSYVSWFAYWGQ | FGGGTKLTVL |
| | | | GTLVTVSS (SEQ ID | (SEQ ID |
| | | | NO: 874) | NO: 875) |
| Tigatuzumab | EVQLVESGGGLVQPGGSL | DIQMTQSPSSLSASVGD | na | na |
| (Format: Whole | RLSCAASGFTFSSYVMSW | RVTITCKASQDVGTAVA | | |
| mAb) | VRQAPGKGLEWVATISSG | WYQQKPGKAPKLLIYWA | | |
| | GSYTYYPDSVKGRFTISRD | STRHTGVPSRFSGSGSGT | | |
| | NAKNTLYLQMNSLRAEDT | DFTLTISSLQPEDFATYYC | | |
| | AVYYCARRGDSMITTDYW | QQYSSYRTFGQGTKVEIK | | |
| | GQGTLVTVSS (SEQ ID | (SEQ ID NO: 877) | | |
| | NO: 876) | | | |
| Tilavonemab | EVKVVESGGGLVQPGGS | DIVLTQSPDSLAVSLGER | na | na |
| (Format: Whole | MKLSCVVSGFTFSNYWVN | ATISCRASQSVSTSRYSYI | | |
| mAb) | WVRQAPGKGLEWVAQIR | HWYQQKPGQPPKLLIKY | | |
| | LKSDNYATHYEESVKGRFT | ASNLESGVPSRFGSGSGT | | |
| | ISRDDSKSSVYLQMNNLR | DFTLNIHPLEPEDFATYYC | | |
| | AEDSGIYYCTNWEDYWG | HHSWEIPLTFGQGTKLEI | | |
| | QGTTVTVSS (SEQ ID NO: | K (SEQ ID NO: 879) | | |
| | 878) | | | |
| Tildrakizumab | QVQLVQSGAEVKKPGASV | DIQMTQSPSSLSASVGD | na | na |
| (Format: Whole | KVSCKASGYIFITYWMTW | RVTITCRTSENIYSYLAWY | | |
| mAb) | VRQAPGQGLEWMGQIFP | QQKPGKAPKLLIYNAKTL | | |
| | ASGSADYNEKFEGRVTMT | AEGVPSRFSGSGSGTDFT | | |
| | TDTSTSTAYMELRSLRSDD | LTISSLQPEDFATYYCQH | | |
| | TAVYYCARGGGGFAYWG | HYGIPFTFGQGTKVEIK | | |
| | QGTLVTVSS (SEQ ID NO: | (SEQ ID NO: 881) | | |
| | 880) | | | |
| Timolumab | QVQLVESGGGVVQPGRSL | VIQLTQSPSSLSASVGDR | na | na |
| (Format: Whole | RLSCAASGFTFFSYAMHW | VTITCRASQGISRALAWY | | |
| mAb) | VRQTPGKGLEWVAVIWF | QQKPGKGPKLLIYDASSL | | |
| | DGSNENYVDSVKGRFTISR | ESGVPSRFSGSGSGTDFT | | |
| | DNSKNTLYLQMNTLRAED | LTISSLQPEDFATYYCQQF | | |
| | TAVYYCARDAWSYFDYW | NSYPLTFGGGTKVEIK | | |
| | GQGTLVTVSS (SEQ ID | (SEQ ID NO: 883) | | |
| | NO: 882) | | | |
| Tiragolumab | EVQLQQSGPGLVKPSQTL | DIVMTQSPDSLAVSLGER | na | na |
| (Format: Whole | SLTCAISGDSVSSNSAAW | ATINCKSSQTVLYSSNNK | | |
| mAb) | NWIRQSPSRGLEWLGKTY | KYLAWYQQKPGQPPNLL | | |
| | YRFKWYSDYAVSVKGRITI | IYWASTRESGVPDRFSGS | | |
| | NPDTSKNQFSLQLNSVTPE | GSGTDFTLTISSLQAEDV | | |
| | DTAVFYCTRESTTYDLLAG | AVYYCQQYYSTPFTFGPG | | |
| | PFDYWGQGTLVTVSS | TKVEIK (SEQ ID NO: | | |
| | (SEQ ID NO: 884) | 885) | | |
| Tislelizumab | QVQLQESGPGLVKPSETLS | DIVMTQSPDSLAVSLGER | na | na |
| (Format: Whole | LTCTVSGFSLTSYGVHWIR | ATINCKSSESVSNDVAWY | | |
| mAb) | QPPGKGLEWIGVIYADGS | QQKPGQPPKLLINYAFHR | | |
| | TNYNPSLKSRVTISKDTSK | FTGVPDRFSGSGYGTDFT | | |
| | NQVSLKLSSVTAADTAVYY | LTISSLQAEDVAVYYCHQ | | |
| | CARAYGNYWYIDVWGQG | AYSSPYTFGQGTKLEIK | | |
| | TTVTVSS (SEQ ID NO: | (SEQ ID NO: 887) | | |
| | 886) | | | |
| Tisotumab | EVQLLESGGGLVQPGGSL | DIQMTQSPPSLSASAGD | na | na |
| (Format: Whole | RLSCAASGFTFSNYAMSW | RVTITCRASQGISSRLAW | | |
| mAb) | VRQAPGKGLEWVSSISGS | YQQKPEKAPKSLIYAASSL | | |
| | GDYTYYTDSVKGRFTISRD | QSGVPSRFSGSGSGTDFT | | |
| | NSKNTLYLQMNSLRAEDT | LTISSLQPEDFATYYCQQY | | |
| | AVYYCARSPWGYYLDSW | NSYPYTFGQGTKLEIK | | |
| | GQGTLVTVSS (SEQ ID | (SEQ ID NO: 889) | | |
| | NO: 888) | | | |
| Tocilizumab | QVQLQESGPGLVRPSQTL | DIQMTQSPSSLSASVGD | na | na |
| (Format: Whole | SLTCTVSGYSITSDHAWS | RVTITCRASQDISSYLNW | | |
| mAb) | WVRQPPGRGLEWIGYISY | YQQKPGKAPKLLIYYTSRL | | |
| | SGITTYNPSLKSRVTMLRD | HSGVPSRFSGSGSGTDFT | | |
| | TSKNQFSLRLSSVTAADTA | FTISSLQPEDIATYYCQQG | | |
| | VYYCARSLARTTAMDYW | NTLPYTFGQGTKVEIK | | |
| | GQGSLVTVSS (SEQ ID | (SEQ ID NO: 891) | | |
| | NO: 890) | | | |
| Tomaralimab | EVQLVQSGSELKKPGASVK | DIVLTQSPATLSLSPGERA | na | na |
| (Format: Whole | LSCKASGFTFTTYGINWVR | TLSCRASESVEYYGTSLM | | |
| mAb) | QAPGQGLEWIGWIYPRD | QWYQQKPGQPPKLLIFG | | |
| | GSTNFNENFKDRATITVDT | ASNVESGVPDRFSGSGS | | |
| | SASTAYMELSSLRSEDTAV | GTDFTLKISRVEAEDVGM | | |
| | YFCARLTGGTFLDYWGQG | YFCQQSRKLPWTFGGGT | | |
| | TTVTVSS (SEQ ID NO: | KVEIK | | |
| | 892) | (SEQ ID NO: 893) | | |
| Tomuzotuximab | QVQLKQSGPGLVQPSQSL | DILLTQSPVILSVSPGERV | na | na |
| (Format: Whole | SITCTVSGFSLTNYGVHWV | SFSCRASQSIGTNIHWYQ | | |
| mAb) | RQSPGKGLEWLGVIWSG | QRTNGSPRLLIKYASESIS | | |
| | GNTDYNTPFTSRLSINKDN | GIPSRFSGSGSGTDFTLSI | | |
| | SKSQVFFKMNSLQSNDTA | NSVESEDIADYYCQQNN | | |
| | IYYCARALTYYDYEFAYWG | NWPTTFGAGTKLELK | | |
| | QGTLVTVST (SEQ ID NO: | (SEQ ID NO: 895) | | |
| | 894) | | | |
| Toripalimab | QGQLVQSGAEVKKPGASV | DVVMTQSPLSLPVTLGQ | na | na |
| (Format: Whole | KVSCKASGYTFTDYEMHW | PASISCRSSQSIVHSNGNT | | |
| mAb) | VRQAPIHGLEWIGVIESET | YLEWYLQKPGQSPQLLIY | | |
| | GGTAYNQKFKGRVTITAD | KVSNRFSGVPDRFSGSGS | | |
| | KSTSTAYMELSSLRSEDTA | GTDFTLKISRVEAEDVGV | | |
| | VYYCAREGITTVATTYYWY | YYCFQGSHVPLTFGQGT | | |
| | FDVWGQGTTVTVSS (SEQ | KLEIK | | |
| | ID NO: 896) | (SEQ ID NO: 897) | | |
| Tosatoxumab | EVQMVQSGAEVKKPGEPL | QSVLTQSPSASGTPGQR | na | na |
| (Format: Whole | KISCKGSGYKFGTHWIGW | VTISCSGGSSNIGSNTVN | | |
| mAb) | VRQRPGKGLEWMGIIHPA | WYQQFPGAAPKLLIYTN | | |
| | DSETKYSPSFQGQVSFSAD | NQRPSGVPDRFSGSKSG | | |
| | KSSNTAYLHWSTLRASDT | TSASLAISGLQSEDEADYY | | |
| | AMYYCARRSGSSSWYALD | CATWDDSLNGLYVFGTG | | |
| | FWGQGTMVTVSS (SEQ | TKVTVL (SEQ ID NO: | | |
| | ID NO: 898) | 899) | | |
| Tovetumab | QVQLVESGGGLVKPGGSL | DIQMTQSPSSLSASVGD | na | na |
| (Format: Whole | RLSCAASGFTFSDYYMNW | RVSITCRPSQSFSRYINWY | | |
| mAb) | IRQAPGKGLEWVSYISSSG | QQKPGKAPKLLIHAASSL | | |
| | SIIYYADSVKGRFTISRDNA | VGGVPSRFSGSGSGTDFT | | |
| | KNSLYLQMNSLRAEDTAV | LTISSLQPEDFATYYCQQT | | |
| | YYCAREGRIAARGMDVW | YSNPPITFGQGTRLEMK | | |
| | GQGTTVTVSS (SEQ ID | (SEQ ID NO: 901) | | |
| | NO: 900) | | | |
| Tralokinumab | QVQLVQSGAEVKKPGASV | SYVLTQPPSVSVAPGKTA | na | na |
| (Format: Whole | KVSCKASGYTFTNYGLSW | RITCGGNIIGSKLVHWYQ | | |
| mAb) | VRQAPGQGLEWMGWIS | QKPGQAPVLVIYDDGDR | | |
| | ANNGDTNYGQEFQGRVT | PSGIPERFSGSNSGNTAT | | |
| | MTTDTSTSTAYMELRSLRS | LTISRVEAGDEADYYCQV | | |
| | DDTAVYYCARDSSSSWAR | WDTGSDPVVFGGGTKLT | | |
| | WFFDLWGRGTLVTVSS | VL (SEQ ID NO: 903) | | |
| | (SEQ ID NO: 902) | | | |
| Trastuzumab | EVQLVESGGGLVQPGGSL | DIQMTQSPSSLSASVGD | na | na |
| (Timigutuzumab) | RLSCAASGFNIKDTYIHWV | RVTITCRASQDVNTAVA | | |
| (Format: Whole | RQAPGKGLEWVARIYPTN | WYQQKPGKAPKLLIYSAS | | |
| mAb) | GYTRYADSVKGRFTISADT | FLYSGVPSRFSGSRSGTD | | |
| | SKNTAYLQMNSLRAEDTA | FTLTISSLQPEDFATYYCQ | | |
| | VYYCSRWGGDGFYAMDY | QHYTTPPTFGQGTKVEIK | | |
| | WGQGTLVTVSS (SEQ ID | (SEQ ID NO: 905) | | |
| | NO: 904) | | | |
| Tregalizumab | EEQLVESGGGLVKPGGSL | DIVMTQSPDSLAVSLGER | na | na |
| (Format: Whole | RLSCAASGFSFSDCRMYW | ATINCRASKSVSTSGYSYI | | |
| mAb) | LRQAPGKGLEWIGVISVKS | YWYQQKPGQPPKLLIYLA | | |
| | ENYGANYAESVRGRFTISR | SILESGVPDRFSGSGSGT | | |
| | DDSKNTVYLQMNSLKTED | DFTLTISSLQAEDVAVYYC | | |
| | TAVYYCSASYYRYDVGAW | QHSRELPWTFGQGTKVE | | |
| | FAYWGQGTLVTVSS (SEQ | IK (SEQ ID NO: 907) | | |
| | ID NO: 906) | | | |
| Tremelimumab | QVQLVESGGGVVQPGRSL | DIQMTQSPSSLSASVGD | na | na |
| (Ticilimumab) | RLSCAASGFTFSSYGMHW | RVTITCRASQSINSYLDW | | |
| (Format: Whole | VRQAPGKGLEWVAVIWY | YQQKPGKAPKLLIYAASSL | | |
| mAb) | DGSNKYYADSVKGRFTISR | QSGVPSRFSGSGSGTDFT | | |
| | DNSKNTLYLQMNSLRAED | LTISSLQPEDFATYYCQQY | | |
| | TAVYYCARDPRGATLYYYY | YSTPFTFGPGTKVEIK | | |
| | YGMDVWGQGTTVTVSS | (SEQ ID NO: 909) | | |
| | (SEQ ID NO: 908) | | | |
| Trevogrumab | EVQVLESGGDLVQPGGSL | DIQMTQSPASLSASVGD | na | na |
| (Format: Whole | RLSCAASGFTFSAYAMTW | RVTITCRASQDISDYLAW | | |
| mAb) | VRQAPGKGLEWVSAISGS | YQQKPGKIPRLLIYTTSTL | | |
| | GGSAYYADSVKGRFTISRD | QSGVPSRFRGSGSGTDFT | | |
| | NSKNTVYLQMNSLRAEDT | LTISSLQPEDVATYYCQKY | | |
| | AVYYCAKDGAWKMSGLD | DSAPLTFGGGTKVEIK | | |
| | VWGQGTTVIVSS (SEQ ID | (SEQ ID NO: 911) | | |
| | NO: 910) | | | |
| Ublituximab | QAYLQQSGAELVRPGASV | QIVLSQSPAILSASPGEKV | na | na |
| (Format: Whole | KMSCKASGYTFTSYNMH | TMTCRASSSVSYMHWY | | |
| mAb) | WVKQTPRQGLEWIGGIYP | QQKPGSSPKPWIYATSNL | | |
| | GNGDTSYNQKFKGKATLT | ASGVPARFSGSGSGTSYS | | |
| | VGKSSSTAYMQLSSLTSED | FTISRVEAEDAATYYCQQ | | |
| | SAVYFCARYDYNYAMDY | WTFNPPTFGGGTRLEIK | | |
| | WGQGTSVTVSS (SEQ ID | (SEQ ID NO: 913) | | |
| | NO: 912) | | | |
| Ulocuplumab | EVQLVESGGGLVQPGGSL | DIQMTQSPSSLSASVGD | na | na |
| (Format: Whole | RLSCAAAGFTFSSYSMNW | RVTITCRASQGISSWLAW | | |
| mAb) | VRQAPGKGLEWVSYISSRS | YQQKPEKAPKSLIYAASSL | | |
| | RTIYYADSVKGRFTISRDN | QSGVPSRFSGSGSGTDFT | | |
| | AKNSLYLQMNSLRDEDTA | LTISSLQPEDFVTYYCQQY | | |
| | VYYCARDYGGQPPYYYYY | NSYPRTFGQGTKVEIK | | |
| | GMDVWGQGTTVTVSS | (SEQ ID NO: 915) | | |
| | (SEQ ID NO: 914) | | | |
| Urelumab | QVQLQQWGAGLLKPSETL | EIVLTQSPATLSLSPGERA | na | na |
| (Format: Whole | SLTCAVYGGSFSGYYWSW | TLSCRASQSVSSYLAWYQ | | |
| mAb) | IRQSPEKGLEWIGEINHGG | QKPGQAPRLLIYDASNRA | | |
| | YVTYNPSLESRVTISVDTSK | TGIPARFSGSGSGTDFTLT | | |
| | NQFSLKLSSVTAADTAVYY | ISSLEPEDFAVYYCQQRS | | |
| | CARDYGPGNYDWYFDLW | NWPPALTFGGGTKVEIK | | |
| | GRGTLVTVSS (SEQ ID | (SEQ ID NO: 917) | | |
| | NO: 916) | | | |
| Ustekinumab | EVQLVQSGAEVKKPGESL | DIQMTQSPSSLSASVGD | na | na |
| (Format: Whole | KISCKGSGYSFTTYWLGW | RVTITCRASQGISSWLAW | | |
| mAb) | VRQMPGKGLDWIGIMSP | YQQKPEKAPKSLIYAASSL | | |
| | VDSDIRYSPSFQGQVTMS | QSGVPSRFSGSGSGTDFT | | |
| | VDKSITTAYLQWNSLKASD | LTISSLQPEDFATYYCQQY | | |
| | TAMYYCARRRPGQGYFDF | NIYPYTFGQGTKLEIK | | |
| | WGQGTLVTVSS (SEQ ID | (SEQ ID NO: 919) | | |
| | NO: 918) | | | |
| Utomilumab | EVQLVQSGAEVKKPGESL | SYELTQPPSVSVSPGQTA | na | na |
| (Format: Whole | RISCKGSGYSFSTYWISWV | SITCSGDNIGDQYAHWY | | |
| mAb) | RQMPGKGLEWMGKIYPG | QQKPGQSPVLVIYQDKN | | |
| | DSYTNYSPSFQGQVTISAD | RPSGIPERFSGSNSGNTA | | |
| | KSISTAYLQWSSLKASDTA | TLTISGTQAMDEADYYCA | | |
| | MYYCARGYGIFDYWGQG | TYTGFGSLAVFGGGTKLT | | |
| | TLVTVSS (SEQ ID NO: | VL (SEQ ID NO: 921) | | |
| | 920) | | | |
| Vadastuximab | QVQLVQSGAEVKKPGASV | DIQMTQSPSSLSASVGD | na | na |
| (Format: Whole | KVSCKASGYTFTNYDINW | RVTINCKASQDINSYLSW | | |
| mAb ADC) | VRQAPGQGLEWIGWIYP | FQQKPGKAPKTLIYRANR | | |
| | GDGSTKYNEKFKAKATLTA | LVDGVPSRFSGSGSGQD | | |
| | DTSTSTAYMELRSLRSDDT | YTLTISSLQPEDFATYYCL | | |
| | AVYYCASGYEDAMDYWG | QYDEFPLTFGGGTKVEIK | | |
| | QGTTVTVSS (SEQ ID NO: | (SEQ ID NO: 923) | | |
| | 922) | | | |
| Valanafusp | QVQLQQSGPELVKPGALV | DIQMTQSPSSLSASLGER | na | na |
| (Format: Whole | KISCKASGYTFTNYDIHWV | VSLTCRASQDIGGNLYWL | | |
| mAb Fusion) | KQRPGQGLEWIGWIYPG | QQGPDGTIKRLIYATSSL | | |
| | DGSTKYNEKFKGKATLTAD | DSGVPKRFSGSRSGSDYS | | |
| | KSSSTAYMHLSSLTSEKSA | LTISSLESEDFVDYYCLQY | | |
| | VYFCAREWAYWGQGTLV | SSSPWTFGGGTKMEIK | | |
| | TVSA (SEQ ID NO: 924) | (SEQ ID NO: 925) | | |
| Vantictumab | EVQLVESGGGLVQPGGSL | DIELTQPPSVSVAPGQTA | na | na |
| (Format: Whole | RLSCAASGFTFSHYTLSWV | RISCSGDNIGSFYVHWYQ | | |
| mAb) | RQAPGKGLEWVSVISGDG | QKPGQAPVLVIYDKSNRP | | |
| | SYTYYADSVKGRFTISSDNS | SGIPERFSGSNSGNTATL | | |
| | KNTLYLQMNSLRAEDTAV | TISGTQAEDEADYYCQSY | | |
| | YYCARNFIKYVFANWGQG | ANTLSLVFGGGTKLTVL | | |
| | TLVTVSS (SEQ ID NO: | (SEQ ID NO: 927) | | |
| | 926) | | | |
| Vanucizumab | QVQLVQSGAEVKKPGASV | QPGLTQPPSVSVAPGQT | EVQLVESGGGLVQ | DIQMTQSPSSLSA |
| (Format: | KVSCKASGYTFTGYYMHW | ARITCGGNNIGSKSVHW | PGGSLRLSCAASGY | SVGDRVTITCSAS |
| Bispecific mAb) | VRQAPGQGLEWMGWIN | YQQKPGQAPVLVVYDDS | TFTNYGMNWVRQ | QDISNYLNWYQQ |
| | PNSGGTNYAQKFQGRVT | DRPSGIPERFSGSNSGNT | APGKGLEWVGWI | KPGKAPKVLIYFTS |
| | MTRDTSISTAYMELSRLRS | ATLTISRVEAGDEADYYC | NTYTGEPTYAADFK | SLHSGVPSRFSGS |
| | DDTAVYYCARSPNPYYYDS | QVWDSSSDHYVFGTGTK | RRFTFSLDTSKSTAY | GSGTDFTLTISSLQ |
| | SGYYYPGAFDIWGQGTM | VTVL (SEQ ID NO: 929) | LQMNSLRAEDTAV | PEDFATYYCQQYS |
| | VTVSS (SEQ ID NO: 928) | | YYCAKYPHYYGSSH | TVPWTFGQGTKV |
| | | | WYFDVWGQGTLV | EIK (SEQ ID NO: |
| | | | TVSS (SEQ ID NO: | 931) |
| | | | 930) | |
| Varisacumab | QVQLVQSGAEVKKPGASV | DIRMTQSPSSLSASVGDR | na | na |
| (Format: Whole | KVSCKASGGTFSSYAISWV | VTITCRASQSISSYLNWY | | |
| mAb) | RQAPGQGLEWMGGFDP | QQKPGKAPKLLIYAASSL | | |
| | EDGETIYAQKFQGRVTMT | QSGVPSRFSGSGSGTDFT | | |
| | EDTSTDTAYMELSSLRSED | LTISSLQPEDFATYYCQQS | | |
| | TAVYYCATGRSMVRGVIIP | YSTPLTFGGGTKVEIK | | |
| | FNGMDVWGQGTTVTVSS | (SEQ ID NO: 933) | | |
| | (SEQ ID NO: 932) | | | |
| Varlilumab | QVQLVESGGGVVQPGRSL | DIQMTQSPSSLSASVGD | na | na |
| (Format: Whole | RLSCAASGFTFSSYDMHW | RVTITCRASQGISRWLA | | |
| mAb) | VRQAPGKGLEWVAVIWY | WYQQKPEKAPKSLIYAAS | | |
| | DGSNKYYADSVKGRFTISR | SLQSGVPSRFSGSGSGTD | | |
| | DNSKNTLYLQMNSLRAED | FTLTISSLQPEDFATYYCQ | | |
| | TAVYYCARGSGNWGFFD | QYNTYPRTFGQGTKVEIK | | |
| | YWGQGTLVTVSS (SEQ ID | (SEQ ID NO: 935) | | |
| | NO: 934) | | | |
| Vatelizumab | QVQLQESGPGLVKPSETLS | DFVMTQSPAFLSVTPGE | na | na |
| (Format: Whole | LTCTVSGFSLTNYGIHWIR | KVTITCSAQSSVNYIHWY | | |
| mAb) | QPPGKGLEWLGVIWARG | QQKPDQAPKKLIYDTSKL | | |
| | FTNYNSALMSRLTISKDNS | ASGVPSRFSGSGSGTDYT | | |
| | KNQVSLKLSSVTAADTAVY | FTISSLEAEDAATYYCQQ | | |
| | YCARANDGVYYAMDYW | WTTNPLTFGQGTKVEIK | | |
| | GQGTLVTVSS (SEQ ID | (SEQ ID NO: 937) | | |
| | NO: 936) | | | |
| Vedolizumab | QVQLVQSGAEVKKPGASV | DVVMTQSPLSLPVTPGE | na | na |
| (Format: Whole | KVSCKGSGYTFTSYWMH | PASISCRSSQSLAKSYGNT | | |
| mAb) | WVRQAPGQRLEWIGEIDP | YLSWYLQKPGQSPQLLIY | | |
| | SESNTNYNQKFKGRVTLT | GISNRFSGVPDRFSGSGS | | |
| | VDISASTAYMELSSLRSED | GTDFTLKISRVEAEDVGV | | |
| | TAVYYCARGGYDGWDYAI | YYCLQGTHQPYTFGQGT | | |
| | DYWGQGTLVTVSS (SEQ | KVEIK (SEQ ID NO: 939) | | |
| | ID NO: 938) | | | |
| Veltuzumab | QVQLQQSGAEVKKPGSSV | DIQLTQSPSSLSASVGDR | na | na |
| (Format: Whole | KVSCKASGYTFTSYNMHW | VTMTCRASSSVSYIHWF | | |
| mAb) | VKQAPGQGLEWIGAIYPG | QQKPGKAPKPWIYATSN | | |
| | MGDTSYNQKFKGKATLTA | LASGVPVRFSGSGSGTDY | | |
| | DESTNTAYMELSSLRSEDT | TFTISSLQPEDIATYYCQQ | | |
| | AFYYCARSTYYGGDWYFD | WTSNPPTFGGGTKLEIK | | |
| | VWGQGTTVTVSS (SEQ ID | (SEQ ID NO: 941) | | |
| | NO: 940) | | | |
| Vesencumab | EVQLVESGGGLVQPGGSL | DIQMTQSPSSLSASVGD | na | na |
| (Format: Whole | RLSCAASGFTFSSYAMSW | RVTITCRASQYFSSYLAW | | |
| mAb) | VRQAPGKGLEWVSQISPA | YQQKPGKAPKLLIYGASS | | |
| | GGYTNYADSVKGRFTISAD | RASGVPSRFSGSGSGTDF | | |
| | TSKNTAYLQMNSLRAEDT | TLTISSLQPEDFATYYCQQ | | |
| | AVYYCARGELPYYRMSKV | YLGSPPTFGQGTKVEIK | | |
| | MDVWGQGTLVTVSS | (SEQ ID NO: 943) | | |
| | (SEQ ID NO: 942) | | | |
| Vibecotamab | QVQLQQSGAEVKKPGAS | DFVMTQSPDSLAVSLGE | EVQLVESGGGLVQ | QAVVTQEPSLTVS |
| (Format: | VKVSCKASGYTFTDYYMK | RATINCKSSQSLLNTGNQ | PGGSLRLSCAASGF | PGGTVTLTCGSST |
| Bispecific | WVKQSHGKSLEWMGDII | KNYLTWYQQKPGQPPKL | TFSTYAMNWVRQ | GAVTTSNYANW |
| Mixed | PSNGATFYNQKFKGKATL | LIYWASTRESGVPDRFTG | APGKGLEWVGRIR | VQQKPGKSPRGLI |
| mAb and scFV) | TVDRSTSTAYMELSSLRSE | SGSGTDFTLTISSLQAEDV | SKYNNYATYYADS | GGTNKRAPGVPA |
| | DTAVYYCARSHLLRASWF | AVYYCQNDYSYPYTFGG | VKGRFTISRDDSKN | RFSGSLLGGKAAL |
| | AYWGQGTLVTVSS (SEQ | GTKLEIK (SEQ ID NO: | TLYLQMNSLRAED | TISGAQPEDEADY |
| | ID NO: 944) | 945) | TAVYYCVRHGNFG | YCALWYSNHWV |
| | | | DSYVSWFAYWGQ | FGGGTKLTVLEPK |
| | | | GTLVTVSS (SEQ ID | (SEQ ID NO: 947) |
| | | | NO: 946) | |
| Visilizumab | QVQLVQSGAEVKKPGASV | DIQMTQSPSSLSASVGD | na | na |
| (Format: Whole | KVSCKASGYTFISYTMHW | RVTITCSASSSVSYMNWY | | |
| mAb) | VRQAPGQGLEWMGYINP | QQKPGKAPKRLIYDTSKL | | |
| | RSGYTHYNQKLKDKATLT | ASGVPSRFSGSGSGTDFT | | |
| | ADKSASTAYMELSSLRSED | LTISSLQPEDFATYYCQQ | | |
| | TAVYYCARSAYYDYDGFAY | WSSNPPTFGGGTKVEIK | | |
| | WGQGTLVTVSS (SEQ ID | (SEQ ID NO: 949) | | |
| | NO: 948) | | | |
| Vobarilizumab | EVQLVESGGGLVQPGGSL | na | EVQLVESGGGLVQ | na |
| (Format: | RLSCAASGSVFKINVMAW | | PGNSLRLSCAASGF | |
| Bispecific | YRQAPGKGRELVAGIISGG | | TFSSFGMSWVRQ | |
| Single | STSYADSVKGRFTISRDNA | | APGKGLEWVSSIS | |
| Domains (VH- | KNTLYLQMNSLRPEDTAV | | GSGSDTLYADSVK | |
| VH′) | YYCAFITTESDYDLGRRYW | | GRFTISRDNAKTTL | |
| | GQGTLVTVSS (SEQ ID | | YLQMNSLRPEDTA | |
| | NO: 950) | | VYYCTIGGSLSRSS | |
| | | | QGTLVTVSS (SEQ | |
| | | | ID NO: 951) | |
| Vofatamab | EVQLVESGGGLVQPGGSL | DIQMTQSPSSLSASVGD | na | na |
| (Format: Whole | RLSCAASGFTFTSTGISWV | RVTITCRASQDVDTSLA | | |
| mAb) | RQAPGKGLEWVGRIYPTS | WYKQKPGKAPKLLIYSAS | | |
| | GSTNYADSVKGRFTISADT | FLYSGVPSRFSGSGSGTD | | |
| | SKNTAYLQMNSLRAEDTA | FTLTISSLQPEDFATYYCQ | | |
| | VYYCARTYGIYDLYVDYTE | QSTGHPQTFGQGTKVEI | | |
| | YVMDYWGQGTLVTVSS | K (SEQ ID NO: 953) | | |
| | (SEQ ID NO: 952) | | | |
| Volagidemab | QVQLVESGGGVVQPGRSL | DIQMTQSPSSLSASVGD | na | na |
| (Format: Whole | RLSCAASGFTFSSYGMHW | RVTITCRASQGIRNDLG | | |
| mAb) | VRQAPGKGLEWVAVMW | WYQQKPGKAPKRLIYAA | | |
| | YDGSNKDYVDSVKGRFTIS | SSLQSGVPSRFSGSGSGT | | |
| | RDNSKNTLYLQMNRLRAE | EFTLTISSVQPEDFVTYYC | | |
| | DTAVYYCAREKDHYDILTG | LQHNSNPLTFGGGTKVEI | | |
| | YNYYYGLDVWGQGTTVT | K (SEQ ID NO: 955) | | |
| | VSS (SEQ ID NO: 954) | | | |
| Vonlerolizumab | EVQLVQSGAEVKKPGASV | DIQMTQSPSSLSASVGD | na | na |
| (Pogalizumab) | KVSCKASGYTFTDSYMSW | RVTITCRASQDISNYLNW | | |
| (Format: Whole | VRQAPGQGLEWIGDMYP | YQQKPGKAPKLLIYYTSRL | | |
| mAb) | DNGDSSYNQKFRERVTITR | RSGVPSRFSGSGSGTDFT | | |
| | DTSTSTAYLELSSLRSEDTA | LTISSLQPEDFATYYCQQ | | |
| | VYYCVLAPRWYFSVWGQ | GHTLPPTFGQGTKVEIK | | |
| | GTLVTVSS (SEQ ID NO: | (SEQ ID NO: 957) | | |
| | 956) | | | |
| Vopratelimab | EVQLVESGGGLVQPGGSL | DIVMTQSPDSLAVSLGER | na | na |
| (Format: Whole | RLSCAASGFTFSDYWMD | ATINCKSSQSLLSGSFNYL | | |
| mAb) | WVRQAPGKGLVWVSNID | TWYQQKPGQPPKLLIFY | | |
| | EDGSITEYSPFVKGRFTISR | ASTRHTGVPDRFSGSGS | | |
| | DNAKNTLYLQMNSLRAED | GTDFTLTISSLQAEDVAV | | |
| | TAVYYCTRWGRFGFDSW | YYCHHHYNAPPTFGPGT | | |
| | GQGTLVTVSS (SEQ ID | KVDIK | | |
| | NO: 958) | (SEQ ID NO: 959) | | |
| Vorsetuzumab | QVQLVQSGAEVKKPGASV | DIVMTQSPDSLAVSLGER | na | na |
| (Format: Whole | KVSCKASGYTFTNYGMN | ATINCRASKSVSTSGYSF | | |
| mAb ADC) | WVRQAPGQGLKWMGWI | MHWYQQKPGQPPKLLIY | | |
| | NTYTGEPTYADAFKGRVT | LASNLESGVPDRFSGSGS | | |
| | MTRDTSISTAYMELSRLRS | GTDFTLTISSLQAEDVAV | | |
| | DDTAVYYCARDYGDYGM | YYCQHSREVPWTFGQGT | | |
| | DYWGQGTTVTVSS (SEQ | KVEIK | | |
| | ID NO: 960) | (SEQ ID NO: 961) | | |
| Vunakizumab | EVQLVQSGAEVKKPGASV | EIVLTQSPDFQSVTPKEK | na | na |
| (Format: Whole | KVSCKASGYTFTDYEVHW | VTITCSASSSVNYMHWF | | |
| mAb) | VRQAPGQGLEWMGVIDP | QQKPDQSPKLWIYRTSN | | |
| | GTGGVAYNQKFEGRVTM | LASGVPSRFSGSGSGTDY | | |
| | TADTSTSTAYMELRSLRSD | TLTINSLEAEDAATYYCQ | | |
| | DTAVYYCTRYSLFYGSSPY | QRSSYPWTFGQGTKLEIK | | |
| | AMDYWGQGTLVTVSS | (SEQ ID NO: 963) | | |
| | (SEQ ID NO: 962) | | | |
| Xentuzumab | QVELVESGGGLVQPGGSL | DIVLTQPPSVSGAPGQRV | na | na |
| (Format: Whole | RLSCAASGFTFTSYWMSW | TISCSGSSSNIGSNSVSWY | | |
| mAb) | VRQAPGKGLELVSSITSYG | QQLPGTAPKLLIYDNSKR | | |
| | SFTYYADSVKGRFTISRDN | PSGVPDRFSGSKSGTSAS | | |
| | SKNTLYLQMNSLRAEDTA | LAITGLQSEDEADYYCQS | | |
| | VYYCARNMYTHFDSWGQ | RDTYGYYWVFGGGTKLT | | |
| | GTLVTVSS (SEQ ID NO: | VL (SEQ ID NO: 965) | | |
| | 964) | | | |
| Zalifrelimab | EVQLVESGGGLVKPGGSL | EIVLTQSPGTLSLSPGERA | na | na |
| (Format: Whole | RLSCAASGFTFSSYSMNW | TLSCRASQSVSRYLGWY | | |
| mAb) | VRQAPGKGLEWVSSISSSS | QQKPGQAPRLLIYGASTR | | |
| | SYIYYADSVKGRFTISRDNA | ATGIPDRFSGSGSGTDFT | | |
| | KNSLYLQMNSLRAEDTAV | LTITRLEPEDFAVYYCQQY | | |
| | YYCARVGLMGPFDIWGQ | GSSPWTFGQGTKVEIK | | |
| | GTMVTVSS (SEQ ID NO: | (SEQ ID NO: 967) | | |
| | 966) | | | |
| Zalutumumab | QVQLVESGGGVVQPGRSL | AIQLTQSPSSLSASVGDR | na | na |
| (Format: Whole | RLSCAASGFTFSTYGMHW | VTITCRASQDISSALVWY | | |
| mAb) | VRQAPGKGLEWVAVIWD | QQKPGKAPKLLIYDASSL | | |
| | DGSYKYYGDSVKGRFTISR | ESGVPSRFSGSESGTDFT | | |
| | DNSKNTLYLQMNSLRAED | LTISSLQPEDFATYYCQQF | | |
| | TAVYYCARDGITMVRGV | NSYPLTFGGGTKVEIK | | |
| | MKDYFDYWGQGTLVTVS | (SEQ ID NO: 969) | | |
| | S (SEQ ID NO: 968) | | | |
| Zampilimab | EVQLLESGGGLVQPGGSL | DITMTQSPSSLSASVGDR | na | na |
| (Format: Whole | RLSCAASGFTLSTHAMSW | VTITCKASQDINSYLTWF | | |
| mAb) | VRQAPGKGLEWVATISSG | QQKPGKAPKILIYLVNRL | | |
| | GRSTYYPDSVKGRFTISRD | VDGVPSRFSGSGSGQDY | | |
| | NSKNTLYLQMNSLRAEDT | ALTISSLQPEDFATYYCLQ | | |
| | AVYFCARLISTYWGQGTLV | YDDFPYTFGQGTKVEIK | | |
| | TVSS (SEQ ID NO: 970) | (SEQ ID NO: 971) | | |
| Zanolimumab | QVQLQQWGAGLLKPSETL | DIQMTQSPSSVSASVGD | na | na |
| (Format: Whole | SLTCAVYGGSFSGYYWSW | RVTITCRASQDISSWLAW | | |
| mAb) | IRQPPGKGLEWIGEINHSG | YQHKPGKAPKLLIYAASSL | | |
| | STNYNPSLKSRVTISVDTSK | QSGVPSRFSGSGSGTDFT | | |
| | NQFSLKLSSVTAADTAVYY | LTISSLQPEDFATYYCQQ | | |
| | CARVINWFDPWGQGTLV | ANSFPYTFGQGTKLEIK | | |
| | TVSS (SEQ ID NO: 972) | (SEQ ID NO: 973) | | |
| Zenocutuzumab | QVQLVQSGAEVKKPGASV | DIQMTQSPSSLSASVGD | QVQLVQSGAEVKK | DIQMTQSPSSLSA |
| (Format: | KVSCKASGYTFTGYYMHW | RVTITCRASQSISSYLNWY | PGASVKLSCKASGY | SVGDRVTITCRAS |
| Bispecific mAb) | VRQAPGQGLEWMGWIN | QQKPGKAPKLLIYAASSL | TFTAYYINWVRQA | QSISSYLNWYQQ |
| | PNSGGTNYAQKFQGRVT | QSGVPSRFSGSGSGTDFT | PGQGLEWIGRIYP | KPGKAPKLLIYAAS |
| | MTRDTSISTAYMELSRLRS | LTISSLQPEDFATYYCQQS | GSGYTSYAQKFQG | SLQSGVPSRFSGS |
| | DDTAVYYCARDHGSRHF | YSTPPTFGQGTKVEIK | RATLTADESTSTAY | GSGTDFTLTISSLQ |
| | WSYWGFDYWGQGTLVT | (SEQ ID NO: 975) | MELSSLRSEDTAVY | PEDFATYYCQQSY |
| | VSS (SEQ ID NO: 974) | | FCARPPVYYDSAW | STPPTFGQGTKVE |
| | | | FAYWGQGTLVTVS | IK (SEQ ID NO: |
| | | | S (SEQ ID NO: | 977) |
| | | | 976) | |
| Zolbetuximab | QVQLQQPGAELVRPGAS | DIVMTQSPSSLTVTAGEK | na | na |
| (Claudiximab) | VKLSCKASGYTFTSYWIN | VTMSCKSSQSLLNSGNQ | | |
| (Format: Whole | WVKQRPGQGLEWIGNIY | KNYLTWYQQKPGQPPKL | | |
| mAb) | PSDSYTNYNQKFKDKATLT | LIYWASTRESGVPDRFTG | | |
| | VDKSSSTAYMQLSSPTSED | SGSGTDFTLTISSVQAEDL | | |
| | SAVYYCTRSWRGNSFDY | AVYYCQNDYSYPFTFGSG | | |
| | WGQGTTLTVSS (SEQ ID | TKLEIK (SEQ ID NO: | | |
| | NO: 978) | 979) | | |

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated within the scope of the invention without limitation thereto.

### REFERENCES

1. Maude, S. L., et al., Chimeric antigen receptor T cells for sustained remissions in leukemia. N Engl J Med, 2014. 371(16): p. 1507-17.
2. Neelapu, S. S., et al., Axicabtagene Ciloleucel CAR T-Cell Therapy in Refractory Large B-Cell Lymphoma. N Engl J Med, 2017. 377(26): p. 2531-2544.
3. Kantarjian, H., et al., Results of inotuzumab ozogamicin, a CD22 monoclonal antibody, in refractory and relapsed acute lymphocytic leukemia. Cancer, 2013. 119(15): p. 2728-36.
4. Fry, T. J., et al., CD22-targeted CAR T cells induce remission in B-ALL that is naive or resistant to CD19-targeted CAR immunotherapy. Nat Med, 2018. 24(1): p. 20-28.
5. Vogler, I., et al., An improved bicistronic CD20/tCD34 vector for efficient purification and in vivo depletion of gene-modified T cells for adoptive immunotherapy. Mol Ther, 2010. 18(7): p. 1330-8.
6. Berger, C., et al., Analysis of transgene-specific immune responses that limit the in vivo persistence of adoptively transferred HSV-TK-modified donor T cells after allogeneic hematopoietic cell transplantation. Blood, 2006. 107(6): p. 2294-302.
7. Rodgers, D. T., et al., Switch-mediated activation and retargeting of CAR-T cells for B-cell malignancies. Proc Natl Acad Sci USA, 2016. 113(4): p. E459-68.
8. Majzner, R. G. and C. L. Mackall, Tumor Antigen Escape from CAR T-cell Therapy. Cancer Discov, 2018. 8(10): p. 1219-1226.
9. Shah, N. N., et al., Multi Targeted CAR-T Cell Therapies for B-Cell Malignancies. Frontiers in Oncology, 2019. 9(146).
10. Jia, H., et al., Haploidentical CD19/CD22 bispecific CAR-T cells induced MRD-negative remission in a patient with relapsed and refractory adult BALL after haploidentical hematopoietic stem cell transplantation. J Hematol Oncol, 2019. 12(1): p. 57.
11. Qin, H., et al., Preclinical Development of Bivalent Chimeric Antigen Receptors Targeting Both CD19 and CD22. Mol Ther Oncolytics, 2018. 11: p. 127-137.
12. Raney, K. D., et al., Hepatitis C virus non-structural protein 3 (HCV NS3): a multifunctional antiviral target. J Biol Chem, 2010. 285(30): p. 22725-31.
13. Jacobs, C. L., R. K. Badiee, and M. Z. Lin, StaPLs: versatile genetically encoded modules for engineering drug-inducible proteins. Nat Methods, 2018. 15(7): p. 523-526.
14. Jones, K. A., et al., Orthogonal Luciferase-Luciferin Pairs for Bioluminescence Imaging. Journal of the American Chemical Society, 2017. 139(6): p. 2351-2358.

1-43. (canceled)

44. A chimeric antigen receptor (CAR) comprising:
a single light chain variable fragment;
a single heavy chain variable fragment;
a single chain variable fragment linker;
at least one non-structural protein 3 (NS3) protease domain;
at least one cleavage site;
a hinge region;
a transmembrane region;
a CD3-zeta signaling domain; and, optionally,
an intracellular signaling domain, such as an intracellular signaling domain of CD27, CD28, 4-1BB (CD137), OX40, CD30, CD40, PD-1, ICOS (CD278), lymphocyte function-associated antigen-1 (LFA-1), CD2, CD7, TNFSF14, NKG2C, B7-H3, CD132, or ILRβ (CD122).

45. The CAR according to claim 44, wherein the at least one NS3 protease domain is selected from a wild-type HCV NS3 protease domain, a T54A NS3 protease domain, and a NS3 protease domain sensitive to inhibition by a small molecule inhibitor.

46. The CAR according to claim 44, wherein the NS3 protease domain is located between the single light and heavy chain variable fragments and the hinge region or between the hinge region and the transmembrane region.

47. The CAR, according to claim 44, wherein the NS3 protease domain is flanked by one or two cleavage sites.

48. A nucleic acid encoding the CAR according to claim 44.

49. A nucleic acid encoding
a first CAR comprising:
a single light chain variable fragment;
a single heavy chain variable fragment;
a single chain variable fragment linker;
a first non-structural protein 3 (NS3) protease domain;
at least one first cleavage site;
a first hinge region;
a first transmembrane region;
a first CD3-zeta signaling domain; and, optionally,
a first intracellular signaling domain; and
a second CAR comprising:
a single light chain variable fragment;
a single heavy chain variable fragment;
a single chain variable fragment linker;
a second non-structural protein 3 (NS3) protease domain;
at least one second cleavage site;
a second hinge region;
a second transmembrane region;
a second CD3-zeta signaling domain; and, optionally,
a second intracellular signaling domain.

50. The nucleic acid according to claim 49, wherein the first CAR comprises CD19 single light and heavy chain variable fragments and the second CAR comprises CD22 single light and heavy chain variable fragments.

51. The nucleic acid according to claim 49, wherein the first and the second NS3 protease domains are selected from a wild-type HCV NS3 protease domain, a T54A NS3 protease domain, and a NS3 protease domain sensitive to inhibition by a small molecule inhibitor.

52. The nucleic acid according to claim 51, wherein the first and the second NS3 protease domains are NS3 protease domains sensitive to inhibition by small molecule inhibitors.

53. The nucleic acid according to claim 52, wherein the first NS3 protease domain is sensitive to inhibition by a first small molecule inhibitor and the second NS3 protease domain is sensitive to inhibition by a second small molecule inhibitor.

54. A T cell comprising the nucleic acid according to claim 48, wherein the T cell does not express the CAR on the cell surface in the absence of a small molecule protease inhibitor.

55. A T cell comprising the nucleic acid according to claim 49, wherein the T cell does not express the first CAR and/or the second CAR on the cell surface in the absence of a small molecule protease inhibitor.

56. The T cell according to claim 55, wherein the T cell expresses the first CAR and/or the second CAR on the cell surface when contacted with at least one small molecule protease inhibitor.

57. A pharmaceutical composition comprising a T cell according to claim 54.

58. A pharmaceutical composition comprising a T cell according to claim 55.

59. A method of producing a T cell according to claim 54.

60. A method of producing a T cell according to claim 55.

61. A method of treatment of a cancer comprising administering a T cell according to claim 54 or a pharmaceutical composition thereof.

62. A method of treatment of a cancer or an immune disease comprising administering a T cell according to claim 55 or a pharmaceutical composition thereof to a subject suffering from said cancer or immune disease.

63. A method of treatment of an immune disease comprising administering a T cell according to claim 54 to a subject suffering from said immune disease.
